(19) United States
(12) Patent Application Publication
(10) Pub. No.: US 2020/0255844 A1
(43) Pub. Date: Aug. 13, 2020

(54) MULTIPLEXED RECEPTOR-LIGAND INTERACTION SCREENS

(71) Applicant: THE REGENTS OF THE UNIVERSITY OF CALIFORNIA, Oakland, CA (US)

(72) Inventors: Sriram KOSURI, Los Angeles, CA (US); Eric JONES, Los Angeles, CA (US)

(73) Assignee: THE REGENTS OF THE UNIVERSITY OF CALIFORNIA, Oakland, CA (US)

(21) Appl. No.: 16/628,348

(22) PCT Filed: Jul. 5, 2018
(86) PCT No.: PCT/US18/40866

§ 371 Date: Jan. 3, 2020

Related U.S. Application Data
(60) Provisional application No. 62/528,833, filed on Jul. 5, 2017.

(51) Int. Cl. C12N 15/79; C12N 15/10; C40B 40/08

(52) U.S. Cl. CPC C12N 15/79; C12N 2830/002; C40B 40/08; C12N 15/1086

(57) ABSTRACT

Aspects of the disclosure relate to a population of cells, wherein each cell comprises: i.) a heterologous receptor gene; ii.) an inducible reporter comprising a receptor-responsive element; wherein expression of the reporter is dependent on the activation of the activity of the receptor encoded by the receptor gene, and wherein the reporter comprises a barcode comprising an index region that is unique to the heterologous receptor gene; and wherein the cells express different heterologous receptors and wherein each single cell expresses one or more copies of one specific heterologous receptor and one or more copies of one specific reporter.

### CROSS-REFERENCE TO RELATED APPLICATIONS

This application claims the benefit of priority of U.S. Provisional Patent Application No. 62/528,833, filed Jul. 5, 2017, which is hereby incorporated by reference in its entirety.

This invention was made with Government support under 1555952, awarded by the National Science Foundation. The Government has certain rights in the invention.

### BACKGROUND

### 1. Field of the Invention

The current disclosure relates to the field of medicine and drug discovery.

### 2. Description of Related Art

G protein-coupled receptors (GPCRs) are one of the most important classes of drug targets, with approximately one-third of currently marketed drugs having their effect through GPCRs. G protein-coupled receptors (GPCRs) represent 50-60% of the current drug targets. This family of membrane proteins plays a crucial role in drug discovery today. Classically, a number of drugs based on GPCRs have been developed for such different indications as cardiovascular, metabolic, neurodegenerative, psychiatric, and oncologic diseases.

Moreover, there are currently few, if any methods that allow for an effective and efficient large-scale screen of thousands and even tens of thousands of receptors in a single assay platform. There is a significant need in the art for improvements in receptor and ligand interaction screens.

### SUMMARY OF THE DISCLOSURE

The current disclosure relates to nucleic acids, vectors, cells, viral particles, and methods that can be used to determine specific receptor activation. Accordingly, certain embodiments relate to nucleic acids comprising i.) a heterologous receptor gene; and ii.) an inducible reporter comprising a receptor-responsive element; wherein the expression of the reporter is dependent on the activation of the activity of the receptor encoded by the receptor gene, and wherein the reporter comprises a barcode comprising an index region that is uniquely identifiable to the heterologous receptor gene. Further aspects relate to a vector comprising nucleic acids of the disclosure. Further aspects relate to a vector comprising a heterologous receptor gene. The term “heterologous,” in the context of polynucleotides, refers to a gene or polynucleotide that has been transferred to a cell by gene transfer methods known in the art or described herein; progeny of such cells may also be referred to as containing the heterologous nucleic acid sequence if the exogenously derived sequence remains in the descendant cells. The cell may already contain an endogenous gene that is identical to the heterologous receptor gene or the cell may lack any endogenous genes that are related or identical to the heterologous gene. The term “heterologous cell” or “host cell” refers to a cell intentionally containing a heterologous nucleic acid sequence

The term “encode” as it is applied to polynucleotides refers to a polynucleotide which is said to “encode” a polypeptide if, in its native state or when manipulated by methods well known to those skilled in the art, it can be transcribed and/or translated to produce the mRNA for the polypeptide and/or a fragment thereof. The antisense strand is the complement of such a nucleic acid, and the encoding sequence can be deduced therefrom.

In some embodiments, the vector further comprises an inducible reporter; wherein expression of the reporter is dependent on the activation of the activity of the receptor encoded by the receptor gene, and wherein the reporter comprises a barcode comprising an index region that is unique to the heterologous receptor gene. Further aspects relate to a vector comprising an inducible reporter comprising a barcode.

Further aspects relate to a population of cells, wherein each cell comprises: i.) a heterologous receptor gene; ii.) an inducible reporter comprising a receptor-responsive element; wherein expression of the reporter is dependent on the activation of the activity of the receptor encoded by the receptor gene, and wherein the reporter comprises a barcode comprising an index region that is unique to the heterologous receptor gene; and wherein the cells express different heterologous receptors and wherein each single cell expresses one or more copies of one specific heterologous receptor and one or more copies of one specific reporter. For example, the population of cells may comprise at least a first cell with a first receptor gene and a first inducible reporter, a second cell with a second receptor gene and a second inducible reporter, a third cell with a third receptor gene and an inducible reporter, a fourth cell with a fourth receptor gene and a fourth inducible reporter . . . and a 1000th cell with a 1000th receptor gene and a 1000th inducible reporter . . . etc. The population of cells may comprise cells, each of which contains only one receptor and an associated inducible reporter comprising a barcode comprising an index region that can be used to identify the heterologous receptor that is activated in the same cell. The population of cells may comprise at least or at most 5, 10, 20, 30, 40, 50, 60, 70, 80, 90, 100, 150, 200, 250, 300, 350, 400, 450, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4500, 5000, 6000, 7000, 8000, 9000, 10⁴, 10⁵, 10⁶, 10^{7,}, 10⁸, 10⁹, or 10¹⁰ cells (or any derivable range therein), which represents the number of different receptor genes and their associated inducible reporter. Furthermore, in some embodiments, the inducible reporter produces an expressed nucleic acid that uniquely identifies the heterologous receptor gene that was expressed in that cell. The different receptor genes may be receptors belonging to a class of receptors, such as olfactory receptors, hormone receptors, adrenoceptors, drug-responsive receptors, and the like. Accordingly, the population of cells may comprise cells that express one and only one receptor gene (although it may be expressed from multiple copies of the same gene) and one and only one associated inducible reporter (although there may be multiple copies of the inducible reporter). In some embodiments, the cells each express one variant of the same receptor gene. It is contemplated that a single screen may involve the number of cells/receptors discussed herein. This differs in scale than other screens, which may involve employing screens serially in order to have the magnitude of some embodiments provided by this disclosure.

Further embodiments relate to a cell comprising i.) a heterologous receptor gene; and ii.) an inducible reporter comprising a receptor-responsive element; wherein expression of the reporter is dependent on the activation of the activity of the receptor encoded by the receptor gene, and wherein the reporter comprises a barcode comprising an index region that is unique to the heterologous receptor gene. In some embodiments, expression of the heterologous gene is “sustainable,” meaning expression of the heterologous gene remains at level that is within about or within at least about 10, 20, 30, 40, 50, 60, 70, 80, 90, or 100% of an expression level of cells from 1, 2, 3, 4, 5, 6, 7, 8, 9, 10, 15, 20, 25, 30, 35, 40, 45, 50 passages or more (or any range derivable therein) prior to the later cells or from 1, 2, 3, 4, 5, 6, 7 days and/or 1, 2, 3, 4, 5 weeks and/or 1, 2, 3, 4, 5, 6, 7, 8, 9, 10, 11, 12 months (or any range derivable therein) at a point in time prior to those later cells. In certain embodiments, the cells exhibit sustainable expression of the receptors to be tested. In some embodiments, cells express the receptors at a level that is within 2× of the level first measured following 1, 2, 3, 4, 5, 6, 7, 8, 9, 10, 15, 20, 25, 30, 35, 40, 45, 50 passages or more (or any range derivable therein).

In some embodiments, the receptor gene encodes for a G-protein coupled receptor (GPCR). In some embodiments, the reporter is induced upon signal transduction by the activated receptor protein. In some embodiments, activation of the receptor protein comprises binding of the receptor to a ligand. In some embodiments, the receptor gene further comprises one or more additional polynucleotides encoding for an auxiliary polypeptide. In some embodiments, the auxiliary polypeptide comprises a selectable or screenable protein. In some embodiments, the auxiliary polypeptide comprises a protein or peptide tag. In some embodiments, the auxiliary polypeptide comprises a transcription factor. In some embodiments, the auxiliary polypeptide comprises one or more trafficking tags. In some embodiments, the auxiliary polypeptide comprises two trafficking tags. In some embodiments, the auxiliary polypeptide comprises at least, at most, or exactly 1, 2, 3, 4, or 5 (or any derivable range therein) trafficking tags. In some embodiments, the trafficking tags comprise a Lucy and/or Rho trafficking tags. In some embodiments, the trafficking tag comprises a signal peptide. In some embodiments, the signal peptide is a cleavable peptide cleaved in vivo by endogenous proteins. Exemplary auxiliary polypeptides are described herein. In some embodiments, the receptor gene encodes for a fusion protein comprising the receptor gene and the auxiliary polypeptide. In some embodiments, the fusion protein comprises a protease site between the receptor gene and the auxiliary polypeptide.

In some embodiments, the reporter is induced by signal transduction upon activation of the GPCR. In some embodiments, the receptor-responsive element comprises one or more of a cAMP response element (CRE), a nuclear factor of activated T-cells response element (NFAT-RE), serum response element (SRE), and serum response factor response element (SRF-RE). In some embodiments, the receptor-responsive element comprises a DNA element that is bound by the auxiliary polypeptide transcription factor. In some embodiments, the auxiliary polypeptide transcription factor comprises reverse tetracycline-controlled transactivator (rtTA), and the receptor-responsive element comprises a tetracycline responsive element (TRE).

In some embodiments, the receptor-response element comprises CRE. In some embodiments, the CRE comprises at least 5 repeats of tgacgtca (SEQ ID NO:1). In some embodiments, the CRE comprises at least, at most, or exactly 3, 4, 5, 6, 7, 8, 9, or 10 repeats of SEQ ID NO:1 (or any derivable range therein). In some embodiments, the CRE comprises cgtcgtgacgtcagacagaccacgcgatcgctcgagtccgccggtcaatccggtgacgtcacgggcctcttcgctattacgccagct ggcgaaagggggttgacgtcacattaaatcggccaacgcgcggggagaggcggtgacgtcaacaggcatcgtggtgtcacgctcg tcgtgacgtcagtcgctttaactggccctggctttggcagcctgtagcctgacgtcagagagcctgacgtcaGagagcggagactcta gagggtatataatggaagctcgaattccagcttggcattccggtactgttggtaaa (SEQ ID NO:2) or a sequence that is at least, at most, or exactly 70, 75, 80, 85, 90, 95, 96, 97, 98, or 99% identical to SEQ ID NO:2 or a fragment thereof, for example, a fragment of 5, 6, 7, 8, 9, 10, 11, 12, 13, 14, 15, 16, 17, 18, 19, 20, 25, 30, 35, 40, 45, 50, 60, 70, 80, 90, 100, 125, 150, 175, 200, 225 250, 275, 300, 301, 302, 304, 305, 306, 307, 308, 309, 310, 312, 313, 314, or 315 contiguous nucleic acids of SEQ ID NO:2 (or any derivable range therein).

In some embodiments, the GPCR is an olfactory receptor (OR). ORs are known in the art and further described herein. In some embodiments, the receptor gene comprises a nuclear hormone receptor gene. In some embodiments, the receptor gene comprises a receptor tyrosine kinase gene. In some embodiments, the receptor comprises an adrenoceptor. In some embodiments, the adrenoceptor comprises a beta-2 adrenergic receptor. In some embodiments, the receptor comprises a receptor described herein. In some embodiments, the receptor is a transmembrane receptor. In some embodiments, the receptor is an intracellular receptor.

In some embodiments, the vector is a viral vector. In further embodiments, the vector is one known in the art and/or described herein. In some embodiments, the vector comprises a lentiviral vector.

In some embodiments, the receptor gene comprises a constitutive promoter. Exemplary constitutive promoters include, CMV, RSV, SV40 and the like. In some embodiments, the receptor gene comprises a conditional promoter. The term “conditional promoter” as used herein refers to a promoter that can be induced by the addition of an inducer and/or switched from the “off” state to the “on” state or the “on” state to the “off” state by the change of conditions, such as the change of temperature or the addition of a molecule such as an activator, a co-activator, or a ligand. Examples of a conditional promoter includes a “Tet-on” or “Tet-off” system, which can be used to inducible express proteins in cells.

In some embodiments, the reporter comprises an expressed RNA. In some embodiments, the reporter comprises a barcode of at least 10 nucleic acids. The barcode may be, be at least, or be at most, 3, 4, 5, 6, 7, 8, 9, 10, 12, 14, 16, 18, 20, 25, 30, 35, 40, 45, 50, 55, 60, 65, 70, 75, 80, 85, 90, 95, 100 or more nucleic acids (or any derivable range therein) in length. In some embodiments, the reporter comprises or further comprises an open reading frame (ORF); wherein the gene comprises a 3′ untranslated region (UTR). In some embodiments, the barcode is located in the 3′UTR of a gene, reporter, or other nucleic acid segment, such as for a gene encoding a fluorescent protein. In some embodiments, the ORF encodes a selectable or screenable protein. In some embodiments, the ORF encodes a fluorescent protein. In some embodiments, the ORF encodes a luciferase protein.

In some embodiments, the receptor gene is flanked at the 5′ and/or 3′ end by insulator sequences. In some embodiments, the reporter is flanked at the 5′ and/or 3′ end by insulator sequences. In some embodiments, the reporter gene is flanked at only the 5′ end or at only the 3′ end. In some embodiments, the reporter gene is not flanked at the 3′ end by an insulator. In some embodiments, the reporter gene is not flanked at the 5′ end by an insulator. In some embodiments, the receptor gene is flanked at only the 5′ end or at only the 3′ end. In some embodiments, the receptor gene is not flanked at the 3′ end by an insulator. In some embodiments, the receptor gene is not flanked at the 5′ end by an insulator.

In some embodiments, the insulator comprises a cHS4 insulator. In some embodiments, the insulator comprises GAGGGACAGCCCCCCCCCAAAGCCCCCAGGGATGTAATTACGTCCCTCCCCCGCT AGGGGGCAGCAGCGAGCCGCCCGGGGCTCCGCTCCGGTCCGGCGCTCCCCCCGC ATCCCCGAGCCGGCAGCGTGCGGGGACAGCCCGGGCACGGGGAAGGTGGCACG GGATCGCTTTCCTCTGAACGCTTCTCGCTGCTCTTTGAGCCTGCAGACACCTGGG GGGATACGGGGAAAA (SEQ ID NO:3) or a sequence that is at least, at most, or exactly 70, 75, 80, 85, 90, 95, 96, 97, 98, or 99% identical to SEQ ID NO:3 or a fragment thereof, for example, a fragment of 5, 6, 7, 8, 9, 10, 11, 12, 13, 14, 15, 16, 17, 18, 19, 20, 25, 30, 35, 40, 45, 50, 60, 70, 80, 90, 100, 125, 150, 175, 200, 205, 210, 215, 216, 217, 218, 219, 220, 221, 222, 223, 224, 225, 226, 227, 228, 229, 230, or 231 contiguous nucleic acids of SEQ ID NO:3 (or any derivable range therein).

In some embodiments, the insulator is a CTCF insulator, which is regulated by the CTCF repressor, or gypsy insulator, which is found in the gypsy retrotransposon of Drosophila.

In some embodiments, the vector comprises a second, third, fourth, or fifth barcode. In some embodiments, at least one of the second, third, or fourth barcode comprises an index region that is unique to one or more of: an assay condition or a position on a microplate. Assay conditions may include the addition of a specific ligand, the addition of a specific concentration of a ligand, or variant of a ligand, or concentration or variant of a metabolite, small molecule, polypeptide, inhibitor, repressor, or nucleic acid. In some embodiments, the additional barcode may be used to identify where the cell was positioned on a microplate, so that the assay conditions at that particular position may be identified and connected to the barcode.

Further aspects of the disclosure relate to a viral particle comprising one or more vectors or nucleic acids of the disclosure. Yet further aspects of the disclosure relate to a cell comprising a nucleic acid, vector, or viral particle of the disclosure. Further embodiments relate to a cell comprising a plurality of copies of a vector of the disclosure. In some embodiments, the cell comprises at least three copies of the vector. In some embodiments, the cell comprises at least four copies of the vector. In some embodiments, the cell comprises at least, at most, or exactly 3, 4, 5, 6, 7, 8, 9, 10, 12, 14, 16, or 20 copies (or any derivable range therein) of the vector.

In some embodiments, the cell or cells of the disclosure further comprises one or more genes encoding for one or more accessory proteins. In some embodiments, the one or more accessory proteins comprises one or more of a G α-subunit, Ric-8B, RTP1L, RTP2, RTP3, RTP4, CHMR3, and RTP1S. In some embodiments, the one or more accessory proteins comprises an arrestin protein. In some embodiments, the one or more accessory proteins comprises a Gi or Gq protein. In some embodiments, the arrestin protein is fused to a protease. In some embodiments, the one or more accessory proteins comprises one or more of a chaperone protein, a G protein, and a guanine nucleotide exchange factor. In some embodiments, the accessory proteins are integrated into the genome of the cell. As shown in the examples of the application, stable integration of the accessory factors provides for surprisingly good results, compared to transient expression. In some embodiments, the accessory proteins are transiently expressed. In some embodiments, the cell comprises stable integration of one or more exogenous nucleotides encoding one or more accessory factor genes, wherein the accessory factor genes comprise RTP1S, RTP2, G α-subunit (NCBI gene ID:2774), or Ric-8b (NCBI Gene ID 237422).

In some embodiments, the cell further comprises a receptor protein expressed from the heterologous receptor gene. In some embodiments, the receptor protein is localized intracellularly. In some embodiments, the cell lacks an endogenous gene that encodes for a protein that is at least 80% identical to the heterologous receptor gene. In some embodiments, the cell lacks an endogenous gene that encodes for a protein that is at least, at most, or exactly 65, 70, 75, 80, 85, 90, 95, 96, 97, 98, 99, or 100% identical (or any derivable range therein) to the heterologous receptor gene. In some embodiments, the receptor gene is integrated into the cell's genome. In some embodiments, the inducible reporter is integrated into the cell's genome. In some embodiments, the receptor gene and/or the inducible reporter is/are transiently expressed.

In some embodiments, the receptor gene and inducible reporter are genetically linked. In some embodiments, the receptor gene and inducible reporter are genetically unlinked. In some embodiments, the receptor gene and inducible reporter are inserted into the cell's genome and are within or separated by at least 10, 50, 100, 200, 500, 1000, 2000, 3000, 5000, or 10000 base pairs (bp) (or any range derivable therein) from each other. In further embodiments, the receptor gene and the inducible reporter are on separate genetic elements, such as separate chromosomes and/or extrachromosomal molecules.

In some embodiments, the integrated receptor gene and/or inducible reporter are integrated into the cellular genome by targeted integration. In some embodiments, the integrated receptor gene and/or inducible reporter are randomly integrated into the genome. In some embodiments, the random integration comprises transposition of the receptor gene and/or inducible reporter. In some embodiments, the cell comprises at least 2 copies of the receptor gene and/or inducible reporter. In other methods of random integration, DNA can be introduced into a cell and allowed to randomly integrate through recombination. In some embodiments, the integration is into the H11 safe harbor locus. In some embodiments, the integration is targeted integration into the H11 safe harbor locus.

In some embodiments, the receptor gene comprises a constitutive promoter. In some embodiments, the expression of the receptor is constitutive. In some embodiments, the receptor gene comprises a conditional promoter. In some embodiments, the expression of the receptor is conditional or inducible. In some embodiments, the heterologous receptor gene is operatively coupled to an inducible promoter. In some embodiments, the inducible or conditional promoter is a tetracycline response element.

In some embodiments, the expression level of the heterologous receptor is at a physiologically relevant expression level. The term “physiologically relevant expression level” refers to an expression level that is similar or equivalent to the endogenous expression level of the receptor in a cell. In other embodiments, the level of expression may below a physiologically relevant level. It is contemplated that in some embodiments, the sensitivity of sequencing a barcode allows for expression levels that are lower than what is needed for less sensitive assays. In some embodiments, the level of RNA transcripts is, is at least, or is at most about 10, 10², 10³, 10⁴, 10⁵, 10⁶, 10^{7,}, 10⁸, 10⁹, or 10¹⁰ or any range derivable therein.

In some embodiments, the cell or cells are frozen. In some embodiments, the cell is a mammalian cell. In some embodiments, the cell is a human embryonic kidney 293T (HEK293T) cells.

Further aspects relate to an assay system comprising the cells or population of cells described herein.

Further aspects relate to a method for screening for ligand and receptor binding, the method comprising: contacting the cell or cells of the disclosure with a ligand; detecting one or more reporters; and determining the identity of the one or more reporters; wherein the identity of the reporter indicates the identity of the bound receptor. Methods may involve screening some number of receptors and/or some number of ligands within a certain time period. In some embodiments, a single screen involves assaying about, at least about, or at most about 10, 10², 10³, 10⁴, 10⁵, 10⁶, 10^{7,}, 10⁸, 10⁹, or 10¹⁰ different cells and/or receptors (or any range derivable therein) with about, about at least, or about at most 5, 10, 20, 30, 40, 50, 60, 70, 80, 90, 100, 150, 200, 250, 300, 350, 400, 450, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4500, 5000, 6000, 7000, 8000, 9000, 10⁴, 10⁵, 10⁶, 10^{7,}, 10⁸, 10⁹, or 10¹⁰ ligands or potential ligands (or any range derivable therein) in a matter of 2, 3, 4, 5, 6, 7 days and/or 1, 2, 3, 4, 5 weeks and/or 1, 2, 3, 4, 5, or 6 months (and any range deriveable therein), where the screen begins when cells are contacted with a candidate ligand and the screen ends when a receptor is identified by its sequenced barcode.

In some embodiments, at least 300 different heterologous receptors are expressed in a population of cells. In some embodiments, at least 2, 5, 10, 50, 100, 200, 300, 400, 500, 600, 700, 800, 900, 1000, 2000, 3000, 4000, 5000, 6000, 7000, 8000, 9000, 10000, 20000, 30000, 40000, 50000, or more receptors are expressed in a population of cells. In some embodiments, the population of cells comprises at least or at most 10⁴, 10⁵, 10⁶, 10^{7,}, 10⁸, 10⁹, 10¹⁰, 10¹¹, or 10¹² cells (or any range derivable therein). In some embodiments, the population of cells are co-mixed in one composition. The composition may be a suspended composition of cells or a plated composition of cells. In some embodiments, the population of cells are adhered to a substrate, such as a cell culture dish. In some embodiments, the population of cells are contained within one well of a substrate or within one cell culture dish.

In some embodiments, determining the identity of the reporter comprises isolating nucleic acids from the cell. In some embodiments, the nucleic acids comprise RNA. In some embodiments, the method further comprises performing a reverse transcriptase reaction on the isolated RNA to make a cDNA. In some embodiments, the method further comprises amplifying the isolated nucleic acids. In some embodiments, the method further comprises sequencing the isolated nucleic acids. In some embodiments, the reverse transcriptase reaction is performed in the lysate. In some embodiments, detecting one or more reporters comprises detecting the level of fluorescence from the cell or cells. In some embodiments, the method further comprises plating the cells. In some embodiments, the cells are plated onto a 96-well cell culture plate. In some embodiments, the cells or cells are frozen and the method further comprises thawing frozen cells.

Certain aspects of the disclosure relate to a method for screening for ligand and receptor binding comprising: contacting a population of cells with a ligand; wherein each cell of the population of cells comprises: i.) a heterologous receptor gene; and ii.) an inducible reporter comprising a receptor-responsive element; wherein expression of the reporter is dependent on the activation of the activity of the receptor encoded by the receptor gene, and wherein the reporter comprises a barcode comprising an index region that is unique to the heterologous receptor gene; and wherein the population of cells express at least 2 different receptors from the heterologous receptor genes and wherein each single cell has one or more copies of one specific heterologous receptor and one or more copies of one specific reporter; detecting one or more reporters; and determining the identity of the one or more reporters; wherein the identity of the reporter indicates the identity of the bound receptor.

Methods further involve expressing in a cell any receptor identified in a screen. The receptor may be purified or isolated. One or more identified receptors may also be cloned. It may then be transfected into a different host cell for expression.

Further aspects relate to a vector library comprising at least two different vectors, wherein the vectors comprise different heterologous receptor genes and different inducible reporters. The vectors may be a vector described herein. Further aspects relate to a cell library comprising the population of cells of the disclosure. Further aspects relate to a viral library comprising at least two viral particles of the disclosure, wherein the viral particles comprise different heterologous receptor genes and different inducible reporters.

Further aspects relate to a method for making a library of cells comprising receptor proteins, the method comprising: i.) expressing a nucleic acid or vector of the disclosure in cells or ii.) infecting the cells with a viral particle of the disclosure; wherein the cells express different heterologous receptors and wherein each single cell has one or more copies of one specific heterologous receptor and one or more copies of one specific reporter. Each cell may have at least, at most, or exactly 1, 2, 3, 4, 5, 6, 7, 8, 9, or 10 copies (or any derivabley range therein) of the heterologous receptor gene and/or inducible reporter. In certain embodiments, the cell comprises at least 2, 3, 4, 5, 6, 7, 8, 9, or 10 copies (or any derivable range therein) of a nucleic acid encoding the receptor gene and/or inducible reporter.

Further aspects relate to kits comprising vectors, cells, nucleic acids, libraries, primers, probes, sequencing reagents and/or buffers as described herein.

Further aspects relate to a nucleic acid comprising: i.) a heterologous receptor gene operatively coupled to an inducible promoter; and ii.) a reporter comprising a receptor-responsive element; wherein the expression of the reporter is dependent on the activation of the activity of the receptor encoded by the heterologous receptor gene, and wherein the reporter comprises a barcode comprising an index region that is unique to the heterologous receptor gene. In some embodiments, the comprises at least 2 copies to at least 6 copies of the nucleic acid.

The term “an equivalent nucleic acid” refers to a nucleic acid having a nucleotide sequence having a certain degree of homology with the nucleotide sequence of the nucleic acid or complement thereof. A homolog of a double stranded nucleic acid is intended to include nucleic acids having a nucleotide sequence which has a certain degree of homology with or with the complement thereof. In one aspect, homologs of nucleic acids are capable of hybridizing to the nucleic acid or complement thereof. Nucleic acids of the disclosure also include equivalent nucleic acids.

A polynucleotide or polynucleotide region (or a polypeptide or polypeptide region) may have at least, at more, or exactly, 60%, 65%, 70%, 75%, 80%, 85%, 90%, 95%, 98% or 99% (or any derivable range therein) of “sequence identity” or “homology” to another sequence means that, when aligned, that percentage of bases (or amino acids) are the same in comparing the two sequences. This alignment and the percent homology or sequence identity can be determined using software programs known in the art, for example those described in Ausubel et al. eds. (2007) Current Protocols in Molecular Biology.

Biologically equivalent polynucleotides are those having the specified percent homology and encoding a polypeptide having the same or similar biological activity.

“About” and “approximately” shall generally mean an acceptable degree of error for the quantity measured given the nature or precision of the measurements. Typically, exemplary degrees of error are within 20 percent (%), preferably within 10%, and more preferably within 5% of a given value or range of values. Alternatively, and particularly in biological systems, the terms “about” and “approximately” may mean values that are within an order of magnitude, preferably within 5-fold and more preferably within 2-fold of a given value. In some embodiments it is contemplated that an numerical value discussed herein may be used with the term “about” or “approximately.”

As used herein, the term “comprising” is intended to mean that the compositions and methods include the recited elements, but not excluding others. “Consisting essentially of” when used to define compositions and methods, shall mean excluding other elements of any essential significance to the combination for the stated purpose. “Consisting essentially of” in the context of pharmaceutical compositions of the disclosure is intended to include all the recited active agents and excludes any additional non-recited active agents, but does not exclude other components of the composition that are not active ingredients. Thus, a composition consisting essentially of the elements as defined herein would not exclude trace contaminants from the isolation and purification method and pharmaceutically acceptable carriers, such as phosphate buffered saline, preservatives and the like. “Consisting of” shall mean excluding more than trace elements of other ingredients and substantial method steps for administering the compositions of this invention or process steps to produce a composition or achieve an intended result. Embodiments defined by each of these transition terms are within the scope of this invention.

The terms “protein”, “polypeptide” and “peptide” are used interchangeably herein when referring to a gene product or functional protein.

The terms “contacted” and “exposed,” when applied to a cell, are used herein to describe the process by which an agent is delivered to a target cell or are placed in direct juxtaposition with the target cell or target molecule.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives as well as “and/or.” As used herein “another” may mean at least a second or more.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment set forth with the term “comprising” may also be substituted with the word “consisting of” for “comprising.”

It is contemplated that any method or composition described herein can be implemented with respect to any other method or composition described herein and that different embodiments may be combined.

Use of the one or more compositions may be employed based on methods described herein. Use of one or more compositions may be employed in the preparation of medicaments for treatments according to the methods described herein. Other embodiments are discussed throughout this application. Any embodiment discussed with respect to one aspect of the disclosure applies to other aspects of the disclosure as well and vice versa. The embodiments in the Example section are understood to be embodiments that are applicable to all aspects of the technology described herein.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Overview of Multiplexed Reporter Scheme. Diagram detailing multiplexed scheme. Diagram detailing the barcoding strategy for the OR library. Each OR is linked to a unique barcode in the 3′ UTR of the reporter gene. Mukku3a cells are clonally integrated with each OR, pooled, and seeded for odorant induction. After induction, the barcoded transcripts are sequenced and quantified to determine the relative affinity for each odorant-receptor pair.

FIG. 2. Ind. Cell Line Luc/RNA and Pilot Screen. a) Show Ind. Luc for Stable Cell Line b) Show Ind. RNA for Stable Cell Line a) Individual, stable OR activation with known ligands measured via a cAMP responsive luciferase genetic reporter in Mukku3a cells. b) Individual, stable OR activation with known ligands measured via Q-RTPCR of the barcoded genetic reporter in Mukku3a cells.

FIG. 3. Combined v. Sep Genetic Reporter. a) Schematic of Sep v. Comb b) Sep v. Comb Transient Data. a) Plasmid configuration for encoding the OR and the reporter separately and together. b) Comparison of transient OR activation (MOR42-3 and MOR9-1) with known ligands measured via a cAMP responsive luciferase genetic reporter in the separate and combined configurations.

FIG. 4. Landing Pad. a) Schematic of Bxb1 b) Integration Efficiency c) B2 and OR int Luc. a) Schematic of Bxb1 recombination into a landing pad. HEK293T cells were pre-engineered to contain a single copy of the landing pad the safe harbor locus H11 (Mukku1a cells). The landing pad contains the Bxb1 recombinase recognition site attp. Co-expression of the recombinase and a plasmid containing the corresponding attb recognition site leads to a single, irreversible site-specific integration event. This integration strategy enables the clonal integration of a heterogeneous library in a single pot. b) Evaluation of the integration efficiency of the Bxb1 landing pad using flow cytometry. Cells were co-transfected with plasmids expressing the recombinase and a plasmid that conditionally expresses mCherry upon integration as well as solely with the mCherry plasmid. After multiple passages 7-8% of cells transfected with the recombinase as well were fluorescent and no cells without the recombinase were fluorescent. c) Combined genetic reporters encoding an OR (MOR42-3) and the beta-2 adrenergic receptor (ADRB2) were integrated into the landing pad. Both were induced with known agonists and genetic reporter activation was measured with a luciferase assay. Dose dependent activation was observed for ADRB2 but not for MOR42-3.

FIG. 5. Inducible Scheme. a) Schematic b) Trans and Int Ind. a) Mukku1a cells were transduced to constitutively express a reverse tetracycline transactivator (m2rtTA) and the constitutive promoter driving OR expression was replaced with a tetracycline regulated promoter. (Tetracycline responsive GFP was integrated to confirm expression in the landing pad with addition of doxycycline.) b) The inducible combined genetic reporter was screened for OR activation transiently and integrated in the landing pad of Mukku2a cells. Transient activation of MOR42-3 was observed in the presence of dox when stimulated with odorant, but was not observed when integrated in the landing pad. The bars above each concentration of part b represent − Dox (left bar) and + Dox (right bar).

FIG. 6. Copy Number. a) Transposon Scheme b) Cons. Transposon c) Ind. Transposon d) QPCR. a) Diagram of the transposon schematic. The PiggyBac transposase excises the combined genetic reporter flanked by intermediate terminal repeats. Multiple copies of the sequence are then inserted at TTAA loci across the genome. b) When transposed in Mukku1a cells under constitutive expression, MOR42-3 exhibits no dose responsive luciferase production to ligand. c) When transposed in Mukku2a under inducible expression, MOR42-3 exhibits robust dose responsive luciferase production to ligand in the presence of doxycycline. The bars above each concentration of part c represent − Dox (left bar) and + Dox (right bar). d) Copy number of the transposon was determined for transposition of three different ORs by QPCR of genomic DNA. Absolute copy number was determined by comparing the Cq for the transposons relative to the clonally integrated combined genetic reporter in the landing pad. The bars in part d represent (from left to right) control, MOR203-1, MOR9-1, and Olfr62.

FIG. 7. a) Trans AF b) Clone Selection. a) Comparison of transient OR activation (Olfr62 and MOR30-1) with known ligands measured via the combined luciferase genetic reporter in the presence or absence of the accessory factors RTP1S and RTP2. b) Mukku2a cells were transposed with four accessory factors (RTP1S, RTP2, Gαolf, and Ric8b) regulated under inducible expression. Individual clones were isolated and functionally assessed for accessory factor expression. Clones were assayed for transient OR activation (Olfr62 and OR7D4) with known ligands via the separate luciferase genetic reporter. The clone (Mukku3a) that displayed robust activation for both, typical morphology and growth rates was selected for downstream applications.

FIG. 8. Landing Pad Integration.

FIG. 9. A genomically integrated synthetic circuit allows screening of mammalian olfactory receptor activation. a.) Schematic of the synthetic circuit for stable OR expression and function in an engineered HEK293T cell line. b) MOR42-3 reporter activation expressing the receptor transiently or genomically integrated at varying copy number and under constitutive or inducible expression. c) Olfr62 reporter activation with/without accessory factors and transiently expressed/integrated into the engineered cell line. d) Dose-response curves for OR reporter activation integrated into the engineered cell line.

FIG. 10. Large-Scale, Multiplexed Screening of Olfactory Receptor-Odorant Interactions. a) Schematic for the creation of a library of OR reporter cell lines and for multiplexed screening. b) Comparison of MOR30-1 and Olfr62 reporter activation when tested with a transient or genomically integrated luciferase assay or the pooled RNA-seq assay. c) Heatmap of all interactions from the screen clustered by similarity of the odorant and receptor responses and colored by the lowest concentration that triggered reporter activity. d) Hits identified for four ORs (black) mapped onto a PCA projection of the chemical space of our odorant panel (grey).

FIG. 11. Engineering HEK293 Cells for Stable, Functional OR Expression. a) Comparison of MOR42-3 activation from inducibly driven receptor expression that was either transiently transfected or integrated at single copy at the H11 genomic locus. B. Activation from cells with MOR42-3 integrated at multiple copies in the genome under either constitutive or inducible expression. c) Relative receptor/reporter DNA copy number determined with qPCR for three transposed ORs relative to a single copy integrant. d) MOR30-1 and Olfr62 activation (stimulated with Decanoic Acid and 2-Coumaranone respectively) co-transfected with or without accessory factors (AF) Gα olf, Ric8b, RTP1S, and RTP2. e) Cell line generation for stable accessory factor expression. After transfection, clones were isolated and screened for activation of the ORs, Olfr62 and OR7D4, that require accessory factors to functionally express. The dark grey bar represents the clone selected for further experiments.

FIG. 12. Design of a Multiplexed Genetic Reporter for OR Activation. a) Schematic of the vector containing the OR expression cassette and genetic reporter for integration. b) MOR42-3 reporter activation in cells transiently co-expressing the receptor cassette on separate plasmids or together. c) Fold activation of an engineered CRE enhancer compared to Promega's pGL4.19 CRE enhancer. d) Basal activation of genetic reporter upon induction of the inducible OR promoter with or without a DNA insulator upstream of the CRE enhancer.

FIG. 13. Schematic of the Synthetic Olfactory Activation Circuit in the Engineered Cell Line. Full graphical representation of the expressed components for expression/signaling of the ORs and the barcoded reporter system as shown in FIG. 9 and described in Example 2. Receptor expression is controlled by the Tet-On system. After doxycycline induction, the OR is expressed on the cell surface with assistance from two exogenously expressed chaperones, RTP1S and RTP2. Upon odorant activation, g protein signaling triggers cAMP production. Signaling is augmented by transgenic expression of the native OR G alpha subunit, G olf, and its corresponding GEF, Ric8b. cAMP leads to activation of the kinase PKA that phosphorylates the transcription factor CREB leading to expression of the barcoded reporter.

FIG. 14. Pilot-Scale Recapitulation of Odorant Response in Multiplex. a) Heatmap displaying 40 pooled receptors response to 9 odorants and 2 mixtures. Interactions are colored by the log 2-fold activation of the genetic reporter. Odorant interactions previously identified (Saito et al. 2009) are boxed in yellow. b) Dose-response curves for odorants or forskolin (adenylate cyclase stimulator) screened against the OR library at 5 concentrations. Curves for ORs known to interact with the odorant are colored. Stimulation with forskolin does not show substantial differential activity between ORs in our assay.

FIG. 15. Library Representation. Representation of Individual ORs in the OR library. a) Frequency of each OR as a fraction of the library as determined by the relative activation of each reporter incubated with DMSO. b) The relationship between frequency of each OR in the library and the average coefficient of variation between biological replicate measurements of reporter activation for all conditions.

FIG. 16. Replicability of the Large-Scale Multiplexed Screen. a) Histogram displaying the distribution of the coefficient of variation for the OR library when stimulated with DMSO. b) Histogram displaying the distribution of the coefficient of variation for the OR library for all conditions assayed. c) Dose-response curves for the control odorants included on each 96-well plate assayed. Each color represents a different plate.

FIG. 17. Significance and Fold Change of High-Throughput Assay Data a) The False Discovery Rate (FDR)—computed from a generalized linear model with a negative binomial assumption and then multiple hypothesis corrected—plotted against the fold change for each OR-odorant interaction. The dashed line represents the 1% FDR, a conservative cutoff used to identify interactions b) The subset of interactions chosen for an orthogonal individual luciferase assay color indicates whether the interaction was detected. Of the interactions passing a 1% FDR, 21 of 28 also showed interaction in the orthogonal followup assay.

FIG. 18. Recapitulation of the Screen in a Transient, Orthogonal System. Secondary screen of chemicals against cell lines expressing a single olfactory receptor using a luciferase readout. Each plot shows the behavior of a negative control cell line not expressing an OR but treated with odorant (black line), as well as a cell line expressing a specific OR. In addition data from the high throughput sequencing screen (labeled Seq) is plotted for reference.

FIG. 19. Assay Correspondence with Previously Screened Odorant-Receptor Pairs. a) FDR plotted against fold induction for the 540 odorant-OR interactions that were previously tested by Saito et al. Points are colored by the EC50 of the interaction identified by Saito et al. (2009). Grey points represent interactions not identified in the previous screen. Comparing transient versus integrated luciferase assays revealed that, in some cases, the integrated system required a higher concentration of odorant to achieve significant activation, likely because of the lower DNA copy number of the CRE-driven luciferase and receptor. Since the highest concentration of odorant assayed was 1 mM, low affinity interactions may be not have been detectable in this screen. b) The FDR in the assay related to the EC50 of the hit from the previous screen colored by the fold activation from the multiplexed screen.

FIG. 20. Clustering of Odorant Response for Receptors. Here we plot the locations of any hits (black) with respect to the other chemicals tested (grey) on the same coordinates as FIG. 20. This provides a visualization of the breadth of activity for a given OR with respect to the larger chemicals space.

FIG. 21 Deep Mutational Scanning Overview.

FIG. 22. Distribution of Library Activity.

FIG. 23. Variant activity landscape for β2 at 0.625 uM Isoproterenol.

FIG. 24. Comparison to Individually Assayed Mutants

FIG. 25. Ligand Interaction Sites.

FIG. 26. k-means Clustering.

FIG. 27. A) Diagram of how Bxb1 recombination works in the context of a test to ensure only one construct is inserted per cell (cells will be only red or green) B) Flow Results of Two Color Test C) Activity of Reporter when stimulated with B2 agonist, isoproterenol, in the KO or wild type cells. D) When adding transgenic B2 in the single copy locus we can recover the ability to read B2 activity E) can be down on an RNA level as well and fold activation improved with an insulator element.

FIG. 28. Diagram of B2 construct being inserted into H11 locus.

### DESCRIPTION OF ILLUSTRATIVE EMBODIMENTS

Brute-force chemical screens have significant financial costs, scaling issues, and in the case of some receptors, such as olfactory recedptors, the screens also suffer from unreliable functional expression. Recently, a large-scale effort to conduct a comprehensive olfactory screen for human receptors assayed 394 ORs across 73 odorants. The researchers constructed a cell line that in combination with transient transfection allowed expression of all required factors for functional OR expression. Activation of the transiently transfected OR leads to luciferase reporter expression, which they can assay in multi-well plates. This screen required >50,000 individual measurements and took many years. This study alone doubled the known number of ligand-receptor binding pairs, and mapped 27 human OR receptors to their chemical ligands. Despite the success of this approach, the scale required to perform this relatively small chemical screen was so large because every compound had to be tested at a range of concentrations across hundreds of ORs with each test requiring a separate transient transfection. Such methods thus have little chance of scaling to the types of methods of the disclosure.

The methods of the disclosure describe the construction of large libraries of receptors contained within cell lines that can report on their activity in multiplex using detection methods described herein. With this automatable characterization platform, the current methods can be used to investigate ligand and receptor binding on a scale that is much larger that has been performed before. The assays and methods can have a multitude of applications in drug discovery and testing.

### I. RECEPTORS AND INDUCIBLE REPORTER ELEMENTS

The current methods, nucleic acids, vectors, viral particles, and cells of the disclosure relate to receptor proteins that, upon ligand engagement, induce the transcription of a reporter through the receptor-responsive element. Accordingly, the reporter is either under the direct control of the receptor protein or indirectly controlled by the receptor protein. The term “receptor-responsive element” refers to an element in the promoter region of the inducible reporter that is bound by the receptor or a down-stream element of the receptor after receptor and ligand engagement. In some embodiments, the receptor protein is a G-protein coupled receptor (GPCR) or the receptor gene encodes for a GPCR. G Protein Coupled Receptors (GPCRs) regulate a wide variety of normal biological processes and play a role in the pathophysiology of many diseases upon dysregulation of their downstream signaling activities. GPCR ligands include neurotransmitters, hormones, cytokines, and lipid signaling molecules. GPCRs regulate a wide variety of biological processes, such as vision, olfaction, the autonomic nervous system, and behavior. Besides its extracellular ligand, each GPCR binds specific intracellular heterotrimeric G-proteins composed of G-alpha, G-beta, and G-gamma subunits, which activate downstream signaling pathways. These intracellular signaling pathways include cAMP/PKA, calcium/NFAT, phospholipase C, protein tyrosine kinases, MAP kinases, PI-3-kinase, nitric oxide/cGMP, Rho, and JAK/STAT. Disruptions in GPCR function or signaling contribute to pathological conditions as varied as their ligands and the processes they regulate, from neurological to immunological to hormonal disorders. GPCRs represent 30 percent of all current drug development targets. Developing drug screening assays requires a survey of both target and related GPCR expression and function in the chosen cell-based model system as well as expression of related GPCRs to assess both direct and potential off-target side effects.

It is within the skill of one in the art to construct a receptor gene/receptor-responsive element based on the extensive knowledge of receptor signaling and transcriptional regulation effected by the receptor.

In the case of GPCRs, the inducible reporter comprises a response element that directs transcriptional activity of the reporter upon GPCR signal transduction activation by ligand engagement. GPCR response elements include: cAMP response element (CRE), nuclear factor of activated T-cells response element (NFAT-RE), serum response element (SRE) and serum response factor response element (SRF-RE). GPCRs can further be classified as Gₛ, Gᵢ, G_{q}, and G₁₂. Examples of receptor gene/protein and response element is shown in the table below:

| Receptor gene/protein | Response element |
|---|---|
| Gₛ | CRE |
| Gᵢ | SRE |
| G_{q} | NFAT-RE |
| G₁₂ | SRF-RE |

The G_{olf} or G olfactory receptor is a Gₛ GPCR whose signal transduction converts ATP to cAMP. cAMP then directs transcription through the CRE response element. Exemplary olfactory receptors include those tabulated below:

Olfactory receptors, family 1:

| Approved | | Previous | Syn- | |
|---|---|---|---|---|
| Symbol | Approved Name | Symbols | onyms | Chromosome |
| OR1A1 | olfactory receptor family 1 | | OR17-7 | 17p13.3 |
| | subfamily A member 1 | | | |
| OR1A2 | olfactory receptor family 1 | | OR17-6 | 17p13.3 |
| | subfamily A member 2 | | | |
| OR1AA1P | olfactory receptor family 1 | | | Xq26.2 |
| | subfamily AA member 1 pseudogene | | | |
| OR1AB1P | olfactory receptor family 1 | | | 19p13.12 |
| | subfamily AB member 1 pseudogene | | | |
| OR1AC1P | olfactory receptor family 1 | | | 17p13.3 |
| | subfamily AC member 1 pseudogene | | | |
| OR1B1 | olfactory receptor family 1 | | OR9-B | 9q33.2 |
| | subfamily B member 1 | | | |
| | (gene/pseudogene) | | | |
| OR1C1 | olfactory receptor family 1 | | TPCR27, | 1q44 |
| | subfamily C member 1 | | HSTPCR27 | |
| OR1D2 | | | | |
| OR1D3P | olfactory receptor family 1 | OR1D6P, | OR17-23, | 17p13.3 |
| | subfamily D member 3 pseudogene | OR1D7P | OR11-13, | |
| | | | OR11-22 | |
| OR1D4 | olfactory receptor family 1 | | OR17-30 | 17p13.3 |
| | subfamily D member 4 | | | |
| | (gene/pseudogene) | | | |
| OR1D5 | olfactory receptor family 1 | | OR17-31 | 17p13.3 |
| | subfamily D member 5 | | | |
| OR1E1 | olfactory receptor family 1 | OR1E9P, | OR17-2, | 17p13.3 |
| | subfamily E member 1 | OR1E5, | HGM071, | |
| | | OR1E6 | OR17-32, | |
| | | | OR13-66 | |
| OR1E2 | olfactory receptor family 1 | OR1E4 | OR17-93, | 17p13.2 |
| | subfamily E member 2 | | OR17-135 | |
| OR1E3 | olfactory receptor family 1 | OR1E3P | OR17-210 | 17p13.3 |
| | subfamily E member 3 | | | |
| | (gene/pseudogene) | | | |
| OR1F1 | olfactory receptor family 1 | OR1F4, | Olfmf, OR16- | 16p13.3 |
| | subfamily F member 1 | OR1F6, | 36, OR16-37, | |
| | | OR1F7, | OR16-88, | |
| | | OR1F8, | OR16-89, | |
| | | OR1F9, | OR16-90, | |
| | | OR1F5, | OEFMF, OR3- | |
| | | OR1F10, | 145 | |
| | | OR1F13P | | |
| OR1F2P | olfactory receptor family 1 | OR1F3P, | OLFMF2 | 16p13.3 |
| | subfamily F member 2 pseudogene | OR1F2 | | |
| OR1F12 | olfactory receptor family 1 | OR1F12P | hs6M1-35P, | 6p22.1 |
| | subfamily F member 12 | | OR1F12Q | |
| OR1G1 | olfactory receptor family 1 | OR1G2 | OR17-209 | 17p13.3 |
| | subfamily G member 1 | | | |
| OR1H1P | olfactory receptor family 1 | OR1H1 | OST26 | 9q33.2 |
| | subfamily H member 1 pseudogene | | | |
| OR1I1 | olfactory receptor family 1 | | OR1I1P, | 19p13.1 |
| | subfamily I member 1 | | OR19-20, | |
| | | | OR1I1Q | |
| OR1J1 | olfactory receptor family 1 | | hg32 | 9q33.2 |
| | subfamily J member 1 | | | |
| OR1J2 | olfactory receptor family 1 | OR1J3, | OST044 | 9q33.2 |
| | subfamily J member 2 | OR1J5 | | |
| OR1J4 | olfactory receptor family 1 | | HTPCRX01, | 9q33.2 |
| | subfamily J member 4 | | HSHTPCRX01 | |
| OR1K1 | olfactory receptor family 1 | | hg99, MNAB | 9q33 |
| | subfamily K member 1 | | | |
| OR1L1 | olfactory receptor family 1 | OR1L2 | OR9-C | 9q33.2 |
| | subfamily L member 1 | | | |
| OR1L3 | olfactory receptor family 1 | | OR9-D | 9q33.2 |
| | subfamily L member 3 | | | |
| OR1L4 | olfactory receptor family 1 | OR1L5 | OR9-E | 9q33.2 |
| | subfamily L member 4 | | | |
| OR1L6 | olfactory receptor family 1 | OR1L7 | | 9q33.2 |
| | subfamily L member 6 | | | |
| OR1L8 | olfactory receptor family 1 | | | 9q33.2 |
| | subfamily L member 8 | | | |
| OR1M1 | olfactory receptor family 1 | | OR19-6 | 19p13.2 |
| | subfamily M member 1 | | | |
| OR1M4P | olfactory receptor family 1 | | | 19p13.2 |
| | subfamily M member 4 pseudogene | | | |
| OR1N1 | olfactory receptor family 1 | OR1N3 | OR1-26 | 9q33.2 |
| | subfamily N member 1 | | | |
| OR1N2 | olfactory receptor family 1 | | | 9q33.2 |
| | subfamily N member 2 | | | |
| OR1P1 | olfactory receptor family 1 | OR1P1P | OR17-208 | 17p13.3 |
| | subfamily P member 1 | | | |
| | (gene/pseudogene) | | | |
| OR1Q1 | olfactory receptor family 1 | OR1Q2, | OST226, OR9- | 9q33.2 |
| | subfamily Q member 1 | OR1Q3 | A, | |
| | | | HSTPCR106, | |
| | | | OST226OR9- | |
| | | | A, TPCR106 | |
| OR1R1P | olfactory receptor family 1 | OR20A1P, | OR17-1 | 17p13.3 |
| | subfamily R member 1 pseudogene | OR1R2P, | | |
| | | OR1R3P | | |
| OR1S1 | olfactory receptor family 1 | | OST034 | 11q12.1 |
| | subfamily S member 1 | | | |
| | (gene/pseudogene) | | | |
| OR1S2 | olfactory receptor family 1 | | | 11q12.1 |
| | subfamily S member 2 | | | |
| OR1X1P | olfactory receptor family 1 | | | 5q35.2 |
| | subfamily X member 1 pseudogene | | | |
| OR1X5P | olfactory receptor family 1 | | | 5q35.3 |
| | subfamily X member 5 pseudogene | | | |

Olfactory receptors, family 2:

| Approved | | Previous | Syn- | |
|---|---|---|---|---|
| Symbol | Approved Name | Symbols | onyms | Chromosome |
| OR2A1 | olfactory receptor family 2 subfamily | | | 7q35 |
| | A member 1 | | | |
| OR2A2 | olfactory receptor family 2 subfamily | OR2A2P, | OST008 | 7q35 |
| | A member 2 | OR2A17P | | |
| OR2A3P | olfactory receptor family 2 subfamily | | | 7q35 |
| | A member 3 pseudogene | | | |
| OR2A4 | olfactory receptor family 2 subfamily | OR2A10 | | 6q23.2 |
| | A member 4 | | | |
| OR2A5 | olfactory receptor family 2 subfamily | OR2A8, | OR7-138, | 7q35 |
| | A member 5 | OR2A26 | OR7-141 | |
| OR2A7 | olfactory receptor family 2 subfamily | | HSDJ0798C | 7q35 |
| | A member 7 | | 17 | |
| OR2A9P | olfactory receptor family 2 subfamily | OR2A9 | HSDJ0798C | 7q35 |
| | A member 9 pseudogene | | 17 | |
| OR2A12 | olfactory receptor family 2 subfamily | OR2A12P | | 7q35 |
| | A member 12 | | | |
| OR2A13P | olfactory receptor family 2 subfamily | | | 7q35 |
| | A member 13 pseudogene | | | |
| OR2A14 | olfactory receptor family 2 subfamily | OR2A14P, | OST182 | 7q35 |
| | A member 14 | OR2A6 | | |
| OR2A15P | olfactory receptor family 2 subfamily | OR2A28P | | 7q35 |
| | A member 15 pseudogene | | | |
| OR2A20P | olfactory receptor family 2 subfamily | OR2A20 | | 7q35 |
| | A member 20 pseudogene | | | |
| OR2A25 | olfactory receptor family 2 subfamily | OR2A25P, | | 7q35 |
| | A member 25 | OR2A27 | | |
| OR2A41P | olfactory receptor family 2 subfamily | | | 7q35 |
| | A member 41 pseudogene | | | |
| OR2A42 | olfactory receptor family 2 subfamily | | | 7q35 |
| | A member 42 | | | |
| OR2AD1P | olfactory receptor family 2 subfamily | | OR2AD1, | 6p22.1 |
| | AD member 1 pseudogene | | hs6M1-8P | |
| OR2AE1 | olfactory receptor family 2 subfamily | OR2AE2 | | 7q22.1 |
| | AE member 1 | | | |
| OR2AF1P | olfactory receptor family 2 subfamily | OR2AF2P | | Xq26.2 |
| | AF member 1 pseudogene | | | |
| OR2AG1 | olfactory receptor family 2 subfamily | OR2AG3 | | 11p15.4 |
| | AG member 1 (gene/pseudogene) | | | |
| OR2AG2 | olfactory receptor family 2 subfamily | OR2AG2P | | 11p15.4 |
| | AG member 2 | | | |
| OR2AH1P | olfactory receptor family 2 subfamily | | | 11q12.1 |
| | AH member 1 pseudogene | | | |
| OR2AI1P | olfactory receptor family 2 subfamily | | | 5q35.3 |
| | AI member 1 pseudogene | | | |
| OR2AJ1 | olfactory receptor family 2 subfamily | OR2AJ1P | OR2AJ1Q | 1q44 |
| | AJ member 1 | | | |
| OR2AK2 | olfactory receptor family 2 subfamily | OR2AK1P | | 1q44 |
| | AK member 2 | | | |
| OR2AL1P | olfactory receptor family 2 subfamily | | | 11q22.3 |
| | AL member 1 pseudogene | | | |
| OR2AM1P | olfactory receptor family 2 subfamily | | | 9p13.3 |
| | AM member 1 pseudogene | | | |
| OR2AO1P | olfactory receptor family 2 subfamily | | | 7q35 |
| | AO member 1 pseudogene | | | |
| OR2AP1 | olfactory receptor family 2 subfamily | OR2AP1P | | 12q13.2 |
| | AP member 1 | | | |
| OR2AQ1P | olfactory receptor family 2 subfamily | | | 1q23.1 |
| | AQ member 1 pseudogene | | | |
| OR2AS1P | olfactory receptor family 2 subfamily | | | 1q44 |
| | AS member 1 pseudogene | | | |
| OR2AS2P | olfactory receptor family 2 subfamily | | | 1q44 |
| | AS member 2 pseudogene | | | |
| OR2AT1P | olfactory receptor family 2 subfamily | | | 11q13.4 |
| | AT member 1 pseudogene | | | |
| OR2AT2P | olfactory receptor family 2 subfamily | | | 11q13.4 |
| | AT member 2 pseudogene | | | |
| OR2AT4 | olfactory receptor family 2 subfamily | | | 11q13.4 |
| | AT member 4 | | | |
| OR2B2 | olfactory receptor family 2 subfamily | OR2B9 | hs6M1-10, | 6p22.1 |
| | B member 2 | | OR6-1, | |
| | | | OR2B2Q | |
| OR2B3 | olfactory receptor family 2 subfamily | OR2B3P | OR6-4 | 6p22.1 |
| | B member 3 | | | |
| OR2B4P | olfactory receptor family 2 subfamily | | hs6M1-22 | 6p22.2-p21.32 |
| | B member 4 pseudogene | | | |
| OR2B6 | olfactory receptor family 2 subfamily | OR2B6P, | OR6-31, | 6p22.1 |
| | B member 6 | OR2B1, | dJ408B20.2, | |
| | | OR2B1P, | OR5-40, | |
| | | OR2B5 | OR5-41 | |
| OR2B7P | olfactory receptor family 2 subfamily | | hs6M1-31P | 6p22.1 |
| | B member 7 pseudogene | | | |
| OR2B8P | olfactory receptor family 2 subfamily | OR2B8 | hs6M1-29P | 6p22.1 |
| | B member 8 pseudogene | | | |
| OR2B11 | olfactory receptor family 2 subfamily | | | 1q44 |
| | B member 11 | | | |
| OR2BH1P | olfactory receptor family 2 subfamily | | | 11p14.1 |
| | BH member 1 pseudogene | | | |
| OR2C1 | olfactory receptor family 2 subfamily | OR2C2P | OLFmf3 | 16p13.3 |
| | C member 1 | | | |
| OR2C3 | olfactory receptor family 2 subfamily | OR2C4, | OST742 | 1q44 |
| | C member 3 | OR2C5P | | |
| OR2D2 | olfactory receptor family 2 subfamily | OR2D1 | OR11-610, | 11p15.4 |
| | D member 2 | | hg27 | |
| OR2D3 | olfactory receptor family 2 subfamily | | | 11p15.4 |
| | D member 3 | | | |
| OR2E1P | olfactory receptor family 2 subfamily | OR2E1, | hs6M1-9, | 6p22-p21.3 |
| | E member 1 pseudogene | OR2E2 | hs6M1-9p, | |
| | | | HS29K1, | |
| | | | HSNH0569I24 | |
| OR2F1 | olfactory receptor family 2 subfamily | OR2F4, | OLF3, OR7- | 7q35 |
| | F member 1 (gene/pseudogene) | OR2F5, | 140, OR7- | |
| | | OR2F3, | 139, OR14- | |
| | | OR2F3P | 60 | |
| OR2F2 | olfactory receptor family 2 subfamily | | OR7-1 | 7q35 |
| | F member 2 | | | |
| OR2G1P | olfactory receptor family 2 subfamily | | OST619, | 6p22.2-p21.32 |
| | G member 1 pseudogene | | hs6M1-25 | |
| OR2G2 | olfactory receptor family 2 subfamily | | | 1q44 |
| | G member 2 | | | |
| OR2G3 | olfactory receptor family 2 subfamily | | | 1q44 |
| | G member 3 | | | |
| OR2G6 | olfactory receptor family 2 subfamily | | | 1q44 |
| | G member 6 | | | |
| OR2H1 | olfactory receptor family 2 subfamily | OR2H6, | OR6-2 | 6p22.1 |
| | H member 1 | OR2H8 | | |
| OR2H2 | olfactory receptor family 2 subfamily | | hs6Ml-12 | 6p22.1 |
| | H member 2 | | | |
| OR2H4P | olfactory receptor family 2 subfamily | | OR6-3, | 6p22.2-p21.31 |
| | H member 4 pseudogene | | OR2H4, | |
| | | | hs6M1-7, | |
| | | | dJ80I19.6 | |
| OR2H5P | olfactory receptor family 2 subfamily | | OR2H5, | 6p22.2-p21.31 |
| | H member 5 pseudogene | | hs6M1-13, | |
| | | | HS271M21 | |
| OR2I1P | olfactory receptor family 2 subfamily | OR2I1, | HS6M1-14 | 6p22.1 |
| | I member 1 pseudogene | OR2I3P, | | |
| | | OR2I4P, | | |
| | | OR2I2 | | |
| OR2J1 | olfactory receptor family 2 subfamily | OR2J1P | OR6-5, | 6p22.1 |
| | J member 1 (gene/pseudogene) | | hs6M1-4, | |
| | | | dJ80I19.2 | |
| OR2J2 | olfactory receptor family 2 subfamily | | OR6-8, | 6p22.1 |
| | J member 2 | | hs6M1-6, | |
| | | | dJ80I19.4 | |
| OR2J3 | olfactory receptor family 2 subfamily | | OR6-6 | 6p22.1 |
| | J member 3 | | | |
| OR2J4P | olfactory receptor family 2 subfamily | | OR6-9, | 6p22.2-p21.31 |
| | J member 4 pseudogene | | hs6M1-5, | |
| | | | dJ80I19.5 | |
| OR2K2 | olfactory receptor family 2 subfamily | OR2AR1P | HTPCRH06, | 9q31.3 |
| | K member 2 | | HSHTPCRH06 | |
| OR2L1P | olfactory receptor family 2 subfamily | OR2L1, | HTPCRX02, | 1q44 |
| | L member 1 pseudogene | OR2L7P | HSHTPCRX02 | |
| OR2L2 | olfactory receptor family 2 subfamily | OR2L4P, | HTPCRH07, | 1q44 |
| | L member 2 | OR2L12 | HSHTPCRH07 | |
| OR2L3 | olfactory receptor family 2 subfamily | | | 1q44 |
| | L member 3 | | | |
| OR2L5 | olfactory receptor family 2 subfamily | OR2L11, | | 1q44 |
| | L member 5 | OR2L5P | | |
| OR2L6P | olfactory receptor family 2 subfamily | | | 1q44 |
| | L member 6 pseudogene | | | |
| OR2L8 | olfactory receptor family 2 subfamily | | | 1q44 |
| | L member 8 (gene/pseudogene) | | | |
| OR2L9P | olfactory receptor family 2 subfamily | | | 1q44 |
| | L member 9 pseudogene | | | |
| OR2L13 | olfactory receptor family 2 subfamily | OR2L14 | | 1q44 |
| | L member 13 | | | |
| OR2M1P | olfactory receptor family 2 subfamily | OR2M1 | OST037 | 1q44 |
| | M member 1 pseudogene | | | |
| OR2M2 | olfactory receptor family 2 subfamily | | OST423, | 1q44 |
| | M member 2 | | OR2M2Q | |
| OR2M3 | olfactory receptor family 2 subfamily | OR2M6, | OST003 | 1q44 |
| | M member 3 | OR2M3P | | |
| OR2M4 | olfactory receptor family 2 subfamily | | HTPCRX18, | 1q44 |
| | M member 4 | | TPCR100, | |
| | | | HSHTPCRX18, | |
| | | | OST710 | |
| OR2M5 | olfactory receptor family 2 subfamily | OR2M5P | | 1q44 |
| | M member 5 | | | |
| OR2M7 | olfactory receptor family 2 subfamily | | | 1q44 |
| | M member 7 | | | |
| OR2N1P | olfactory receptor family 2 subfamily | | OR6-7 | 6p22.2-p21.31 |
| | N member 1 pseudogene | | | |
| OR2P1P | olfactory receptor family 2 subfamily | | hs6M1-26 | 6p22.1 |
| | P member 1 pseudogene | | | |
| OR2Q1P | olfactory receptor family 2 subfamily | | OR7-2 | 7q33-q35 |
| | Q member 1 pseudogene | | | |
| OR2R1P | olfactory receptor family 2 subfamily | OR2R1 | OST058 | 7q35 |
| | R member 1 pseudogene | | | |
| OR2S1P | olfactory receptor family 2 subfamily | | OST611 | 9pl3.3 |
| | S member 1 pseudogene | | | |
| OR2S2 | olfactory receptor family 2 subfamily | | | 9pl3.3 |
| | S member 2 (gene/pseudogene) | | | |
| OR2T1 | olfactory receptor family 2 subfamily | | OR1-25 | 1q44 |
| | T member 1 | | | |
| OR2T2 | olfactory receptor family 2 subfamily | OR2T2P | | 1q44 |
| | T member 2 | | | |
| OR2T3 | olfactory receptor family 2 subfamily | | | 1q44 |
| | T member 3 | | | |
| OR2T4 | olfactory receptor family 2 subfamily | | OR2T4Q | 1q44 |
| | T member 4 | | | |
| OR2T5 | olfactory receptor family 2 subfamily | | | 1q44 |
| | T member 5 | | | |
| OR2T6 | olfactory receptor family 2 subfamily | OR2T6P, | OST703 | 1q44 |
| | T member 6 | OR2T9 | | |
| OR2T7 | olfactory receptor family 2 subfamily | OR2T7P | OST723 | 1q44 |
| | T member 7 | | | |
| OR2T8 | olfactory receptor family 2 subfamily | OR2T8P | | 1q44 |
| | T member 8 | | | |
| OR2T10 | olfactory receptor family 2 subfamily | | | 1q44 |
| | T member 10 | | | |
| OR2T11 | olfactory receptor family 2 subfamily | | OR2T11Q | 1q44 |
| | T member 11 (gene/pseudogene) | | | |
| OR2T12 | olfactory receptor family 2 subfamily | | | 1q44 |
| | T member 12 | | | |
| OR2T27 | olfactory receptor family 2 subfamily | | | 1q44 |
| | T member 27 | | | |
| OR2T29 | olfactory receptor family 2 subfamily | | | 1q44 |
| | T member 29 | | | |
| OR2T32P | olfactory receptor family 2 subfamily | | | 1q44 |
| | T member 32 pseudogene | | | |
| OR2T33 | olfactory receptor family 2 subfamily | | | 1q44 |
| | T member 33 | | | |
| OR2T34 | olfactory receptor family 2 subfamily | | | 1q44 |
| | T member 34 | | | |
| OR2T35 | olfactory receptor family 2 subfamily | | | 1q44 |
| | T member 35 | | | |
| OR2U1P | olfactory receptor family 2 subfamily | OR2AU1P | hs6M1-24 | 6p22.2-p21.32 |
| | U member 1 pseudogene | | | |
| OR2U2P | olfactory receptor family 2 subfamily | | hs6M1-23 | 6p22.2-p21.32 |
| | U member 2 pseudogene | | | |
| OR2V1 | olfactory receptor family 2 subfamily | OR2V1P | OST265 | 5q35.3 |
| | V member 1 | | | |
| OR2V2 | olfactory receptor family 2 subfamily | OR2V3 | OST713 | 5q35.3 |
| | V member 2 | | | |
| OR2W1 | olfactory receptor family 2 subfamily | | hs6M1-15 | 6p22.1 |
| | W member 1 | | | |
| OR2W2P | olfactory receptor family 2 subfamily | | hs6M1-30P | 6p22.1 |
| | W member 2 pseudogene | | | |
| OR2W3 | olfactory receptor family 2 subfamily | OR2W8P, | OST718 | 1q44 |
| | W member 3 | OR2W3P | | |
| OR2W4P | olfactory receptor family 2 subfamily | | | 6p22.1 |
| | W member 4 pseudogene | | | |
| OR2W5 | olfactory receptor family 2 subfamily | OR2W5P | OST722 | 1q44 |
| | W member 5 (gene/pseudogene) | | | |
| OR2W6P | olfactory receptor family 2 subfamily | OR2W7P | | 6p22.1 |
| | W member 6 pseudogene | | | |
| OR2X1P | olfactory receptor family 2 subfamily | | | 1q44 |
| | X member 1 pseudogene | | | |
| OR2Y1 | olfactory receptor family 2 subfamily | | | 5q35.3 |
| | Y member 1 | | | |
| OR2Z1 | olfactory receptor family 2 subfamily | OR2Z2 | | 19p13.2 |
| | Z member 1 | | | |

Olfactory receptors, family 3:

| Approved | | Previous | Syn- | |
|---|---|---|---|---|
| Symbol | Approved Name | Symbols | onyms | Chromosome |
| OR3A1 | olfactory receptor family 3 | | OLFRA03, | 17p13.3 |
| | subfamily A member 1 | | OR40, | |
| | | | OR17-40 | |
| OR3A2 | olfactory receptor family 3 | | OLFRA04, | 17p13.3 |
| | subfamily A member 2 | | OR228, | |
| | | | OR17-228 | |
| OR3A3 | olfactory receptor family 3 | OR3A6, | OR17-201, | 17p13.2 |
| | subfamily A member 3 | OR3A7, | OR17-137, | |
| | | OR3A8P | OR17-16 | |
| OR3A4P | olfactory receptor family 3 | OR3A4 | | 17p13.3 |
| | subfamily A member 4 pseudogene | | | |
| OR3B1P | olfactory receptor family 3 | | | Xq28 |
| | subfamily B member 1 pseudogene | | | |
| OR3D1P | olfactory receptor family 3 | | | 1q44 |
| | subfamily D member 1 pseudogene | | | |

Olfactory receptors, family 4:

| Approved | | Previous | Syn- | |
|---|---|---|---|---|
| Symbol | Approved Name | Symbols | onyms | Chromosome |
| OR4A1P | olfactory receptor family 4 subfamily | OR4A20P | OR11-30 | 11p11.12 |
| | A member 1 pseudogene | | | |
| OR4A2P | olfactory receptor family 4 subfamily | | | 11q11 |
| | A member 2 pseudogene | | | |
| OR4A3P | olfactory receptor family 4 subfamily | | | 11q11 |
| | A member 3 pseudogene | | | |
| OR4A4P | olfactory receptor family 4 subfamily | OR4A4 | | 11q11 |
| | A member 4 pseudogene | | | |
| OR4A5 | olfactory receptor family 4 subfamily | | | 11q11 |
| | A member 5 | | | |
| OR4A6P | olfactory receptor family 4 subfamily | | | 11q11 |
| | A member 6 pseudogene | | | |
| OR4A7P | olfactory receptor family 4 subfamily | | | 11q11 |
| | A member 7 pseudogene | | | |
| OR4A8 | olfactory receptor family 4 subfamily | OR4A8P | | 11q11 |
| | A member 8 (gene/pseudogene) | | | |
| OR4A9P | olfactory receptor family 4 subfamily | | | 11q11 |
| | A member 9 pseudogene | | | |
| OR4A10P | olfactory receptor family 4 subfamily | OR4A25P | | 11q11 |
| | A member 10 pseudogene | | | |
| OR4A11P | olfactory receptor family 4 subfamily | | | 11q11 |
| | A member 11 pseudogene | | | |
| OR4A12P | olfactory receptor family 4 subfamily | | | 11q11 |
| | A member 12 pseudogene | | | |
| OR4A13P | olfactory receptor family 4 subfamily | | | 11q11 |
| | A member 13 pseudogene | | | |
| OR4A14P | olfactory receptor family 4 subfamily | | | 11q11 |
| | A member 14 pseudogene | | | |
| OR4A15 | olfactory receptor family 4 subfamily | | | 11q11 |
| | A member 15 | | | |
| OR4A16 | olfactory receptor family 4 subfamily | | OR4A16Q | 11q11 |
| | A member 16 | | | |
| OR4A17P | olfactory receptor family 4 subfamily | OR4A22P | | 11q11 |
| | A member 17 pseudogene | | | |
| OR4A18P | olfactory receptor family 4 subfamily | | | 11p11.12 |
| | A member 18 pseudogene | | | |
| OR4A19P | olfactory receptor family 4 subfamily | | | 11p11.12 |
| | A member 19 pseudogene | | | |
| OR4A21P | olfactory receptor family 4 subfamily | | | 11q11 |
| | A member 21 pseudogene | | | |
| OR4A40P | olfactory receptor family 4 subfamily | | | 11p11.2 |
| | A member 40 pseudogene | | | |
| OR4A41P | olfactory receptor family 4 subfamily | | | 11p11.2 |
| | A member 41 pseudogene | | | |
| OR4A42P | olfactory receptor family 4 subfamily | | | 11p11.2 |
| | A member 42 pseudogene | | | |
| OR4A43P | olfactory receptor family 4 subfamily | | | 11p11.2 |
| | A member 43 pseudogene | | | |
| OR4A44P | olfactory receptor family 4 subfamily | | | 11p11.2 |
| | A member 44 pseudogene | | | |
| OR4A45P | olfactory receptor family 4 subfamily | | | 11p11.2 |
| | A member 45 pseudogene | | | |
| OR4A46P | olfactory receptor family 4 subfamily | | | 11p11.2 |
| | A member 46 pseudogene | | | |
| OR4A47 | olfactory receptor family 4 subfamily | | | 11p11.2 |
| | A member 47 | | | |
| OR4A48P | olfactory receptor family 4 subfamily | | | 11p11.2 |
| | A member 48 pseudogene | | | |
| OR4A49P | olfactory receptor family 4 subfamily | | | 11p11.12 |
| | A member 49 pseudogene | | | |
| OR4A50P | olfactory receptor family 4 subfamily | | | 11q11 |
| | A member 50 pseudogene | | | |
| OR4B1 | olfactory receptor family 4 subfamily | | OST208 | 11p11.2 |
| | B member 1 | | | |
| OR4B2P | olfactory receptor family 4 subfamily | | hg449 | 11p11.2 |
| | B member 2 pseudogene | | | |
| OR4C1P | olfactory receptor family 4 subfamily | OR4C1 | HTPCRX11, | 11q11 |
| | C member 1 pseudogene | | HSHTPCRX11 | |
| OR4C2P | olfactory receptor family 4 subfamily | OR4C8P | | 11p11.2 |
| | C member 2 pseudogene | | | |
| OR4C3 | olfactory receptor family 4 subfamily | | | 11p11.2 |
| | C member 3 | | | |
| OR4C4P | olfactory receptor family 4 subfamily | OR4C17P, | OR4C47P | 11q12.1 |
| | C member 4 pseudogene | OR4C17 | | |
| OR4C5 | olfactory receptor family 4 subfamily | OR4C5P | OR4C5Q | 11p11.2 |
| | C member 5 (gene/pseudogene) | | | |
| OR4C6 | olfactory receptor family 4 subfamily | | | 11q11 |
| | C member 6 | | | |
| OR4C7P | olfactory receptor family 4 subfamily | | | 11q11 |
| | C member 7 pseudogene | | | |
| OR4C9P | olfactory receptor family 4 subfamily | | | 11p11.2 |
| | C member 9 pseudogene | | | |
| OR4C10P | olfactory receptor family 4 subfamily | | | 11p11.2 |
| | C member 10 pseudogene | | | |
| OR4C11 | olfactory receptor family 4 subfamily | OR4C11P | | 11q11 |
| | C member 11 | | | |
| OR4C12 | olfactory receptor family 4 subfamily | | | 11p11.12 |
| | C member 12 | | | |
| OR4C13 | olfactory receptor family 4 subfamily | | | 11p11.12 |
| | C member 13 | | | |
| OR4C14P | olfactory receptor family 4 subfamily | | | 11q11 |
| | C member 14 pseudogene | | | |
| OR4C15 | olfactory receptor family 4 subfamily | | | 11q11 |
| | C member 15 | | | |
| OR4C16 | olfactory receptor family 4 subfamily | | | 11q11 |
| | C member 16 (gene/pseudogene) | | | |
| OR4C45 | olfactory receptor family 4 subfamily | | | 11p11.12 |
| | C member 45 (gene/pseudogene) | | | |
| OR4C46 | olfactory receptor family 4 subfamily | | | 11q11 |
| | C member 46 | | | |
| OR4C48P | olfactory receptor family 4 subfamily | | | 11p11.12 |
| | C member 48 pseudogene | | | |
| OR4C49P | olfactory receptor family 4 subfamily | | | 11p11.12 |
| | C member 49 pseudogene | | | |
| OR4C50P | olfactory receptor family 4 subfamily | | | 11q11 |
| | C member 50 pseudogene | | | |
| OR4D1 | olfactory receptor family 4 subfamily | OR4D3 | TPCR16 | 17q22 |
| | D member 1 | | | |
| OR4D2 | olfactory receptor family 4 subfamily | | | 17q22 |
| | D member 2 | | | |
| OR4D5 | olfactory receptor family 4 subfamily | | | 11q24.1 |
| | D member 5 | | | |
| OR4D6 | olfactory receptor family 4 subfamily | | | 11q12.1 |
| | D member 6 | | | |
| OR4D7P | olfactory receptor family 4 subfamily | | OST724 | 11q12.1 |
| | D member 7 pseudogene | | | |
| OR4D8P | olfactory receptor family 4 subfamily | | | 11q12.1 |
| | D member 8 pseudogene | | | |
| OR4D9 | olfactory receptor family 4 subfamily | | | 11q12.1 |
| | D member 9 | | | |
| OR4D10 | olfactory receptor family 4 subfamily | OR4D10P | OST711 | 11q12.1 |
| | D member 10 | | | |
| OR4D11 | olfactory receptor family 4 subfamily | OR4D11P | | 11q12.1 |
| | D member 11 | | | |
| OR4D12P | olfactory receptor family 4 subfamily | OR7E103P | | 4p16.3 |
| | D member 12 pseudogene | | | |
| OR4E1 | olfactory receptor family 4 subfamily | OR4E1P | | 14q11.2 |
| | E member 1 (gene/pseudogene) | | | |
| OR4E2 | olfactory receptor family 4 subfamily | | | 14q11.2 |
| | E member 2 | | | |
| OR4F1P | olfactory receptor family 4 subfamily | OR4F1 | HSDJ0609N19 | 6p25.3 |
| | F member 1 pseudogene | | | |
| OR4F2P | olfactory receptor family 4 subfamily | | OR4F2, | 11p15.5 |
| | F member 2 pseudogene | | hs6M1-11, | |
| | | | S191N21 | |
| OR4F3 | olfactory receptor family 4 subfamily | | | 5q35.3 |
| | F member 3 | | | |
| OR4F4 | olfactory receptor family 4 subfamily | | OR4F18 | 15q26.3 |
| | F member 4 | | | |
| OR4F5 | olfactory receptor family 4 subfamily | | | 1p36.33 |
| | F member 5 | | | |
| OR4F6 | olfactory receptor family 4 subfamily | OR4F12 | | 15q26.3 |
| | F member 6 | | | |
| OR4F7P | olfactory receptor family 4 subfamily | OR4F10 | | 6q27 |
| | F member 7 pseudogene | | | |
| OR4F8P | olfactory receptor family 4 subfamily | OR4F20P, | | 19p13.3 |
| | F member 8 pseudogene | OR4F9P | | |
| OR4F13P | olfactory receptor family 4 subfamily | | | 15q26.3 |
| | F member 13 pseudogene | | | |
| OR4F14P | olfactory receptor family 4 subfamily | OR4F14 | | 15q26.3 |
| | F member 14 pseudogene | | | |
| OR4F15 | olfactory receptor family 4 subfamily | | | 15q26.3 |
| | F member 15 | | | |
| OR4F16 | olfactory receptor family 4 subfamily | | | 1p36.33 |
| | F member 16 | | | |
| OR4F17 | olfactory receptor family 4 subfamily | OR4F19, | | 19p13.3 |
| | F member 17 | OR4F11P, | | |
| | | OR4F18 | | |
| OR4F21 | olfactory receptor family 4 subfamily | OR4F21P | | 8p23.3 |
| | F member 21 | | | |
| OR4F28P | olfactory receptor family 4 subfamily | | | 15q26.3 |
| | F member 28 pseudogene | | | |
| OR4F29 | olfactory receptor family 4 subfamily | | | 1p36.33 |
| | F member 29 | | | |
| OR4G1P | olfactory receptor family 4 subfamily | OR4G8P | OLB | 19p13.3 |
| | G member 1 pseudogene | | | |
| OR4G2P | olfactory receptor family 4 subfamily | OR4G7P | | 15q26.3 |
| | G member 2 pseudogene | | | |
| OR4G3P | olfactory receptor family 4 subfamily | OR4G3, | OLC, | 19p13.3 |
| | G member 3 pseudogene | OR4G5P | OLC-7501 | |
| OR4G4P | olfactory receptor family 4 subfamily | | | 1p36.33 |
| | G member 4 pseudogene | | | |
| OR4G6P | olfactory receptor family 4 subfamily | | | 15q26.3 |
| | G member 6 pseudogene | | | |
| OR4G11P | olfactory receptor family 4 subfamily | | | 1p36.33 |
| | G member 11 pseudogene | | | |
| OR4H6P | olfactory receptor family 4 subfamily | OR4H9P, | OR15-71, | 15q11.2 |
| | H member 6 pseudogene | OR4H10P, | OR4H6, | |
| | | OR4H5P, | OR15-82, | |
| | | OR4H11P, | OR4H9, | |
| | | OR4H5, | OR5-39, | |
| | | OR4H7, | OR5-84, | |
| | | OR4H7P, | OR4-114, | |
| | | OR4H2P, | OR4-115, | |
| | | OR4H3P, | OR4-119, | |
| | | OR4H11, | OR15-69, | |
| | | OR4H2, | OR15-80, | |
| | | OR4H3, | OR15-81, | |
| | | OR4H1P, | OR14-58 | |
| | | OR4H4P, | | |
| | | OR4H10, | | |
| | | OR4H4, | | |
| | | OR4H8P, | | |
| | | OR4H8 | | |
| OR4H12P | olfactory receptor family 4 subfamily | OR4H12 | C14orf14 | 14p13 |
| | H member 12 pseudogene | | | |
| OR4K1 | olfactory receptor family 4 subfamily | | | 14q11.2 |
| | K member 1 | | | |
| OR4K2 | olfactory receptor family 4 subfamily | | | 14q11.2 |
| | K member 2 | | | |
| OR4K3 | olfactory receptor family 4 subfamily | OR4K3P | | 14q11.2 |
| | K member 3 (gene/pseudogene) | | | |
| OR4K4P | olfactory receptor family 4 subfamily | | | 14q11.2 |
| | K member 4 pseudogene | | | |
| OR4K5 | olfactory receptor family 4 subfamily | | | 14q11.22 |
| | K member 5 | | | |
| OR4K6P | olfactory receptor family 4 subfamily | | | 14q11.2 |
| | K member 6 pseudogene | | | |
| OR4K7P | olfactory receptor family 4 subfamily | OR4K10P | | 18p11.21 |
| | K member 7 pseudogene | | | |
| OR4K8P | olfactory receptor family 4 subfamily | OR4K9P | | 18p11.21 |
| | K member 8 pseudogene | | | |
| OR4K11P | olfactory receptor family 4 subfamily | | OR21-1 | 21q11.2 |
| | K member 11 pseudogene | | | |
| OR4K12P | olfactory receptor family 4 subfamily | | OR21-2 | 21q11.2 |
| | K member 12 pseudogene | | | |
| OR4K13 | olfactory receptor family 4 subfamily | | | 14q11.2 |
| | K member 13 | | | |
| OR4K14 | olfactory receptor family 4 subfamily | | | 14q11.2 |
| | K member 14 | | | |
| OR4K15 | olfactory receptor family 4 subfamily | | OR4K15Q | 14q11.2 |
| | K member 15 | | | |
| OR4K16P | olfactory receptor family 4 subfamily | | | 14q11.2 |
| | K member 16 pseudogene | | | |
| OR4K17 | olfactory receptor family 4 subfamily | | | 14q11.2 |
| | K member 17 | | | |
| OR4L1 | olfactory receptor family 4 subfamily | OR4L2P | | 14q11.2 |
| | L member 1 | | | |
| OR4M1 | olfactory receptor family 4 subfamily | | | 14q11.2 |
| | M member 1 | | | |
| OR4M2 | olfactory receptor family 4 subfamily | | | 15q11.2 |
| | M member 2 | | | |
| OR4N1P | olfactory receptor family 4 subfamily | | | 14q11.2 |
| | N member 1 pseudogene | | | |
| OR4N2 | olfactory receptor family 4 subfamily | | | 14q11.2 |
| | N member 2 | | | |
| OR4N3P | olfactory receptor family 4 subfamily | | | 15q11.2 |
| | N member 3 pseudogene | | | |
| OR4N4 | olfactory receptor family 4 subfamily | | | 15q11.2 |
| | N member 4 | | | |
| OR4N5 | olfactory receptor family 4 subfamily | | | 14q11.2 |
| | N member 5 | | | |
| OR4P1P | olfactory receptor family 4 subfamily | | | 11q11 |
| | P member 1 pseudogene | | | |
| OR4P4 | olfactory receptor family 4 subfamily | OR4P3P | | 11q11 |
| | P member 4 | | | |
| OR4Q1P | olfactory receptor family 4 subfamily | | | 15q11.2 |
| | Q member 1 pseudogene | | | |
| OR4Q2 | olfactory receptor family 4 subfamily | OR4Q2P | | 14q11.2 |
| | Q member 2 (gene/pseudogene) | | | |
| OR4Q3 | olfactory receptor family 4 subfamily | OR4Q4 | C14orf13 | 14p13 |
| | Q member 3 | | | |
| OR4R1P | olfactory receptor family 4 subfamily | | | 11p11.2 |
| | R member 1 pseudogene | | | |
| OR4R2P | olfactory receptor family 4 subfamily | | | 11q11 |
| | R member 2 pseudogene | | | |
| OR4R3P | olfactory receptor family 4 subfamily | | | 11p11.12 |
| | R member 3 pseudogene | | | |
| OR4S1 | olfactory receptor family 4 subfamily | | | 11p11.2 |
| | S member 1 | | | |
| OR4S2 | olfactory receptor family 4 subfamily | OR4S2P | OST725 | 11q11 |
| | S member 2 | | | |
| OR4T1P | olfactory receptor family 4 subfamily | | | 14q11.2 |
| | T member 1 pseudogene | | | |
| OR4U1P | olfactory receptor family 4 subfamily | | | 14q11.2 |
| | U member 1 pseudogene | | | |
| OR4V1P | olfactory receptor family 4 subfamily | | | 11q11 |
| | V member 1 pseudogene | | | |
| OR4W1P | olfactory receptor family 4 subfamily | | | Xq25 |
| | W member 1 pseudogene | | | |
| OR4X1 | olfactory receptor family 4 subfamily | | | 11p11.2 |
| | X member 1 (gene/pseudogene) | | | |
| OR4X2 | olfactory receptor family 4 subfamily | | | 11p11.2 |
| | X member 2 (gene/pseudogene) | | | |
| OR4X7P | olfactory receptor family 4 subfamily | | | 11q11 |
| | X member 7 pseudogene | | | |

Olfactory receptors, family 5

| Approved | | Previous | Syn- | |
|---|---|---|---|---|
| Symbol | Approved Name | Symbols | onyms | Chromosome |
| OR4A1P | olfactory receptor family 5 subfamily | OR5A1P | OST181 | 11q12.1 |
| | A member 1 | | | |
| OR4A2P | olfactory receptor family 5 subfamily | | | 11q12.1 |
| | A member 2 | | | |
| OR4A3P | olfactory receptor family 5 subfamily | OR5AC1P | | 3q11.2 |
| | AC member 1 (gene/pseudogene) | | | |
| OR4A4P | olfactory receptor family 5 subfamily | | HSA1 | 3q11.2 |
| | AC member 2 | | | |
| OR4A5 | olfactory receptor family 5 subfamily | | | 3q11.2 |
| | AC member 4 pseudogene | | | |
| OR4A6P | olfactory receptor family 5 subfamily | | | 19q13.43 |
| | AH member 1 pseudogene | | | |
| OR4A7P | olfactory receptor family 5 subfamily | OR5AK5P | | 11q12.1 |
| | AK member 1 pseudogene | | | |
| OR4A8 | olfactory receptor family 5 subfamily | | | 11q12.1 |
| | AK member 2 | | | |
| OR4A9P | olfactory receptor family 5 subfamily | OR5AK3 | | 11q12.1 |
| | AK member 3 pseudogene | | | |
| OR4A10P | olfactory receptor family 5 subfamily | | | 11q12.1 |
| | AK member 4 pseudogene | | | |
| OR4A11P | olfactory receptor family 5 subfamily | OR5AL1P | | 11q12.1 |
| | AL member 1 (gene/pseudogene) | | | |
| OR4A12P | olfactory receptor family 5 subfamily | | | 11q12.1 |
| | AL member 2 pseudogene | | | |
| OR4A13P | olfactory receptor family 5 subfamily | | | 11q12.1 |
| | AM member 1 pseudogene | | | |
| OR4A14P | olfactory receptor family 5 subfamily | | | 11q12.1 |
| | AN member 1 | | | |
| OR4A15 | olfactory receptor family 5 subfamily | | | 11q12.1 |
| | AN member 2 pseudogene | | | |
| OR4A16 | olfactory receptor family 5 subfamily | | | 11q |
| | AO member 1 pseudogene | | | |
| OR4A17P | olfactory receptor family 5 subfamily | | | 11q12.1 |
| | AP member 1 pseudogene | | | |
| OR4A18P | olfactory receptor family 5 subfamily | | | 11q12.1 |
| | AP member 2 | | | |
| OR4A19P | olfactory receptor family 5 subfamily | | | 11q12.1 |
| | AQ member 1 pseudogene | | | |
| OR4A21P | olfactory receptor family 5 subfamily | | | 11q12.1 |
| | AR member 1 (gene/pseudogene) | | | |
| OR4A40P | olfactory receptor family 5 subfamily | | | 11q12.1 |
| | AS member 1 | | | |
| OR4A41P | olfactory receptor family 5 subfamily | | | 14q11.2 |
| | AU member 1 | | | |
| OR4A42P | olfactory receptor family 5 subfamily | | | Xq26.2 |
| | AW member 1 pseudogene | | | |
| OR4A43P | olfactory receptor family 5 subfamily | | | 11q12.1 |
| | AZ member 1 pseudogene | | | |
| OR4A44P | olfactory receptor family 5 subfamily | OR5B9P, | OR8-122, | 11q12.1 |
| | B member 1 pseudogene | OR5B9, | OR8-123, | |
| | | OR5B5P, | OR6-57, | |
| | | OR5B14P, | OR6-55, | |
| | | OR5B7P, | OR3-144, | |
| | | OR5B7, | OR912-92 | |
| | | OR5B8, | | |
| | | OR5B8P, | | |
| | | OR5B5, | | |
| | | OR5B6, | | |
| | | OR5B6P | | |
| OR4A45P | olfactory receptor family 5 subfamily | | OST073 | 11q12.1 |
| | B member 2 | | | |
| OR4A46P | olfactory receptor family 5 subfamily | OR5B13 | OST129 | 11q12.1 |
| | B member 3 | | | |
| OR4A47 | olfactory receptor family 5 subfamily | OR5B11P, | OR13-67, | 11q12.1 |
| | B member 10 pseudogene | OR5B4P, | OR13-34, | |
| | | OR5B10, | OR13-64 | |
| | | OR5B11, | | |
| | | OR5B18P | | |
| OR4A48P | olfactory receptor family 5 subfamily | OR5B12P, | OST743 | 11q12.1 |
| | B member 12 | OR5B16 | | |
| OR4A49P | olfactory receptor family 5 subfamily | | | 11q12.1 |
| | B member 15 pseudogene | | | |
| OR4A50P | olfactory receptor family 5 subfamily | OR5B20P | | 11q12.1 |
| | B member 17 | | | |
| OR4B1 | olfactory receptor family 5 subfamily | | | 11q12.1 |
| | B member 19 pseudogene | | | |
| OR4B2P | olfactory receptor family 5 subfamily | | | 11q12.1 |
| | B member 21 | | | |
| OR4C1P | olfactory receptor family 5 subfamily | | | 11q12.1 |
| | BA member 1 pseudogene | | | |
| OR4C2P | olfactory receptor family 5 subfamily | | | 11q12.1 |
| | BB member 1 pseudogene | | | |
| OR4C3 | olfactory receptor family 5 subfamily | | | 11q12.1 |
| | BC member 1 pseudogene | | | |
| OR4C4P | olfactory receptor family 5 subfamily | | | 11q12.1 |
| | BD member 1 pseudogene | | | |
| OR4C5 | olfactory receptor family 5 subfamily | | | 11q12.1 |
| | BE member 1 pseudogene | | | |
| OR4C6 | olfactory receptor family 5 subfamily | OR5BH2P | | Xq26.2 |
| | BH member 1 pseudogene | | | |
| OR4C7P | olfactory receptor family 5 subfamily | | OST740 | 12q13.11 |
| | BJ member 1 pseudogene | | | |
| OR4C9P | olfactory receptor family 5 subfamily | | | 12q13.11 |
| | BK member 1 pseudogene | | | |
| OR4C10P | olfactory receptor family 5 subfamily | | | 11q12.1 |
| | BL member 1 pseudogene | | | |
| OR4C11 | olfactory receptor family 5 subfamily | | | 3q11.2 |
| | BM member 1 pseudogene | | | |
| OR4C12 | olfactory receptor family 5 subfamily | | | 11q12.1 |
| | BN member 1 pseudogene | | | |
| OR4C13 | olfactory receptor family 5 subfamily | | | 11q12 |
| | BN member 2 pseudogene | | | |
| OR4C14P | olfactory receptor family 5 subfamily | | | 11q12.1 |
| | BP member 1 pseudogene | | | |
| OR4C15 | olfactory receptor family 5 subfamily | OR5BQ2P | | 11q12.1 |
| | BQ member 1 pseudogene | | | |
| OR4C16 | olfactory receptor family 5 subfamily | | | 11q12.1 |
| | BR member 1 pseudogene | | | |
| OR4C45 | olfactory receptor family 5 subfamily | OR5BS1 | | 12q13.2 |
| | BS member 1 pseudogene | | | |
| OR4C46 | olfactory receptor family 5 subfamily | | | 12q13.2 |
| | BT member 1 pseudogene | | | |
| OR4C48P | olfactory receptor family 5 subfamily | OR5C2P | OR9-F, | 9q33.2 |
| | C member 1 | | hRPK-465_F_21 | |
| OR4C49P | olfactory receptor family 5 subfamily | OR5D6P, | OR11-7a, | 11q11 |
| | D member 2 pseudogene | OR5D10P, | OR912-91, | |
| | | OR5D1P, | OR8-127, | |
| | | OR5D5P, | OR912-47, | |
| | | OR5D12P, | OR18-44, | |
| | | OR5D8P, | R5D9P, | |
| | | OR5D7P, | OR18-17, | |
| | | OR5D9P, | OR18-42, | |
| | | OR5D12, | OR18-43, | |
| | | OR5D11P, | OR912-94, | |
| | | OR5D11 | OR8-125 | |
| OR4C50P | olfactory receptor family 5 subfamily | OR5D3, | OR11-8b, | 11q12 |
| | D member 3 pseudogene | OR5D4 | OR11-8c | |
| OR4D1 | olfactory receptor family 5 subfamily | | | 11q11 |
| | D member 13 (gene/pseudogene) | | | |
| OR4D2 | olfactory receptor family 5 subfamily | | | 11q11 |
| | D member 14 | | | |
| OR4D5 | olfactory receptor family 5 subfamily | | | 11q11 |
| | D member 15 pseudogene | | | |
| OR4D6 | olfactory receptor family 5 subfamily | | | 11q12.1 |
| | D member 16 | | | |
| OR4D7P | olfactory receptor family 5 subfamily | | | 11q11 |
| | D member 17 pseudogene | | | |
| OR4D8P | olfactory receptor family 5 subfamily | | | 11q12.1 |
| | D member 18 | | | |
| OR4D9 | olfactory receptor family 5 subfamily | OR5E1 | TPCR24, | 11p15.4 |
| | E member 1 pseudogene | | HSTPCR24 | |
| OR4D10 | olfactory receptor family 5 subfamily | | OR11-10 | 11q12.1 |
| | F member 1 | | | |
| OR4D11 | olfactory receptor family 5 subfamily | | | 11q12.1 |
| | F member 2 pseudogene | | | |
| OR4D12P | olfactory receptor family 5 subfamily | OR5G2P | OR11-104, | 11q12.1 |
| | G member 1 pseudogene | | OR93 | |
| OR4E1 | olfactory receptor family 5 subfamily | OR5G6P, | | 11q12.1 |
| | G member 3 (gene/pseudogene) | OR5G3P | | |
| OR4E2 | olfactory receptor family 5 subfamily | | | 11q12.1 |
| | G member 4 pseudogene | | | |
| OR4F1P | olfactory receptor family 5 subfamily | | | 11q12.1 |
| | G member 5 pseudogene | | | |
| OR4F2P | olfactory receptor family 5 subfamily | | HTPCRX14, | 3q11.2 |
| | H member 1 | | HSHTPCRX14 | |
| OR4F3 | olfactory receptor family 5 subfamily | | | 3q11.2 |
| | H member 2 | | | |
| OR4F4 | olfactory receptor family 5 subfamily | | | 3q11.2 |
| | H member 3 pseudogene | | | |
| OR4F5 | olfactory receptor family 5 subfamily | | | 3q11.2 |
| | H member 4 pseudogene | | | |
| OR4F6 | olfactory receptor family 5 subfamily | | | 3q11.2 |
| | H member 5 pseudogene | | | |
| OR4F7P | olfactory receptor family 5 subfamily | | | 3q11.2 |
| | H member 6 (gene/pseudogene) | | | |
| OR4F8P | olfactory receptor family 5 subfamily | | | 3q11.2 |
| | H member 7 pseudogene | | | |
| OR4F13P | olfactory receptor family 5 subfamily | OR5H8P | | 3q11.2 |
| | H member 8 (gene/pseudogene) | | | |
| OR4F14P | olfactory receptor family 5 subfamily | | | 3q11.2 |
| | H member 14 | | | |
| OR4F15 | olfactory receptor family 5 subfamily | | | 3q11.2 |
| | H member 15 | | | |
| OR4F16 | olfactory receptor family 5 subfamily | | HSOlf1, | 11q12.1 |
| | I member 1 | | OLF1 | |
| OR4F17 | olfactory receptor family 5 subfamily | OR5J1 | HTPCRH02 | 11q12.1 |
| | J member 1 pseudogene | | | |
| OR4F21 | olfactory receptor family 5 subfamily | | | 11q12.1 |
| | J member 2 | | | |
| OR4F28P | olfactory receptor family 5 subfamily | | | 11q |
| | J member 7 pseudogene | | | |
| OR4F29 | olfactory receptor family 5 subfamily | | HTPCRX10, | 3q11.2 |
| | K member 1 | | HSHTPCRX10 | |
| OR4G1P | olfactory receptor family 5 subfamily | | | 3q11.2 |
| | K member 2 | | | |
| OR4G2P | olfactory receptor family 5 subfamily | | | 3q11.2 |
| | K member 3 | | | |
| OR4G3P | olfactory receptor family 5 subfamily | | | 3q11.2 |
| | K member 4 | | | |
| OR4G4P | olfactory receptor family 5 subfamily | | OST262 | 11q12.1 |
| | L member 1 (gene/pseudogene) | | | |
| OR4G6P | olfactory receptor family 5 subfamily | | HTPCRX16, | 11q12.1 |
| | L member 2 | | HSHTPCRX16 | |
| OR4G11P | olfactory receptor family 5 subfamily | | OST050 | 11q11 |
| | M member 1 | | | |
| OR4H6P | olfactory receptor family 5 subfamily | | | 11q12.1 |
| | M member 2 pseudogene | | | |
| OR4H12P | olfactory receptor family 5 subfamily | | | 11q12.1 |
| | M member 3 | | | |
| OR4K1 | olfactory receptor family 5 subfamily | | | 11q12.1 |
| | M member 4 pseudogene | | | |
| OR4K2 | olfactory receptor family 5 subfamily | | | 11q12.1 |
| | M member 5 pseudogene | | | |
| OR4K3 | olfactory receptor family 5 subfamily | | | 11q12.1 |
| | M member 6 pseudogene | | | |
| OR4K4P | olfactory receptor family 5 subfamily | | | 11q12.1 |
| | M member 7 pseudogene | | | |
| OR4K5 | olfactory receptor family 5 subfamily | | | 11q12.1 |
| | M member 8 | | | |
| OR4K6P | olfactory receptor family 5 subfamily | | | 11q12.1 |
| | M member 9 | | | |
| OR4K7P | olfactory receptor family 5 subfamily | | | 11q11 |
| | M member 10 | | | |
| OR4K8P | olfactory receptor family 5 subfamily | | OR11-199 | 11q11 |
| | M member 11 | | | |
| OR4K11P | olfactory receptor family 5 subfamily | | | 11q12.1 |
| | M member 12 pseudogene | | | |
| OR4K12P | olfactory receptor family 5 subfamily | | | 11q12.1 |
| | M member 13 pseudogene | | | |
| OR4K13 | olfactory receptor family 5 subfamily | OR5M15P | | 4p13 |
| | M member 14 pseudogene | | | |
| OR4K14 | olfactory receptor family 5 subfamily | | | 11p15.4 |
| | P member 1 pseudogene | | | |
| OR4K15 | olfactory receptor family 5 subfamily | | JCG3 | 11p15.4 |
| | P member 2 | | | |
| OR4K16P | olfactory receptor family 5 subfamily | | JCG1 | 11p15.4 |
| | P member 3 | | | |
| OR4K17 | olfactory receptor family 5 subfamily | | OST730 | 11p15.4 |
| | P member 4 pseudogene | | | |
| OR4L1 | olfactory receptor family 5 subfamily | OR5R1P | | 11q12.1 |
| | R member 1 (gene/pseudogene) | | | |
| OR4M1 | olfactory receptor family 5 subfamily | | | 2q37.3 |
| | S member 1 pseudogene | | | |
| OR4M2 | olfactory receptor family 5 subfamily | OR5T1P | | 11q12.1 |
| | T member 1 | | | |
| OR4N1P | olfactory receptor family 5 subfamily | | | 11q12.1 |
| | T member 2 | | | |
| OR4N2 | olfactory receptor family 5 subfamily | | OR5T3Q | 11q12.1 |
| | T member 3 | | | |
| OR4N3P | olfactory receptor family 5 subfamily | | hs6M1-21 | 6p22.1 |
| | V member 1 | | | |
| OR4N4 | olfactory receptor family 5 subfamily | | | 11q12.1 |
| | W member 1 pseudogene | | | |
| OR4N5 | olfactory receptor family 5 subfamily | OR5W2P, | | 11q12.1 |
| | W member 2 | OR5W3P | | |

Olfactory receptors, family 6:

| Approved | | Previous | Syn- | |
|---|---|---|---|---|
| Symbol | Approved Name | Symbols | onyms | Chromosome |
| OR6A2 | olfactory receptor family 6 subfamily | OR6A2P, | OR11-55 | 11p15.4 |
| | A member 2 | OR6A1 | | |
| OR6B1 | olfactory receptor family 6 subfamily | | OR7-3 | 7q35 |
| | B member 1 | | | |
| OR6B2 | olfactory receptor family 6 subfamily | OR6B2P | | 2q37.3 |
| | B member 2 | | | |
| OR6B3 | olfactory receptor family 6 subfamily | OR6B3P | OR6B3Q | 2q37.3 |
| | B member 3 | | | |
| OR6C1 | olfactory receptor family 6 subfamily | | OST267 | 12q13.2 |
| | C member 1 | | | |
| OR6C2 | olfactory receptor family 6 subfamily | | OR6C67 | 12q13.2 |
| | C member 2 | | | |
| OR6C3 | olfactory receptor family 6 subfamily | | OST709 | 12q13.2 |
| | C member 3 | | | |
| OR6C4 | olfactory receptor family 6 subfamily | | | 12q13.2 |
| | C member 4 | | | |
| OR6C5P | olfactory receptor family 6 subfamily | | | 12q13.2 |
| | C member 5 pseudogene | | | |
| OR6C6 | olfactory receptor family 6 subfamily | | | 12q13.2 |
| | C member 6 | | | |
| OR6C7P | olfactory receptor family 6 subfamily | | | 12q13.2 |
| | C member 7 pseudogene | | | |
| OR6C64P | olfactory receptor family 6 subfamily | | | 12q13.2 |
| | C member 64 pseudogene | | | |
| OR6C65 | olfactory receptor family 6 subfamily | | | 12q13.2 |
| | C member 65 | | | |
| OR6C66P | olfactory receptor family 6 subfamily | | | 12q13.2 |
| | C member 66 pseudogene | | | |
| OR6C68 | olfactory receptor family 6 subfamily | | | 12q13.2 |
| | C member 68 | | | |
| OR6C69P | olfactory receptor family 6 subfamily | | | 12q13.2 |
| | C member 69 pseudogene | | | |
| OR6C70 | olfactory receptor family 6 subfamily | | | 12q13.2 |
| | C member 70 | | | |
| OR6C71P | olfactory receptor family 6 subfamily | | | 12q13.2 |
| | C member 71 pseudogene | | | |
| OR6C72P | olfactory receptor family 6 subfamily | | | 12q13.2 |
| | C member 72 pseudogene | | | |
| OR6C73P | olfactory receptor family 6 subfamily | | | 12q13.2 |
| | C member 73 pseudogene | | | |
| OR6C74 | olfactory receptor family 6 subfamily | | | 12q13.2 |
| | C member 74 | | | |
| OR6C75 | olfactory receptor family 6 subfamily | | | 12q13.2 |
| | C member 75 | | | |
| OR6C76 | olfactory receptor family 6 subfamily | | | 12q13.2 |
| | C member 76 | | | |
| OR6D1P | olfactory receptor family 6 subfamily | | | 10q11.21 |
| | D member 1 pseudogene | | | |
| OR6E1P | olfactory receptor family 6 subfamily | | | 14q11.2 |
| | E member 1 pseudogene | | | |
| OR6F1 | olfactory receptor family 6 subfamily | | OST731 | 1q44 |
| | F member 1 | | | |
| OR6J1 | olfactory receptor family 6 subfamily | OR6J2, | | 14q11.2 |
| | J member 1 (gene/pseudogene) | OR6J1P | | |
| OR6K1P | olfactory receptor family 6 subfamily | | | 1q23.1 |
| | K member 1 pseudogene | | | |
| OR6K2 | olfactory receptor family 6 subfamily | | | 1q23.1 |
| | K member 2 | | | |
| OR6K3 | olfactory receptor family 6 subfamily | | | 1q23.1 |
| | K member 3 | | | |
| OR6K4P | olfactory receptor family 6 subfamily | | | 1q23.1 |
| | K member 4 pseudogene | | | |
| OR6K5P | olfactory receptor family 6 subfamily | | | 1q23.1 |
| | K member 5 pseudogene | | | |
| OR6K6 | olfactory receptor family 6 subfamily | | | 1q23.1 |
| | K member 6 | | | |
| OR6L1P | olfactory receptor family 6 subfamily | | | 10q26.3 |
| | L member 1 pseudogene | | | |
| OR6L2P | olfactory receptor family 6 subfamily | | | 10q26.3 |
| | L member 2 pseudogene | | | |
| OR6M1 | olfactory receptor family 6 subfamily | | | 11q24.1 |
| | M member 1 | | | |
| OR6M2P | olfactory receptor family 6 subfamily | | | 11q24.1 |
| | M member 2 pseudogene | | | |
| OR6M3P | olfactory receptor family 6 subfamily | | | 11q24.1 |
| | M member 3 pseudogene | | | |
| OR6N1 | olfactory receptor family 6 subfamily | | | 1q23.1 |
| | N member 1 | | | |
| OR6N2 | olfactory receptor family 6 subfamily | | | 1q23.1 |
| | N member 2 | | | |
| OR6P1 | olfactory receptor family 6 subfamily | | | 1q23.1 |
| | P member 1 | | | |
| OR6Q1 | olfactory receptor family 6 subfamily | | | 11q12.1 |
| | Q member 1 (gene/pseudogene) | | | |
| OR6R1P | olfactory receptor family 6 subfamily | | | 1q44 |
| | R member 1 pseudogene | | | |
| OR6R2P | olfactory receptor family 6 subfamily | | | 8p21.3 |
| | R member 2 pseudogene | | | |
| OR6S1 | olfactory receptor family 6 subfamily | | OR6S1Q | 14q11.2 |
| | S member 1 | | | |
| OR6T1 | olfactory receptor family 6 subfamily | | | 11q24.1 |
| | T member 1 | | | |
| OR6U2P | olfactory receptor family 6 subfamily | OR6U1P | | 12q14.2 |
| | U member 2 pseudogene | | | |
| OR6V1 | olfactory receptor family 6 subfamily | | GPR138 | 7q34 |
| | V member 1 | | | |
| OR6W1P | olfactory receptor family 6 subfamily | OR6W1 | sdolf | 7q34 |
| | W member 1 pseudogene | | | |
| OR6X1 | olfactory receptor family 6 subfamily | | | 11q24.1 |
| | X member 1 | | | |
| OR6Y1 | olfactory receptor family 6 subfamily | OR6Y2 | | 1q23.1 |
| | Y member 1 | | | |

Olfactory receptors, family 7:

| Approved | | Previous | Syn- | |
|---|---|---|---|---|
| Symbol | Approved Name | Symbols | onyms | Chromosome |
| OR7A1P | olfactory receptor family 7 | OR7A6P | OR 19-3, | 19p13.12 |
| | subfamily A member 1 pseudogene | | OLF4p, | |
| | | | hg513 | |
| OR7A2P | olfactory receptor family 7 | OR7A7, | hg1003, | 19p13.12 |
| | subfamily A member 2 pseudogene | OR7A2 | OR19-18, | |
| | | | OLF4p | |
| OR7A3P | olfactory receptor family 7 | OR7A12P, | OR 11-7b, | 19p13.12 |
| | subfamily A member 3 pseudogene | OR7A14P, | OR19-12, | |
| | | OR7A14, | OR14-59, | |
| | | OR7A13P | OR14-11 | |
| OR7A5 | olfactory receptor family 7 | | HTPCR2 | 19p13.1 |
| | subfamily A member 5 | | | |
| OR7A8P | olfactory receptor family 7 | OR7A9P | OST042, | 19p13.12 |
| | subfamily A member 8 pseudogene | | HG83, | |
| | | | OR19-11 | |
| OR7A10 | olfactory receptor family 7 | | | 19p13.1 |
| | subfamily A member 10 | | | |
| OR7A11P | olfactory receptor family 7 | OR7A11 | OST527 | 19p13.12 |
| | subfamily A member 11 pseudogene | | | |
| OR7A15P | olfactory receptor family 7 | OR7A4P, | OR19-134, | 19p13.12 |
| | subfamily A member 15 pseudogene | OR7A16P, | OR19-1, | |
| | | OR7A20P | OR19-146 | |
| OR7A17 | olfactory receptor family 7 | | HTPCRX19 | 19p13.12 |
| | subfamily A member 17 | | | |
| OR7A18P | olfactory receptor family 7 | | | 19p13.12 |
| | subfamily A member 18 pseudogene | | | |
| OR7A19P | olfactory receptor family 7 | | | 12q13.11 |
| | subfamily A member 19 pseudogene | | | |
| OR7C1 | olfactory receptor family 7 | OR7C4 | OR19-5 | 19p13.1 |
| | subfamily C member 1 | | | |
| OR7C2 | olfactory receptor family 7 | OR7C3 | OR19-18 | 19p13.1 |
| | subfamily C member 2 | | | |
| OR7D1P | olfactory receptor family 7 | OR7D3P, | OR 19-A | 19p13.2 |
| | subfamily D member 1 pseudogene | OR7D3 | | |
| OR7D2 | olfactory receptor family 7 | | OR 19-4, | 19p13.2 |
| | subfamily D member 2 | | HTPCRH03, | |
| | | | FLJ38149 | |
| OR7D4 | olfactory receptor family 7 | OR7D4P | hg105, | 19p13.2 |
| | subfamily D member 4 | | OR19-B | |
| OR7D11P | olfactory receptor family 7 | | | 19p13.2 |
| | subfamily D member 11 pseudogene | | | |
| OR7E1P | olfactory receptor family 7 | | | 11q13.2 |
| | subfamily E member 1 pseudogene | | | |
| OR7E2P | olfactory receptor family 7 | OR7F2P, | OR 11-6, | 11q14.2 |
| | subfamily E member 2 pseudogene | OR7E51P | hg94 | |
| OR7E4P | olfactory receptor family 7 | OR7F4P | OR11-11a | 11q13.4 |
| | subfamily E member 4 pseudogene | | | |
| OR7E5P | olfactory receptor family 7 | OR7F5P | OR11-12, | 11q12.1 |
| | subfamily E member 5 pseudogene | | FLJ31393 | |
| OR7E7P | olfactory receptor family 7 | | | 7q21.3 |
| | subfamily E member 7 pseudogene | | | |
| OR7E8P | olfactory receptor family 7 | | OR11-11a | 8p23.1 |
| | subfamily E member 8 pseudogene | | | |
| OR7E10P | olfactory receptor family 7 | | OR11-1 | 8p23.1 |
| | subfamily E member 10 pseudogene | | | |
| OR7E11P | olfactory receptor family 7 | OR7E144P | OR11-2 | 11q13.2 |
| | subfamily E member 11 pseudogene | | | |
| OR7E12P | olfactory receptor family 7 | OR7E58P, | OR11-3 | 11p15.4 |
| | subfamily E member 12 pseudogene | OR7E79P | | |
| OR7E13P | olfactory receptor family 7 | | OR11-4 | 11q14.2 |
| | subfamily E member 13 pseudogene | | | |
| OR7E14P | olfactory receptor family 7 | OR7E151P | OR11-5 | 11p15.1 |
| | subfamily E member 14 pseudogene | | | |
| OR7E15P | olfactory receptor family 7 | OR7E80P, | OR11-392, | 8p23.1 |
| | subfamily E member 15 pseudogene | OR7E42P | OST001 | |
| OR7E16P | olfactory receptor family 7 | OR7E60P, | OR19-133, | 19p13.2 |
| | subfamily E member 16 pseudogene | OR7E17P | OR19-9 | |
| OR7E18P | olfactory receptor family 7 | OR7E61, | OR19-14, | 19p13.2 |
| | subfamily E member 18 pseudogene | OR7E98P | TPCR26 | |
| OR7E19P | olfactory receptor family 7 | OR7E65 | OR 19-7 | 19p13.2 |
| | subfamily E member 19 pseudogene | | | |
| OR7E21P | olfactory receptor family 7 | OR7E49P, | OR4DG, | 3p13 |
| | subfamily E member 21 pseudogene | OR7E127P | OST035 | |
| OR7E22P | olfactory receptor family 7 | | OR6DG, | 3p12.3 |
| | subfamily E member 22 pseudogene | | OR3.6 | |
| OR7E23P | olfactory receptor family 7 | OR7E92P | OR21-3 | 21q22.11 |
| | subfamily E member 23 pseudogene | | | |
| OR7E24 | olfactory receptor family 7 | OR7E24P | OR19-8, | 19p13.2 |
| | subfamily E member 24 | | HSHT2, | |
| | | | OR7E24Q | |
| OR7E25P | olfactory receptor family 7 | | OR19-C, | 19p13.2 |
| | subfamily E member 25 pseudogene | | CIT-B-440L2 | |
| OR7E26P | olfactory receptor family 7 | OR7E67P, | OR1-51, | 10p13 |
| | subfamily E member 26 pseudogene | OR7E69P, | OR1-72, | |
| | | OR7E70P, | OR1-73, | |
| | | OR7E68P | OR912-95 | |
| OR7E28P | olfactory receptor family 7 | OR7E133P, | OST128, | 2q24.1 |
| | subfamily E member 28 pseudogene | OR7E107P, | hg616 | |
| | | OR7E27P | | |
| OR7E29P | olfactory receptor family 7 | | OST032 | 3q21.2 |
| | subfamily E member 29 pseudogene | | | |
| OR7E31P | olfactory receptor family 7 | OR7E32P | OST205 | 9q22.2 |
| | subfamily E member 31 pseudogene | | | |
| OR7E33P | olfactory receptor family 7 | | hg688 | 13q21.32 |
| | subfamily E member 33 pseudogene | | | |
| OR7E35P | olfactory receptor family 7 | OR7E120 | OST018 | 4p16.1 |
| | subfamily E member 35 pseudogene | | | |
| OR7E36P | olfactory receptor family 7 | OR7E119P | OST024 | 13q14.11 |
| | subfamily E member 36 pseudogene | | | |
| OR7E37P | olfactory receptor family 7 | | hg533 | 13q14.11 |
| | subfamily E member 37 pseudogene | | | |
| OR7E38P | olfactory receptor family 7 | OR7E76 | OST127 | 7q21.3 |
| | subfamily E member 38 pseudogene | | | |
| OR7E39P | olfactory receptor family 7 | OR7E138P | hg611 | 7p22.1 |
| | subfamily E member 39 pseudogene | | | |
| OR7E41P | olfactory receptor family 7 | OR7F6P, | OR11-20, | 11p15.2 |
| | subfamily E member 41 pseudogene | OR7E50P, | hg84, | |
| | | OR7E95P | OR8-126 | |
| OR7E43P | olfactory receptor family 7 | | OR4-116 | 4p16.3 |
| | subfamily E member 43 pseudogene | | | |
| OR7E46P | olfactory receptor family 7 | | OST379, | 2p13.3 |
| | subfamily E member 46 pseudogene | | MCEEP | |
| OR7E47P | olfactory receptor family 7 | | OR7E141 | 12q13.13 |
| | subfamily E member 47 pseudogene | | | |
| OR7E53P | olfactory receptor family 7 | OR7E78P, | OR3-143, | 3q21.2 |
| | subfamily E member 53 pseudogene | OR7E78, | OR3-142 | |
| | | OR7E132P | | |
| OR7E55P | olfactory receptor family 7 | OR7E56P | OR2DG, | 3p13 |
| | subfamily E member 55 pseudogene | | OR3.2, | |
| | | | OST013 | |
| OR7E59P | olfactory receptor family 7 | OR7E59, | OST119 | 7p22.1 |
| | subfamily E member 59 pseudogene | OR7E137P | | |
| OR7E62P | olfactory receptor family 7 | OR7E63P, | OR7E62, | 2p13.3 |
| | subfamily E member 62 pseudogene | OR7E64P, | OR2-53, | |
| | | OR7E82P | OR7E63, | |
| | | | OR7E64 | |
| OR7E66P | olfactory receptor family 7 | OR7E6P, | hg630, | 3p13 |
| | subfamily E member 66 pseudogene | OR7E20P | HG630, | |
| | | | OR3DG, | |
| | | | OR3.3 | |
| OR7E83P | olfactory receptor family 7 | OR7E134P | | 4p16.1 |
| | subfamily E member 83 pseudogene | | | |
| OR7E84P | olfactory receptor family 7 | OR7E54P | OST185 | 4p16.1 |
| | subfamily E member 84 pseudogene | | | |
| OR7E85P | olfactory receptor family 7 | OR7E88P | | 4p16.1 |
| | subfamily E member 85 pseudogene | | | |
| OR7E86P | olfactory receptor family 7 | | | 4p16.1 |
| | subfamily E member 86 pseudogene | | | |
| OR7E87P | olfactory receptor family 7 | OR7E3P, | OR11-9 | 11q13.4 |
| | subfamily E member 87 pseudogene | OR7F3P | | |
| OR7E89P | olfactory receptor family 7 | | | 2q24.1 |
| | subfamily E member 89 pseudogene | | | |
| OR7E90P | olfactory receptor family 7 | OR7E123P | OST705 | 2q24.1 |
| | subfamily E member 90 pseudogene | | | |
| OR7E91P | olfactory receptor family 7 | | | 2p13.3 |
| | subfamily E member 91 pseudogene | | | |
| OR7E93P | olfactory receptor family 7 | OR7E131P | | 3q21.2 |
| | subfamily E member 93 pseudogene | | | |
| OR7E94P | olfactory receptor family 7 | | | 4q21.21 |
| | subfamily E member 94 pseudogene | | | |
| OR7E96P | olfactory receptor family 7 | | | 8p23.1 |
| | subfamily E member 96 pseudogene | | | |
| OR7E97P | olfactory receptor family 7 | | | 3q21.2 |
| | subfamily E member 97 pseudogene | | | |
| OR7E99P | olfactory receptor family 7 | | | 4p16.3 |
| | subfamily E member 99 pseudogene | | | |
| OR7E100P | olfactory receptor family 7 | | | 3q13.2 |
| | subfamily E member 100 | | | |
| | pseudogene | | | |
| OR7E101P | olfactory receptor family 7 | | | 13q14.13 |
| | subfamily E member 101 | | | |
| | pseudogene | | | |
| OR7E102P | olfactory receptor family 7 | OR7E102 | | 2q11.1 |
| | subfamily E member 102 | | | |
| | pseudogene | | | |
| OR7E104P | olfactory receptor family 7 | | | 13q21.31 |
| | subfamily E member 104 | | | |
| | pseudogene | | | |
| OR7E105P | olfactory receptor family 7 | | | 14q22.1 |
| | subfamily E member 105 | | | |
| | pseudogene | | | |
| OR7E106P | olfactory receptor family 7 | OR7E40P | OST215 | 14q22.1 |
| | subfamily E member 106 | | | |
| | pseudogene | | | |
| OR7E108P | olfactory receptor family 7 | | OST726 | 9q22.2 |
| | subfamily E member 108 | | | |
| | pseudogene | | | |
| OR7E109P | olfactory receptor family 7 | | OST721 | 9q22.2 |
| | subfamily E member 109 | | | |
| | pseudogene | | | |
| OR7E110P | olfactory receptor family 7 | OR7E68P, | hg674, | 10p13 |
| | subfamily E member 110 | OR7E71P, | OR912-109, | |
| | pseudogene | OR7E72P, | OR912-46, | |
| | | OR7E73P, | OR912-108, | |
| | | OR7E74P, | OR912-110, | |
| | | OR7E75P | hg523 | |
| OR7E111P | olfactory receptor family 7 | | | 13q21.32 |
| | subfamily E member 111 | | | |
| | pseudogene | | | |
| OR7E115P | olfactory receptor family 7 | | OST704 | 10p13 |
| | subfamily E member 115 | | | |
| | pseudogene | | | |
| OR7E116P | olfactory receptor family 7 | | OST733 | 9q22.2 |
| | subfamily E member 116 | | | |
| | pseudogene | | | |
| OR7E117P | olfactory receptor family 7 | | OST716 | 11p15.4 |
| | subfamily E member 117 | | | |
| | pseudogene | | | |
| OR7E121P | olfactory receptor family 7 | | | 3p12.3 |
| | subfamily E member 121 | | | |
| | pseudogene | | | |
| OR7E122P | olfactory receptor family 7 | | OST719 | 3p25.3 |
| | subfamily E member 122 | | | |
| | pseudogene | | | |
| OR7E125P | olfactory receptor family 7 | | PJCG6 | 8p23.1 |
| | subfamily E member 125 | | | |
| | pseudogene | | | |
| OR7E126P | olfactory receptor family 7 | | hg500, | 11q13.4 |
| | subfamily E member 126 | | OR11-1 | |
| | pseudogene | | | |
| OR7E128P | olfactory receptor family 7 | | | 11q13.4 |
| | subfamily E member 128 | | | |
| | pseudogene | | | |
| OR7E129P | olfactory receptor family 7 | | | 3q22.1 |
| | subfamily E member 129 | | | |
| | pseudogene | | | |
| OR7E130P | olfactory receptor family 7 | | OST702 | 3q21.2 |
| | subfamily E member 130 | | | |
| | pseudogene | | | |
| OR7E136P | olfactory receptor family 7 | OR7E147P, | | 7p22.1 |
| | subfamily E member 136 | OR7E139P | | |
| | pseudogene | | | |
| OR7E140P | olfactory receptor family 7 | | | 12p13.31 |
| | subfamily E member 140 | | | |
| | pseudogene | | | |
| OR7E145P | olfactory receptor family 7 | | | 11q13.4 |
| | subfamily E member 145 | | | |
| | pseudogene | | | |
| OR7E148P | olfactory receptor family 7 | OR7E150P | | 12p13 |
| | subfamily E member 148 | | | |
| | pseudogene | | | |
| OR7E149P | olfactory receptor family 7 | | | 12p13.31 |
| | subfamily E member 149 | | | |
| | pseudogene | | | |
| OR7E154P | olfactory receptor family 7 | | | 8p23.1 |
| | subfamily E member 154 | | | |
| | pseudogene | | | |
| OR7E155P | olfactory receptor family 7 | | | 13q14.11 |
| | subfamily E member 155 | | | |
| | pseudogene | | | |
| OR7E156P | olfactory receptor family 7 | | | 13q21.31 |
| | subfamily E member 156 | | | |
| | pseudogene | | | |
| OR7E157P | olfactory receptor family 7 | | | 8p23.1 |
| | subfamily E member 157 | | | |
| | pseudogene | | | |
| OR7E158P | olfactory receptor family 7 | | | 8p23.1 |
| | subfamily E member 158 | | | |
| | pseudogene | | | |
| OR7E159P | olfactory receptor family 7 | | | 14q22.1 |
| | subfamily E member 159 | | | |
| | pseudogene | | | |
| OR7E160P | olfactory receptor family 7 | | | 8p23.1 |
| | subfamily E member 160 | | | |
| | pseudogene | | | |
| OR7E161P | olfactory receptor family 7 | | | 8p23.1 |
| | subfamily E member 161 | | | |
| | pseudogene | | | |
| OR7E162P | olfactory receptor family 7 | | | 4p16.3 |
| | subfamily E member 162 | | | |
| | pseudogene | | | |
| OR7E163P | olfactory receptor family 7 | | | 4p16.3 |
| | subfamily E member 163 | | | |
| | pseudogene | | | |
| OR7G1 | olfactory receptor family 7 | OR7G1P | OR19-15 | 19p13.2 |
| | subfamily G member 1 | | | |
| OR7G2 | olfactory receptor family 7 | | OST260 | 19p13.2 |
| | subfamily G member 2 | | | |
| OR7G3 | olfactory receptor family 7 | | OST085 | 19p13.2 |
| | subfamily G member 3 | | | |
| OR7G15P | olfactory receptor family 7 | | | 19p13.2 |
| | subfamily G member 15 pseudogene | | | |
| OR7H1P | olfactory receptor family 7 | | OR7H1 | 19p13.2 |
| | subfamily H member 1 pseudogene | | | |
| OR7H2P | olfactory receptor family 7 | | | 5q21.1 |
| | subfamily H member 2 pseudogene | | | |
| OR7K1P | olfactory receptor family 7 | | | 14q12 |
| | subfamily K member 1 pseudogene | | | |
| OR7L1P | olfactory receptor family 7 | | | Xq26.2 |
| | subfamily L member 1 pseudogene | | | |
| OR7M1P | olfactory receptor family 7 | | | 10q26.3 |
| | subfamily M member 1 pseudogene | | | |

http://www.genenames.org/cgi-bin/download?title=Genefam+data&submit=submit&hgnc_dbtag=on&preset=genefarn& status=Approved&status=Entry+Withdrawn&status_opt=2&=on&format=text&limit=&.cgifields=&.cgifields=chr&.cgifields=status&.cgifields=hgnc_dbtage&where=gd_gene_fam_name%20RLIKE%20‘(%5e|%20)OR7($1,)’&order_by=gd_app_sym_sort

Olfactory receptors, family 8:

| Approved | | Previous | | |
|---|---|---|---|---|
| Symbol | Approved Name | Symbols | Synonyms | Chromosome |
| OR8A1 | olfactory receptor family 8 subfamily | | OST025 | 11q24.2 |
| | A member 1 | | | |
| OR8A2P | olfactory receptor family 8 subfamily | | | 11q24.2 |
| | A member 2 pseudogene | | | |
| OR8A3P | olfactory receptor family 8 subfamily | | | 11q |
| | A member 3 pseudogene | | | |
| OR8B1P | olfactory receptor family 8 subfamily | OR8B11P | OR11-561 | 11q24.2 |
| | B member 1 pseudogene | | | |
| OR8B2 | olfactory receptor family 8 subfamily | | | 11q24.2 |
| | B member 2 | | | |
| OR8B3 | olfactory receptor family 8 subfamily | | | 11q24.2 |
| | B member 3 | | | |
| OR8B4 | olfactory receptor family 8 subfamily | OR8B4P | | 11q24.2 |
| | B member 4 (gene/pseudogene) | | | |
| OR8B5P | olfactory receptor family 8 subfamily | | | 11q25 |
| | B member 5 pseudogene | | | |
| OR8B6P | olfactory receptor family 8 subfamily | | | 11q25 |
| | B member 6 pseudogene | | | |
| OR8B7P | olfactory receptor family 8 subfamily | OR8B13P | | 11q25 |
| | B member 7 pseudogene | | | |
| OR8B8 | olfactory receptor family 8 subfamily | | TPCR85 | 11q24.2 |
| | B member 8 | | | |
| OR8B9P | olfactory receptor family 8 subfamily | | | 11q24.2 |
| | B member 9 pseudogene | | | |
| OR8B10P | olfactory receptor family 8 subfamily | | | 11q24.2 |
| | B member 10 pseudogene | | | |
| OR8B12 | olfactory receptor family 8 subfamily | | | 11q24.2 |
| | B member 12 | | | |
| OR8C1P | olfactory receptor family 8 subfamily | OR8C3P, | OR11-175, | 11q24.2 |
| | C member 1 pseudogene | OR8C4P | OR912-45, | |
| | | | OR912-106 | |
| OR8D1 | olfactory receptor family 8 subfamily | OR8D3 | OST004 | 11q24.2 |
| | D member 1 | | | |
| OR8D2 | olfactory receptor family 8 subfamily | | | 11q24.2 |
| | D member 2 (gene/pseudogene) | | | |
| OR8D4 | olfactory receptor family 8 subfamily | | | 11q24.1 |
| | D member 4 | | | |
| OR8F1P | olfactory receptor family 8 subfamily | | | 11q24.2 |
| | F member 1 pseudogene | | | |
| OR8G1 | olfactory receptor family 8 subfamily | OR8G1P | TPCR25, | 11q24.2 |
| | G member 1 (gene/pseudogene) | | HSTPCR25 | |
| OR8G2P | olfactory receptor family 8 subfamily | OR8G4, | TPCR120, | 11q24.2 |
| | G member 2 pseudogene | OR8G2 | HSTPCR120, | |
| | | | ORL206, | |
| | | | ORL486 | |
| OR8G3P | olfactory receptor family 8 subfamily | | | 11q24.2 |
| | G member 3 pseudogene | | | |
| OR8G5 | olfactory receptor family 8 subfamily | OR8G5P, | | 11q24.2 |
| | G member 5 | OR8G6 | | |
| OR8G7P | olfactory receptor family 8 subfamily | | | 11q24.2 |
| | G member 7 pseudogene | | | |
| OR8H1 | olfactory receptor family 8 subfamily | | | 11q12.1 |
| | H member 1 | | | |
| OR8H2 | olfactory receptor family 8 subfamily | | | 11q12.1 |
| | H member 2 | | | |
| OR8H3 | olfactory receptor family 8 subfamily | | | 11q12.1 |
| | H member 3 | | | |
| OR8I1P | olfactory receptor family 8 subfamily | | | 11q12.1 |
| | I member 1 pseudogene | | | |
| OR8I2 | olfactory receptor family 8 subfamily | | | 11q12.1 |
| | I member 2 | | | |
| OR8I4P | olfactory receptor family 8 subfamily | | | 11q |
| | I member 4 pseudogene | | | |
| OR8J1 | olfactory receptor family 8 subfamily | | | 11q12.1 |
| | J member 1 | | | |
| OR8J2 | olfactory receptor family 8 subfamily | OR8J2P | | 11q12.1 |
| | J member 2 (gene/pseudogene) | | | |
| OR8J3 | olfactory receptor family 8 subfamily | | | 11q12.1 |
| | J member 3 | | | |
| OR8K1 | olfactory receptor family 8 subfamily | | | 11q12.1 |
| | K member 1 | | | |
| OR8K2P | olfactory receptor family 8 subfamily | | | 11q12.1 |
| | K member 2 pseudogene | | | |
| OR8K3 | olfactory receptor family 8 subfamily | | | 11q12.1 |
| | K member 3 (gene/pseudogene) | | | |
| OR8K4P | olfactory receptor family 8 subfamily | | | 11q12.1 |
| | K member 4 pseudogene | | | |
| OR8K5 | olfactory receptor family 8 subfamily | | | 11q12.1 |
| | K member 5 | | | |
| OR8L1P | olfactory receptor family 8 subfamily | | | 11q12.1 |
| | L member 1 pseudogene | | | |
| OR8Q1P | olfactory receptor family 8 subfamily | | | 11q24.2 |
| | Q member 1 pseudogene | | | |
| OR8R1P | olfactory receptor family 8 subfamily | | | 11q13.4 |
| | R member 1 pseudogene | | | |
| OR8S1 | olfactory receptor family 8 subfamily | | | 12q13.2 |
| | S member 1 | | | |
| OR8S21P | olfactory receptor family 8 subfamily | | | 12q13.11 |
| | S member 21 pseudogene | | | |
| OR8T1P | olfactory receptor family 8 subfamily | | | 12q13.11 |
| | T member 1 pseudogene | | | |
| OR8U1 | olfactory receptor family 8 subfamily | | | 11q12.1 |
| | U member 1 | | | |
| OR8U8 | olfactory receptor family 8 subfamily | | | 11q1 alternate |
| | U member 8 | | | reference |
| | | | | locus |
| OR8U9 | olfactory receptor family 8 subfamily | | | 11q1 alternate |
| | U member 9 | | | reference |
| | | | | locus |
| OR8V1P | olfactory receptor family 8 subfamily | | | 11q12.1 |
| | V member 1 pseudogene | | | |
| OR8X1P | olfactory receptor family 8 subfamily | | | 11q24.2 |
| | X member 1 pseudogene | | | |

Olfactory receptors, family 9:

| Approved | | Previous | | |
|---|---|---|---|---|
| Symbol | Approved Name | Symbols | Synonyms | Chromosome |
| OR9A1P | olfactory receptor family 9 subfamily | OR9A1 | HTPCRX06, | 7q34 |
| | A member 1 pseudogene | | HSHTPCRX06 | |
| OR9A2 | olfactory receptor family 9 subfamily | | | 7q34 |
| | A member 2 | | | |
| OR9A3P | olfactory receptor family 9 subfamily | OR9A6P | | 7q34 |
| | A member 3 pseudogene | | | |
| OR9A4 | olfactory receptor family 9 subfamily | | | 7q34 |
| | A member 4 | | | |
| OR9G1 | olfactory receptor family 9 subfamily | OR9G5 | | 11q12.1 |
| | G member 1 | | | |
| OR9G2P | olfactory receptor family 9 subfamily | OR9G6 | | 11q12.1 |
| | G member 2 pseudogene | | | |
| OR9G3P | olfactory receptor family 9 subfamily | | | 11q12.1 |
| | G member 3 pseudogene | | | |
| OR9G4 | olfactory receptor family 9 subfamily | | | 11q12.1 |
| | G member 4 | | | |
| OR9G9 | olfactory receptor family 9 subfamily | | | 11q11 |
| | G member 9 | | | alternate |
| | | | | reference |
| | | | | locus |
| OR9H1P | olfactory receptor family 9 subfamily | | | 1q44 |
| | H member 1 pseudogene | | | |
| OR9I1 | olfactory receptor family 9 subfamily | | | 11q12.1 |
| | I member 1 | | | |
| OR9I2P | olfactory receptor family 9 subfamily | | | 11q12.1 |
| | I member 2 pseudogene | | | |
| OR9I3P | olfactory receptor family 9 subfamily | | OST714 | 11q12.1 |
| | I member 3 pseudogene | | | |
| OR9K1P | olfactory receptor family 9 subfamily | | | 12q13.2 |
| | K member 1 pseudogene | | | |
| OR9K2 | olfactory receptor family 9 subfamily | | | 12q13.2 |
| | K member 2 | | | |
| OR9L1P | olfactory receptor family 9 subfamily | OR9L2P | | 11q12.1 |
| | L member 1 pseudogene | | | |
| OR9M1P | olfactory receptor family 9 subfamily | OR5BG1P | | 11q12.1 |
| | M member 1 pseudogene | | | |
| OR9N1P | olfactory receptor family 9 subfamily | | | 7q34 |
| | N member 1 pseudogene | | | |
| OR9P1P | olfactory receptor family 9 subfamily | OR9P2P | | 7q34 |
| | P member 1 pseudogene | | | |
| OR9Q1 | olfactory receptor family 9 subfamily | | | 11q12.1 |
| | Q member 1 | | | |
| OR9Q2 | olfactory receptor family 9 subfamily | OR9Q2P | | 11q12.1 |
| | Q member 2 | | | |
| OR9R1P | olfactory receptor family 9 subfamily | | | 12q13.2 |
| | R member 1 pseudogene | | | |
| OR9S24P | olfactory receptor family 9 subfamily | OR5J6P | | 2q37.3 |
| | S member 24 pseudogene | | | |

Olfactory receptors, family 10:

| Approved | | Previous | | |
|---|---|---|---|---|
| Symbol | Approved Name | Symbols | Synonyms | Chromosome |
| OR10A2 | olfactory receptor family 10 | OR10A2P | OST363 | 11p15.4 |
| | subfamily A member 2 | | | |
| OR10A3 | olfactory receptor family 10 | | HTPCRX12, | 11p15.4 |
| | subfamily A member 3 | | HSHTPCRX12 | |
| OR10A4 | olfactory receptor family 10 | OR10A4P | | 11p15.4 |
| | subfamily A member 4 | | | |
| OR10A5 | olfactory receptor family 10 | OR10A1 | OR11-403, | 11p15.4 |
| | subfamily A member 5 | | JCG6 | |
| OR10A6 | olfactory receptor family 10 | | | 11p15.4 |
| | subfamily A member 6 | | | |
| | (gene/pseudogene) | | | |
| OR10A7 | olfactory receptor family 10 | | | 12q13.2 |
| | subfamily A member 7 | | | |
| OR10AA1P | olfactory receptor family 10 | | | 1q23.1 |
| | subfamily AA member 1 pseudogene | | | |
| OR10AB1P | olfactory receptor family 10 | | | 11p15.4 |
| | subfamily AB member 1 pseudogene | | | |
| OR10AC1 | olfactory receptor family 10 | OR10AC1P | | 7q35 |
| | subfamily AC member 1 | | | |
| | (gene/pseudogene) | | | |
| OR10AD1 | olfactory receptor family 10 | OR10AD1P | | 12q13.11 |
| | subfamily AD member 1 | | | |
| OR10AE1P | olfactory receptor family 10 | OR10AE2P | | 1q23.2 |
| | subfamily AE member 1 pseudogene | | | |
| OR10AE3P | olfactory receptor family 10 | | | 12q13.2 |
| | subfamily AE member 3 pseudogene | | | |
| OR10AF1P | olfactory receptor family 10 | | | 11q12 |
| | subfamily AF member 1 pseudogene | | | |
| OR10AG1 | olfactory receptor family 10 | | | 11q12.1 |
| | subfamily AG member 1 | | | |
| OR10AH1P | olfactory receptor family 10 | | | 7p22.1 |
| | subfamily AH member 1 pseudogene | | | |
| OR10AK1P | olfactory receptor family 10 | | | 11q |
| | subfamily AK member 1 pseudogene | | | |
| OR10B1P | olfactory receptor family 10 | OR10B2 | | 19p13.12 |
| | subfamily B member 1 pseudogene | | | |
| OR10C1 | olfactory receptor family 10 | OR10C2 | hs6M1-17, | 6p22.1 |
| | subfamily C member 1 | | OR10C1P | |
| | (gene/pseudogene) | | | |
| OR10D1P | olfactory receptor family 10 | OR10D2P | OST074, | 11q24.2 |
| | subfamily D member 1 pseudogene | | HTPCRX03 | |
| OR10D3 | olfactory receptor family 10 | OR10D3P | HTPCRX09 | 11q24.2 |
| | subfamily D member 3 (putative) | | | |
| OR10D4P | olfactory receptor family 10 | OR10D4, | | 11q24.2 |
| | subfamily D member 4 pseudogene | OR10D6P | | |
| OR10D5P | olfactory receptor family 10 | | | 11q24.2 |
| | subfamily D member 5 pseudogene | | | |
| OR10G1P | olfactory receptor family 10 | | | 14q11.2 |
| | subfamily G member 1 pseudogene | | | |
| OR10G2 | olfactory receptor family 10 | | | 14q11.2 |
| | subfamily G member 2 | | | |
| OR10G3 | olfactory receptor family 10 | | | 14q11.2 |
| | subfamily G member 3 | | | |
| OR10G4 | olfactory receptor family 10 | | | 11q24.2 |
| | subfamily G member 4 | | | |
| OR10G5P | olfactory receptor family 10 | | | 11q24.2 |
| | subfamily G member 5 pseudogene | | | |
| OR10G6 | olfactory receptor family 10 | OR10G6P | OR10G6Q | 11q24.1 |
| | subfamily G member 6 | | | |
| OR10G7 | olfactory receptor family 10 | | | 11q24.2 |
| | subfamily G member 7 | | | |
| OR10G8 | olfactory receptor family 10 | | | 11q24.2 |
| | subfamily G member 8 | | | |
| OR10G9 | olfactory receptor family 10 | OR10G10P | | 11q24.2 |
| | subfamily G member 9 | | | |
| OR10H1 | olfactory receptor family 10 | | | 19p13.1 |
| | subfamily H member 1 | | | |
| OR10H2 | olfactory receptor family 10 | | | 19p13.1 |
| | subfamily H member 2 | | | |
| OR10H3 | olfactory receptor family 10 | | | 19p13.1 |
| | subfamily H member 3 | | | |
| OR10H4 | olfactory receptor family 10 | | | 19p13.12 |
| | subfamily H member 4 | | | |
| OR10H5 | olfactory receptor family 10 | | | 19p13.12 |
| | subfamily H member 5 | | | |
| OR10J1 | olfactory receptor family 10 | | HGMP07J, | 1q23.2 |
| | subfamily J member 1 | | HSHGMP07J | |
| OR10J2P | olfactory receptor family 10 | | | 1q23.2 |
| | subfamily J member 2 pseudogene | | | |
| OR10J3 | olfactory receptor family 10 | OR10J3P | | 1q23.2 |
| | subfamily J member 3 | | | |
| OR10J4 | olfactory receptor family 10 | OR10J4P | OST717 | 1q23.2 |
| | subfamily J member 4 | | | |
| | (gene/pseudogene) | | | |
| OR10J5 | olfactory receptor family 10 | | | 1q23.2 |
| | subfamily J member 5 | | | |
| OR10J6P | olfactory receptor family 10 | OR10J6 | | 1q23.2 |
| | subfamily J member 6 pseudogene | | | |
| OR10J7P | olfactory receptor family 10 | | | 1q23.2 |
| | subfamily J member 7 pseudogene | | | |
| OR10J8P | olfactory receptor family 10 | | | 1q23.2 |
| | subfamily J member 8 pseudogene | | | |
| OR10J9P | olfactory receptor family 10 | | | 1q23.2 |
| | subfamily J member 9 pseudogene | | | |
| OR10K1 | olfactory receptor family 10 | | | 1q23.1 |
| | subfamily K member 1 | | | |
| OR10K2 | olfactory receptor family 10 | | | 1q23.1 |
| | subfamily K member 2 | | | |
| OR10N1P | olfactory receptor family 10 | | | 11q24.2 |
| | subfamily N member 1 pseudogene | | | |
| OR10P1 | olfactory receptor family 10 | OR10P1P, | OST701 | 12q13.2 |
| | subfamily P member 1 | OR10P2P, | | |
| | | OR10P3P | | |
| OR10Q1 | olfactory receptor family 10 | | | 11q12.1 |
| | subfamily Q member 1 | | | |
| OR10Q2P | olfactory receptor family 10 | | | 11q12.1 |
| | subfamily Q member 2 pseudogene | | | |
| OR10R1P | olfactory receptor family 10 | | | 1q23.1 |
| | subfamily R member 1 pseudogene | | | |
| OR10R2 | olfactory receptor family 10 | | OR10R2Q | 1q23.1 |
| | subfamily R member 2 | | | |
| OR10R3P | olfactory receptor family 10 | | | 1q23.1 |
| | subfamily R member 3 pseudogene | | | |
| OR10S1 | olfactory receptor family 10 | | | 11q24.1 |
| | subfamily S member 1 | | | |
| OR10T1P | olfactory receptor family 10 | | | 1q23.1 |
| | subfamily T member 1 pseudogene | | | |
| OR10T2 | olfactory receptor family 10 | | | 1q23.1 |
| | subfamily T member 2 | | | |
| OR10U1P | olfactory receptor family 10 | | | 12q13.2 |
| | subfamily U member 1 pseudogene | | | |
| OR10V1 | olfactory receptor family 10 | | | 11q12.1 |
| | subfamily V member 1 | | | |
| OR10V2P | olfactory receptor family 10 | | | 11q12.1 |
| | subfamily V member 2 pseudogene | | | |
| OR10V3P | olfactory receptor family 10 | | | 11q12.1 |
| | subfamily V member 3 pseudogene | | | |
| OR10V7P | olfactory receptor family 10 | | | 14q21.2 |
| | subfamily V member 7 pseudogene | | | |
| OR10W1 | olfactory receptor family 10 | OR10W1P | | 11q12.1 |
| | subfamily W member 1 | | | |
| OR10X1 | olfactory receptor family 10 | OR10X1P | | 1q23.1 |
| | subfamily X member 1 | | | |
| | (gene/pseudogene) | | | |
| OR10Y1P | olfactory receptor family 10 | | | 11q12.1 |
| | subfamily Y member 1 pseudogene | | | |
| OR10Z1 | olfactory receptor family 10 | | | 1q23.1 |
| | subfamily Z member 1 | | | |

Olfactory receptors, family 11:

| Approved | | Previous | | |
|---|---|---|---|---|
| Symbol | Approved Name | Symbols | Synonyms | Chromosome |
| OR11A1 | olfactory receptor family 11 | OR11A2 | hs6M1-18 | 6p22.2-p21.31 |
| | subfamily A member 1 | | | |
| OR11G1P | olfactory receptor family 11 | | | 14q11.2 |
| | subfamily G member 1 pseudogene | | | |
| OR11G2 | olfactory receptor family 11 | | | 14q11.2 |
| | subfamily G member 2 | | | |
| OR11H1 | olfactory receptor family 11 | | OR22-1 | 22q11.1 |
| | subfamily H member 1 | | | |
| OR11H2 | olfactory receptor family 11 | OR11H2P, | | 14q11.2 |
| | subfamily H member 2 | OR11H8P, | | |
| | | C14orf15 | | |
| OR11H3P | olfactory receptor family 11 | | | 15q11.2 |
| | subfamily H member 3 pseudogene | | | |
| OR11H4 | olfactory receptor family 11 | | | 14q11.2 |
| | subfamily H member 4 | | | |
| OR11H5P | olfactory receptor family 11 | | | 14q11.2 |
| | subfamily H member 5 pseudogene | | | |
| OR11H6 | olfactory receptor family 11 | | | 14q11.2 |
| | subfamily H member 6 | | | |
| OR11H7 | olfactory receptor family 11 | OR11H7P | | 14q11.2 |
| | subfamily H member 7 | | | |
| | (gene/pseudogene) | | | |
| OR11H12 | olfactory receptor family 11 | | | 14q11.2 |
| | subfamily H member 12 | | | |
| OR11H13P | olfactory receptor family 11 | | | 14q11.2 |
| | subfamily H member 13 pseudogene | | | |
| OR11I1P | olfactory receptor family 11 | OR11I2P | | 1p13.3 |
| | subfamily I member 1 pseudogene | | | |
| OR11J1P | olfactory receptor family 11 | | | 15q11.2 |
| | subfamily J member 1 pseudogene | | | |
| OR11J2P | olfactory receptor family 11 | | | 15q11.2 |
| | subfamily J member 2 pseudogene | | | |
| OR11J5P | olfactory receptor family 11 | | | 15q11.2 |
| | subfamily J member 5 pseudogene | | | |
| OR11K1P | olfactory receptor family 11 | | | 15q11.2 |
| | subfamily K member 1 pseudogene | | | |
| OR11K2P | olfactory receptor family 11 | | | 14p13 |
| | subfamily K member 2 pseudogene | | | |
| OR11L1 | olfactory receptor family 11 | | | 1q44 |
| | subfamily L member 1 | | | |
| OR11M1P | olfactory receptor family 11 | | | 12q13.1 |
| | subfamily M member 1 pseudogene | | | |
| OR11N1P | olfactory receptor family 11 | | | Xq26.2 |
| | subfamily N member 1 pseudogene | | | |
| OR11P1P | olfactory receptor family 11 | | | 14q1 |
| | subfamily P member 1 pseudogene | | | |
| OR11Q1P | olfactory receptor family 11 | | | Xq26.1 |
| | subfamily Q member 1 pseudogene | | | |

Olfactory receptors, family 12:

| Approved | | Previous | | |
|---|---|---|---|---|
| Symbol | Approved Name | Symbols | Synonyms | Chromosome |
| OR11A1 | olfactory receptor family 12 subfamily | OR12D1P | hs6M1-19 | 6p22.1 |
| | D member 1 (gene/pseudogene) | | | |
| OR11G1P | olfactory receptor family 12 subfamily | | hs6M1-20 | 6p22.1 |
| | D member 2 (gene/pseudogene) | | | |
| OR11G2 | olfactory receptor family 12 subfamily | | hs6M1-27 | 6p22.1 |
| | D member 3 | | | |

Olfactory receptors, family 13:

| Approved | | Previous | | |
|---|---|---|---|---|
| Symbol | Approved Name | Symbols | Synonyms | Chromosome |
| OR13A1 | olfactory receptor family 13 subfamily | | | 10q11.21 |
| | A member 1 | | | |
| OR13C1P | olfactory receptor family 13 subfamily | | | 9q31.1 |
| | C member 1 pseudogene | | | |
| OR13C2 | olfactory receptor family 13 subfamily | | | 9q31.1 |
| | C member 2 | | | |
| OR13C3 | olfactory receptor family 13 subfamily | | | 9q31.1 |
| | C member 3 | | | |
| OR13C4 | olfactory receptor family 13 subfamily | | | 9q31.1 |
| | C member 4 | | | |
| OR13C5 | olfactory receptor family 13 subfamily | | | 9q31.1 |
| | C member 5 | | | |
| OR13C6P | olfactory receptor family 13 subfamily | | | 9p13.3 |
| | C member 6 pseudogene | | | |
| OR13C7 | olfactory receptor family 13 subfamily | OR13C7P | OST706 | 9p13.3 |
| | C member 7 (gene/pseudogene) | | | |
| OR13C8 | olfactory receptor family 13 subfamily | | | 9q31.1 |
| | C member 8 | | | |
| OR13C9 | olfactory receptor family 13 subfamily | | | 9q31.1 |
| | C member 9 | | | |
| OR13D1 | olfactory receptor family 13 subfamily | | | 9q31.1 |
| | D member 1 | | | |
| OR13D2P | olfactory receptor family 13 subfamily | | | 9q31.1 |
| | D member 2 pseudogene | | | |
| OR13D3P | olfactory receptor family 13 subfamily | | | 9q31.1 |
| | D member 3 pseudogene | | | |
| OR13E1P | olfactory receptor family 13 subfamily | OR13E2 | OST741 | 9p13.3 |
| | E member 1 pseudogene | | | |
| OR13F1 | olfactory receptor family 13 subfamily | | | 9q31.1 |
| | F member 1 | | | |
| OR13G1 | olfactory receptor family 13 subfamily | | | 1q44 |
| | G member 1 | | | |
| OR13H1 | olfactory receptor family 13 subfamily | | | Xq26.2 |
| | H member 1 | | | |
| OR13I1P | olfactory receptor family 13 subfamily | OR13I2P | | 9q31.1 |
| | I member 1 pseudogene | | | |
| OR13J1 | olfactory receptor family 13 subfamily | | | 9p13.3 |
| | J member 1 | | | |
| OR13K1P | olfactory receptor family 13 subfamily | | | Xq26.2 |
| | K member 1 pseudogene | | | |
| OR13Z1P | olfactory receptor family 13 subfamily | | | 1q21.1 |
| | Z member 1 pseudogene | | | |
| OR13Z2P | olfactory receptor family 13 subfamily | | | 1q21.1 |
| | Z member 2 pseudogene | | | |
| OR13Z3P | olfactory receptor family 13 subfamily | | | 1q21.1 |
| | Z member 3 pseudogene | | | |

Olfactory receptors, family 14:

| Approved | | Previous | | |
|---|---|---|---|---|
| Symbol | Approved Name | Symbols | Synonyms | Chromosome |
| OR13A1 | olfactory receptor family 14 | OR5AX1P, | | 1q44 |
| | subfamily A member 2 | OR5AX1 | | |
| OR13C1P | olfactory receptor family 14 | OR5AT1 | | 1q44 |
| | subfamily A member 16 | | | |
| OR13C2 | olfactory receptor family 14 | OR5BF1 | | 1q44 |
| | subfamily C member 36 | | | |
| OR13C3 | olfactory receptor family 14 | OR5BU1P, | | 1q44 |
| | subfamily I member 1 | OR5BU1 | | |
| OR13C4 | olfactory receptor family 14 | OR5U1 | hs6M1-28 | 6p22.1 |
| | subfamily J member 1 | | | |
| OR13C5 | olfactory receptor family 14 | OR5AY1 | | 1q44 |
| | subfamily K member 1 | | | |
| OR13C6P | olfactory receptor family 14 | OR5AV1, | | 1q44 |
| | subfamily L member 1 pseudogene | OR5AV1P | | |

Olfactory receptors, family 51:

| Approved | | Previous | | |
|---|---|---|---|---|
| Symbol | Approved Name | Symbols | Synonyms | Chromosome |
| OR51A1P | olfactory receptor family 51 | | | 11p15.4 |
| | subfamily A member 1 pseudogene | | | |
| OR51A2 | olfactory receptor family 51 | | | 11p15.4 |
| | subfamily A member 2 | | | |
| OR51A3P | olfactory receptor family 51 | | | 11p15.4 |
| | subfamily A member 3 pseudogene | | | |
| OR51A4 | olfactory receptor family 51 | | | 11p15.4 |
| | subfamily A member 4 | | | |
| OR51A5P | olfactory receptor family 51 | | | 11p15.4 |
| | subfamily A member 5 pseudogene | | | |
| OR51A6P | olfactory receptor family 51 | | | 11p15.4 |
| | subfamily A member 6 pseudogene | | | |
| OR51A7 | olfactory receptor family 51 | | | 11p15.4 |
| | subfamily A member 7 | | | |
| OR51A8P | olfactory receptor family 51 | | | 11p15.4 |
| | subfamily A member 8 pseudogene | | | |
| OR51A9P | olfactory receptor family 51 | | | 11p15.4 |
| | subfamily A member 9 pseudogene | | | |
| OR51A10P | olfactory receptor family 51 | OR51A11P, | | 11p15.4 |
| | subfamily A member 10 | OR51A13 | | |
| | pseudogene | | | |
| OR51AB1P | olfactory receptor family 51 | | | 11p15.4 |
| | subfamily AB member 1 | | | |
| | pseudogene | | | |
| OR51B2 | olfactory receptor family 51 | OR51B1P | | 11p15.4 |
| | subfamily B member 2 | | | |
| | (gene/pseudogene) | | | |
| OR51B3P | olfactory receptor family 51 | | | 11p15.4 |
| | subfamily B member 3 pseudogene | | | |
| OR51B4 | olfactory receptor family 51 | | | 11p15.4 |
| | subfamily B member 4 | | | |
| OR51B5 | olfactory receptor family 51 | | | 11p15.4 |
| | subfamily B member 5 | | | |
| OR51B6 | olfactory receptor family 51 | | | 11p15.4 |
| | subfamily B member 6 | | | |
| OR51B8P | olfactory receptor family 51 | | | 11p15.4 |
| | subfamily B member 8 pseudogene | | | |
| OR51C1P | olfactory receptor family 51 | OR51C3P, | OST734 | 11p15.4 |
| | subfamily C member 1 pseudogene | OR51C2P | | |
| OR51C4P | olfactory receptor family 51 | | | 11p15.4 |
| | subfamily C member 4 pseudogene | | | |
| OR51D1 | olfactory receptor family 51 | | OR51D1Q | 11p15.4 |
| | subfamily D member 1 | | | |
| OR51E1 | olfactory receptor family 51 | OR51E1P, | GPR136 | 11p15.4 |
| | subfamily E member 1 | OR52A3P, | | |
| | | GPR164 | | |
| OR51E2 | olfactory receptor family 51 | | PSGR | 11p15.4 |
| | subfamily E member 2 | | | |
| OR51F1 | olfactory receptor family 51 | OR51F1P | | 11p15.4 |
| | subfamily F member 1 | | | |
| | (gene/pseudogene) | | | |
| OR51F2 | olfactory receptor family 51 | | | 11p15.4 |
| | subfamily F member 2 | | | |
| OR51F3P | olfactory receptor family 51 | | | 11p15.4 |
| | subfamily F member 3 pseudogene | | | |
| OR51F4P | olfactory receptor family 51 | | | 11p15.4 |
| | subfamily F member 4 pseudogene | | | |
| OR51F5P | olfactory receptor family 51 | | | 11p15.4 |
| | subfamily F member 5 pseudogene | | | |
| OR51G1 | olfactory receptor family 51 | OR51G3P | | 11p15.4 |
| | subfamily G member 1 | | | |
| | (gene/pseudogene) | | | |
| OR51G2 | olfactory receptor family 51 | | | 11p15.4 |
| | subfamily G member 2 | | | |
| OR51H1 | olfactory receptor family 51 | OR51H1P | | 11p15.4 |
| | subfamily H member 1 | | | |
| OR51H2P | olfactory receptor family 51 | | | 11p15.4 |
| | subfamily H member 2 pseudogene | | | |
| OR51I1 | olfactory receptor family 51 | | | 11p15.4 |
| | subfamily I member 1 | | | |
| OR51I2 | olfactory receptor family 51 | | | 11p15.4 |
| | subfamily I member 2 | | | |
| OR51J1 | olfactory receptor family 51 | OR51J2, | | 11p15.4 |
| | subfamily J member 1 | OR51J1P | | |
| | (gene/pseudogene) | | | |
| OR51K1P | olfactory receptor family 51 | | | 11p15.4 |
| | subfamily K member 1 pseudogene | | | |
| OR51L1 | olfactory receptor family 51 | | | 11p15.4 |
| | subfamily L member 1 | | | |
| OR51M1 | olfactory receptor family 51 | | | 11p15.4 |
| | subfamily M member 1 | | | |
| OR51N1P | olfactory receptor family 51 | | | 11p15.4 |
| | subfamily N member 1 pseudogene | | | |
| OR51P1P | olfactory receptor family 51 | OR51P2P | | 11p15.4 |
| | subfamily P member 1 pseudogene | | | |
| OR51Q1 | olfactory receptor family 51 | | | 11p15.4 |
| | subfamily Q member 1 | | | |
| | (gene/pseudogene) | | | |
| OR51R1P | olfactory receptor family 51 | | | 11p15.4 |
| | subfamily R member 1 pseudogene | | | |
| OR51S1 | olfactory receptor family 51 | | | 11p15.4 |
| | subfamily S member 1 | | | |
| OR51T1 | olfactory receptor family 51 | | | 11p15.4 |
| | subfamily T member 1 | | | |
| OR51V1 | olfactory receptor family 51 | OR51A12 | | 11p15.4 |
| | subfamily V member 1 | | | |

Olfactory receptors, family 52:

| Approved | | Previous | | |
|---|---|---|---|---|
| Symbol | Approved Name | Symbols | Synonyms | Chromosome |
| OR52A1 | olfactory receptor family 52 subfamily | | HPFH1OR | 11p15.4 |
| | A member 1 | | | |
| OR52A4P | olfactory receptor family 52 subfamily | OR52A4 | | 11p15.4 |
| | A member 4 pseudogene | | | |
| OR52A5 | olfactory receptor family 52 subfamily | | | 11p15.4 |
| | A member 5 | | | |
| OR52B1P | olfactory receptor family 52 subfamily | | | 11p15.4 |
| | B member 1 pseudogene | | | |
| OR52B2 | olfactory receptor family 52 subfamily | | | 11p15.4 |
| | B member 2 | | | |
| OR52B3P | olfactory receptor family 52 subfamily | | | 11p15.4 |
| | B member 3 pseudogene | | | |
| OR52B4 | olfactory receptor family 52 subfamily | | | 11p15.4 |
| | B member 4 (gene/pseudogene) | | | |
| OR52B5P | olfactory receptor family 52 subfamily | | | 11p15.4 |
| | B member 5 pseudogene | | | |
| OR52B6 | olfactory receptor family 52 subfamily | | | 11p15.4 |
| | B member 6 | | | |
| OR52D1 | olfactory receptor family 52 subfamily | | | 11p15.4 |
| | D member 1 | | | |
| OR52E1 | olfactory receptor family 52 subfamily | OR52E1P | | 11p15.4 |
| | E member 1 (gene/pseudogene) | | | |
| OR52E2 | olfactory receptor family 52 subfamily | | | 11p15.4 |
| | E member 2 | | | |
| OR52E3P | olfactory receptor family 52 subfamily | | | 11p15.4 |
| | E member 3 pseudogene | | | |
| OR52E4 | olfactory receptor family 52 subfamily | | | 11p15.4 |
| | E member 4 | | | |
| OR52E5 | olfactory receptor family 52 subfamily | | | 11p15.4 |
| | E member 5 | | | |
| OR52E6 | olfactory receptor family 52 subfamily | | | 11p15.4 |
| | E member 6 | | | |
| OR52E7P | olfactory receptor family 52 subfamily | | | 11p15.4 |
| | E member 7 pseudogene | | | |
| OR52E8 | olfactory receptor family 52 subfamily | | | 11p15.4 |
| | E member 8 | | | |
| OR52H1 | olfactory receptor family 52 subfamily | | | 11p15.4 |
| | H member 1 | | | |
| OR52H2P | olfactory receptor family 52 subfamily | | | 11p15.4 |
| | H member 2 pseudogene | | | |
| OR52I1 | olfactory receptor family 52 subfamily I | | | 11p15.4 |
| | member 1 | | | |
| OR52I2 | olfactory receptor family 52 subfamily I | | | 11p15.4 |
| | member 2 | | | |
| OR52J1P | olfactory receptor family 52 subfamily J | | | 11p15.4 |
| | member 1 pseudogene | | | |
| OR52J2P | olfactory receptor family 52 subfamily J | OR52J4P | | 11p15.4 |
| | member 2 pseudogene | | | |
| OR52J3 | olfactory receptor family 52 subfamily J | | | 11p15.4 |
| | member 3 | | | |
| OR52K1 | olfactory receptor family 52 subfamily | | | 11p15.4 |
| | K member 1 | | | |
| OR52K2 | olfactory receptor family 52 subfamily | | | 11p15.4 |
| | K member 2 | | | |
| OR52K3P | olfactory receptor family 52 subfamily | | | 11p15.4 |
| | K member 3 pseudogene | | | |
| OR52L1 | olfactory receptor family 52 subfamily | | | 11p15.4 |
| | L member 1 | | | |
| OR52L2P | olfactory receptor family 52 subfamily | OR52L2 | | 11p15.4 |
| | L member 2 pseudogene | | | |
| OR52M1 | olfactory receptor family 52 subfamily | OR52M1P | | 11p15.4 |
| | M member 1 | | | |
| OR52M2P | olfactory receptor family 52 subfamily | OR52M4 | | 11p15.4 |
| | M member 2 pseudogene | | | |
| OR52N1 | olfactory receptor family 52 subfamily | | | 11p15.4 |
| | N member 1 | | | |
| OR52N2 | olfactory receptor family 52 subfamily | | | 11p15.4 |
| | N member 2 | | | |
| OR52N3P | olfactory receptor family 52 subfamily | | | 11p15.4 |
| | N member 3 pseudogene | | | |
| OR52N4 | olfactory receptor family 52 subfamily | | | 11p15.4 |
| | N member 4 (gene/pseudogene) | | | |
| OR52N5 | olfactory receptor family 52 subfamily | | OR52N5Q | 11p15.4 |
| | N member 5 | | | |
| OR52P1P | olfactory receptor family 52 subfamily | OR52P1 | | 11p15.4 |
| | P member 1 pseudogene | | | |
| OR52P2P | olfactory receptor family 52 subfamily | | | 11p15.4 |
| | P member 2 pseudogene | | | |
| OR52Q1P | olfactory receptor family 52 subfamily | | | 11p15.4 |
| | Q member 1 pseudogene | | | |
| OR52R1 | olfactory receptor family 52 subfamily | | | 11p15.4 |
| | R member 1 (gene/pseudogene) | | | |
| OR52S1P | olfactory receptor family 52 subfamily | | | 11p15.4 |
| | S member 1 pseudogene | | | |
| OR52T1P | olfactory receptor family 52 subfamily | | | 11p15.4 |
| | T member 1 pseudogene | | | |
| OR52U1P | olfactory receptor family 52 subfamily | | | 11p15.4 |
| | U member 1 pseudogene | | | |
| OR52V1P | olfactory receptor family 52 subfamily | | | 11p15.4 |
| | V member 1 pseudogene | | | |
| OR52W1 | olfactory receptor family 52 subfamily | OR52W1P | | 11p15.4 |
| | W member 1 | | | |
| OR52X1P | olfactory receptor family 52 subfamily | | | 11p15.4 |
| | X member 1 pseudogene | | | |
| OR52Y1P | olfactory receptor family 52 subfamily | OR52Y2P | | 11p15.4 |
| | Y member 1 pseudogene | | | |
| OR52Z1 | olfactory receptor family 52 subfamily | OR52Z1P | | 11p15.4 |
| | Z member 1 (gene/pseudogene) | | | |

Olfactory receptors, family 55:

| | | | | |
|---|---|---|---|---|
| Approved | | Previous | Syn- | |
| Symbol | Approved Name | Symbols | onyms | Chromosome |

| | | | |
|---|---|---|---|
| OR55B1P | olfactory receptor | OR55B2P, | 11p15.4 |
| | family 55 | OR55C1P | |
| | subfamily B | | |
| | member 1 pseudogene | | |

Olfactory receptors, family 56:

| Approved | | Previous | Syn- | |
|---|---|---|---|---|
| Symbol | Approved Name | Symbols | onyms | Chromosome |
| OR56A1 | olfactory receptor | | | 11p15.4 |
| | family 56 | | | |
| | subfamily A | | | |
| | member 1 | | | |
| OR56A3 | olfactory receptor | OR56A6, | | 11p15.4 |
| | family 56 | OR56A3P | | |
| | subfamily A | | | |
| | member 3 | | | |
| OR56A4 | olfactory receptor | | | 11p15.4 |
| | family 56 | | | |
| | subfamily A | | | |
| | member 4 | | | |
| OR56A5 | olfactory receptor | OR56A5P | | 11p15.4 |
| | family 56 | | | |
| | subfamily A | | | |
| | member 5 | | | |
| OR56A7P | olfactory receptor | | | 11p15.4 |
| | family 56 | | | |
| | subfamily A | | | |
| | member 7 pseudogene | | | |
| OR56B1 | olfactory receptor | OR56B1P | | 11p15.4 |
| | family 56 | | | |
| | subfamily B | | | |
| | member 1 | | | |
| OR56B2P | olfactory receptor | OR56B2 | | 11p15.4 |
| | family 56 | | | |
| | subfamily B | | | |
| | member 2 pseudogene | | | |
| OR56B3P | olfactory receptor | | | 11p15.4 |
| | family 56 | | | |
| | subfamily B | | | |
| | member 3 pseudogene | | | |
| OR56B4 | olfactory receptor | | | 11p15.4 |
| | family 56 | | | |
| | subfamily B | | | |
| | member 4 | | | |

| Receptor gene/protein | Response element |
|---|---|
| Nuclear Hormone | HRE (hormone re- |
| Receptors | sponse element) |
| Estrogen receptor | ERE (estrogen re- |
| | sponse element) |
| μ-opioid receptor | CRE |
| 5-HT receptor | CRE, SRE, and NFAT |
| Glucocorticoid receptor | Glucocorticoid re- |
| | sponse element (GRE) |
| Adrenergic receptor | CRE |
| Androgen receptor | Androgen response |
| | element (ARE) |
| Thyroid hormone receptor | HRE |

Further exemplary receptor genes/proteins useful as heterologous receptors according to the methods and compositions of the disclosure include receptors such as those listed in the table below:

GPCR Receptors

| | HGNC |
|---|---|
| Family name | symbol |
| 5-Hydroxytryptamine receptors | HTR1A |
| 5-Hydroxytryptamine receptors | HTR1B |
| 5-Hydroxytryptamine receptors | HTR1D |
| 5-Hydroxytryptamine receptors | HTR1E |
| 5-Hydroxytryptamine receptors | HTR1F |
| 5-Hydroxytryptamine receptors | HTR2A |
| 5-Hydroxytryptamine receptors | HTR2B |
| 5-Hydroxytryptamine receptors | HTR2C |
| 5-Hydroxytryptamine receptors | HTR4 |
| 5-Hydroxytryptamine receptors | HTR5A |
| 5-Hydroxytryptamine receptors | HTR5BP |
| 5-Hydroxytryptamine receptors | HTR6 |
| 5-Hydroxytryptamine receptors | HTR7 |
| Acetylcholine receptors (muscarinic) | CHRM1 |
| Acetylcholine receptors (muscarinic) | CHRM2 |
| Acetylcholine receptors (muscarinic) | CHRM3 |
| Acetylcholine receptors (muscarinic) | CHRM4 |
| Acetylcholine receptors (muscarinic) | CHRM5 |
| Adenosine receptors | ADORA1 |
| Adenosine receptors | ADORA2A |
| Adenosine receptors | ADORA2B |
| Adenosine receptors | ADORA3 |
| Adhesion Class GPCRs | ADGRA1 |
| Adhesion Class GPCRs | ADGRA2 |
| Adhesion Class GPCRs | ADGRA3 |
| Adhesion Class GPCRs | ADGRB1 |
| Adhesion Class GPCRs | ADGRB2 |
| Adhesion Class GPCRs | ADGRB3 |
| Adhesion Class GPCRs | CELSR1 |
| Adhesion Class GPCRs | CELSR2 |
| Adhesion Class GPCRs | CELSR3 |
| Adhesion Class GPCRs | ADGRD1 |
| Adhesion Class GPCRs | ADGRD2 |
| Adhesion Class GPCRs | ADGRE1 |
| Adhesion Class GPCRs | ADGRE2 |
| Adhesion Class GPCRs | ADGRE3 |
| Adhesion Class GPCRs | ADGRE4P |
| Adhesion Class GPCRs | ADGRE5 |
| Adhesion Class GPCRs | ADGRF1 |
| Adhesion Class GPCRs | ADGRF2 |
| Adhesion Class GPCRs | ADGRF3 |
| Adhesion Class GPCRs | ADGRF4 |
| Adhesion Class GPCRs | ADGRF5 |
| Adhesion Class GPCRs | ADGRG1 |
| Adhesion Class GPCRs | ADGRG2 |
| Adhesion Class GPCRs | ADGRG3 |
| Adhesion Class GPCRs | ADGRG4 |
| Adhesion Class GPCRs | ADGRG5 |
| Adhesion Class GPCRs | ADGRG6 |
| Adhesion Class GPCRs | ADGRG7 |
| Adhesion Class GPCRs | ADGRL1 |
| Adhesion Class GPCRs | ADGRL2 |
| Adhesion Class GPCRs | ADGRL3 |
| Adhesion Class GPCRs | ADGRL4 |
| Adhesion Class GPCRs | ADGRV1 |
| Adrenoceptors | ADRA1A |
| Adrenoceptors | ADRA1B |
| Adrenoceptors | ADRA1D |
| Adrenoceptors | ADRA2A |
| Adrenoceptors | ADRA2B |
| Adrenoceptors | ADRA2C |
| Adrenoceptors | ADRB1 |
| Adrenoceptors | ADRB2 |
| Adrenoceptors | ADRB3 |
| Angiotensin receptors | AGTR1 |
| Angiotensin receptors | AGTR2 |
| Apelin receptor | APLNR |
| Bile acid receptor | GPBAR1 |
| Bombesin receptors | NMBR |
| Bombesin receptors | GRPR |
| Bombesin receptors | BRS3 |
| Bradykinin receptors | BDKRB1 |
| Bradykinin receptors | BDKRB2 |
| Calcitonin receptors | CALCR |
| Calcitonin receptors | |
| Calcitonin receptors | |
| Calcitonin receptors | |
| Calcitonin receptors | CALCRL |
| Calcitonin receptors | |
| Calcitonin receptors | |
| Calcitonin receptors | |
| Calcium-sensing receptor | CASR |
| Cannabinoid receptors | CNR1 |
| Cannabinoid receptors | CNR2 |
| Chemerin receptor | CMKLR1 |
| Chemokine receptors | CCR1 |
| Chemokine receptors | CCR2 |
| Chemokine receptors | CCR3 |
| Chemokine receptors | CCR4 |
| Chemokine receptors | CCR5 |
| Chemokine receptors | CCR6 |
| Chemokine receptors | CCR7 |
| Chemokine receptors | CCR8 |
| Chemokine receptors | CCR9 |
| Chemokine receptors | CCR10 |
| Chemokine receptors | CXCR1 |
| Chemokine receptors | CXCR2 |
| Chemokine receptors | CXCR3 |
| Chemokine receptors | CXCR4 |
| Chemokine receptors | CXCR5 |
| Chemokine receptors | CXCR6 |
| Chemokine receptors | CX3CR1 |
| Chemokine receptors | XCR1 |
| Chemokine receptors | ACKR1 |
| Chemokine receptors | ACKR2 |
| Chemokine receptors | ACKR3 |
| Chemokine receptors | ACKR4 |
| Chemokine receptors | CCRL2 |
| Cholecystokinin receptors | CCKAR |
| Cholecystokinin receptors | CCKBR |
| Class A Orphans | GPR1 |
| Class A Orphans | BRS3 |
| Class A Orphans | GPR3 |
| Class A Orphans | GPR4 |
| Class A Orphans | GPR42 |
| Class A Orphans | GPR6 |
| Class A Orphans | GPR12 |
| Class A Orphans | GPR15 |
| Class A Orphans | GPR17 |
| Class A Orphans | GPR18 |
| Class A Orphans | GPR19 |
| Class A Orphans | GPR20 |
| Class A Orphans | GPR21 |
| Class A Orphans | GPR22 |
| Class A Orphans | GPR25 |
| Class A Orphans | GPR26 |
| Class A Orphans | GPR27 |
| Class A Orphans | GPR31 |
| Class A Orphans | GPR32 |
| Class A Orphans | GPR33 |
| Class A Orphans | GPR34 |
| Class A Orphans | GPR35 |
| Class A Orphans | GPR37 |
| Class A Orphans | GPR37L1 |
| Class A Orphans | GPR39 |
| Class A Orphans | GPR45 |
| Class A Orphans | GPR50 |
| Class A Orphans | GPR52 |
| Class A Orphans | GPR55 |
| Class A Orphans | GPR61 |
| Class A Orphans | GPR62 |
| Class A Orphans | GPR63 |
| Class A Orphans | GPR65 |
| Class A Orphans | GPR68 |
| Class A Orphans | GPR75 |
| Class A Orphans | GPR78 |
| Class A Orphans | GPR79 |
| Class A Orphans | GPR82 |
| Class A Orphans | GPR83 |
| Class A Orphans | GPR84 |
| Class A Orphans | GPR85 |
| Class A Orphans | GPR87 |
| Class A Orphans | GPR88 |
| Class A Orphans | GPR101 |
| Class A Orphans | GPR119 |
| Class A Orphans | GPR132 |
| Class A Orphans | GPR135 |
| Class A Orphans | GPR139 |
| Class A Orphans | GPR141 |
| Class A Orphans | GPR142 |
| Class A Orphans | GPR146 |
| Class A Orphans | GPR148 |
| Class A Orphans | GPR149 |
| Class A Orphans | GPR150 |
| Class A Orphans | GPR151 |
| Class A Orphans | GPR152 |
| Class A Orphans | GPR153 |
| Class A Orphans | GPR160 |
| Class A Orphans | GPR161 |
| Class A Orphans | GPR162 |
| Class A Orphans | GPR171 |
| Class A Orphans | GPR173 |
| Class A Orphans | GPR174 |
| Class A Orphans | GPR176 |
| Class A Orphans | GPR182 |
| Class A Orphans | GPR183 |
| Class A Orphans | LGR4 |
| Class A Orphans | LGR5 |
| Class A Orphans | LGR6 |
| Class A Orphans | MAS1 |
| Class A Orphans | MAS1L |
| Class A Orphans | MRGPRD |
| Class A Orphans | MRGPRE |
| Class A Orphans | MRGPRF |
| Class A Orphans | MRGPRG |
| Class A Orphans | MRGPRX1 |
| Class A Orphans | MRGPRX2 |
| Class A Orphans | MRGPRX3 |
| Class A Orphans | MRGPRX4 |
| Class A Orphans | OPN3 |
| Class A Orphans | OPN4 |
| Class A Orphans | OPN5 |
| Class A Orphans | P2RY8 |
| Class A Orphans | P2RY10 |
| Class A Orphans | TAAR2 |
| Class A Orphans | TAAR3P |
| Class A Orphans | TAAR4P |
| Class A Orphans | TAAR5 |
| Class A Orphans | TAAR6 |
| Class A Orphans | TAAR8 |
| Class A Orphans | TAAR9 |
| Class C Orphans | GPR156 |
| Class C Orphans | GPR158 |
| Class C Orphans | GPR179 |
| Class C Orphans | GPRC5A |
| Class C Orphans | GPRC5B |
| Class C Orphans | GPRC5C |
| Class C Orphans | GPRC5D |
| Class C Orphans | GPRC6A |
| Class Frizzled GPCRs | FZD1 |
| Class Frizzled GPCRs | FZD2 |
| Class Frizzled GPCRs | FZD3 |
| Class Frizzled GPCRs | FZD4 |
| Class Frizzled GPCRs | FZD5 |
| Class Frizzled GPCRs | FZD6 |
| Class Frizzled GPCRs | FZD7 |
| Class Frizzled GPCRs | FZD8 |
| Class Frizzled GPCRs | FZD9 |
| Class Frizzled GPCRs | FZD10 |
| Class Frizzled GPCRs | SMO |
| Complement peptide receptors | C3AR1 |
| Complement peptide receptors | C5AR1 |
| Complement peptide receptors | C5AR2 |
| Corticotropin-releasing factor receptors | CRHR1 |
| Corticotropin-releasing factor receptors | CRHR2 |
| Dopamine receptors | DRD1 |
| Dopamine receptors | DRD2 |
| Dopamine receptors | DRD3 |
| Dopamine receptors | DRD4 |
| Dopamine receptors | DRD5 |
| Endothelin receptors | EDNRA |
| Endothelin receptors | EDNRB |
| Formylpeptide receptors | FPR1 |
| Formylpeptide receptors | FPR2 |
| Formylpeptide receptors | FPR3 |
| Free fatty acid receptors | FFAR1 |
| Free fatty acid receptors | FFAR2 |
| Free fatty acid receptors | FFAR3 |
| Free fatty acid receptors | FFAR4 |
| Free fatty acid receptors | GPR42 |
| GABA<sub>B</sub> receptors | |
| GABA<sub>B</sub> receptors | GABBR1 |
| GABA<sub>B</sub> receptors | GABBR2 |
| Galanin receptors | GALR1 |
| Galanin receptors | GALR2 |
| Galanin receptors | GALR3 |
| Ghrelin receptor | GHSR |
| Glucagon receptor family | GHRHR |
| Glucagon receptor family | GIPR |
| Glucagon receptor family | GLP1R |
| Glucagon receptor family | GLP2R |
| Glucagon receptor family | GCGR |
| Glucagon receptor family | SCTR |
| Glycoprotein hormone receptors | FSHR |
| Glycoprotein hormone receptors | LHCGR |
| Glycoprotein hormone receptors | TSHR |
| Gonadotrophin-releasing hormone receptors | GNRHR |
| Gonadotrophin-releasing hormone receptors | GNRHR2 |
| GPR18, GPR55 and GPR119 | GPR18 |
| GPR18, GPR55 and GPR119 | GPR55 |
| GPR18, GPR55 and GPR119 | GPR119 |
| G protein-coupled estrogen receptor | GPER1 |
| Histamine receptors | HRH1 |
| Histamine receptors | HRH2 |
| Histamine receptors | HRH3 |
| Histamine receptors | HRH4 |
| Hydroxycarboxylic acid receptors | HCAR1 |
| Hydroxycarboxylic acid receptors | HCAR2 |
| Hydroxycarboxylic acid receptors | HCAR3 |
| Kisspeptin receptor | KISS1R |
| Leukotriene receptors | LTB4R |
| Leukotriene receptors | LTB4R2 |
| Leukotriene receptors | CYSLTR1 |
| Leukotriene receptors | CYSLTR2 |
| Leukotriene receptors | OXER1 |
| Leukotriene receptors | FPR2 |
| Lysophospholipid (LPA) receptors | LPAR1 |
| Lysophospholipid (LPA) receptors | LPAR2 |
| Lysophospholipid (LPA) receptors | LPAR3 |
| Lysophospholipid (LPA) receptors | LPAR4 |
| Lysophospholipid (LPA) receptors | LPAR5 |
| Lysophospholipid (LPA) receptors | LPAR6 |
| Lysophospholipid (S1P) receptors | S1PR1 |
| Lysophospholipid (S1P) receptors | S1PR2 |
| Lysophospholipid (S1P) receptors | S1PR3 |
| Lysophospholipid (S1P) receptors | S1PR4 |
| Lysophospholipid (S1P) receptors | S1PR5 |
| Melanin-concentrating hormone receptors | MCHR1 |
| Melanin-concentrating hormone receptors | MCHR2 |
| Melanocortin receptors | MC1R |
| Melanocortin receptors | MC2R |
| Melanocortin receptors | MC3R |
| Melanocortin receptors | MC4R |
| Melanocortin receptors | MC5R |
| Melatonin receptors | MTNR1A |
| Melatonin receptors | MTNR1B |
| Metabotropic glutamate receptors | GRM1 |
| Metabotropic glutamate receptors | GRM2 |
| Metabotropic glutamate receptors | GRM3 |
| Metabotropic glutamate receptors | GRM4 |
| Metabotropic glutamate receptors | GRM5 |
| Metabotropic glutamate receptors | GRM6 |
| Metabotropic glutamate receptors | GRM7 |
| Metabotropic glutamate receptors | GRM8 |
| Motilin receptor | MLNR |
| Neuromedin U receptors | NMUR1 |
| Neuromedin U receptors | NMUR2 |
| Neuropeptide FF/neuropeptide AF receptors | NPFFR1 |
| Neuropeptide FF/neuropeptide AF receptors | NPFFR2 |
| Neuropeptide S receptor | NPSR1 |
| Neuropeptide W/neuropeptide B receptors | NPBWR1 |
| Neuropeptide W/neuropeptide B receptors | NPBWR2 |
| Neuropeptide Y receptors | NPY1R |
| Neuropeptide Y receptors | NPY2R |
| Neuropeptide Y receptors | NPY4R |
| Neuropeptide Y receptors | NPY5R |
| Neuropeptide Y receptors | NPY6R |
| Neurotensin receptors | NTSR1 |
| Neurotensin receptors | NTSR2 |
| Opioid receptors | OPRD1 |
| Opioid receptors | OPRK1 |
| Opioid receptors | OPRM1 |
| Opioid receptors | OPRL1 |
| Orexin receptors | HCRTR1 |
| Orexin receptors | HCRTR2 |
| Other 7TM proteins | GPR107 |
| Other 7TM proteins | GPR137 |
| Other 7TM proteins | OR51E1 |
| Other 7TM proteins | TPRA1 |
| Other 7TM proteins | GPR143 |
| Other 7TM proteins | GPR157 |
| Oxoglutarate receptor | OXGR1 |
| P2Y receptors | P2RY1 |
| P2Y receptors | P2RY2 |
| P2Y receptors | P2RY4 |
| P2Y receptors | P2RY6 |
| P2Y receptors | P2RY11 |
| P2Y receptors | P2RY12 |
| P2Y receptors | P2RY13 |
| P2Y receptors | P2RY14 |
| Parathyroid hormone receptors | PTH1R |
| Parathyroid hormone receptors | PTH2R |
| Platelet-activating factor receptor | PTAFR |
| Prokineticin receptors | PROKR1 |
| Prokineticin receptors | PROKR2 |
| Prolactin-releasing peptide receptor | PRLHR |
| Prostanoid receptors | PTGDR |
| Prostanoid receptors | PTGDR2 |
| Prostanoid receptors | PTGER1 |
| Prostanoid receptors | PTGER2 |
| Prostanoid receptors | PTGER3 |
| Prostanoid receptors | PTGER4 |
| Prostanoid receptors | PTGFR |
| Prostanoid receptors | PTGIR |
| Prostanoid receptors | TBXA2R |
| Proteinase-activated receptors | F2R |
| Proteinase-activated receptors | F2RL1 |
| Proteinase-activated receptors | F2RL2 |
| Proteinase-activated receptors | F2RL3 |
| QRFP receptor | QRFPR |
| Relaxin family peptide receptors | RXFP1 |
| Relaxin family peptide receptors | RXFP2 |
| Relaxin family peptide receptors | RXFP3 |
| Relaxin family peptide receptors | RXFP4 |
| Somatostatin receptors | SSTR1 |
| Somatostatin receptors | SSTR2 |
| Somatostatin receptors | SSTR3 |
| Somatostatin receptors | SSTR4 |
| Somatostatin receptors | SSTR5 |
| Succinate receptor | SUCNR1 |
| Tachykinin receptors | TACR1 |
| Tachykinin receptors | TACR2 |
| Tachykinin receptors | TACR3 |
| Taste 1 receptors | TAS1R1 |
| Taste 1 receptors | TAS1R2 |
| Taste 1 receptors | TAS1R3 |
| Taste 2 receptors | TAS2R1 |
| Taste 2 receptors | TAS2R3 |
| Taste 2 receptors | TAS2R4 |
| Taste 2 receptors | TAS2R5 |
| Taste 2 receptors | TAS2R7 |
| Taste 2 receptors | TAS2R8 |
| Taste 2 receptors | TAS2R9 |
| Taste 2 receptors | TAS2R10 |
| Taste 2 receptors | TAS2R13 |
| Taste 2 receptors | TAS2R14 |
| Taste 2 receptors | TAS2R16 |
| Taste 2 receptors | TAS2R19 |
| Taste 2 receptors | TAS2R20 |
| Taste 2 receptors | TAS2R30 |
| Taste 2 receptors | TAS2R31 |
| Taste 2 receptors | TAS2R38 |
| Taste 2 receptors | TAS2R39 |
| Taste 2 receptors | TAS2R40 |
| Taste 2 receptors | TAS2R41 |
| Taste 2 receptors | TAS2R42 |
| Taste 2 receptors | TAS2R43 |
| Taste 2 receptors | TAS2R45 |
| Taste 2 receptors | TAS2R46 |
| Taste 2 receptors | TAS2R50 |
| Taste 2 receptors | TAS2R60 |
| Thyrotropin-releasing hormone receptors | TRHR |
| Thyrotropin-releasing hormone receptors | |
| Trace amine receptor | TAAR1 |
| Urotensin receptor | UTS2R |
| Vasopressin and oxytocin receptors | AVPR1A |
| Vasopressin and oxytocin receptors | AVPR1B |
| Vasopressin and oxytocin receptors | AVPR2 |
| Vasopressin and oxytocin receptors | OXTR |
| VIP and PACAP receptors | ADCYAP1R1 |
| VIP and PACAP receptors | VIPR1 |
| VIP and PACAP receptors | VIPR2 |

Nuclear Hormone Receptors:

| Family name | HGNCsymbol |
|---|---|
| 0B. DAX-like receptors | NR0B1 |
| 0B. DAX-like receptors | NR0B2 |
| 1A. Thyroid hormone receptors | THRA |
| 1A. Thyroid hormone receptors | THRB |
| 1B. Retinoic acid receptors | RARA |
| 1B. Retinoic acid receptors | RARB |
| 1B. Retinoic acid receptors | RARG |
| 1C. Peroxisome proliferator-activated receptors | PPARA |
| 1C. Peroxisome proliferator-activated receptors | PPARD |
| 1C. Peroxisome proliferator-activated receptors | PPARG |
| 1D. Rev-Erb receptors | NR1D1 |
| 1D. Rev-Erb receptors | NR1D2 |
| 1F. Retinoic acid-related orphans | RORA |
| 1F. Retinoic acid-related orphans | RORB |
| 1F. Retinoic acid-related orphans | RORC |
| 1H. Liver X receptor-like receptors | NR1H4 |
| 1H. Liver X receptor-like receptors | NR1H5P |
| 1H. Liver X receptor-like receptors | NR1H3 |
| 1H. Liver X receptor-like receptors | NR1H2 |
| 1I. Vitamin D receptor-like receptors | VDR |
| 1I. Vitamin D receptor-like receptors | NR1I2 |
| 1I. Vitamin D receptor-like receptors | NR1I3 |
| 2A. Hepatocyte nuclear factor-4 receptors | HNF4A |
| 2A. Hepatocyte nuclear factor-4 receptors | HNF4G |
| 2B. Retinoid X receptors | RXRA |
| 2B. Retinoid X receptors | RXRB |
| 2B. Retinoid X receptors | RXRG |
| 2C. Testicular receptors | NR2C1 |
| 2C. Testicular receptors | NR2C2 |
| 2E. Tailless-like receptors | NR2E1 |
| 2E. Tailless-like receptors | NR2E3 |
| 2F. COUP-TF-like receptors | NR2F1 |
| 2F. COUP-TF-like receptors | NR2F2 |
| 2F. COUP-TF-like receptors | NR2F6 |
| 3A. Estrogen receptors | ESR1 |
| 3A. Estrogen receptors | ESR2 |
| 3B. Estrogen-related receptors | ESRRA |
| 3B. Estrogen-related receptors | ESRRB |
| 3B. Estrogen-related receptors | ESRRG |
| 3C. 3-Ketosteroid receptors | AR |
| 3C. 3-Ketosteroid receptors | NR3C1 |
| 3C. 3-Ketosteroid receptors | NR3C2 |
| 3C. 3-Ketosteroid receptors | PGR |
| 4A. Nerve growth factor IB-like receptors | NR4A1 |
| 4A. Nerve growth factor IB-like receptors | NR4A2 |
| 4A. Nerve growth factor IB-like receptors | NR4A3 |
| 5A. Fushi tarazu F1 -like receptors | NR5A1 |
| 5A. Fushi tarazu F1 -like receptors | NR5A2 |
| 6A. Germ cell nuclear factor receptors | NR6A1 |

Catalytic Receptors

| | HGNC |
|---|---|
| Family name | symbol |
| GDNF receptor family | GFRA1 |
| GDNF receptor family | GFRA2 |
| GDNF receptor family | GFRA3 |
| GDNF receptor family | GFRA4 |
| IL-10 receptor family | IL22RA2 |
| IL-10 receptor family | IL10RA |
| IL-10 receptor family | IL10RB |
| IL-10 receptor family | IL20RA |
| IL-10 receptor family | IL20RB |
| IL-10 receptor family | IL22RA1 |
| IL-10 receptor family | IFNLR1 |
| IL-12 receptor family | IL12RB1 |
| IL-12 receptor family | IL12RB2 |
| IL-12 receptor family | IL23R |
| IL-17 receptor family | IL17RA |
| IL-17 receptor family | IL17RB |
| IL-17 receptor family | IL17RC |
| IL-17 receptor family | IL17RD |
| IL-17 receptor family | IL17RE |
| IL-2 receptor family | IL13RA2 |
| IL-2 receptor family | IL2RA |
| IL-2 receptor family | IL2RB |
| IL-2 receptor family | IL2RG |
| IL-2 receptor family | IL4R |
| IL-2 receptor family | IL7R |
| IL-2 receptor family | IL9R |
| IL-2 receptor family | IL13RA1 |
| IL-2 receptor family | IL15RA |
| IL-2 receptor family | IL21R |
| IL-2 receptor family | CRLF2 |
| IL-3 receptor family | IL3RA |
| IL-3 receptor family | IL5RA |
| IL-3 receptor family | CSF2RA |
| IL-3 receptor family | CSF2RB |
| IL-6 receptor family | IL6R |
| IL-6 receptor family | IL6ST |
| IL-6 receptor family | IL11RA |
| IL-6 receptor family | IL27RA |
| IL-6 receptor family | IL31RA |
| IL-6 receptor family | CNTFR |
| IL-6 receptor family | LEPR |
| IL-6 receptor family | LIFR |
| IL-6 receptor family | OSMR |
| Immunoglobulin-like family of IL-1 receptors | IL1R1 |
| Immunoglobulin-like family of IL-1 receptors | IL1R2 |
| Immunoglobulin-like family of IL-1 receptors | IL1RL1 |
| Immunoglobulin-like family of IL-1 receptors | IL1RL2 |
| Immunoglobulin-like family of IL-1 receptors | IL18R1 |
| Integrins | ITGA1 |
| Integrins | ITGA2 |
| Integrins | ITGA2B |
| Integrins | ITGA3 |
| Integrins | ITGA4 |
| Integrins | ITGA5 |
| Integrins | ITGA6 |
| Integrins | ITGA7 |
| Integrins | ITGA8 |
| Integrins | ITGA9 |
| Integrins | ITGA10 |
| Integrins | ITGA11 |
| Integrins | ITGAD |
| Integrins | ITGAE |
| Integrins | ITGAL |
| Integrins | ITGAM |
| Integrins | ITGAV |
| Integrins | ITGAX |
| Integrins | ITGB1 |
| Integrins | ITGB2 |
| Integrins | ITGB3 |
| Integrins | ITGB4 |
| Integrins | ITGB5 |
| Integrins | ITGB6 |
| Integrins | ITGB7 |
| Integrins | ITGB8 |
| Interferon receptor family | IFNAR1 |
| Interferon receptor family | IFNAR2 |
| Interferon receptor family | IFNGR1 |
| Interferon receptor family | IFNGR2 |
| Natriuretic peptide receptor family | NPR1 |
| Natriuretic peptide receptor family | NPR2 |
| Natriuretic peptide receptor family | GUCY2C |
| Natriuretic peptide receptor family | NPR3 |
| NOD-like receptor family | NOD1 |
| NOD-like receptor family | NOD2 |
| NOD-like receptor family | NLRC3 |
| NOD-like receptor family | NLRC4 |
| NOD-like receptor family | NLRC5 |
| NOD-like receptor family | NLRX1 |
| NOD-like receptor family | CIITA |
| NOD-like receptor family | NLRP1 |
| NOD-like receptor family | NLRP2 |
| NOD-like receptor family | NLRP3 |
| NOD-like receptor family | NLRP4 |
| NOD-like receptor family | NLRP5 |
| NOD-like receptor family | NLRP6 |
| NOD-like receptor family | NLRP7 |
| NOD-like receptor family | NLRP8 |
| NOD-like receptor family | NLRP9 |
| NOD-like receptor family | NLRP10 |
| NOD-like receptor family | NLRP11 |
| NOD-like receptor family | NLRP12 |
| NOD-like receptor family | NLRP13 |
| NOD-like receptor family | NLRP14 |
| Prolactin receptor family | EPOR |
| Prolactin receptor family | CSF3R |
| Prolactin receptor family | GHR |
| Prolactin receptor family | PRLR |
| Prolactin receptor family | MPL |
| Receptor Guanylyl Cyclase (RGC) family | NPR1 |
| Receptor Guanylyl Cyclase (RGC) family | NPR2 |
| Receptor Guanylyl Cyclase (RGC) family | GUCY2C |
| Receptor Guanylyl Cyclase (RGC) family | GUCY2D |
| Receptor Guanylyl Cyclase (RGC) family | GUCY2F |
| Receptor Guanylyl Cyclase (RGC) family | GUCY2GP |
| Receptor tyrosine phosphatase (RTP) family | PTPRA |
| Receptor tyrosine phosphatase (RTP) family | PTPRB |
| Receptor tyrosine phosphatase (RTP) family | PTPRC |
| Receptor tyrosine phosphatase (RTP) family | PTPRD |
| Receptor tyrosine phosphatase (RTP) family | PTPRE |
| Receptor tyrosine phosphatase (RTP) family | PTPRF |
| Receptor tyrosine phosphatase (RTP) family | PTPRG |
| Receptor tyrosine phosphatase (RTP) family | PTPRH |
| Receptor tyrosine phosphatase (RTP) family | PTPRJ |
| Receptor tyrosine phosphatase (RTP) family | PTPRK |
| Receptor tyrosine phosphatase (RTP) family | PTPRM |
| Receptor tyrosine phosphatase (RTP) family | PTPRN |
| Receptor tyrosine phosphatase (RTP) family | PTPRN2 |
| Receptor tyrosine phosphatase (RTP) family | PTPRO |
| Receptor tyrosine phosphatase (RTP) family | PTPRQ |
| Receptor tyrosine phosphatase (RTP) family | PTPRR |
| Receptor tyrosine phosphatase (RTP) family | PTPRS |
| Receptor tyrosine phosphatase (RTP) family | PTPRT |
| Receptor tyrosine phosphatase (RTP) family | PTPRU |
| Receptor tyrosine phosphatase (RTP) family | PTPRZ1 |
| RIG-I-like receptor family | DDX58 |
| RIG-I-like receptor family | IFIH1 |
| RIG-I-like receptor family | DHX58 |
| Toll-like receptor family | TLR1 |
| Toll-like receptor family | TLR2 |
| Toll-like receptor family | TLR3 |
| Toll-like receptor family | TLR4 |
| Toll-like receptor family | TLR5 |
| Toll-like receptor family | TLR6 |
| Toll-like receptor family | TLR7 |
| Toll-like receptor family | TLR8 |
| Toll-like receptor family | TLR9 |
| Toll-like receptor family | TLR10 |
| Tumour necrosis factor (TNF) receptor family | TNFRSF1A |
| Tumour necrosis factor (TNF) receptor family | TNFRSF1B |
| Tumour necrosis factor (TNF) receptor family | LTBR |
| Tumour necrosis factor (TNF) receptor family | TNFRSF4 |
| Tumour necrosis factor (TNF) receptor family | CD40 |
| Tumour necrosis factor (TNF) receptor family | FAS |
| Tumour necrosis factor (TNF) receptor family | TNFRSF6B |
| Tumour necrosis factor (TNF) receptor family | CD27 |
| Tumour necrosis factor (TNF) receptor family | TNFRSF8 |
| Tumour necrosis factor (TNF) receptor family | TNFRSF9 |
| Tumour necrosis factor (TNF) receptor family | TNFRSF10A |
| Tumour necrosis factor (TNF) receptor family | TNFRSF10B |
| Tumour necrosis factor (TNF) receptor family | TNFRSF10C |
| Tumour necrosis factor (TNF) receptor family | TNFRSF10D |
| Tumour necrosis factor (TNF) receptor family | TNFRSF11A |
| Tumour necrosis factor (TNF) receptor family | TNFRSF11B |
| Tumour necrosis factor (TNF) receptor family | TNFRSF25 |
| Tumour necrosis factor (TNF) receptor family | TNFRSF12A |
| Tumour necrosis factor (TNF) receptor family | TNFRSF13B |
| Tumour necrosis factor (TNF) receptor family | TNFRSF13C |
| Tumour necrosis factor (TNF) receptor family | TNFRSF14 |
| Tumour necrosis factor (TNF) receptor family | NGFR |
| Tumour necrosis factor (TNF) receptor family | TNFRSF17 |
| Tumour necrosis factor (TNF) receptor family | TNFRSF18 |
| Tumour necrosis factor (TNF) receptor family | TNFRSF19 |
| Tumour necrosis factor (TNF) receptor family | RELT |
| Tumour necrosis factor (TNF) receptor family | TNFRSF21 |
| Tumour necrosis factor (TNF) receptor family | EDA2R |
| Tumour necrosis factor (TNF) receptor family | EDAR |
| Type III receptor serine/threonine kinases | TGFBR3 |
| Type III RTKs: PDGFR, CSFR, Kit, FLT3 | PDGFRA |
| receptor family | |
| Type III RTKs: PDGFR, CSFR, Kit, FLT3 | PDGFRB |
| receptor family | |
| Type III RTKs: PDGFR, CSFR, Kit, FLT3 | KIT |
| receptor family | |
| Type III RTKs: PDGFR, CSFR, Kit, FLT3 | CSF1R |
| receptor family | |
| Type III RTKs: PDGFR, CSFR, Kit, FLT3 | FLT3 |
| receptor family | |
| Type II receptor serine/threonine kinases | ACVR2A |
| Type II receptor serine/threonine kinases | ACVR2B |
| Type II receptor serine/threonine kinases | AMHR2 |
| Type II receptor serine/threonine kinases | BMPR2 |
| Type II receptor serine/threonine kinases | TGFBR2 |
| Type II RTKs: Insulin receptor family | INSR |
| Type II RTKs: Insulin receptor family | IGF1R |
| Type II RTKs: Insulin receptor family | INSRR |
| Type I receptor serine/threonine kinases | ACVRL1 |
| Type I receptor serine/threonine kinases | ACVR1 |
| Type I receptor serine/threonine kinases | BMPR1A |
| Type I receptor serine/threonine kinases | ACVR1B |
| Type I receptor serine/threonine kinases | TGFBR1 |
| Type I receptor serine/threonine kinases | BMPR1B |
| Type I receptor serine/threonine kinases | ACVR1C |
| Type I RTKs: ErbB (epidermal growth factor) | EGFR |
| receptor family | |
| Type I RTKs: ErbB (epidermal growth factor) | ERBB2 |
| receptor family | |
| Type I RTKs: ErbB (epidermal growth factor) | ERBB3 |
| receptor family | |
| Type I RTKs: ErbB (epidermal growth factor) | ERBB4 |
| receptor family | |
| Type IV RTKs: VEGF (vascular endothelial | FLT1 |
| growth factor) receptor family | |
| Type IV RTKs: VEGF (vascular endothelial | KDR |
| growth factor) receptor family | |
| Type IV RTKs: VEGF (vascular endothelial | FLT4 |
| growth factor) receptor family | |
| Type IX RTKs: MuSK | MUSK |
| Type VIII RTKs: ROR family | ROR1 |
| Type VIII RTKs: ROR family | ROR2 |
| Type VII RTKs: Neurotrophin receptor/Trk family | NTRK1 |
| Type VII RTKs: Neurotrophin receptor/Trk family | NTRK2 |
| Type VII RTKs: Neurotrophin receptor/Trk family | NTRK3 |
| Type VI RTKs: PTK7/CCK4 | PTK7 |
| Type V RTKs: FGF (fibroblast growth factor) | FGFR1 |
| receptor family | |
| Type V RTKs: FGF (fibroblast growth factor) | FGFR2 |
| receptor family | |
| Type V RTKs: FGF (fibroblast growth factor) | FGFR3 |
| receptor family | |
| Type V RTKs: FGF (fibroblast growth factor) | FGFR4 |
| receptor family | |
| Type XIII RTKs: Ephrin receptor family | EPHA1 |
| Type XIII RTKs: Ephrin receptor family | EPHA2 |
| Type XIII RTKs: Ephrin receptor family | EPHA3 |
| Type XIII RTKs: Ephrin receptor family | EPHA4 |
| Type XIII RTKs: Ephrin receptor family | EPHA5 |
| Type XIII RTKs: Ephrin receptor family | EPHA6 |
| Type XIII RTKs: Ephrin receptor family | EPHA7 |
| Type XIII RTKs: Ephrin receptor family | EPHA8 |
| Type XIII RTKs: Ephrin receptor family | EPHA10 |
| Type XIII RTKs: Ephrin receptor family | EPHB1 |
| Type XIII RTKs: Ephrin receptor family | EPHB2 |
| Type XIII RTKs: Ephrin receptor family | EPHB3 |
| Type XIII RTKs: Ephrin receptor family | EPHB4 |
| Type XIII RTKs: Ephrin receptor family | EPHB6 |
| Type XII RTKs: TIE family of angiopoietin | TIE1 |
| receptors | |
| Type XII RTKs: TIE family of angiopoietin | TEK |
| receptors | |
| Type XI RTKs: TAM (TYRO3-, AXL- and MER-TK) | AXL |
| receptor family | |
| Type XI RTKs: TAM (TYRO3-, AXL- and MER-TK) | TYRO3 |
| receptor family | |
| Type XI RTKs: TAM (TYRO3-, AXL- and MER-TK) | MERTK |
| receptor family | |
| Type XIV RTKs: RET | RET |
| Type XIX RTKs: Leukocyte tyrosine kinase (LTK) | LTK |
| receptor family | |
| Type XIX RTKs: Leukocyte tyrosine kinase (LTK) | ALK |
| receptor family | |
| Type X RTKs: HGF (hepatocyte growth factor) | MET |
| receptor family | |
| Type X RTKs: HGF (hepatocyte growth factor) | MST1R |
| receptor family | |
| Type XVIII RTKs: LMR family | AATK |
| Type XVIII RTKs: LMR family | LMTK2 |
| Type XVIII RTKs: LMR family | LMTK3 |
| Type XVII RTKs: ROS receptors | ROS1 |
| Type XVI RTKs: DDR (collagen receptor) family | DDR1 |
| Type XVI RTKs: DDR (collagen receptor) family | DDR2 |
| Type XV RTKs: RYK | RYK |
| Type XX RTKs: STYK1 | STYK1 |

The ligands may be a known ligand for the receptor or a test compound. For example, in the case of olfactory receptors, the ligand may be an odorant. Exemplary odorants include Geranyl acetate, Methyl formate, Methyl acetate, Methyl propionate, Methyl propanoate, Methyl butyrate, Methyl butanoate, Ethyl acetate, Ethyl butyrate, Ethyl butanoate, Isoamyl acetate, Pentyl butyrate, Pentyl butanoate, Pentyl pentanoate, Octyl acetate, Benzyl acetate, and Methyl anthranilate.

In some embodiments, the ligand comprises a small molecule, a polypeptide, or a nucleic acid ligand. Methods of the disclosure relate to screening procedures that detect ligand engagement with a receptor. Accordingly, the ligand may be a test compound or a drug. The methods of the disclosure can be utilized to determine ligand and receptor engagement for the purposes of determining ligand/drug efficacy and/or off-target effects. A polypeptide ligand may be a peptide, which is fewer than 100 amino acids in length.

Chemical agents are “small molecule” compounds that are typically organic, non-peptide molecules, having a molecular weight less than 10,000 Da. In some embodiments, they are less than 5,000 Da, less than 1,000 Da, or less than 500 Da (and any range derivable therein). This class of modulators includes chemically synthesized molecules, for example, compounds from combinatorial chemical libraries. Synthetic compounds may be rationally designed or identified from screening methods described herein. Methods for generating and obtaining small molecules are well known in the art (Schreiber, Science 2000; 151:1964-1969; Radmann et al., Science 2000; 151:1947-1948, which are hereby incorporated by reference).

### II. REPORTER SYSTEMS

A. Nucleic Acid Reporter

The reporter comprises a barcode region, which comprises an index region that can identify the activated receptor. The index region can be a polynucleotide of at least, at most, or exactly 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200 or more (or any range derivable therein) nucleotides in length. The barcode may comprise one or more universal PCR regions, adaptors, linkers, or a combination thereof.

The index region of the barcode is a polynucleotide sequence that can be used to identify the heterologous receptor that is activated and/or expressed in the same cell as the barcode because it is unique to a particular heterologous receptor in the context of the screen being utilized. In embodiments relating to a populations of cells, determining the identity of the barcode is done by determining the nucleotide sequence of the index region in order to identify which receptor(s) has been activated in a population of cells. As discussed herein, methods may involve sequencing one or more index regions or having such index regions sequenced.

Nucleic acid constructs are generated by any means known in the art, including through the use of polymerases and solid state nucleic acid synthesis (e.g., on a column, multiwall plate, or microarray). The invention provides for the inclusion of barcodes, to facilitate the determination of the activity of specific nucleic acid regulatory elements (i.e. receptor-responsive elements), which may be an indication of an activated receptor. These barcodes are included in the nucleic acid constructs and expression vectors containing the nucleic acid regulatory elements. Each index region of the barcode is unique to the corresponding heterologous receptor gene (i.e., although a particular nucleic acid regulatory element may have more than one barcodes or index regions (e.g., 2, 3, 4, 5, 10, or more), each barcode is indicative of the activation of a single receptor). These barcodes are oriented in the expression vector such that they are transcribed in the same mRNA transcript as the associated open reading frame. The barcodes may be oriented in the mRNA transcript 5′ to the open reading frame, 3′ to the open reading frame, immediately 5′ to the terminal poly-A tail, or somewhere in-between. In some embodiments, the barcodes are in the 3′ untranslated region.

The unique portions of the barcodes may be continuous along the length of the barcode sequence or the barcode may include stretches of nucleic acid sequence that is not unique to any one barcode. In one application, the unique portions of the barcodes (i.e. index region(s)) may be separated by a stretch of nucleic acids that is removed by the cellular machinery during transcription into mRNA (e.g., an intron).

The inducible reporter includes a regulatory element, such as a promoter, and a barcode. In some embodiments, the regulatory element further includes an open reading frame. The open reading frame may encode for a selectable or screenable marker, as described herein. The nucleic acid regulatory element may be 5′, 3′, or within the open reading frame. The barcode may be located anywhere within the region to be transcribed into mRNA (e.g., upstream of the open reading frame, downstream of the open reading frame, or within the open reading frame). Importantly, the barcode is located 5′ to the transcription termination site.

The barcodes and/or index regions are quantified or determined by methods known in the art, including quantitative sequencing (e.g., using an Illumina® sequencer) or quantitative hybridization techniques (e.g., microarray hybridization technology or using a Luminex® bead system). Sequencing methods are further described herein.

B. Sequencing Methods to Detect Barcodes

1. Massively Parallel Signature Sequencing (MPSS).

The first of the next-generation sequencing technologies, massively parallel signature sequencing (or MPSS), was developed in the 1990s at Lynx Therapeutics. MPSS was a bead-based method that used a complex approach of adapter ligation followed by adapter decoding, reading the sequence in increments of four nucleotides. This method made it susceptible to sequence-specific bias or loss of specific sequences. Because the technology was so complex, MPSS was only performed ‘in-house’ by Lynx Therapeutics and no DNA sequencing machines were sold to independent laboratories. Lynx Therapeutics merged with Solexa (later acquired by Illumina) in 2004, leading to the development of sequencing-by-synthesis, a simpler approach acquired from Manteia Predictive Medicine, which rendered MPSS obsolete. However, the essential properties of the MPSS output were typical of later “next-generation” data types, including hundreds of thousands of short DNA sequences. In the case of MPSS, these were typically used for sequencing cDNA for measurements of gene expression levels. Indeed, the powerful Illumina HiSeq2000, HiSeq2500 and MiSeq systems are based on MPSS.

2. Polony Sequencing.

The Polony sequencing method, developed in the laboratory of George M. Church at Harvard, was among the first next-generation sequencing systems and was used to sequence a full genome in 2005. It combined an in vitro paired-tag library with emulsion PCR, an automated microscope, and ligation-based sequencing chemistry to sequence an E. coli genome at an accuracy of >99.9999% and a cost approximately 1/9 that of Sanger sequencing. The technology was licensed to Agencourt Biosciences, subsequently spun out into Agencourt Personal Genomics, and eventually incorporated into the Applied Biosystems SOLiD platform, which is now owned by Life Technologies.

3. 454 Pyrosequencing.

A parallelized version of pyrosequencing was developed by 454 Life Sciences, which has since been acquired by Roche Diagnostics. The method amplifies DNA inside water droplets in an oil solution (emulsion PCR), with each droplet containing a single DNA template attached to a single primer-coated bead that then forms a clonal colony. The sequencing machine contains many picoliter-volume wells each containing a single bead and sequencing enzymes. Pyrosequencing uses luciferase to generate light for detection of the individual nucleotides added to the nascent DNA, and the combined data are used to generate sequence read-outs. This technology provides intermediate read length and price per base compared to Sanger sequencing on one end and Solexa and SOLiD on the other.

4. Illumina (Solexa) Sequencing.

Solexa, now part of Illumina, developed a sequencing method based on reversible dye-terminators technology, and engineered polymerases, that it developed internally. The terminated chemistry was developed internally at Solexa and the concept of the Solexa system was invented by Balasubramanian and Klennerman from Cambridge University's chemistry department. In 2004, Solexa acquired the company Manteia Predictive Medicine in order to gain a massively parallel sequencing technology based on “DNA Clusters”, which involves the clonal amplification of DNA on a surface. The cluster technology was co-acquired with Lynx Therapeutics of California. Solexa Ltd. later merged with Lynx to form Solexa Inc.

In this method, DNA molecules and primers are first attached on a slide and amplified with polymerase so that local clonal DNA colonies, later coined “DNA clusters”, are formed. To determine the sequence, four types of reversible terminator bases (RT-bases) are added and non-incorporated nucleotides are washed away. A camera takes images of the fluorescently labeled nucleotides, then the dye, along with the terminal 3′ blocker, is chemically removed from the DNA, allowing for the next cycle to begin. Unlike pyrosequencing, the DNA chains are extended one nucleotide at a time and image acquisition can be performed at a delayed moment, allowing for very large arrays of DNA colonies to be captured by sequential images taken from a single camera.

Decoupling the enzymatic reaction and the image capture allows for optimal throughput and theoretically unlimited sequencing capacity. With an optimal configuration, the ultimately reachable instrument throughput is thus dictated solely by the analog-to-digital conversion rate of the camera, multiplied by the number of cameras and divided by the number of pixels per DNA colony required for visualizing them optimally (approximately 10 pixels/colony). In 2012, with cameras operating at more than 10 MHz A/D conversion rates and available optics, fluidics and enzymatics, throughput can be multiples of 1 million nucleotides/second, corresponding roughly to one human genome equivalent at 1× coverage per hour per instrument, and one human genome re-sequenced (at approx. 30×) per day per instrument (equipped with a single camera).

5. Solid Sequencing.

Applied Biosystems' (now a Life Technologies brand) SOLiD technology employs sequencing by ligation. Here, a pool of all possible oligonucleotides of a fixed length are labeled according to the sequenced position. Oligonucleotides are annealed and ligated; the preferential ligation by DNA ligase for matching sequences results in a signal informative of the nucleotide at that position. Before sequencing, the DNA is amplified by emulsion PCR. The resulting beads, each containing single copies of the same DNA molecule, are deposited on a glass slide. The result is sequences of quantities and lengths comparable to Illumina sequencing. This sequencing by ligation method has been reported to have some issue sequencing palindromic sequences.

6. Ion Torrent Semiconductor Sequencing.

Ion Torrent Systems Inc. (now owned by Life Technologies) developed a system based on using standard sequencing chemistry, but with a novel, semiconductor based detection system. This method of sequencing is based on the detection of hydrogen ions that are released during the polymerization of DNA, as opposed to the optical methods used in other sequencing systems. A microwell containing a template DNA strand to be sequenced is flooded with a single type of nucleotide. If the introduced nucleotide is complementary to the leading template nucleotide it is incorporated into the growing complementary strand. This causes the release of a hydrogen ion that triggers a hypersensitive ion sensor, which indicates that a reaction has occurred. If homopolymer repeats are present in the template sequence multiple nucleotides will be incorporated in a single cycle. This leads to a corresponding number of released hydrogens and a proportionally higher electronic signal.

7. DNA Nanoball Sequencing.

DNA nanoball sequencing is a type of high throughput sequencing technology used to determine the entire genomic sequence of an organism. The company Complete Genomics uses this technology to sequence samples submitted by independent researchers. The method uses rolling circle replication to amplify small fragments of genomic DNA into DNA nanoballs. Unchained sequencing by ligation is then used to determine the nucleotide sequence. This method of DNA sequencing allows large numbers of DNA nanoballs to be sequenced per run and at low reagent costs compared to other next generation sequencing platforms. However, only short sequences of DNA are determined from each DNA nanoball which makes mapping the short reads to a reference genome difficult. This technology has been used for multiple genome sequencing projects and is scheduled to be used for more.

8. Heliscope Single Molecule Sequencing.

Heliscope sequencing is a method of single-molecule sequencing developed by Helicos Biosciences. It uses DNA fragments with added poly-A tail adapters which are attached to the flow cell surface. The next steps involve extension-based sequencing with cyclic washes of the flow cell with fluorescently labeled nucleotides (one nucleotide type at a time, as with the Sanger method). The reads are performed by the Heliscope sequencer. The reads are short, up to 55 bases per run, but recent improvements allow for more accurate reads of stretches of one type of nucleotides. This sequencing method and equipment were used to sequence the genome of the M13 bacteriophage.

9. Single Molecule Real Time (SMRT) Sequencing.

SMRT sequencing is based on the sequencing by synthesis approach. The DNA is synthesized in zero-mode wave-guides (ZMWs)—small well-like containers with the capturing tools located at the bottom of the well. The sequencing is performed with use of unmodified polymerase (attached to the ZMW bottom) and fluorescently labelled nucleotides flowing freely in the solution. The wells are constructed in a way that only the fluorescence occurring by the bottom of the well is detected. The fluorescent label is detached from the nucleotide at its incorporation into the DNA strand, leaving an unmodified DNA strand. According to Pacific Biosciences, the SMRT technology developer, this methodology allows detection of nucleotide modifications (such as cytosine methylation). This happens through the observation of polymerase kinetics. This approach allows reads of 20,000 nucleotides or more, with average read lengths of 5 kilobases.

C. Measurement of Gene or Barcode Expression

Embodiments of the disclosure relate to determining the expression of a reporter barcode and/or reporter gene or open reading frame. The expression of the reporter can be determined by measuring the levels of RNA transcripts of the barcode or index region and any other polynucleotides expressed from the reporter construct. Suitable methods for this purpose include, but are not limited to, RT-PCR, Northern Blot, in situ hybridization, Southern Blot, slot-blotting, nuclease protection assay and oligonucleotide arrays.

In certain aspects, RNA isolated from cells can be amplified to cDNA or cRNA before detection and/or quantitation. The isolated RNA can be either total RNA or mRNA. The RNA amplification can be specific or non-specific. In some embodiments, the amplification is specific in that it specifically amplifies reporter barcodes or regions thereof, such as an index region. In some embodiments, the amplification and/or reverse transcriptase step excludes random priming. Suitable amplification methods include, but are not limited to, reverse transcriptase PCR, isothermal amplification, ligase chain reaction, and Qbeta replicase. The amplified nucleic acid products can be detected and/or quantitated through hybridization to labeled probes. In some embodiments, detection may involve fluorescence resonance energy transfer (FRET) or some other kind of quantum dots.

Amplification primers or hybridization probes for a reporter barcode can be prepared from the sequence of the expressed portion of the reporter. The term “primer” or “probe” as used herein, is meant to encompass any nucleic acid that is capable of priming the synthesis of a nascent nucleic acid in a template-dependent process. Typically, primers are oligonucleotides from ten to twenty and/or thirty base pairs in length, but longer sequences can be employed. Primers may be provided in double-stranded and/or single-stranded form, although the single-stranded form is preferred.

The use of a probe or primer of between 13 and 100 nucleotides, particularly between 17 and 100 nucleotides in length, or in some aspects up to 1-2 kilobases or more in length, allows the formation of a duplex molecule that is both stable and selective. Molecules having complementary sequences over contiguous stretches greater than 20 bases in length may be used to increase stability and/or selectivity of the hybrid molecules obtained. One may design nucleic acid molecules for hybridization having one or more complementary sequences of 20 to 30 nucleotides, or even longer where desired. Such fragments may be readily prepared, for example, by directly synthesizing the fragment by chemical means or by introducing selected sequences into recombinant vectors for recombinant production.

In one embodiment, each probe/primer comprises at least 15 nucleotides. For instance, each probe can comprise at least or at most 20, 25, 50, 75, 100, 125, 150, 175, 200, 225, 250, 275, 300, 325, 350, 400 or more nucleotides (or any range derivable therein). They may have these lengths and have a sequence that is identical or complementary to a gene described herein. Particularly, each probe/primer has relatively high sequence complexity and does not have any ambiguous residue (undetermined “n” residues). The probes/primers can hybridize to the target gene, including its RNA transcripts, under stringent or highly stringent conditions. In some embodiments, because each of the biomarkers has more than one human sequence, it is contemplated that probes and primers may be designed for use with each of these sequences. For example, inosine is a nucleotide frequently used in probes or primers to hybridize to more than one sequence. It is contemplated that probes or primers may have inosine or other design implementations that accommodate recognition of more than one human sequence for a particular biomarker.

For applications requiring high selectivity, one will typically desire to employ relatively high stringency conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe or primers and the template or target strand and would be particularly suitable for isolating specific genes or for detecting specific mRNA transcripts. It is generally appreciated that conditions can be rendered more stringent by the addition of increasing amounts of formamide.

In one embodiment, quantitative RT-PCR (such as TaqMan, ABI) is used for detecting and comparing the levels of RNA transcripts in samples. Quantitative RT-PCR involves reverse transcription (RT) of RNA to cDNA followed by relative quantitative PCR (RT-PCR). The concentration of the target DNA in the linear portion of the PCR process is proportional to the starting concentration of the target before the PCR was begun. By determining the concentration of the PCR products of the target DNA in PCR reactions that have completed the same number of cycles and are in their linear ranges, it is possible to determine the relative concentrations of the specific target sequence in the original DNA mixture. If the DNA mixtures are cDNAs synthesized from RNAs isolated from different tissues or cells, the relative abundances of the specific mRNA from which the target sequence was derived may be determined for the respective tissues or cells. This direct proportionality between the concentration of the PCR products and the relative mRNA abundances is true in the linear range portion of the PCR reaction. The final concentration of the target DNA in the plateau portion of the curve is determined by the availability of reagents in the reaction mix and is independent of the original concentration of target DNA. Therefore, the sampling and quantifying of the amplified PCR products may be carried out when the PCR reactions are in the linear portion of their curves. In addition, relative concentrations of the amplifiable cDNAs may be normalized to some independent standard, which may be based on either internally existing RNA species or externally introduced RNA species. The abundance of a particular mRNA species may also be determined relative to the average abundance of all mRNA species in the sample.

In one embodiment, the PCR amplification utilizes one or more internal PCR standards. The internal standard may be an abundant housekeeping gene in the cell or it can specifically be GAPDH, GUSB and β-2 microglobulin. These standards may be used to normalize expression levels so that the expression levels of different gene products can be compared directly. A person of ordinary skill in the art would know how to use an internal standard to normalize expression levels.

A problem inherent in some samples is that they are of variable quantity and/or quality. This problem can be overcome if the RT-PCR is performed as a relative quantitative RT-PCR with an internal standard in which the internal standard is an amplifiable cDNA fragment that is similar or larger than the target cDNA fragment and in which the abundance of the mRNA encoding the internal standard is roughly 5-100 fold higher than the mRNA encoding the target. This assay measures relative abundance, not absolute abundance of the respective mRNA species.

In another embodiment, the relative quantitative RT-PCR uses an external standard protocol. Under this protocol, the PCR products are sampled in the linear portion of their amplification curves. The number of PCR cycles that are optimal for sampling can be empirically determined for each target cDNA fragment. In addition, the reverse transcriptase products of each RNA population isolated from the various samples can be normalized for equal concentrations of amplifiable cDNAs.

A nucleic acid array can comprise at least 1, 2, 3, 4, 5, 6, 7, 8, 9, 10, 15, 20, 25, 30, 35, 40, 45, 50, 60, 70, 80, 90, 100, 150, 200, 250 or more different polynucleotide probes, which may hybridize to different and/or the same biomarkers. Multiple probes for the same gene can be used on a single nucleic acid array. Probes for other disease genes can also be included in the nucleic acid array. The probe density on the array can be in any range. In some embodiments, the density may be 50, 100, 200, 300, 400, 500 or more probes/cm².

Specifically contemplated are chip-based nucleic acid technologies such as those described by Hacia et al. (1996) and Shoemaker et al. (1996). Briefly, these techniques involve quantitative methods for analyzing large numbers of genes rapidly and accurately. By tagging genes with oligonucleotides or using fixed probe arrays, one can employ chip technology to segregate target molecules as high density arrays and screen these molecules on the basis of hybridization (see also, Pease et al., 1994; and Fodor et al, 1991). It is contemplated that this technology may be used in conjunction with evaluating the expression level of one or more cancer biomarkers with respect to diagnostic, prognostic, and treatment methods.

Certain embodiments may involve the use of arrays or data generated from an array. Data may be readily available. Moreover, an array may be prepared in order to generate data that may then be used in correlation studies.

An array generally refers to ordered macroarrays or microarrays of nucleic acid molecules (probes) that are fully or nearly complementary or identical to a plurality of mRNA molecules or cDNA molecules and that are positioned on a support material in a spatially separated organization. Macroarrays are typically sheets of nitrocellulose or nylon upon which probes have been spotted. Microarrays position the nucleic acid probes more densely such that up to 10,000 nucleic acid molecules can be fit into a region typically 1 to 4 square centimeters. Microarrays can be fabricated by spotting nucleic acid molecules, e.g., genes, oligonucleotides, etc., onto substrates or fabricating oligonucleotide sequences in situ on a substrate. Spotted or fabricated nucleic acid molecules can be applied in a high density matrix pattern of up to about 30 non-identical nucleic acid molecules per square centimeter or higher, e.g. up to about 100 or even 1000 per square centimeter. Microarrays typically use coated glass as the solid support, in contrast to the nitrocellulose-based material of filter arrays. By having an ordered array of complementing nucleic acid samples, the position of each sample can be tracked and linked to the original sample. A variety of different array devices in which a plurality of distinct nucleic acid probes are stably associated with the surface of a solid support are known to those of skill in the art. Useful substrates for arrays include nylon, glass and silicon. Such arrays may vary in a number of different ways, including average probe length, sequence or types of probes, nature of bond between the probe and the array surface, e.g. covalent or non-covalent, and the like. The labeling and screening methods and the arrays are not limited in its utility with respect to any parameter except that the probes detect expression levels; consequently, methods and compositions may be used with a variety of different types of genes.

Representative methods and apparatus for preparing a microarray have been described, for example, in U.S. Pat. Nos. 5,143,854; 5,202,231; 5,242,974; 5,288,644; 5,324,633; 5,384,261; 5,405,783; 5,412,087; 5,424,186; 5,429,807; 5,432,049; 5,436,327; 5,445,934; 5,468,613; 5,470,710; 5,472,672; 5,492,806; 5,525,464; 5,503,980; 5,510,270; 5,525,464; 5,527,681; 5,529,756; 5,532,128; 5,545,531; 5,547,839; 5,554,501; 5,556,752; 5,561,071; 5,571,639; 5,580,726; 5,580,732; 5,593,839; 5,599,695; 5,599,672; 5,610,287; 5,624,711; 5,631,134; 5,639,603; 5,654,413; 5,658,734; 5,661,028; 5,665,547; 5,667,972; 5,695,940; 5,700,637; 5,744,305; 5,800,992; 5,807,522; 5,830,645; 5,837,196; 5,871,928; 5,847,219; 5,876,932; 5,919,626; 6,004,755; 6,087,102; 6,368,799; 6,383,749; 6,617,112; 6,638,717; 6,720,138, as well as WO 93/17126; WO 95/11995; WO 95/21265; WO 95/21944; WO 95/35505; WO 96/31622; WO 97/10365; WO 97/27317; WO 99/35505; WO 09923256; WO 09936760; WO0138580; WO 0168255; WO 03020898; WO 03040410; WO 03053586; WO 03087297; WO 03091426; WO03100012; WO 04020085; WO 04027093; EP 373 203; EP 785 280; EP 799 897 and UK 8 803 000; the disclosures of which are all herein incorporated by reference.

It is contemplated that the arrays can be high density arrays, such that they contain 100 or more different probes. It is contemplated that they may contain 1000, 16,000, 65,000, 250,000 or 1,000,000 or more different probes. The oligonucleotide probes range from 5 to 50, 5 to 45, 10 to 40, or 15 to 40 nucleotides in length in some embodiments. In certain embodiments, the oligonucleotide probes are 20 to 25 nucleotides in length.

The location and sequence of each different probe sequence in the array are generally known. Moreover, the large number of different probes can occupy a relatively small area providing a high density array having a probe density of generally greater than about 60, 100, 600, 1000, 5,000, 10,000, 40,000, 100,000, or 400,000 different oligonucleotide probes per cm2. The surface area of the array can be about or less than about 1, 1.6, 2, 3, 4, 5, 6, 7, 8, 9, or 10 cm2.

Moreover, a person of ordinary skill in the art could readily analyze data generated using an array. Such protocols include information found in WO 9743450; WO 03023058; WO 03022421; WO 03029485; WO 03067217; WO 03066906; WO 03076928; WO 03093810; WO 03100448A1, all of which are specifically incorporated by reference.

In one embodiment, nuclease protection assays are used to quantify RNAs derived from the cancer samples. There are many different versions of nuclease protection assays known to those practiced in the art. The common characteristic that these nuclease protection assays have is that they involve hybridization of an antisense nucleic acid with the RNA to be quantified. The resulting hybrid double-stranded molecule is then digested with a nuclease that digests single-stranded nucleic acids more efficiently than double-stranded molecules. The amount of antisense nucleic acid that survives digestion is a measure of the amount of the target RNA species to be quantified. An example of a nuclease protection assay that is commercially available is the RNase protection assay manufactured by Ambion, Inc. (Austin, Tex.).

### III. RECEPTOR GENE AND INDUCIBLE REPORTER ADDITIONS

In certain embodiments, the receptor gene and or inducible reporter system comprises one or more polynucleotide sequences encoding for one or more auxiliary polypeptides. Exemplary auxiliary polypeptides include transcription factors, protein or peptide tag, and screenable or selectable genes.

A. Selection and Screening Genes

In certain embodiments of the disclosure, the inducible reporter and/or the receptor gene may comprise or further comprise a selection or screening gene. Furthermore, the cells, vectors, and viral particles of the disclosure may further comprise a selection or screening gene. In some embodiments, the selection or screening gene is fused to the receptor gene such that one fusion protein comprising a receptor protein fused to a selection or screening protein is present in the cell. Such genes would confer an identifiable change to the cell permitting easy identification of cells that have activation of the heterologous receptor gene. Generally, a selectable (i.e. selection gene) gene is one that confers a property that allows for selection. A positive selectable gene is one in which the presence of the gene or gene product allows for its selection, while a negative selectable gene is one in which its presence of the gene or gene product prevents its selection. An example of a positive selectable gene is an antibiotic resistance gene.

Usually the inclusion of a drug selection gene aids in the cloning and identification of cells that have an activated receptor gene through, for example, successful ligand engagement. The selection gene may be a gene that confers resistance to neomycin, puromycin, hygromycin, DHFR, GPT, zeocin, G418, phleomycin, blasticidin, and histidinol, for example. In addition to genes conferring a phenotype that allows for the discrimination of receptor activation based on the implementation of conditions, other types of genes, including screenable genes such as GFP, whose gene product provides for colorimetric analysis, are also contemplated. Alternatively, screenable enzymes such as herpes simplex virus thymidine kinase (tk) or chloramphenicol acetyltransferase (CAT) may be utilized. One of skill in the art would also know how to employ screenable genes and their protein products, possibly in conjunction with FACS analysis. Further examples of selectable and screenable genes are well known to one of skill in the art. In certain embodiments, the gene produces a fluorescent protein, an enzymatically active protein, a luminescent protein, a photoactivatable protein, a photoconvertible protein, or a colorimetric protein. Fluorescent markers include, for example, GFP and variants such as YFP, RFP etc., and other fluorescent proteins such as DsRed, mPlum, mCherry, YPet, Emerald, CyPet, T-Sapphire, Luciferase, and Venus. Photoactivatable markers include, for example, KFP, PA-mRFP, and Dronpa. Photoconvertible markers include, for example, mEosFP, KikGR, and PS-CFP2. Luminescent proteins include, for example, Neptune, FP595, and phialidin.

B. Protein or Peptide Tags

Exemplary protein/peptide tags include AviTag, a peptide allowing biotinylation by the enzyme BirA and so the protein can be isolated by streptavidin (GLNDIFEAQKIEWHE, SEQ ID NO:4), Calmodulin-tag, a peptide bound by the protein calmodulin (KRRWKKNFIAVSAANRFKKISSSGAL, SEQ ID NO:5), polyglutamate tag, a peptide binding efficiently to anion-exchange resin such as Mono-Q (EEEEEE, SEQ ID NO:6), E-tag, a peptide recognized by an antibody (GAPVPYPDPLEPR, SEQ ID NO:7), FLAG-tag, a peptide recognized by an antibody (DYKDDDDK, SEQ ID NO:8), HA-tag, a peptide from hemagglutinin recognized by an antibody (YPYDVPDYA, SEQ ID NO:9), His-tag, 5-10 histidines bound by a nickel or cobalt chelate (HHHHHH, SEQ ID NO:10), Myc-tag, a peptide derived from c-myc recognized by an antibody (EQKLISEEDL, SEQ ID NO:11), NE-tag, a novel 18-amino-acid synthetic peptide (TKENPRSNQEESYDDNES, SEQ ID NO:12) recognized by a monoclonal IgG1 antibody, which is useful in a wide spectrum of applications including Western blotting, ELISA, flow cytometry, immunocytochemistry, immunoprecipitation, and affinity purification of recombinant proteins, S-tag, a peptide derived from Ribonuclease A (KETAAAKFERQHMDS, SEQ ID NO:13), SBP-tag, a peptide which binds to streptavidin (MDEKTTGWRGGHVVEGLAGELEQLRARLEHHPQGQREP, SEQ ID NO:14), Softag 1, for mammalian expression (SLAELLNAGLGGS, SEQ ID NO:15), Softag 3, for prokaryotic expression (TQDPSRVG, SEQ ID NO:16), Strep-tag, a peptide which binds to streptavidin or the modified streptavidin called streptactin (Strep-tag II: WSHPQFEK, SEQ ID NO:17), TC tag, a tetracysteine tag that is recognized by FlAsH and ReAsH biarsenical compounds (CCPGCC, SEQ ID NO:18), V5 tag, a peptide recognized by an antibody (GKPIPNPLLGLDST, SEQ ID NO:19), VSV-tag, a peptide recognized by an antibody (YTDIEMNRLGK, SEQ ID NO:20), Xpress tag (DLYDDDDK, SEQ ID NO:21), Covalent peptide tags, Isopeptag, a peptide which binds covalently to pilin-C protein (TDKDMTITFTNKKDAE, SEQ ID NO:22), SpyTag, a peptide which binds covalently to SpyCatcher protein (AHIVMVDAYKPTK, SEQ ID NO:23), SnoopTag, a peptide which binds covalently to SnoopCatcher protein (KLGDIEFIKVNK, SEQ ID NO:24), BCCP (Biotin Carboxyl Carrier Protein), a protein domain biotinylated by BirA enabling recognition by streptavidin, Glutathione-S-transferase-tag, a protein which binds to immobilized glutathione, Green fluorescent protein-tag, a protein which is spontaneously fluorescent and can be bound by nanobodies, HaloTag, a mutated bacterial haloalkane dehalogenase that covalently attaches to a reactive haloalkane substrate, this allows attachment to a wide variety of substrates. Maltose binding protein-tag, a protein which binds to amylose agarose, Nus-tag, Thioredoxin-tag, Fc-tag, derived from immunoglobulin Fc domain, allow dimerization and solubilization. Can be used for purification on Protein-A Sepharose, Designed Intrinsically Disordered tags containing disorder promoting amino acids (P, E, S, T, A, Q, G, . . . ), and Ty-tag

C. Transcription Factors

In some embodiments, the receptor gene encodes for a fusion protein comprising the receptor protein and an auxiliary polypeptide. In some embodiments, the auxiliary polypeptide is a transcription factor. In related embodiments, the inducible reporter comprises a receptor-responsive element, wherein the receptor-responsive element is bound by the transcription factor. Such transcription factors and responsive elements are known in the art and include, for example, reverse tetracycline-controlled transactivator (rtTA), which can induce transcription through a tetracycline-responsive element (TRE), Gal4p, which induces transcription through the GAL1 promoter, and estrogen receptor, which, when bound to a ligand, induces expression through the estrogen response element. Accordingly, related embodiments include administering a ligand to activate transcription of an auxiliary polypeptide transcription factor.

### IV. VECTORS AND NUCLEIC ACIDS

The current disclosure includes embodiments of nucleic acids comprising one or more of a heterologous receptor gene and an inducible reporter. The terms “oligonucleotide,” “polynucleotide,” and “nucleic acid are used interchangeable and include linear oligomers of natural or modified monomers or linkages, including deoxyribonucleosides, ribonucleosides, α-anomeric forms thereof, peptide nucleic acids (PNAs), and the like, capable of specifically binding to a target polynucleotide by way of a regular pattern of monomer-to-monomer interactions, such as Watson-Crick type of base pairing, base stacking, Hoogsteen or reverse Hoogsteen types of base pairing, or the like. Usually monomers are linked by phosphodiester bonds or analogs thereof to form oligonucleotides ranging in size from a few monomeric units, e.g. 3-4, to several tens of monomeric units. Whenever an oligonucleotide is represented by a sequence of letters, such as “ATGCCTG,” it will be understood that the nucleotides are in 5′→3′ order from left to right and that “A” denotes deoxyadenosine, “C” denotes deoxycytidine, “G” denotes deoxyguanosine, and “T” denotes thymidine, unless otherwise noted. Analogs of phosphodiester linkages include phosphorothioate, phosphorodithioate, phosphoranilidate, phosphoramidate, and the like. It is clear to those skilled in the art when oligonucleotides having natural or non-natural nucleotides may be employed, e.g. where processing by enzymes is called for, usually oligonucleotides consisting of natural nucleotides are required.

The nucleic acid may be an “unmodified oligonucleotide” or “unmodified nucleic acid,” which refers generally to an oligomer or polymer of ribonucleic acid (RNA) or deoxyribonucleic acid (DNA). In some embodiments a nucleic acid molecule is an unmodified oligonucleotide. This term includes oligonucleotides composed of naturally occurring nucleobases, sugars and covalent internucleoside linkages. The term “oligonucleotide analog” refers to oligonucleotides that have one or more non-naturally occurring portions which function in a similar manner to oligonucleotides. Such non-naturally occurring oligonucleotides are often selected over naturally occurring forms because of desirable properties such as, for example, enhanced cellular uptake, enhanced affinity for other oligonucleotides or nucleic acid targets and increased stability in the presence of nucleases. The term “oligonucleotide” can be used to refer to unmodified oligonucleotides or oligonucleotide analogs.

Specific examples of nucleic acid molecules include nucleic acid molecules containing modified, i.e., non-naturally occurring internucleoside linkages. Such non-naturally internucleoside linkages are often selected over naturally occurring forms because of desirable properties such as, for example, enhanced cellular uptake, enhanced affinity for other oligonucleotides or nucleic acid targets and increased stability in the presence of nucleases. In a specific embodiment, the modification comprises a methyl group.

Nucleic acid molecules can have one or more modified internucleoside linkages. As defined in this specification, oligonucleotides having modified internucleoside linkages include internucleoside linkages that retain a phosphorus atom and internucleoside linkages that do not have a phosphorus atom. For the purposes of this specification, and as sometimes referenced in the art, modified oligonucleotides that do not have a phosphorus atom in their internucleoside backbone can also be considered to be oligonucleosides.

Modifications to nucleic acid molecules can include modifications wherein one or both terminal nucleotides is modified.

One suitable phosphorus-containing modified internucleoside linkage is the phosphorothioate internucleoside linkage. A number of other modified oligonucleotide backbones (internucleoside linkages) are known in the art and may be useful in the context of this embodiment.

Representative U.S. patents that teach the preparation of phosphorus-containing internucleoside linkages include, but are not limited to, U.S. Pat. Nos. 3,687,808; 4,469,863; 4,476,301; 5,023,243, 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 5,625,050, 5,489,677, and 5,602,240 each of which is herein incorporated by reference.

Modified oligonucleoside backbones (internucleoside linkages) that do not include a phosphorus atom therein have internucleoside linkages that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having amide backbones; and others, including those having mixed N, O, S and CH2 component parts.

Representative U.S. patents that teach the preparation of the above non-phosphorous-containing oligonucleosides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, each of which is herein incorporated by reference.

Oligomeric compounds can also include oligonucleotide mimetics. The term mimetic as it is applied to oligonucleotides is intended to include oligomeric compounds wherein only the furanose ring or both the furanose ring and the internucleotide linkage are replaced with novel groups, replacement of only the furanose ring with for example a morpholino ring, is also referred to in the art as being a sugar surrogate. The heterocyclic base moiety or a modified heterocyclic base moiety is maintained for hybridization with an appropriate target nucleic acid.

Oligonucleotide mimetics can include oligomeric compounds such as peptide nucleic acids (PNA) and cyclohexenyl nucleic acids (known as CeNA, see Wang et al., J. Am. Chem. Soc., 2000, 122, 8595-8602). Representative U.S. patents that teach the preparation of oligonucleotide mimetics include, but are not limited to, U.S. Pat. Nos. 5,539,082; 5,714,331; and 5,719,262, each of which is herein incorporated by reference. Another class of oligonucleotide mimetic is referred to as phosphonomonoester nucleic acid and incorporates a phosphorus group in the backbone. This class of olignucleotide mimetic is reported to have useful physical and biological and pharmacological properties in the areas of inhibiting gene expression (antisense oligonucleotides, ribozymes, sense oligonucleotides and triplex-forming oligonucleotides), as probes for the detection of nucleic acids and as auxiliaries for use in molecular biology. Another oligonucleotide mimetic has been reported wherein the furanosyl ring has been replaced by a cyclobutyl moiety.

Nucleic acid molecules can also contain one or more modified or substituted sugar moieties. The base moieties are maintained for hybridization with an appropriate nucleic acid target compound. Sugar modifications can impart nuclease stability, binding affinity or some other beneficial biological property to the oligomeric compounds.

Representative modified sugars include carbocyclic or acyclic sugars, sugars having substituent groups at one or more of their 2′, 3′ or 4′ positions, sugars having substituents in place of one or more hydrogen atoms of the sugar, and sugars having a linkage between any two other atoms in the sugar. A large number of sugar modifications are known in the art, sugars modified at the 2′ position and those which have a bridge between any 2 atoms of the sugar (such that the sugar is bicyclic) are particularly useful in this embodiment. Examples of sugar modifications useful in this embodiment include, but are not limited to compounds comprising a sugar substituent group selected from: OH; F; O-, S-, or N-alkyl; or O-alkyl-O-alkyl, wherein the alkyl, alkenyl and alkynyl may be substituted or unsubstituted C1 to C10 alkyl or C2 to C10 alkenyl and alkynyl. Particularly suitable are: 2-methoxyethoxy (also known as 2′-O-methoxyethyl, 2′-MOE, or 2′-OCH2CH2OCH3), 2′-O-methyl (2′-O—CH3), 2′-fluoro (2′-F), or bicyclic sugar modified nucleosides having a bridging group connecting the 4′ carbon atom to the 2′ carbon atom wherein example bridge groups include —CH2-O—, —(CH2)2-O—or —CH2-N(R3)-O wherein R3 is H or C1-C12 alkyl.

One modification that imparts increased nuclease resistance and a very high binding affinity to nucleotides is the 2′-MOE side chain (Baker et al., J. Biol. Chem., 1997, 272, 11944-12000). One of the immediate advantages of the 2′-MOE substitution is the improvement in binding affinity, which is greater than many similar 2′ modifications such as O-methyl, O-propyl, and O-aminopropyl. Oligonucleotides having the 2′-MOE substituent also have been shown to be antisense inhibitors of gene expression with promising features for in vivo use (Martin, P., Helv. Chim. Acta, 1995, 78, 486-504; Altmann et al., Chimia, 1996, 50, 168-176; Altmann et al., Biochem. Soc. Trans., 1996, 24, 630-637; and Altmann et al., Nucleosides Nucleotides, 1997, 16, 917-926).

2′-Sugar substituent groups may be in the arabino (up) position or ribo (down) position. One 2′-arabino modification is 2′-F. Similar modifications can also be made at other positions on the oligomeric compound, particularly the 3′ position of the sugar on the 3′ terminal nucleoside or in 2′-5′ linked oligonucleotides and the 5′ position of 5′ terminal nucleotide. Oligomeric compounds may also have sugar mimetics such as cyclobutyl moieties in place of the pentofuranosyl sugar. Representative U.S. patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920, each of which is herein incorporated by reference in its entirety.

Nucleic acid molecules can also contain one or more nucleobase (often referred to in the art simply as “base”) modifications or substitutions which are structurally distinguishable from, yet functionally interchangeable with, naturally occurring or synthetic unmodified nucleobases. Such nucleobase modifications can impart nuclease stability, binding affinity or some other beneficial biological property to the oligomeric compounds. As used herein, “unmodified” or “natural” nucleobases include the purine bases adenine (A) and guanine (G), and the pyrimidine bases thymine (T), cytosine (C) and uracil (U). Modified nucleobases also referred to herein as heterocyclic base moieties include other synthetic and natural nucleobases, many examples of which such as 5-methylcytosine (5-me-C), 5-hydroxymethyl cytosine, 7-deazaguanine and 7-deazaadenine among others.

Heterocyclic base moieties can also include those in which the purine or pyrimidine base is replaced with other heterocycles, for example 7-deaza-adenine, 7-deazaguanosine, 2-aminopyridine and 2-pyridone. Some nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Certain of these nucleobases are particularly useful for increasing the binding affinity of the oligomeric compounds. These include 5-substituted pyrimidines, 6-azapyrimidines and N-2, N-6 and O-6 substituted purines, including 2 aminopropyladenine, 5-propynyluracil and 5-propynylcytosine.

Additional modifications to nucleic acid molecules are disclosed in U.S. Patent Publication 2009/0221685, which is hereby incorporated by reference. Also disclosed herein are additional suitable conjugates to the nucleic acid molecules.

The heterologous receptor gene and inducible reporter may be encoded by a nucleic acid molecule, such as a vector. In some embodiments, they are encoded on the same nucleic acid molecule. In some embodiments, they are encoded on separate nucleic acid molecules. In certain embodiments, the nucleic acid molecule can be in the form of a nucleic acid vector. The term “vector” is used to refer to a carrier nucleic acid molecule into which a heterologous nucleic acid sequence can be inserted for introduction into a cell where it can be replicated and expressed and/or integrated into the host cell's genome. A nucleic acid sequence can be “heterologous,” which means that it is in a context foreign to the cell in which the vector is being introduced or to the nucleic acid in which is incorporated, which includes a sequence homologous to a sequence in the cell or nucleic acid but in a position within the host cell or nucleic acid where it is ordinarily not found. Vectors include DNAs, RNAs, plasmids, cosmids, viruses (bacteriophage, animal viruses, and plant viruses), and artificial chromosomes (e.g., YACs). One of skill in the art would be well equipped to construct a vector through standard recombinant techniques (for example Sambrook et al., 2001; Ausubel et al., 1996, both incorporated herein by reference). Vectors may be used in a host cell to produce an antibody.

The term “expression vector” refers to a vector containing a nucleic acid sequence coding for at least part of a gene product capable of being transcribed or stably integrate into a host cell's genome and subsequently be transcribed. In some cases, RNA molecules are then translated into a protein, polypeptide, or peptide. Expression vectors can contain a variety of “control sequences,” which refer to nucleic acid sequences necessary for the transcription and possibly translation of an operably linked coding sequence in a particular host organism. In addition to control sequences that govern transcription and translation, vectors and expression vectors may contain nucleic acid sequences that serve other functions as well and are described herein.

The vectors disclosed herein can be any nucleic acid vector known in the art. Exemplary vectors include plasmids, cosmids, bacterial artificial chromosomes (BACs) and viral vectors.

Any expression vector for animal cell can be used. Examples of suitable vectors include pAGE107 (Miyaji et al., 1990), pAGE103 (Mizukami and Itoh, 1987), pHSG274 (Brady et al., 1984), pKCR (O'Hare et al., 1981), pSG1 beta d2-4 (Miyaji et al., 1990) and the like.

Other examples of plasmids include replicating plasmids comprising an origin of replication, or integrative plasmids, such as for instance pUC, pcDNA, pBR, and the like.

Other examples of viral vectors include adenoviral, lentiviral, retroviral, herpes virus and AAV vectors. Such recombinant viruses may be produced by techniques known in the art, such as by transfecting packaging cells or by transient transfection with helper plasmids or viruses. Typical examples of virus packaging cells include PA317 cells, PsiCRIP cells, GPenv+ cells, 293 cells, etc. Detailed protocols for producing such replication-defective recombinant viruses may be found for instance in WO 95/14785, WO 96/22378, U.S. Pat. Nos. 5,882,877, 6,013,516, 4,861,719, 5,278,056 and WO 94/19478.

A “promoter” is a control sequence. The promoter is typically a region of a nucleic acid sequence at which initiation and rate of transcription are controlled. It may contain genetic elements at which regulatory proteins and molecules may bind such as RNA polymerase and other transcription factors. The phrases “operatively positioned,” “operatively linked,” “under control,” and “under transcriptional control” mean that a promoter is in a correct functional location and/or orientation in relation to a nucleic acid sequence to control transcriptional initiation and expression of that sequence. A promoter may or may not be used in conjunction with an “enhancer,” which refers to a cis-acting regulatory sequence involved in the transcriptional activation of a nucleic acid sequence.

Examples of promoters and enhancers used in the expression vector for animal cell include early promoter and enhancer of SV40 (Mizukami and Itoh, 1987), LTR promoter and enhancer of Moloney mouse leukemia virus (Kuwana et al., 1987), promoter (Mason et al., 1985) and enhancer (Gillies et al., 1983) of immunoglobulin H chain and the like.

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals.

Vectors can include a multiple cloning site (MCS), which is a nucleic acid region that contains multiple restriction enzyme sites, any of which can be used in conjunction with standard recombinant technology to digest the vector. (See Carbonelli et al., 1999, Levenson et al., 1998, and Cocea, 1997, incorporated herein by reference.)

Most transcribed eukaryotic RNA molecules will undergo RNA splicing to remove introns from the primary transcripts. Vectors containing genomic eukaryotic sequences may require donor and/or acceptor splicing sites to ensure proper processing of the transcript for protein expression. (See Chandler et al., 1997, incorporated herein by reference.)

The vectors or constructs will generally comprise at least one termination signal. A “termination signal” or “terminator” is comprised of the DNA sequences involved in specific termination of an RNA transcript by an RNA polymerase. Thus, in certain embodiments a termination signal that ends the production of an RNA transcript is contemplated. A terminator may be necessary in vivo to achieve desirable message levels. In eukaryotic systems, the terminator region may also comprise specific DNA sequences that permit site-specific cleavage of the new transcript so as to expose a polyadenylation site. This signals a specialized endogenous polymerase to add a stretch of about 200 A residues (polyA) to the 3′ end of the transcript. RNA molecules modified with this polyA tail appear to more stable and are translated more efficiently. Thus, in other embodiments involving eukaryotes, it is preferred that that terminator comprises a signal for the cleavage of the RNA, and it is more preferred that the terminator signal promotes polyadenylation of the message.

In expression, particularly eukaryotic expression, one will typically include a polyadenylation signal to effect proper polyadenylation of the transcript.

In order to propagate a vector in a host cell, it may contain one or more origins of replication sites (often termed “ori”), which is a specific nucleic acid sequence at which replication is initiated. Alternatively an autonomously replicating sequence (ARS) can be employed if the host cell is yeast.

Some vectors may employ control sequences that allow it to be replicated and/or expressed in both prokaryotic and eukaryotic cells. One of skill in the art would further understand the conditions under which to incubate all of the above described host cells to maintain them and to permit replication of a vector. Also understood and known are techniques and conditions that would allow large-scale production of vectors, as well as production of the nucleic acids encoded by vectors and their cognate polypeptides, proteins, or peptides.

A further aspect of the disclosure relates to a cell or cells comprising a receptor gene and inducible reporter, as described herein. In some embodiments, a prokaryotic or eukaryotic cell is genetically transformed or transfected with at least one nucleic acid molecule or vector according to the disclosure. In some embodiments, the cells are infected with a viral particle of the current disclosure.

The term “transformation” or “transfection” means the introduction of a “foreign” (i.e. extrinsic or extracellular) gene, DNA or RNA sequence to a host cell, so that the host cell will express the introduced gene or sequence to produce a desired substance, typically a protein or enzyme coded by the introduced gene or sequence. A host cell that receives and expresses introduced DNA or RNA has been “transformed” or “transfected.” The construction of expression vectors in accordance with the current disclosure, and the transformation or transfection of the host cells can be carried out using conventional molecular biology techniques.

Suitable methods for nucleic acid delivery for transformation/transfection of a cell, a tissue or an organism for use with the current invention are believed to include virtually any method by which a nucleic acid (e.g., DNA) can be introduced into a cell, a tissue or an organism, as described herein or as would be known to one of ordinary skill in the art (e.g., Stadtfeld and Hochedlinger, Nature Methods 6(5):329-330 (2009); Yusa et al., Nat. Methods 6:363-369 (2009); Woltjen et al., Nature 458, 766-770 (9 Apr. 2009)). Such methods include, but are not limited to, direct delivery of DNA such as by ex vivo transfection (Wilson et al., Science, 244:1344-1346, 1989, Nabel and Baltimore, Nature 326:711-713, 1987), optionally with Fugene6 (Roche) or Lipofectamine (Invitrogen), by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, J. Cell Biol., 101:1094-1099, 1985; U.S. Pat. No. 5,789,215, incorporated herein by reference); by electroporation (U.S. Pat. No. 5,384,253, incorporated herein by reference; Tur-Kaspa et al., Mol. Cell Biol., 6:716-718, 1986; Potter et al., Proc. Nat'l Acad. Sci. USA, 81:7161-7165, 1984); by calcium phosphate precipitation (Graham and Van Der Eb, Virology, 52:456-467, 1973; Chen and Okayama, Mol. Cell Biol., 7(8):2745-2752, 1987; Rippe et al., Mol. Cell Biol., 10:689-695, 1990); by using DEAE-dextran followed by polyethylene glycol (Gopal, Mol. Cell Biol., 5:1188-1190, 1985); by direct sonic loading (Fechheimer et al., Proc. Nat'l Acad. Sci. USA, 84:8463-8467, 1987); by liposome mediated transfection (Nicolau and Sene, Biochim. Biophys. Acta, 721:185-190, 1982; Fraley et al., Proc. Nat'l Acad. Sci. USA, 76:3348-3352, 1979; Nicolau et al., Methods Enzymol., 149:157-176, 1987; Wong et al., Gene, 10:87-94, 1980; Kaneda et al., Science, 243:375-378, 1989; Kato et al., J Biol. Chem., 266:3361-3364, 1991) and receptor-mediated transfection (Wu and Wu, Biochemistry, 27:887-892, 1988; Wu and Wu, J. Biol. Chem., 262:4429-4432, 1987); and any combination of such methods, each of which is incorporated herein by reference.

### V. CELLS

As used herein, the terms “cell,” “cell line,” and “cell culture” may be used interchangeably. All of these terms also include both freshly isolated cells and in vitro cultured or expanded cells. All of these terms also include their progeny, which is any and all subsequent generations. It is understood that all progeny may not be identical due to deliberate or inadvertent mutations. In the context of expressing a heterologous nucleic acid sequence, a “host cell” or simply a “cell” refers to a prokaryotic or eukaryotic cell, and it includes any transformable organism that is capable of replicating a vector or expressing a heterologous gene encoded by a vector or integrated nucleic acid. A host cell can, and has been, used as a recipient for vectors, viruses, and nucleic acids. A host cell may be “transfected” or “transformed,” which refers to a process by which exogenous nucleic acid, such as a recombinant protein-encoding sequence, is transferred or introduced into the host cell. A transformed cell includes the primary subject cell and its progeny.

In certain embodiments the nucleic acid transfer can be carried out on any prokaryotic or eukaryotic cell. In some aspects the cells of the disclosure are human cells. In other aspects the cells of the disclosure are an animal cell. In some aspects the cell or cells are cancer cells, tumor cells or immortalized cells. In further aspects, the cells represent a disease-model cell. In certain aspects the cells can be A549, B-cells, B16, BHK-21, C2C12, C6, CaCo-2, CAP/, CAP-T, CHO, CHO2, CHO-DG44, CHO-K1, COS-1, Cos-7, CV-1, Dendritic cells, DLD-1, Embryonic Stem (ES) Cell or derivative, H1299, HEK, 293, 293T, 293FT, Hep G2, Hematopoietic Stem Cells, HOS, Huh-7, Induced Pluripotent Stem (iPS) Cell or derivative, Jurkat, K562, L5278Y, LNCaP, MCF7, MDA-MB-231, MDCK, Mesenchymal Cells, Min-6, Monocytic cell, Neuro2a, NIH 3T3, NIH3T3L1, K562, NK-cells, NSO, Panc-1, PC12, PC-3, Peripheral blood cells, Plasma cells, Primary Fibroblasts, RBL, Renca, RLE, SF21, SF9, SH-SY5Y, SK-MES-1, SK-N-SH, SL3, SW403, Stimulus-triggered Acquisition of Pluripotency (STAP) cell or derivate SW403, T-cells, THP-1, Tumor cells, U20S, U937, peripheral blood lymphocytes, expanded T cells, hematopoietic stem cells, or Vero cells. In some embodiments, the cells are HEK293T cells.

The term “passaged,” as used herein, is intended to refer to the process of splitting cells in order to produce large number of cells from pre-existing ones. Cells may be passaged multiple times prior to or after any step described herein. Passaging involves splitting the cells and transferring a small number into each new vessel. For adherent cultures, cells first need to be detached, commonly done with a mixture of trypsin-EDTA. A small number of detached cells can then be used to seed a new culture, while the rest is discarded. Also, the amount of cultured cells can easily be enlarged by distributing all cells to fresh flasks. Cells may be kept in culture and incubated under conditions to allow cell replication. In some embodiments, the cells are kept in culture conditions that allow the cells to under 1, 2, 3, 4, 5, 6, 7, 8, 9, 10 or more rounds of cell division.

In some embodiments, cells may subjected to limiting dilution methods to enable the expansion of clonal populations of cells. The methods of limiting dilution cloning are well known to those of skill in the art. Such methods have been described, for example for hybridomas but can be applied to any cell. Such methods are described in (Cloning hybridoma cells by limiting dilution, Journal of tissue culture methods, 1985, Volume 9, Issue 3, pp 175-177, by Joan C. Rener, Bruce L. Brown, and Roland M. Nardone) which is incorporated by reference herein.

Methods of the disclosure include the culturing of cells. Methods of culturing suspension and adherent cells are well-known to those skilled in the art. In some embodiments, cells are cultured in suspension, using commercially available cell-culture vessels and cell culture media. Examples of commercially available culturing vessels that may be used in some embodiments including ADME/TOX Plates, Cell Chamber Slides and Coverslips, Cell Counting Equipment, Cell Culture Surfaces, Corning HYPERFlask Cell Culture Vessels, Coated Cultureware, Nalgene Cryoware, Culture Chamber, Culture Dishes, Glass Culture Flasks, Plastic Culture Flasks, 3D Culture Formats, Culture Multiwell Plates, Culture Plate Inserts, Glass Culture Tubes, Plastic Culture Tubes, Stackable Cell Culture Vessels, Hypoxic Culture Chamber, Petri dish and flask carriers, Quickfit culture vessels, Scale-Up Cell Culture using Roller Bottles, Spinner Flasks, 3D Cell Culture, or cell culture bags.

In other embodiments, media may be formulated using components well-known to those skilled in the art. Formulations and methods of culturing cells are described in detail in the following references: Short Protocols in Cell Biology J. Bonifacino, et al., ed., John Wiley & Sons, 2003, 826 pp; Live Cell Imaging: A Laboratory Manual D. Spector & R. Goldman, ed., Cold Spring Harbor Laboratory Press, 2004, 450 pp.; Stem Cells Handbook S. Sell, ed., Humana Press, 2003, 528 pp.; Animal Cell Culture: Essential Methods, John M. Davis, John Wiley & Sons, Mar. 16, 2011; Basic Cell Culture Protocols, Cheryl D. Helgason, Cindy Miller, Humana Press, 2005; Human Cell Culture Protocols, Series: Methods in Molecular Biology, Vol. 806, Mitry, Ragai R.; Hughes, Robin D. (Eds.), 3rd ed. 2012, XIV, 435 p. 89, Humana Press; Cancer Cell Culture: Method and Protocols, Cheryl D. Helgason, Cindy Miller, Humana Press, 2005; Human Cell Culture Protocols, Series: Methods in Molecular Biology, Vol. 806, Mitry, Ragai R.; Hughes, Robin D. (Eds.), 3rd ed. 2012, XIV, 435 p. 89, Humana Press; Cancer Cell Culture: Method and Protocols, Simon P. Langdon, Springer, 2004; Molecular Cell Biology. 4th edition, Lodish H, Berk A, Zipursky S L, et al., New York: W. H. Freeman; 2000, Section 6.2 Growth of Animal Cells in Culture, all of which are incorporated herein by reference.

### VI. GENOMIC INTEGRATION OF NUCLEIC ACIDS

A. Targeted Integration

The current disclosure provides methods for targeting the integration of a nucleic acid. This is also referred to as “gene editing” herein and in the art. In some embodiments, targeted integration is achieved through the use of a DNA digesting agent/polynucleotide modification enzyme, such as a site-specific recombinase and/or a targeting endonuclease. The term “DNA digesting agent” refers to an agent that is capable of cleaving bonds (i.e. phosphodiester bonds) between the nucleotide subunits of nucleic acids.

In one aspect, the current disclosure includes targeted integration. One way of achieving this is through the use of an exogenous nucleic acid sequence (i.e., a landing pad) comprising at least one recognition sequence for at least one polynucleotide modification enzyme, such as a site-specific recombinase and/or a targeting endonuclease. Site-specific recombinases are well known in the art, and may be generally referred to as invertases, resolvases, or integrases. Non-limiting examples of site-specific recombinases may include lambda integrase, Cre recombinase, FLP recombinase, gamma-delta resolvase, Tn3 resolvase, SC31 integrase, Bxb1-integrase, and R4 integrase. Site-specific recombinases recognize specific recognition sequences (or recognition sites) or variants thereof, all of which are well known in the art. For example, Cre recombinases recognize LoxP sites and FLP recombinases recognize FRT sites.

Contemplated targeting endonucleases include zinc finger nucleases (ZFNs), meganucleases, transcription activator-like effector nucleases (TALENs), CRIPSR/Cas-like endonucleases, I-Tev1 nucleases or related monomeric hybrids, or artificial targeted DNA double strand break inducing agents. Exemplary targeting endonucleases is further described below. For example, typically, a zinc finger nuclease comprises a DNA binding domain (i.e., zinc finger) and a cleavage domain (i.e., nuclease), both of which are described below. Also included in the definition of polynucleotide modification enzymes are any other useful fusion proteins known to those of skill in the art, such as may comprise a DNA binding domain and a nuclease.

A landing pad sequence is a nucleotide sequence comprising at least one recognition sequence that is selectively bound and modified by a specific polynucleotide modification enzyme such as a site-specific recombinase and/or a targeting endonuclease. In general, the recognition sequence(s) in the landing pad sequence does not exist endogenously in the genome of the cell to be modified. For example, where the cell to be modified is a CHO cell, the recognition sequence in the landing pad sequence is not present in the endogenous CHO genome. The rate of targeted integration may be improved by selecting a recognition sequence for a high efficiency nucleotide modifying enzyme that does not exist endogenously within the genome of the targeted cell. Selection of a recognition sequence that does not exist endogenously also reduces potential off-target integration. In other aspects, use of a recognition sequence that is native in the cell to be modified may be desirable. For example, where multiple recognition sequences are employed in the landing pad sequence, one or more may be exogenous, and one or more may be native.

One of ordinary skill in the art can readily determine sequences bound and cut by site-specific recombinases and/or targeting endonucleases.

Multiple recognition sequences may be present in a single landing pad, allowing the landing pad to be targeted sequentially by two or more polynucleotide modification enzymes such that two or more unique nucleic acids (comprising, among other things, receptor genes and/or inducible reporters) can be inserted. Alternatively, the presence of multiple recognition sequences in the landing pad, allows multiple copies of the same nucleic acid to be inserted into the landing pad. When two nucleic acids are targeted to a single landing pad, the landing pad includes a first recognition sequence for a first polynucleotide modification enzyme (such as a first ZFN pair), and a second recognition sequence for a second polynucleotide modification enzyme (such as a second ZFN pair). Alternatively, or additionally, individual landing pads comprising one or more recognition sequences may be integrated at multiple locations. Increased protein expression may be observed in cells transformed with multiple copies of a payload Alternatively, multiple gene products may be expressed simultaneously when multiple unique nucleic acid sequences comprising different expression cassettes are inserted, whether in the same or a different landing pad. Regardless of the number and type of nucleic acid, when the targeting endonuclease is a ZFN, exemplary ZFN pairs include hSIRT, hRSK4, and hAAVS 1, with accompanying recognition sequences.

Generally speaking, a landing pad used to facilitate targeted integration may comprise at least one recognition sequence. For example, a landing pad may comprise at least one, at least two, at least three, at least four, at least five, at least six, at least seven, at least eight, at least nine, or at least ten or more recognition sequences. In embodiments comprising more than one recognition sequence, the recognition sequences may be unique from one another (i.e. recognized by different polynucleotide modification enzymes), the same repeated sequence, or a combination of repeated and unique sequences.

One of ordinary skill in the art will readily understand that an exogenous nucleic acid used as a landing pad may also include other sequences in addition to the recognition sequence(s). For example, it may be expedient to include one or more sequences encoding selectable or screenable genes as described herein, such as antibiotic resistance genes, metabolic selection markers, or fluorescence proteins. Use of other supplemental sequences such as transcription regulatory and control elements (i.e., promoters, partial promoters, promoter traps, start codons, enhancers, introns, insulators and other expression elements) can also be present.

In addition to selection of an appropriate recognition sequence(s), selection of a targeting endonuclease with a high cutting efficiency also improves the rate of targeted integration of the landing pad(s). Cutting efficiency of targeting endonucleases can be determined using methods well-known in the art including, for example, using assays such as a CEL-1 assay or direct sequencing of insertions/deletions (Indels) in PCR amplicons.

The type of targeting endonuclease used in the methods and cells disclosed herein can and will vary. The targeting endonuclease may be a naturally-occurring protein or an engineered protein. One example of a targeting endonuclease is a zinc-finger nuclease, which is discussed in further detail below.

Another example of a targeting endonuclease that can be used is an RNA-guided endonuclease comprising at least one nuclear localization signal, which permits entry of the endonuclease into the nuclei of eukaryotic cells. The RNA-guided endonuclease also comprises at least one nuclease domain and at least one domain that interacts with a guiding RNA. An RNA-guided endonuclease is directed to a specific chromosomal sequence by a guiding RNA such that the RNA-guided endonuclease cleaves the specific chromosomal sequence. Since the guiding RNA provides the specificity for the targeted cleavage, the endonuclease of the RNA-guided endonuclease is universal and may be used with different guiding RNAs to cleave different target chromosomal sequences. Discussed in further detail below are exemplary RNA-guided endonuclease proteins. For example, the RNA-guided endonuclease can be a CRISPR/Cas protein or a CRISPR/Cas-like fusion protein, an RNA-guided endonuclease derived from a clustered regularly interspersed short palindromic repeats (CRISPR)/CRIS PR-associated (Cas) system.

The targeting endonuclease can also be a meganuclease. Meganucleases are endodeoxyribonucleases characterized by a large recognition site, i.e., the recognition site generally ranges from about 12 base pairs to about 40 base pairs. As a consequence of this requirement, the recognition site generally occurs only once in any given genome. Among meganucleases, the family of homing endonucleases named LAGLIDADG has become a valuable tool for the study of genomes and genome engineering. Meganucleases may be targeted to specific chromosomal sequence by modifying their recognition sequence using techniques well known to those skilled in the art. See, for example, Epinat et al., 2003, Nuc. Acid Res., 31(11):2952-62 and Stoddard, 2005, Quarterly Review of Biophysics, pp. 1-47.

Yet another example of a targeting endonuclease that can be used is a transcription activator-like effector (TALE) nuclease. TALEs are transcription factors from the plant pathogen Xanthomonas that may be readily engineered to bind new DNA targets. TALEs or truncated versions thereof may be linked to the catalytic domain of endonucleases such as FokI to create targeting endonuclease called TALE nucleases or TALENs. See, e.g., Sanjana et al., 2012, Nature Protocols 7(1):171-192; Bogdanove A J, Voytas D F., 2011, Science, 333(6051):1843-6; Bradley P, Bogdanove A J, Stoddard B L., 2013, Curr Opin Struct Biol., 23(1):93-9.

Another exemplary targeting endonuclease is a site-specific nuclease. In particular, the site-specific nuclease may be a “rare-cutter” endonuclease whose recognition sequence occurs rarely in a genome. Preferably, the recognition sequence of the site-specific nuclease occurs only once in a genome. Alternatively, the targeting nuclease may be an artificial targeted DNA double strand break inducing agent.

In some embodiments, targeted integrated can be achieved through the use of an integrase. For example, The phiC31 integrase is a sequence-specific recombinase encoded within the genome of the bacteriophage phiC31. The phiC31 integrase mediates recombination between two 34 base pair sequences termed attachment sites (att), one found in the phage and the other in the bacterial host. This serine integrase has been show to function efficiently in many different cell types including mammalian cells. In the presence of phiC31 integrase, an attB-containing donor plasmid can be unidirectional integrated into a target genome through recombination at sites with sequence similarity to the native attP site (termed pseudo-attP sites). phiC31 integrase can integrate a plasmid of any size, as a single copy, and requires no cofactors. The integrated transgenes are stably expressed and heritable.

In one embodiment, genomic integration of polynucleotides of the disclosure is achieved through the use of a transposase. For example, a synthetic DNA transposon (e.g. “Sleeping Beauty” transposon system) designed to introduce precisely defined DNA sequences into the chromosome of vertebrate animals can be used. The Sleeping Beauty transposon system is composed of a Sleeping Beauty (SB) transposase and a transposon that was designed to insert specific sequences of DNA into genomes of vertebrate animals. DNA transposons translocate from one DNA site to another in a simple, cut-and-paste manner. Transposition is a precise process in which a defined DNA segment is excised from one DNA molecule and moved to another site in the same or different DNA molecule or genome.

As do all other Tc1/mariner-type transposases, SB transposase inserts a transposon into a TA dinucleotide base pair in a recipient DNA sequence. The insertion site can be elsewhere in the same DNA molecule, or in another DNA molecule (or chromosome). In mammalian genomes, including humans, there are approximately 200 million TA sites. The TA insertion site is duplicated in the process of transposon integration. This duplication of the TA sequence is a hallmark of transposition and used to ascertain the mechanism in some experiments. The transposase can be encoded either within the transposon or the transposase can be supplied by another source, in which case the transposon becomes a non-autonomous element. Non-autonomous transposons are most useful as genetic tools because after insertion they cannot independently continue to excise and re-insert. All of the DNA transposons identified in the human genome and other mammalian genomes are non-autonomous because even though they contain transposase genes, the genes are non-functional and unable to generate a transposase that can mobilize the transposon.

### VII. METHODS OF USE

The assays described herein make large-scale screens both time- and cost-effective. Furthermore, the assays described herein are useful for the screening of a ligand for on and off-target effects, for determining the activity of variants of one or more receptors to a particular ligand or set of ligands, for mapping critical residues required in a receptor required for ligand binding, and for determining which residues in a receptor are non-critical for ligand binding.

In some aspects the assay methods relate to an assay wherein the receptors are variants of one receptor. In some embodiment, each variant comprises or consists of one substitution relative to the wild-type protein sequence. In some embodiments, each variant comprises or consists of at least, at most, or exactly 1, 2, 3, 4, 5, 6, 7, 8, 9, or 10 substitutions (or any derivable range therein), compared to the wild-type amino acid sequence. In some aspects, the methods comprise determining the activity of a population of receptors to a ligand, wherein the population of receptors comprises at least two variants of the same receptor, and wherein the activity is determined in response to a ligand. In some aspects, the population of receptors comprises at least, at most, or about 2, 10, 100, 200, 300, 400, 500, 1000, 1500, 2000, 3000, 4000, or 5000 receptors (or any derivable range therein) are screened. In some aspects at least, at most, or exactly 1, 2, 3, 4, 5, 6, 7, 8, 9, or 10 ligands (or any derivable range therein) are screened. In some aspects, at least, at most, or about 2, 10, 100, 200, 300, 400, 500, 1000, 1500, 2000, 3000, 4000, or 5000 receptors (or any derivable range therein) are screened in response to at least, at most, or exactly 1, 2, 3, 4, 5, 6, 7, 8, 9, or 10 ligands (or any derivable range therein). In some embodiments, the assays may be used to predict a patient's response to a ligand based on the determined activity of a variant receptor to the ligand. For example, the assays described herein may be used to predict a therapeutic response of a variant receptor to a ligand. This information may then be used in a treatment method to treat a patient having the variant receptor. In some embodiments, the methods comprise treating a patient with a ligand, wherein the patient has been determined to have a variant receptor. In some embodiments, the activity of the variant receptor to the ligand has been determined by a method described herein.

In some aspects, the assay is for determining the activity of a class of receptors to one or more ligands.

In some embodiments, the class of receptors are olfactory, GPCR, nuclear hormone, hormone, or catalytic receptors. In some embodiments, the receptor is an adrenoceptor, such as an alpha or beta adrenergic receptor or an alpha-1, alpha-2, beta-1, beta-2, or beta-3 adrenergic receptor, or an alpha-1A, alpha 1B, alpha-1D, alpha-2A, alpha-2B, or alpha-2C adrenergic receptor. In some embodiments, the receptor or class of receptors is one described herein.

### VIII. KITS

Certain aspects of the present disclosure also concern kits containing nucleic acids, vectors, or cells of the disclosure. The kits may be used to implement the methods of the disclosure. In some embodiments, kits can be used to evaluate the activation of a receptor gene or a group of receptor genes. In some embodiments, the kits can be used to evaluate variants of a single gene. In certain embodiments, a kit contains, contains at least or contains at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 500, 1,000 or more nucleic acid probes, primers, or synthetic RNA molecules, or any value or range and combination derivable therein. In some embodiments, there are kits for evaluating the activation of or engagement of a receptor by a ligand. In some embodiments, universal probes or primers are included for amplifying, identifying, or sequencing a barcode or receptor. Such reagents may also be used to generate or test host cells that can be used in screens.

In certain embodiments, the kits may comprise materials for analyzing cell morphology and/or phenotype, such as histology slides and reagents, histological stains, alcohol, buffers, tissue embedding mediums, paraffin, formaldehyde, and tissue dehydrant.

Kits may comprise components, which may be individually packaged or placed in a container, such as a tube, bottle, vial, syringe, or other suitable container means.

Individual components may also be provided in a kit in concentrated amounts; in some embodiments, a component is provided individually in the same concentration as it would be in a solution with other components. Concentrations of components may be provided as 1×, 2×, 5×, 10×, or 20× or more.

Kits for using probes, polypeptide or polynucleotide detecting agents of the disclosure for drug discovery are contemplated.

In certain aspects, negative and/or positive control agents are included in some kit embodiments. The control molecules can be used to verify transfection efficiency and/or control for transfection-induced changes in cells.

Embodiments of the disclosure include kits for analysis of a pathological sample by assessing a nucleic acid or polypeptide profile for a sample comprising, in suitable container means, two or more RNA probes or primers for detecting expressed polynucleotides. Furthermore, the probes or primers may be labeled. Labels are known in the art and also described herein. In some embodiments, the kit can further comprise reagents for labeling probes, nucleic acids, and/or detecting agents. The kit may also include labeling reagents, including at least one of amine-modified nucleotide, poly(A) polymerase, and poly(A) polymerase buffer. Labeling reagents can include an amine-reactive dye. Kits can comprise any one or more of the following materials: enzymes, reaction tubes, buffers, detergent, primers, probes, antibodies. In some embodiments, these kits include the needed apparatus for performing RNA extraction, RT-PCR, and gel electrophoresis. Instructions for performing the assays can also be included in the kits.

The kits may further comprise instructions for using the kit for assessing expression, means for converting the expression data into expression values and/or means for analyzing the expression values to generate ligand/receptor interaction data.

Kits may comprise a container with a label. Suitable containers include, for example, bottles, vials, and test tubes. The containers may be formed from a variety of materials such as glass or plastic. The container may hold a composition which includes a probe that is useful for the methods of the disclosure. The kit may comprise the container described above and one or more other containers comprising materials desirable from a commercial and user standpoint, including buffers, diluents, filters, needles, syringes, and package inserts with instructions for use.

### IX. EXAMPLES

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—A Multiplexed Odorant-Receptor Screening System

Mammalian olfaction is a highly complex process and arguably the least understood sense. Olfactory receptors (ORs) are the first layer of odor perception. Human ORs are a set of 400 G protein-coupled receptors (GPCRs) that are monoallelically expressed in neurons located in the nasal epithelium. Odorants bind receptors in a many-to-many fashion, the pattern is transmitted to the olfactory bulb, and transformed into perception in the cortex. only ˜5% of human ORs have high affinity ligands identified for them, the large number of orphan receptors inhibits one's ability to interrogate the downstream neurobiology that governs olfaction. Previous deorphanization attempts utilized heterologous cell-based assays that screened each odorant-receptor pair individually. The high number of potential receptor-odorant combinations and the difficulty in achieving heterologous OR expression has limited the throughput of “one-at-a-time” approaches. Instead, the inventors have engineered a stable OR expressing cell line that enables multiplexed odorant-receptor screening.

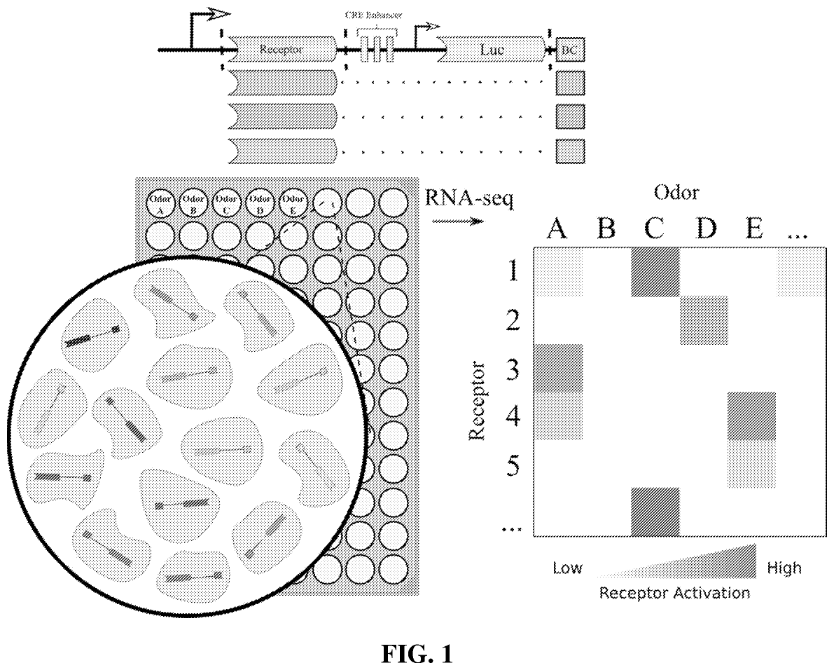

To measure receptor-odorant interactions, the inventors adapted a genetic reporter for cAMP signaling in HEK293T cells. Upon odorant binding, g-protein signaling stimulates cAMP production that leads to phosphorylation of the transcription factor CREB. CREB binds the short, tandem-repeat sequence CRE and turns on transcription of a downstream reporter gene, usually luciferase. The assay was modified to include DNA barcodes into the 3′ UTR of the reporter gene that uniquely associate with one OR in the library expressed on the same plasmid (FIG. 1). Each cell is integrated with a single library member to ensure cAMP signaling does not trigger expression of barcodes corresponding to receptors not bound by odorant but present within the same cell. The inventors seeded the cell line into 96-well plates, induced each well with different odors, and sequenced the barcoded transcripts. The inventors converted the relative abundance of each barcode into a heat map displaying affinity of the odors for each receptor.

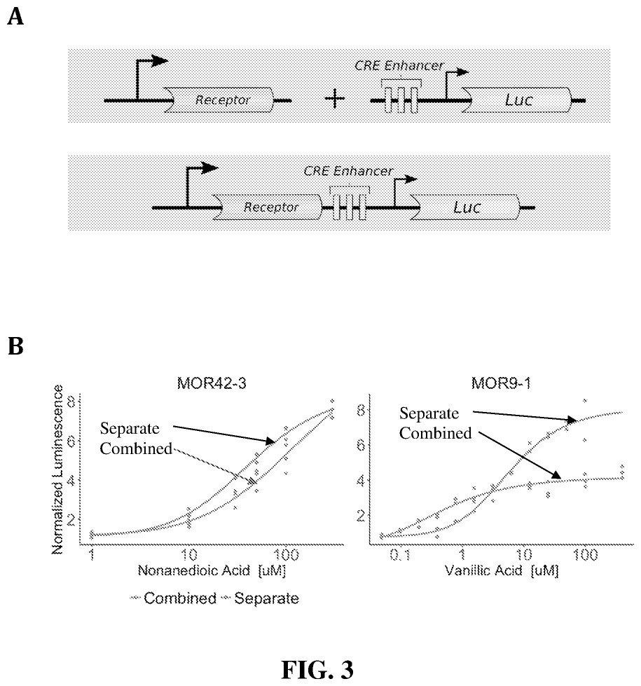

Typical genetic reporter assays for GPCR activation co-transfect the receptor and reporter individually. In order to map each barcode to its corresponding OR, one would need to express all the components for the assay on a single plasmid enabling association of barcode and OR via sequencing. The inventors configured a plasmid to express all necessary components (FIG. 3). The inventors transiently screened a range of concentrations for two ORs, MOR42-3 and MOR9-1, with known, high-affinity ligands against both configurations and observed comparable reporter activation.

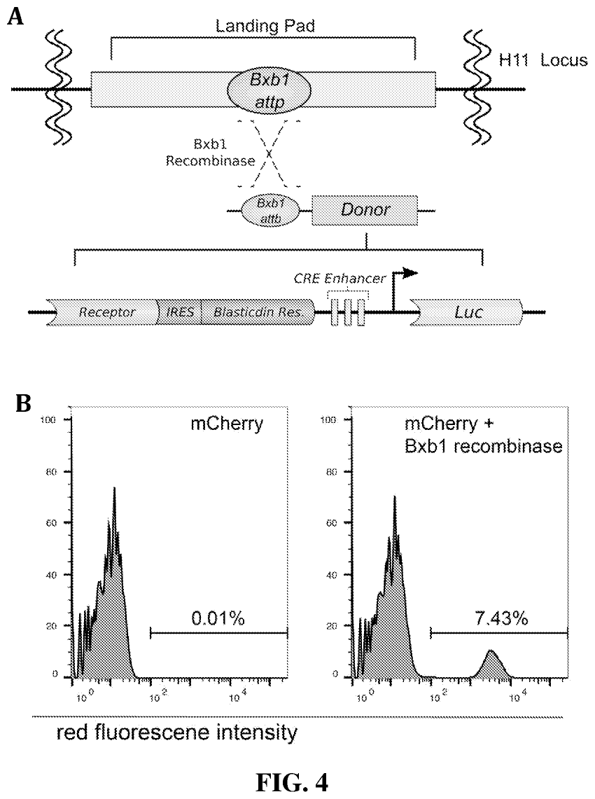

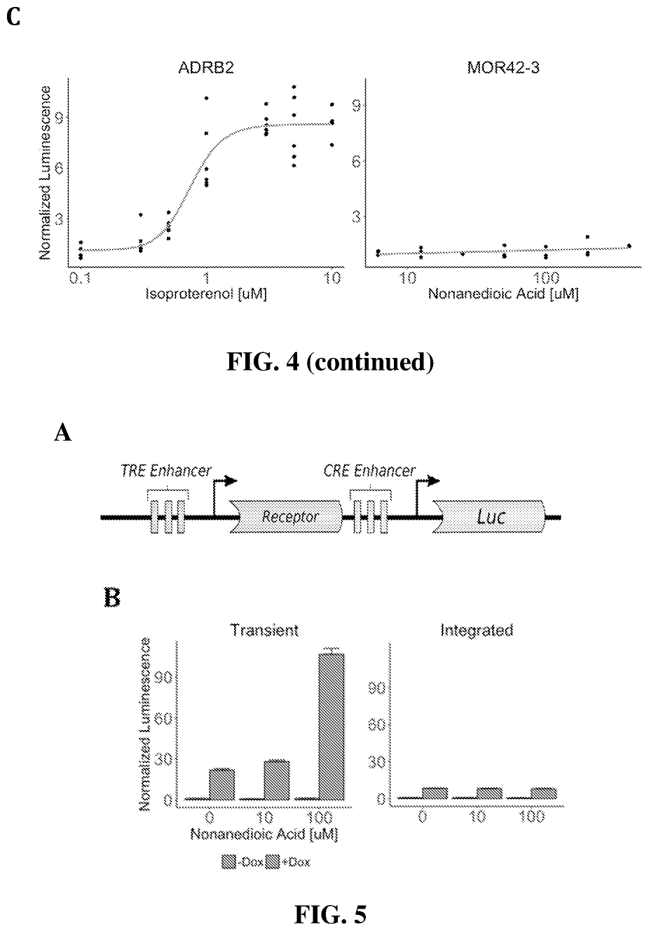

The multiplexing strategy requires stable, clonal integration of the OR library. Initially, the inventors decided to use Bxb1 recombination because it enabled each library member to be integrated at a single copy per cell in a single pot reaction. The inventors engineered a ‘landing pad’ containing the Bxb1 attp recombinase site into the Hl 1 safe harbor locus of HEK293T cells FIG. 4). The engineered cell line is referred to as Mukku1a (Table 1). Bxb1 recombination irreversibly integrates plasmid DNA containing a complementary attb recognition site and disrupts the genomic attp sequence restricting a single recombination per cell. The inventors were unable to observe reporter activation when inducing MOR42-3 in the landing pad. However, the beta-2 adrenergic receptor, a canonical GPCR that also activates adenylate cyclase, robustly activated the reporter upon induction when expressed from the landing pad.

| Modifications | Name |
|---|---|
| Landing Pad | Mukku1a |
| Landing Pad, Tet rTA | Mukku2a |
| Landing Pad, Tet rTA, Accessory Factors | Mukku3a |

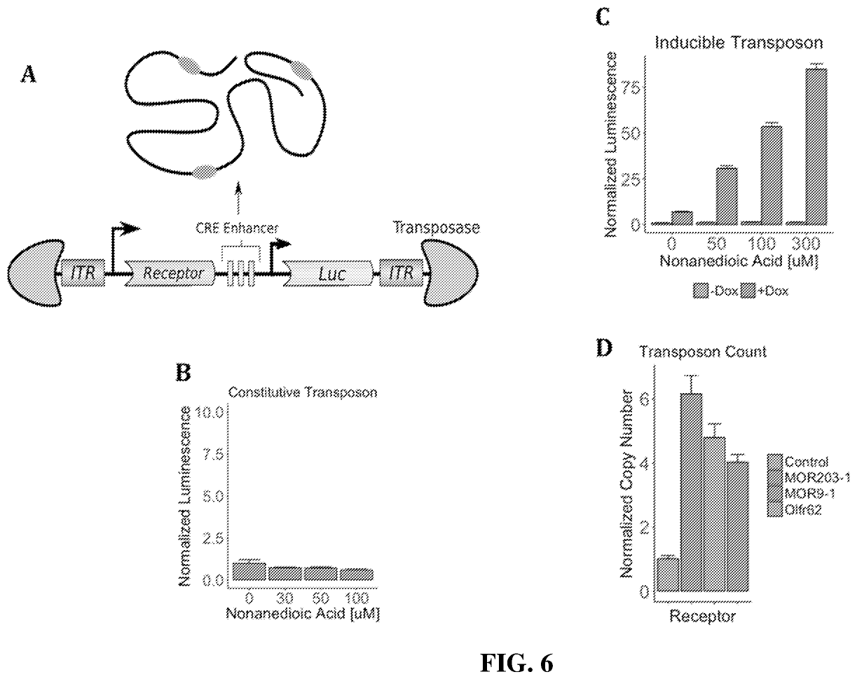

ORs are notoriously difficult to heterologously express and stable, heterologous expression has never been reported. We hypothesized stable, constitutive expression of ORs could lead to many possible avenues of down-regulation and decided to attempt inducible expression. The inventors engineered Mukku1a cells to express the reverse Tet transactivator and replaced the promoter driving OR expression with the Tet-On inducible promoter (FIG. 5). The inducible system achieved comparable reporter activation to the previous system transiently, but the inventors were still unable to observe reporter expression when in the landing pad. The next hypothesis was that a single OR gene was insufficient to achieve the expression necessary to activate the genetic reporter. The inventors flanked the genetic construct with intermediate terminal repeats and integrated the plasmid using a transposase (FIG. 6). Under constitutive OR expression, the reporter still did not respond to odorant. Unexpectedly, the combination of transposing the reporter and controlling OR expression inducibly restored the reporter's odorant response. QPCR confirmed the transposon was integrated at 4-6 copies per cell on average.

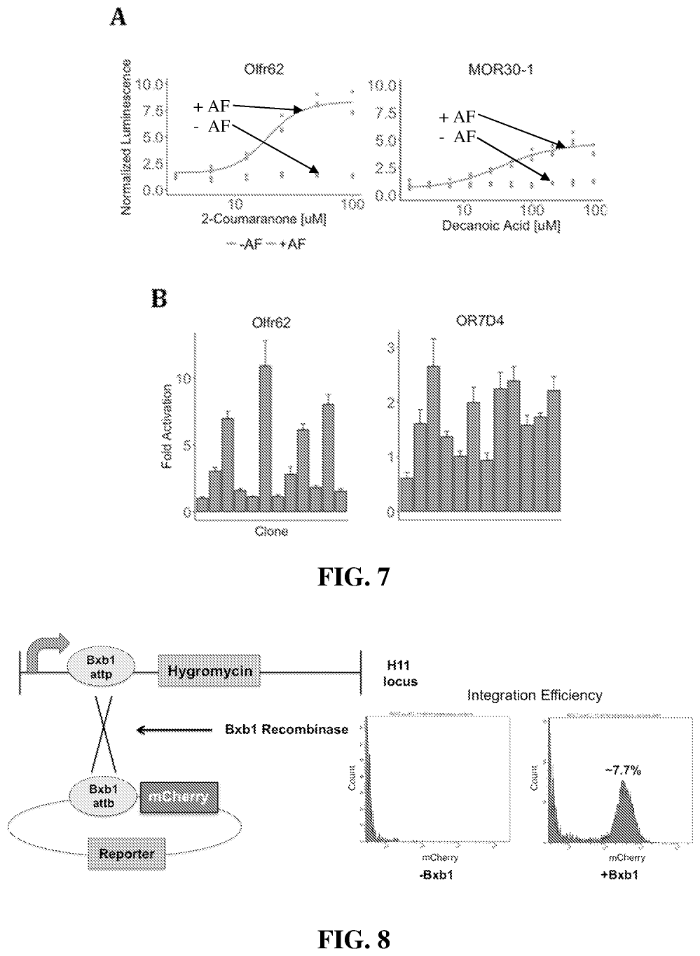

Many ORs require co-expression of accessory factors for cell membrane trafficking and proper signal transduction when transiently expressed in heterologous systems (FIG. 7). It was predicted this would be an issue for stable expression as well and genomically integrated 4 accessory factor transgenes: RTP1S and RTP2 (chaperones that increase surface expression), G_{αolf} (the G protein alpha subunit that natively interacts with ORs), and Ric8b (the guanine nucleotide exchange factor that associates with G_{αolf}). The inventors pooled and transposed these 4 factors under Tet inducible regulation into Mukku2a cells. To create a cell line with potent OR expression capability, the inventors isolated single clones and transiently screened them for genetic reporter activation against 2 ORs, Olfr62 and OR7D4, previously known to require accessory factors for heterologous functional expression.

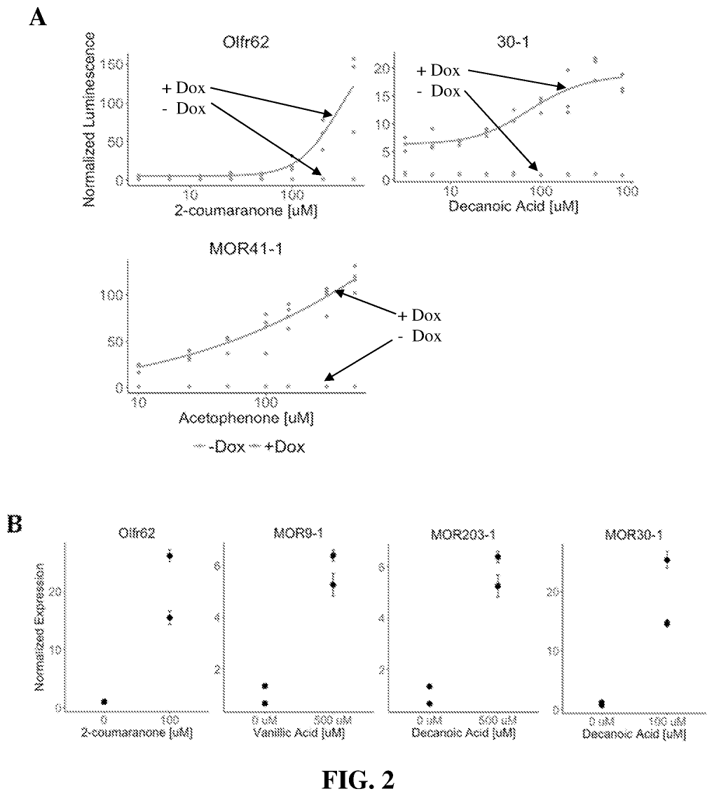

42 mouse ORs were cloned into the transposon vector containing a random barcode in the 3′ UTR of the reporter gene and sequenced clones to map barcodes to each receptor. Next, each construct was individually transposed into Mukku3a cells and then the cells were pooled together post-transposition. Ultimately, the integrated Mukku3a cells inducibly express both the accessory factors and the OR under control of the Tet-On system (data not shown). The inventors tested a handful of receptors with known ligands both at the protein and transcript level to confirm the stable cell line would replicate previous receptor-odorant associations and work reliably for a large receptor cohort (FIG. 2A-B).

In order to make the assay amenable to high throughput screening, a 96-well plate compatible, in-lysate protocol for library preparation (FIG. 8) was developed. Each well of the plate and the plates themselves were barcoded with custom indices. The inventors screened 4 separate concentrations of 96 odorants against our 42-receptor library yielding 16,128 unique receptor-ligand interactions. A heat map was constructed to display the relative activation of each receptor under each condition (FIG. 2C). The odorant-receptor interaction space is complex and difficult to traverse. The inventors have developed a platform that overcomes the challenge of heterologous OR expression and compresses the interaction space through multiplexing. This platform economically and technologically enables large-scale deorphanization of mammalian ORs.

### Example 2—Smell-Seq: A Multiplexed GPCR Activity Assay for Decoding Olfactory Receptor-Ligand Interactions

We developed a platform for multiplex receptor-ligand profiling by building libraries of stable human cell line reporters that can be read in multiplex by next generation sequencing in high-throughput formats. This technology generalizes to many other classes of receptors and allows high throughput screening for drug discovery for medicinally relevant GPCRs.

Interactions between small molecules and receptors underpin an organism's ability to sense and respond to its internal state and environment. For many drugs and natural products, the ability to to modulate the function of many biological targets at once are crucial for their efficacy. Such polypharmacology is difficult to study because we often do not know which chemicals interact with which targets. This many-on-many problem is laborious to study one interaction at a time and is especially manifest in the mammalian sense of smell.

Olfaction is mediated by a class of G protein-coupled receptors (GPCRs) known as olfactory receptors (ORs). GPCRs are a central player in small molecule signaling in mammals and are targeted by over 30% of FDA approved drugs. ORs are a large family of class A GPCRs that have specialized in many different evolutionary contexts with approximately 396, 1130, and 1948 intact receptors in humans, mice, and elephants respectively. Each OR could potentially interact with a near infinite number of odorants and each odorant with many ORs. The vast majority of ORs remain orphan because of this complexity and because recapitulating mammalian GPCR function in vitro is challenging. In addition no crystal structure for any OR exists, hindering computational efforts to predict which odorants activate each OR.

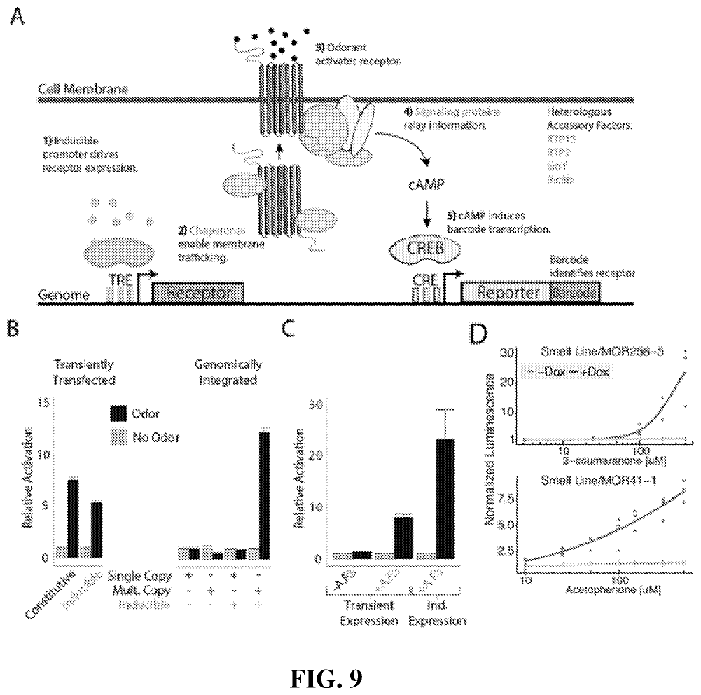

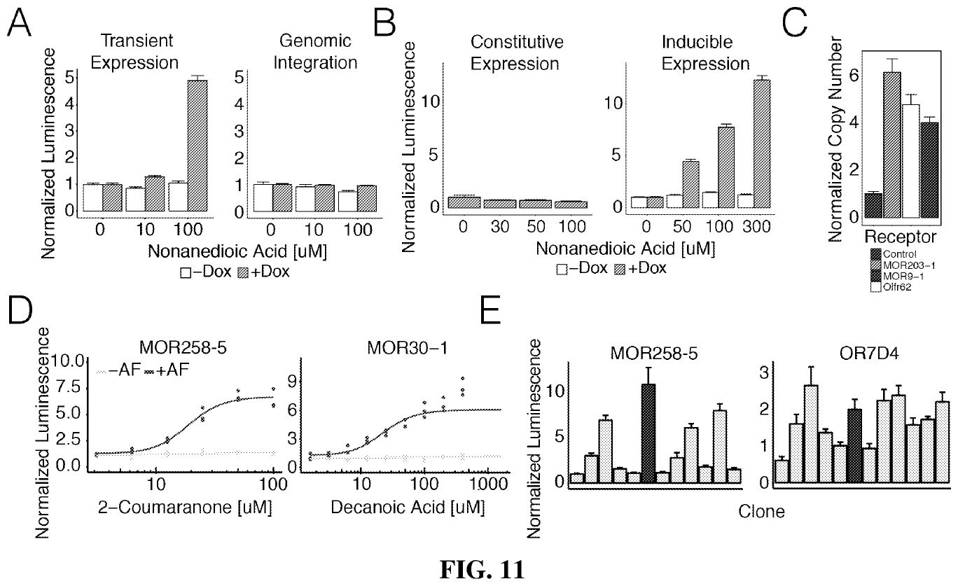

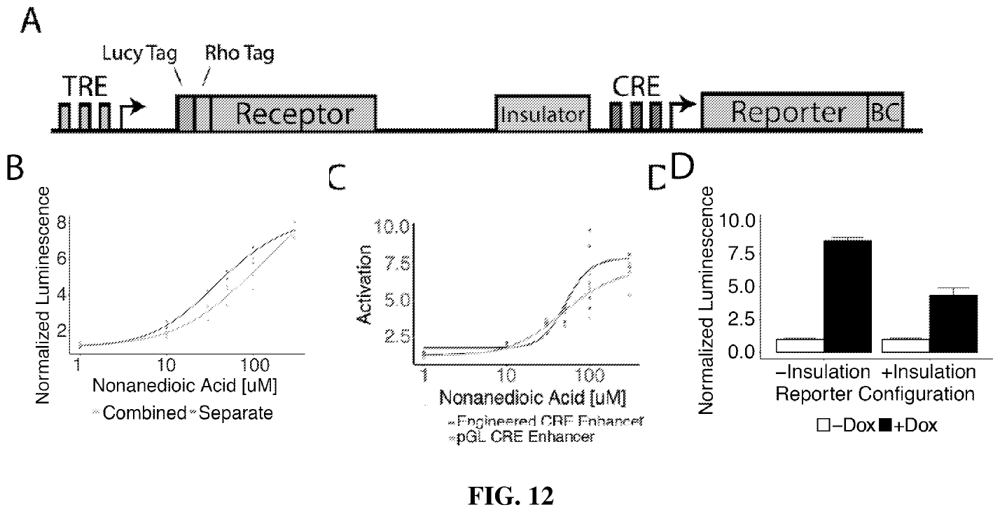

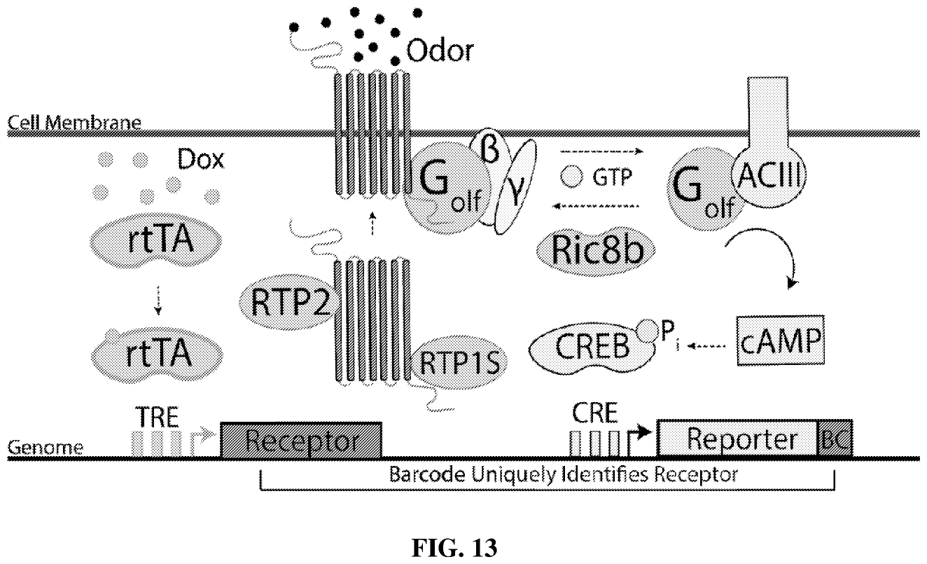

Here we report a new HTS-compatible system to characterize small molecule libraries against mammalian OR libraries in multiplex (FIG. 9A). To do this, we developed both a stable cell line capable of functional OR expression (FIG. 11) and a multiplexed reporter for OR activity (FIG. 12). The final platform comprises a multi-copy, inducibly expressed OR sitting within the context of an engineered cell line with inducibly expressed proteins required for OR trafficking and signaling (FIG. 13). Activation of each OR leads to the expression of a reporter transcript with a unique 15 nucleotide barcode sequence. Each barcode identifies the OR, allowing for the multiplexed readout by amplicon RNA-seq of the barcodes (FIG. 9A, FIG. 13). Using this platform, we have screened at least 42 different receptors, and we have adapted this platform for high-throughput screening that has allowed for the discovery of novel odorant pairs. We found that multi-copy integration and inducible expression allowed for reporter activation. Individually these features yielded no response; however, their combination resulted in a functional OR reporter cell line, which demonstrates a synergistic response not found when either multi-copy integration or inducible expression were used alone. We then inducibly expressed G_alpha_olf, Ric8b, RTP1S, RTP2, (FIG. 9B, FIG. 11). To engineer the reporter construct, we used protein trafficking tags to increase surface expression, added DNA insulator sequences to reduce background reporter activation, modified the cAMP response element (CRE) enhancer to improve reporter signal, and combined these elements into a single transposable vector to speed cell line development (FIG. 12). We validated our system on three murine ORs with known ligands, and observed induction and dose-dependent activation (FIG. 9C), including Olfr62 which has previously been difficult to express.

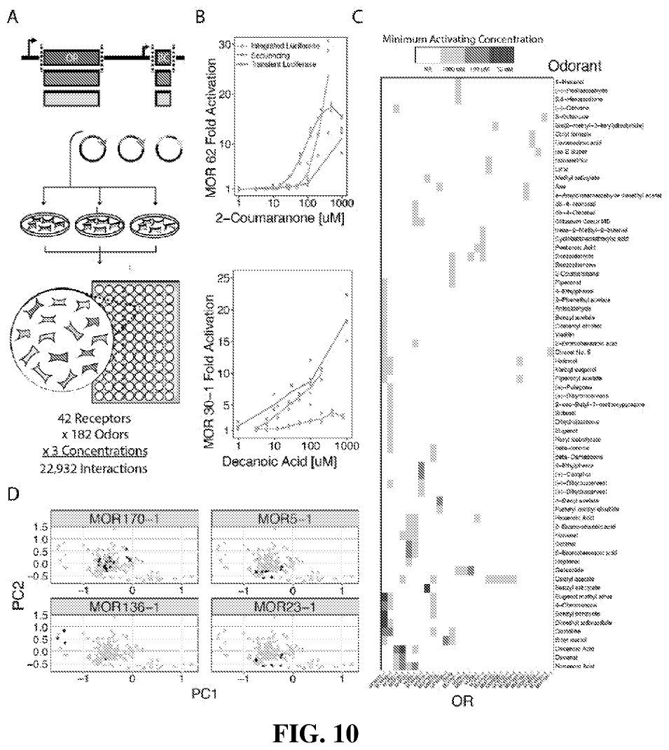

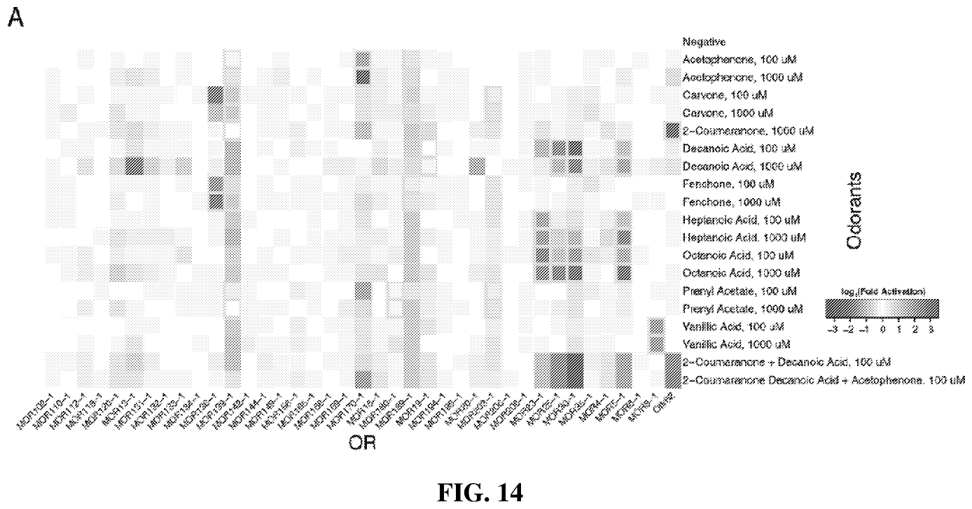

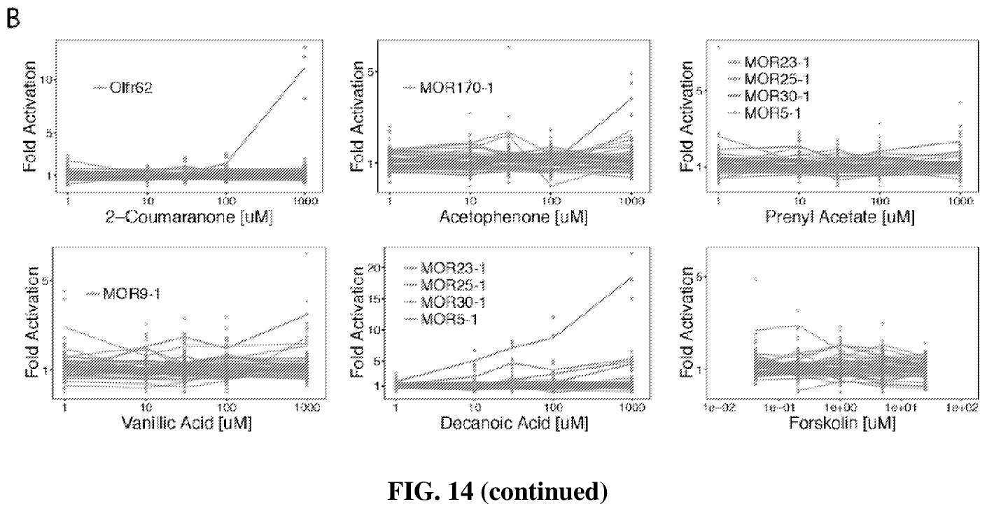

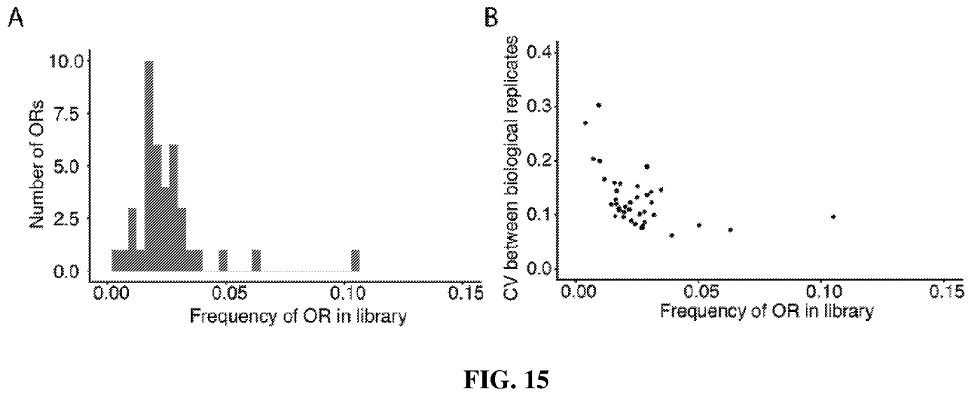

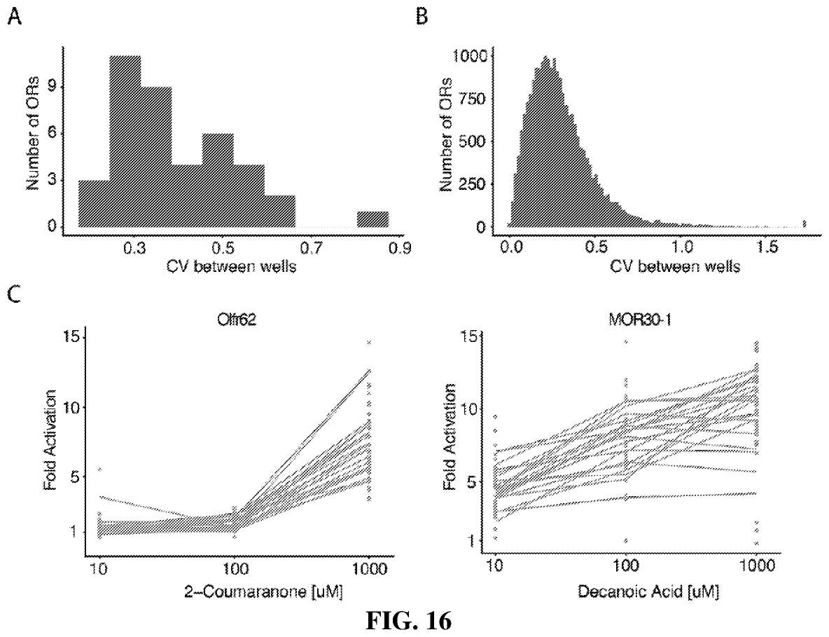

After modifications, we created a library of 42 murine OR-expressing cell lines and tested the multiplexed readout of activation. We first cloned and mapped the ORs to their corresponding barcodes via Sanger sequencing and transposed the plasmids individually into HEK-293T cells, pooling the cell lines together after selection (FIG. 10A). To pilot the multiplexed assay, we plated the cell library in 6-well culture dishes and added odorants known to activate specific ORs (FIG. 14); all but 3 ORs were present in enough cells to obtain reliable estimates of activation. Analysis of the sequencing readout recapitulated previously identified odorant-receptor pairs, and chemical mixtures appropriately activated multiple ORs. Interestingly, we found that the assay was robust to chemicals such as the direct adenylate cyclase stimulator forskolin, which nonspecifically stimulate cells independent of the OR they express. Because such chemicals activate all barcodes equivalently, such nuisance chemicals can easily be filtered out. Next, we adapted the platform for high-throughput screening in 96-well format. To decrease reagent cost and assay time, we developed an in-lysate reverse transcription protocol and used dual indexing to uniquely identify each well (see Methods). Using these improvements, we were able to recapitulate dose-response curves for known odorant-receptor pairs (FIG. 10B, FIG. 14). We observed reproducible results between identically treated but biologically independent wells (FIGS. 15-16).

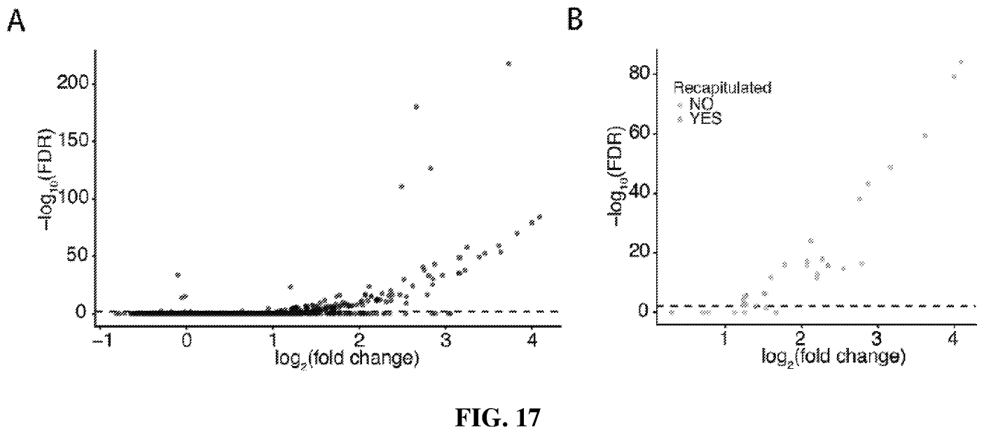

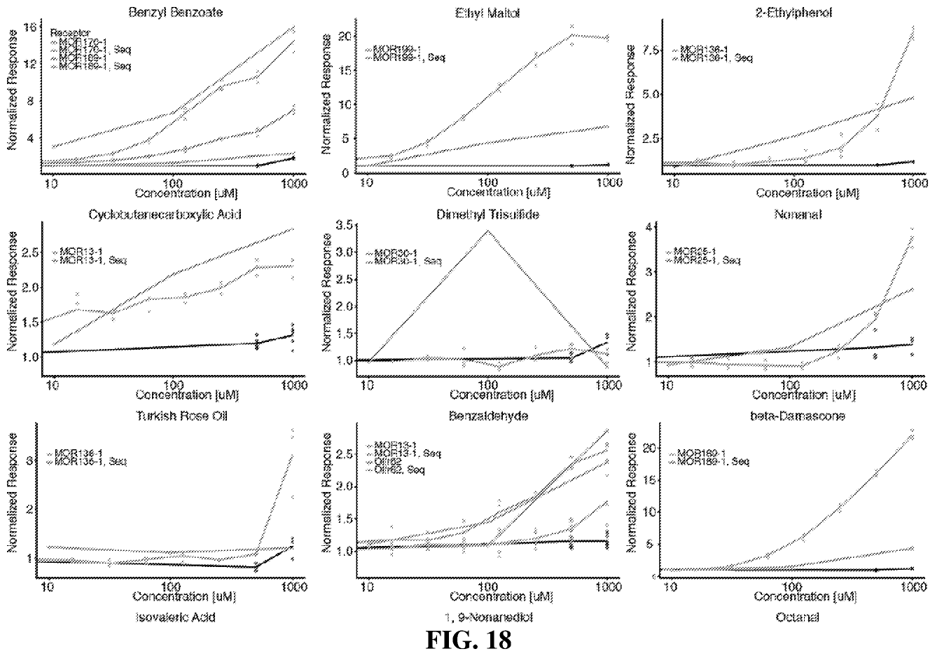

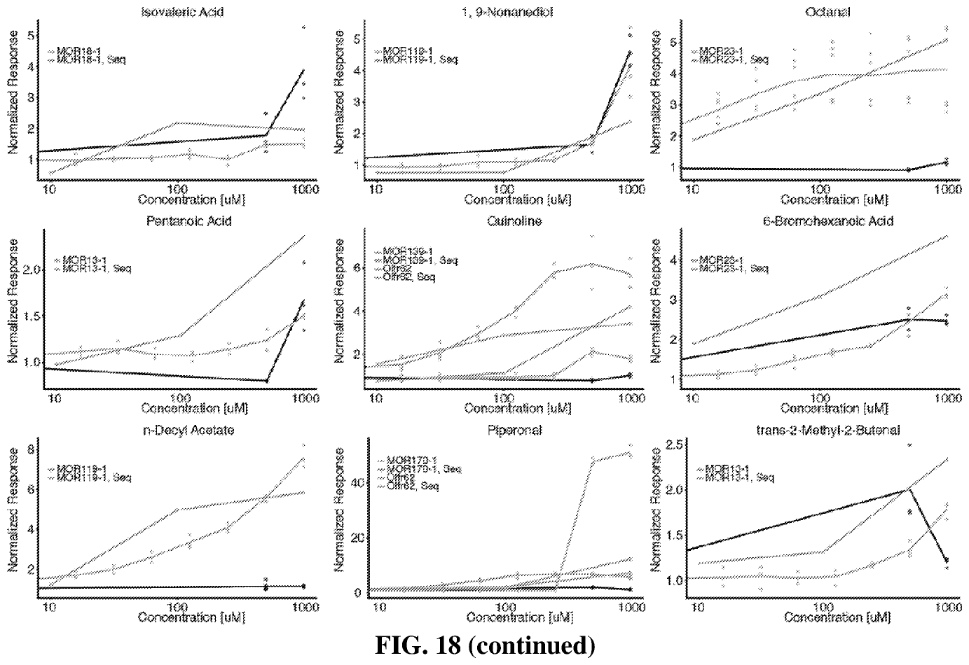

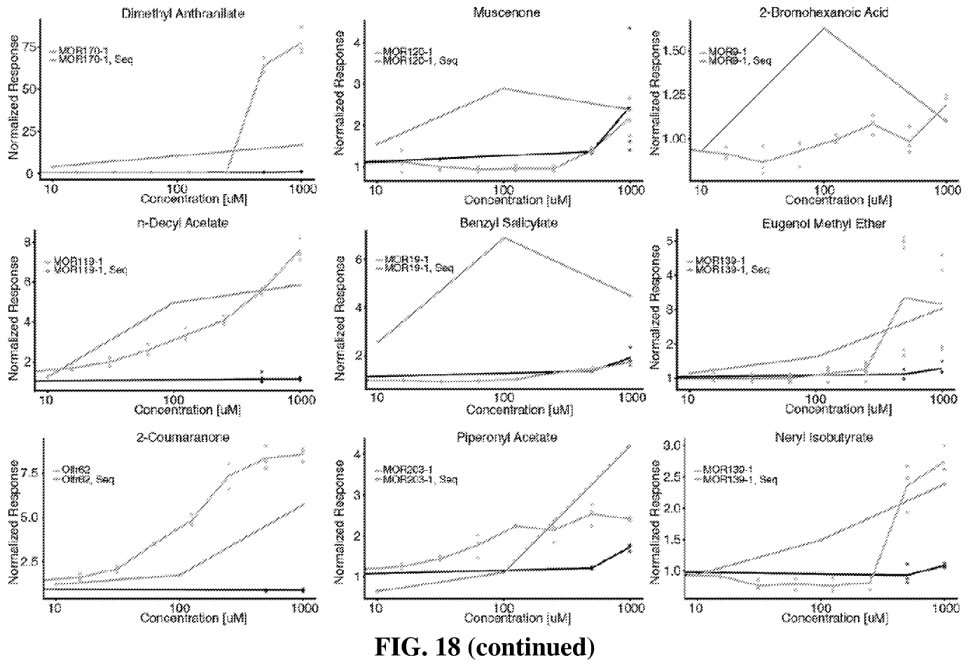

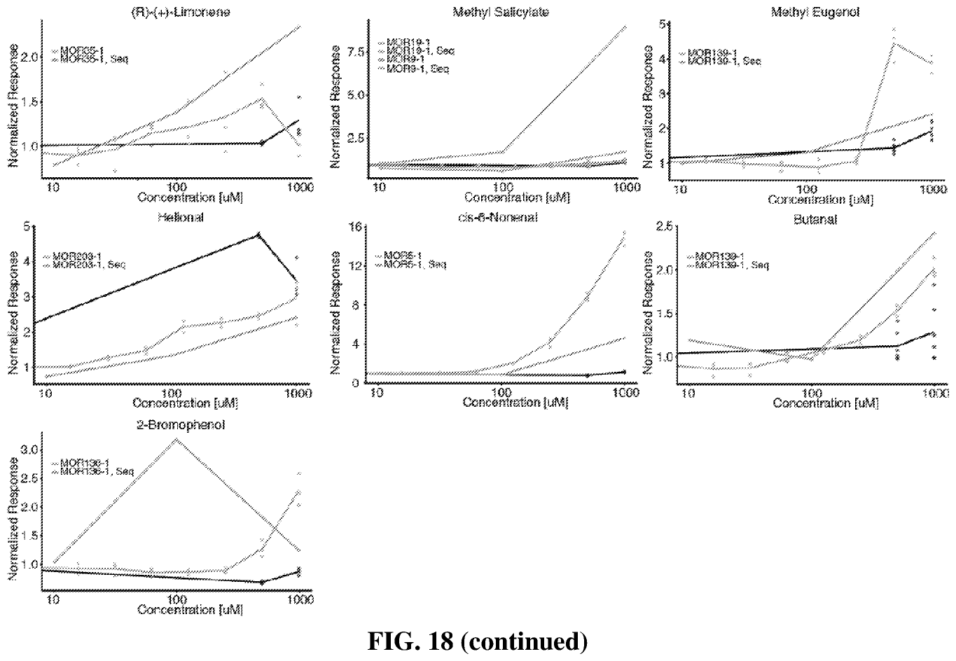

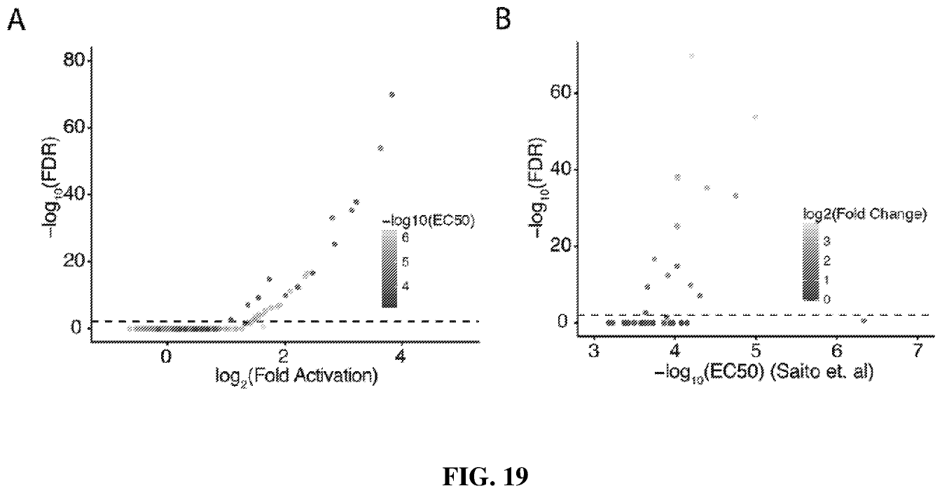

We subsequently screened 182 odorants at three concentrations in triplicate against the OR cell library, the equivalent of ˜85,000 individual luciferase assays including controls (FIG. 10A, Table 2). Each 96-well plate in the assay contained positive control odorants and solvent DMSO wells for normalization (FIG. 16). We used the EdgeR software package to determine differentially responsive ORs based on a negative binomial model of barcode counts. We found 114 OR-odorant interactions (out of 7,200 possible), 81 of which are novel, and 24 interactions with 15 orphan receptors (FIG. 10C, FIG. 17 and Supplementary Table 4) (FDR=1%; Benjamini-Hochberg correction). Overall 28 of 39 receptors were activated by at least one odorant, and 68 of 182 odorants activated at least one OR (Table 4). We chose 37 interactions of at least 1.2 fold induction to test individually with a previously developed transient OR assay that has several important differences (FIG. 18). Of the 28 interactions called as hits at an FDR of 1%, 21 of them replicated in this orthogonal system (FIG. 17). Even some of the seven that did not replicate are likely real. For instance, our assay registered two hits for MOR19-1 with high chemical similarity (methyl salicylate and benzyl salicylate) suggesting they are likely not false positives (FIG. 18). Additionally, three of nine interactions not passing the 1% FDR threshold showed activation in the orthogonal assay, indicating a conservative threshold. A previous large-scale OR deorphanization study used some of the same receptors and chemicals and we found that 9/12 of their reported interactions with EC50 below 100M were also detected in our platform, though we did not identify most of the previous low affinity interactions (FIG. 19). Conversely, we also detect 14 interactions that this previous study tested, but called negative. Finally, our assay mostly recapitulated the combinations of odorant and OR that did not interact (493/507).

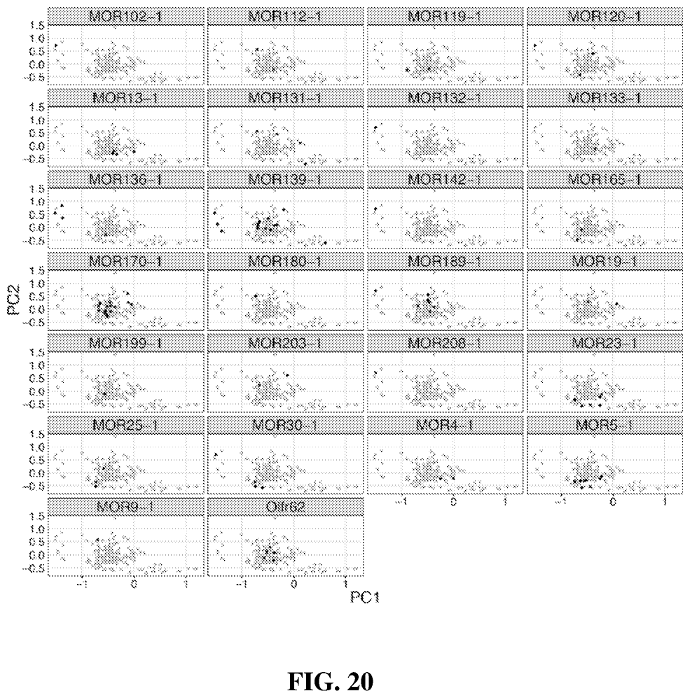

We find that chemicals with similar features activate similar sets of ORs, including those receptors we deorphanize in this study. For example, the previously orphan MOR13-1 is activated by four chemicals with polar groups attached, in three cases, to stiff non-rotatable scaffolds. Another example is, MOR19-1, which has clear affinity for the salicylate functional group. To better understand how chemical similarity relates to receptor activation without relying on incomplete and sometimes arbitrary chemical descriptors, we used a previously validated computational autoencoder to represent each chemical in a ˜292 dimensional latent space, allowing nearly lossless compression of chemical structure (Data not shown). We find chemicals that activate the same OR tend to cluster distinctly (FIG. 10D, FIG. 20). For example, MOR5-1 ligands cluster in latent space, and shows that 10/13 odorants that are long chain (>5 carbons) aldehydes and carboxylic acids activate the receptor. In addition MOR170-1 exhibits a broad activation pattern: binding ˜50% of all odorants containing a benzene ring and either a carbonyl or ether group, and this pattern is also reflected in the latent space. Many, but not all of the receptors. The activation landscape for the entire set of interactions suggest that some ORs are activated by disconnected chemical subspaces (FIG. 20). Understanding the space of chemicals that activates each OR establishes the groundwork for prediction of novel odorant-OR interactions.

Our incomplete understanding for how chemicals, whether they be endogenous ligands, drugs, natural products, or odors, interact with potential targets limits our ability to rationally develop new with the multitude of possible targets and functional pathways is challenging because a particular chemical can interact with multiple targets. This is becoming increasingly apparent in both natural and therapeutic contexts. We anticipate that Smell-seq can be scaled to the 396-member human OR repertoire and comprehensively define OR response to any odorant. The approximate cost per well for Smell-seq is on par with existing assays but multiplexing dramatically reduces cost and labor per interaction interrogated. Efforts to more selectively hit particular targets or broadly activate sets of receptors utilize machine learning methods that rely on massive datasets. Multiplex methods like Smell-seq offer a scalable solution to generate quality data of this magnitude.

### Tables

TABLE 2
Olfactory receptors screened in this study

| | | | |
|---|---|---|---|
| MOR102-1 | MOR20-1 | MOR168-1 | MOR134-1 |
| MOR110-1 | MOR203-1 | MOR169-1 | MOR136-1 |
| MOR112-1 | MOR206-1 | MOR170-1 | MOR139-1 |
| MOR119-1 | MOR208-1 | MOR18-1 | MOR142-1 |
| MOR120-1 | MOR23-1 | MOR180-1 | MOR144-1 |
| MOR13-1 | MOR25-1 | MOR189-1 | MOR149-1 |
| MOR131-1 | MOR30-1 | MOR19-1 | MOR158-1 |
| MOR132-1 | MOR35-1 | MOR194-1 | MOR165-1 |
| MOR133-1 | MOR4-1 | MOR199-1 | MOR9-1 |
| MOR8-1 | MOR5-1 | Olfr62/ | |
| | | MOR258-5 | |

TABLE 3
Odorants screened in this study

| | | | |
|---|---|---|---|
| Pentanoic Acid | Hexanoic Acid | 1-nonanol | Nonanal |
| 4-hydroxycoumarin | Dimedone | 1-decanol | Decanal |
| 4-Chromanone | (−)-Menthone | (+)-2-Heptanol | Citral |
| 2-Butanone | beta-ionone | (+)-2-Octanol | Hydroxycitronellal |
| 2-Hexanone | Pentyl acetate | (−)-B-Citronellol | Lyral |
| 2-Heptanone | Allyl heptanoate | Geraniol | Acetophenone |
| 3-Heptanone | Amyl hexanoate | Linalool | Control_1 |
| 2-Octanone | Nonanoic Acid | 1-Undecanol | Control_2 |
| 3-Octanone | Amyl butyrate | Allyl phenylacetate | Decanoic_Acid |
| Propionic Acid | Butyl heptanoate | Benzene | DMSO |
| 2_coumaranone | Heptyl isobutyrate | Benzyl acetate | Prenyl_Acetate |
| 2-Nonanone | Hexyl acetate | Phenyl acetate | Vanillic_Acid |
| 2,3-Hexanedione | Butyl formate | Octanethiol | a-Amylcinnamaldehyde |
| 3,4-Hexanedione | Ethyl isobutyrate | Nonanedioic Acid | Eucalyptol |
| (−)-Carvone | 1-butanol | Nonanethiol | Pentyl propionate |
| | | | (Amyl propionate) |
| (+)-Dihydrocarvone | Isovaleric Acid | Butanal | Dihydro Myrcenol |
| (+)-Camphor | 1-propanol | Pentanal | Muscenone |
| Dihydrojasmone | 1-hexanol | Hexanal | ethyl maltol |
| Benzophenone | 1-heptanol | Heptanal | calone |
| (+)-Pulegone | 1-octanol | Octanal | Sandalwood Mysone |
| Iso E Super | w-Pentadecalactone | benzyl benzoate | Ethyl 2-methylbutyrate |
| | | (Pentamethylbenzaldehyde) | |
| Olibanum Coeur MD | 2-Phenylethanol | Piperonyl alcohol | trans-2-Dodecenal |
| Turkish Rose Oil | 2-Phenethyl acetate | Piperonyl acetate | Cedryl acetate |
| Angel Eau de parfum (10 uM) | Piperonal | Tetrahydrofuran | l-Octen-3-one |
| a-Hexylcinnamaldehyde | Pyrazine | Tetrahydropyran | 2-Bromohexanoic acid |
| Dior Jadone Eau de | Sassafras oil | Benzaldehyde dimethyl | 6-Bromohexanoic acid |
| parfum | | acetal | |
| Flowerbomb Viktor and | thymol | Â 2-Methyl-1-propanethiol | 2-Bromooctanoic acid |
| Rolf | | | |
| Chanel No 5 | Triethylamine | (+)-Dihydrocarveol | Furfuryl methyl disulfide |
| Axe | L-Turpentine | (−)-Dihydrocarveol | Ethyl isovalerate |
| Aedione | Anisaldehyde | (+)-Perillaaldehyde | Bis(2-methyl-3-furyl)disulphide) |
| Isobornyl acetate | [Di]ethyl sulfide | (−)-Perillaaldehyde | Dimethyl trisulfide |
| a-Amylcinnamaldehyde | Eugenol | Benzyl salicylate | trans-2,cis-6-Nonadienal |
| dimethyl acetal | | | |
| p-Tolyl isobutyrate | Eugenol methyl ether | (+)-Limonene oxide, | trans-2-Nonenal |
| | | mixture of cis and trans | |
| o-Tolyl isobutyrate | 4-Ethylphenol | (−)-Limonene oxide, | Cinnamyl alcohol |
| | | mixture of cis and trans | |
| p-Tolyl phenylacetate | Ethyl vanillin | (R)-(+)-Limonene | n-Decyl acetate |
| 2-Methoxy-3-Methyl-pyrazine | Vanillin | (−)-Camphene | Dimethyl anthranilate |
| 2-Methoxypyrazine | 2-Ethylphenol | (+)-Camphene | trans-2-Undecenal |
| Methyl salicylate | Guaiacol | 2,3-Diethyl-5-methylpyrazine | Neryl isobutyrate |
| Anethole | 2-bromophenol | Ethyl disulfide | cis-4-Decenal |
| Myrcene | Benzaldehyde | Methyl disulfide | Octyl formate |
| (Â±)-2-Butanol | 2,3-Diethylpyrazine | trans-2-Methyl-2-butenal | p-cymene |
| | | (2MB) | |
| 2-Isopropyl-3-methoxypyrazine | 2-Methylbutyric | diacetyl | helional |
| | acid | | |
| 2-sec-Butyl-3-methoxypyrazine | Cyclobutanecarboxylic | galaxolide | 1,9-nonanediol |
| | acid | | |
| cis-6-Nonenal | Isopentylamine | isobutyraldehyde | octanedioic acid |
| | (1-Amino-3-methylbutane, | | (suberic acid) |
| | Isoamylamine) | | |
| Cinnamaldehyde | Quinoline | Ethyl 2-methylpentanoate | decanedioic acid |
| | (1-Benzazine; | | (sebacic acid) |
| | 2,3-Benzopyridine) | | |
| beta-Damascone | Farnesene | e,b,Farnesene | Anisole (Methoxybenzene, |
| | | | Methyl phenyl ether) |

TABLE 4
Odorant-receptor pairs called as hits

| | | Minimum |
|---|---|---|
| | | Activating |
| | | Concentration |
| OR | Odorant | (uM) |
| MOR102-1 | Cedryl acetate | 1000 |
| MOR112-1 | Benzaldehyde | 1000 |
| MOR112-1 | galaxolide | 100 |
| MOR119-1 | Axe (10 uM) | 1000 |
| MOR119-1 | Furfuryl methyl disulfide | 1000 |
| MOR119-1 | n-Decyl acetate | 100 |
| MOR120-1 | Cedryl acetate | 1000 |
| MOR120-1 | Lyral | 1000 |
| MOR120-1 | Nonanethiol | 1000 |
| MOR13-1 | Benzaldehyde | 1000 |
| MOR13-1 | Cyclobutanecarboxylic acid | 1000 |
| MOR13-1 | Pentanoic Acid | 1000 |
| MOR13-1 | trans-2-Methyl-2-butenal (2MB) | 1000 |
| MOR131-1 | (−)-Perillaaldehyde | 1000 |
| MOR131-1 | 1-hexanol | 1000 |
| MOR131-1 | 3,4-Hexanedione | 1000 |
| MOR131-1 | galaxolide | 1000 |
| MOR132-1 | Cedryl acetate | 1000 |
| MOR133-1 | 3-Octanone | 1000 |
| MOR134-1 | Chanel No 5 (10 uM) | 1000 |
| MOR136-1 | (−)-Dihydrocarveol | 1000 |
| MOR136-1 | (+)-Camphor | 100 |
| MOR136-1 | (+)-Dihydrocarveol | 1000 |
| MOR136-1 | 2-Ethylphenol | 100 |
| MOR136-1 | Olibanum Coeur MD | 1000 |
| MOR139-1 | (−)-Dihydrocarveol | 1000 |
| MOR139-1 | (+)-Dihydrocarvone | 1000 |
| MOR139-1 | (+)-Pulegone | 1000 |
| MOR139-1 | 2-sec-Butyl-3-methoxypyrazine | 1000 |
| MOR139-1 | 4-Chromanone | 1000 |
| MOR139-1 | beta-ionone | 1000 |
| MOR139-1 | Butanal | 1000 |
| MOR139-1 | Dihydrojasmone | 1000 |
| MOR139-1 | Dimethyl anthranilate | 1000 |
| MOR139-1 | Eugenol | 1000 |
| MOR139-1 | Eugenol methyl ether | 1000 |
| MOR139-1 | helional | 1000 |
| MOR139-1 | Neryl isobutyrate | 1000 |
| MOR139-1 | Quinoline | 100 |
| | (1-Benzazine; 2,3-Benzopyridine) | |
| MOR142-1 | Bis(2-methyl-3-furyl)disulphide) | 1000 |
| MOR142-1 | Cedryl acetate | 1000 |
| MOR158-1 | Iso E Super | 1000 |
| MOR165-1 | decanedioic acid (sebacic acid) | 1000 |
| MOR165-1 | Octyl formate | 1000 |
| MOR170-1 | 2-Bromohexanoic acid | 1000 |
| MOR170-1 | 2-Phenethyl acetate | 1000 |
| MOR170-1 | 4-Chromanone | 100 |
| MOR170-1 | 4-Ethylphenol | 1000 |
| MOR170-1 | Anisaldehyde | 1000 |
| MOR170-1 | Benzyl acetate | 1000 |
| MOR170-1 | benzyl benzoate | 10 |
| | (Pentamethylbenzaldehyde) | |
| MOR170-1 | Chanel No 5 (10 uM) | 1000 |
| MOR170-1 | Cinnamyl alcohol | 1000 |
| MOR170-1 | Dimethyl anthranilate | 10 |
| MOR170-1 | ethyl maltol | 1000 |
| MOR170-1 | Eugenol methyl ether | 10 |
| MOR170-1 | helional | 1000 |
| MOR170-1 | Piperonal | 1000 |
| MOR170-1 | Piperonyl acetate | 1000 |
| MOR170-1 | Quinoline | 100 |
| | (1-Benzazine; 2,3-Benzopyridine) | |
| MOR170-1 | Vanillin | 1000 |
| MOR180-1 | a-Amylcinnamaldehyde | 1000 |
| | dimethyl acetal | |
| MOR180-1 | Axe (10 uM) | 1000 |
| MOR189-1 | 4-Chromanone | 1000 |
| MOR189-1 | benzyl benzoate | 1000 |
| | (Pentamethylbenzaldehyde) | |
| MOR189-1 | beta-Damascone | 1000 |
| MOR189-1 | beta-ionone | 1000 |
| MOR189-1 | Cedryl acetate | 1000 |
| MOR189-1 | Eugenol methyl ether | 1000 |
| MOR189-1 | Quinoline | 1000 |
| | (1-Benzazine; 2,3-Benzopyridine) | |
| MOR 19-1 | Benzyl salicylate | 10 |
| MOR 19-1 | Methyl salicylate | 1000 |
| MOR 199-1 | ethyl maltol | 100 |
| MOR203-1 | helional | 1000 |
| MOR203-1 | Piperonyl acetate | 1000 |
| MOR208-1 | Cedryl acetate | 1000 |
| MOR23-1 | 2-Bromooctanoic acid | 1000 |
| MOR23-1 | 6-Bromohexanoic acid | 100 |
| MOR23-1 | Heptanal | 1000 |
| MOR23-1 | Hexanoic Acid | 1000 |
| MOR23-1 | Nonanal | 1000 |
| MOR23-1 | Nonanoic Acid | 1000 |
| MOR23-1 | Octanal | 100 |
| MOR25-1 | (−)-Carvone | 1000 |
| MOR25-1 | Decanal | 1000 |
| MOR25-1 | Decanoic-Acid | 100 |
| MOR25-1 | Nonanoic Acid | 1000 |
| MOR30-1 | Cedryl acetate | 1000 |
| MOR30-1 | Decanal | 100 |
| MOR30-1 | Decanoic-Acid | 10 |
| MOR30-1 | Nonanal | 1000 |
| MOR30-1 | Nonanoic Acid | 100 |
| MOR4-1 | Hexanoic Acid | 1000 |
| MOR4-1 | Pentanoic Acid | 1000 |
| MOR5-1 | 2-Bromohexanoic acid | 1000 |
| MOR5-1 | 2-Bromooctanoic acid | 1000 |
| MOR5-1 | 6-Bromohexanoic acid | 1000 |
| MOR5-1 | cis-4-Decenal | 1000 |
| MOR5-1 | cis-6-Nonenal | 1000 |
| MOR5-1 | Decanoic-Acid | 1000 |
| MOR5-1 | Hexanoic Acid | 1000 |
| MOR5-1 | Nonanal | 1000 |
| MOR5-1 | Nonanoic Acid | 100 |
| MOR5-1 | Octanal | 1000 |
| MOR5-1 | Olibanum Coeur MD | 1000 |
| Olfr62 | 2-coumaranone | 1000 |
| Olfr62 | Benzaldehyde | 1000 |
| Olfr62 | Benzophenone | 1000 |
| Olfr62 | ethyl maltol | 1000 |
| Olfr62 | Piperonal | 1000 |
| Olfr62 | Quinoline | 1000 |
| | (1-Benzazine; 2,3-Benzopyridine) | |
| MOR9-1 | galaxolide | 1000 |

TABLE 5
Primers and Sequences Used in This Study

| | SEQ ID | | |
|---|---|---|---|
| Primer | NO: | Sequence | Description |
| OL001 | 25 | CCCTTTAATCAGATGCGT | Gene Specific RT, Reporter Gene, |
| | | CG | for Q-RTPCR |
| OL002 | 25 | CTGCCTGCTTCACCACCT | Gene Specific RT, GAPDH |
| | | TC | |
| OL003 | 27 | AAGTGCCTTCCTGCCCTT | Gene Specific RT, Reporter Gene, |
| | | TAATCAGATGCGTCG | for RNA-seq, Also NGS Read1 |
| | | | Primer |
| OL004F | 28 | CGCCGAAGTGAAAACCA | Pilot-Scale RNA-seq Round 1 |
| | | CCTA | Library Prep Amplification |
| OL004R | 29 | AAGTGCCTTCCTGCCCTT | Pilot-Scale RNA-seq Round 1 |
| | | TAA | Library Prep Amplification |
| OL005F | 30 | CAAGCAGAAGACGGCAT | P7 + i7index + primer for RNAseq |
| | | ACGAGAT NNNNNNNN | library amplification |
| | | CGAAGTGAAAACCACCT | |
| | | A | |
| OL005R | 31 | AATGATACGGCGACCAC | P5 + Read1 + primer for pilot-scale |
| | | CGAGATCTACACAAGTG | RNAseq library amplification |
| | | CCTTCCTGCCCTTTAA | |
| OL006 | 32 | CGGGTTTCTTGGCCTTGT | i7 index read primer, pilot-scale |
| | | AGGTGGTTTTCACTTCG | experiment |
| OL007F | 33 | ggaataACGCGTNNNNNNN | Amplification of fragment |
| | | NNNNNNNNCGACGCATC | containing barcode to be cloned |
| | | TGATTAAAGGG | into reporter plasmid |
| OL007R | 34 | ggaaggACCGGTtctagtcaaggc | Amplification of fragment |
| | | actatacat | containing barcode to be cloned |
| | | | into reporter plasmid |
| OL008F | 35 | tgctcctggccctgctgaccctaggcctg | Amplification of fragment |
| | | gctCATATGAATGGCACAG | containing the OR to be cloned |
| | | AAGGCCC | into the reporter plasmid |
| OL008R | 36 | AGTCGGCCCTGCTGAGG | Amplification of fragment |
| | | AGTCTTTCCACCTGCAGG | containing the OR to be cloned |
| | | TCTTATCATGTCTGCTCG | into the reporter plasmid |
| | | AA | |
| OL009 | 37 | CTTCTACGTGCCCTTCTC | Sequencing and linking |
| | | | barcodes/ORs in the reporter |
| | | | vector |
| OL010 | 38 | CCTGCAGGTCTTATCATG | Sequencing and linking |
| | | TC | barcodes/ORs in the reporter |
| | | | vector |
| OL011 | 39 | TACAGGCGGAATGGACG | Sequencing and linking |
| | | AG | barcodes/ORs in the reporter |
| | | | vector |
| OL012F | 40 | AAGTGAAAACCACCTAC | QPCR of the transposon for copy |
| | | AAGG | number analysis |
| OL012R | 41 | CCCTTTAATCAGATGCGT | QPCR of the transposon for copy |
| | | CG | number analysis |
| OL013 | 42 | AATGATACGGCGACCAC | P5 + i5 + Read1 + primer, for large- |
| | | CGAGATCTACAC | scale library amplification |
| | | NNNNNNNN | |
| | | AAGTGCCTTCCTGCCCTT | |
| | | TAA | |
| LP001F | 43 | TGGGCAGTTCCAGGCTTA | Genomic Amplification of the H11 |
| | | TAGTC | locus with the landing pad |
| LP001R | 44 | GGGCGTACTTGGCATATG | Genomic Amplification of the H11 |
| | | ATACAC | locus with the landing pad |

List of indices used for Pilot-Scale Screen (i7)

| | SEQ ID | |
|---|---|---|
| Name | NO: | Index |
| TBSC01 | 45 | ATCACG |
| TBSC02 | 46 | CGATGT |
| TBSC03 | 47 | TTAGGC |
| TBSC04 | 48 | TGACCA |
| TBSC05 | 49 | ACAGTG |
| TBSC06 | 50 | GCCAAT |
| TBSC07 | 51 | CAGATC |
| TBSC08 | 52 | ACTTGA |
| TBSC09 | 53 | GATCAG |
| TBSC10 | 54 | TAGCTT |
| TBSC11 | 55 | GGCTAC |
| TBSC12 | 56 | CTTGTA |
| TBSC13 | 57 | AGTCAA |
| TBSC14 | 58 | AGTTCC |
| TBSC15 | 59 | ATGTCA |
| TBSC16 | 60 | CCGTCC |
| TBSC17 | 61 | GTAGAG |
| TBSC18 | 62 | GTCCGC |
| TBSC19 | 63 | GTGAAA |
| TBSC20 | 64 | GTGGCC |
| TBSC21 | 65 | GTTTCG |
| TBSC22 | 66 | CGTACG |
| TBSC23 | 67 | GAGTGG |
| TBSC24 | 68 | GGTAGC |
| TBSC25 | 69 | ACTGAT |
| TBSC26 | 70 | ATGAGC |
| TBSC27 | 71 | ATTCCT |
| TBSC28 | 72 | CAAAAG |
| TBSC29 | 73 | CAACTA |
| TBSC30 | 74 | CACCGG |
| TBSC31 | 75 | CACGAT |
| TBSC32 | 76 | CACTCA |
| TBSC33 | 77 | CAGGCG |
| TBSC34 | 78 | CATGGC |

List of Indices Used for Large-Scale Odorant Screen

| | SEQ ID | | |
|---|---|---|---|
| Well | NO: | Plate | SEQ ID NO: |
| Index 1 (i7 | | Index 2 | |
| side) | | (i5 side) | |
| CCTGCGA | 79 | CTCTCTAT | 80 |
| TGCAGAG | 81 | TATCCTCT | 82 |
| ACCTAGG | 83 | GTAAGGAG | 84 |
| TTGATCC | 85 | ACTGCATA | 86 |
| ATCTTGC | 87 | AAGGAGTA | 88 |
| TCTCCAT | 89 | CTAAGCCT | 90 |
| CATCGAG | 91 | CGTCTAAT | 92 |
| TTCGAGC | 93 | TCTCTCCG | 94 |
| AGTTGGT | 95 | CTAGTCGA | 96 |
| GTACCGG | 97 | AGCTAGAA | 98 |
| CGGAGTT | 99 | ACTCTAGG | 100 |
| ACTTCAA | 101 | TCTTACGC | 102 |
| TGATAGT | 103 | CTTAATAG | 104 |
| GATCCAA | 105 | | |
| CAGGTCG | 106 | | |
| CGCATTA | 107 | | |
| GGTACCT | 108 | | |
| GGACGCA | 109 | | |
| GAGATTC | 110 | | |
| GAGCATG | 111 | | |
| GTTGCGT | 112 | | |
| CCAATGC | 113 | | |
| CGAGATC | 114 | | |
| CATATTG | 115 | | |
| GACGTCA | 116 | | |
| TGGCATC | 117 | | |
| GTAATTG | 118 | | |
| CCTATCT | 119 | | |
| CAATCGG | 120 | | |
| GCGGCAT | 121 | | |
| AGTACTG | 122 | | |
| TACTATT | 123 | | |
| CCGGATG | 124 | | |
| ACCATGA | 125 | | |
| CGGTTCT | 126 | | |
| TATTCCA | 127 | | |
| CCTCCTG | 128 | | |
| AGGTATT | 129 | | |
| GCATTCG | 130 | | |
| TTGCGAA | 131 | | |
| TTGAATT | 132 | | |
| CTGCGCG | 133 | | |
| AGACCTT | 134 | | |
| GTCCAGT | 135 | | |
| ACCTGCT | 136 | | |
| CCGGTAC | 137 | | |
| CTTGACC | 138 | | |
| CATCATT | 139 | | |
| TCTGACT | 140 | | |
| TCTAGTT | 141 | | |
| GCCATAG | 142 | | |
| ACCGTCG | 143 | | |
| CTTGGTT | 144 | | |
| TACGCCG | 145 | | |
| GGACTGC | 146 | | |
| GCGCGAG | 147 | | |
| GTCGCAG | 148 | | |
| CATACGT | 149 | | |
| TCAGTAT | 150 | | |
| CTAAGTA | 151 | | |
| TTAGCTT | 152 | | |
| CGCCGTC | 153 | | |
| GTCTTCT | 154 | | |
| GCCGGAC | 155 | | |
| AAGCTGA | 156 | | |
| GCGCTCT | 157 | | |
| CGTAGGC | 158 | | |
| ATGATTA | 159 | | |
| GCAGGTT | 160 | | |
| AATCGTC | 161 | | |
| CGGCCTA | 162 | | |
| CTATGCC | 163 | | |
| GGTTGAA | 164 | | |
| GAGTTAA | 165 | | |
| TAGACTA | 166 | | |
| TCATGCA | 167 | | |
| GCTTATT | 168 | | |
| CAAGGCT | 169 | | |
| AGGTTGG | 170 | | |
| CTTCTGC | 171 | | |
| TAATTCT | 172 | | |
| GATGCTG | 173 | | |
| CCTAGAA | 174 | | |
| CTAGAGG | 175 | | |
| TATCCGG | 176 | | |
| AGGCGGC | 177 | | |
| GGTCGTT | 178 | | |
| CCGCTGG | 179 | | |
| GGAACTA | 180 | | |
| ATTGCCA | 181 | | |
| ATATACG | 182 | | |
| GATTAGC | 183 | | |
| AGAAGTC | 184 | | |
| ATAGTAC | 185 | | |
| GATCTCG | 186 | | |
| GGCTGCG | 187 | | |

### Methods

1. Odorant-Receptor Activation Luciferase Assay (Transient)

The Dual-Glo Luciferase Assay System (Promega) was used to measure OR-odorant responses as previously described (Zhuang and Matsunami 2008). HEK293T cells (ATCC #11268) were plated in poly-D-lysine coated white 96-well plates (Corning) at a density of 7,333 cells per well in 100 ul DMEM (Thermo Fisher Scientific). 24 hours later, cells were transfected using lipofectamine 2000 (Thermo Fisher Scientific) with 5 ng/well of plasmids encoding ORs and 10 ng/well of luciferase driven by a cyclic AMP response element or 10 ng/well of a plasmid encoding both the OR and the luciferase gene, and in both cases 5 ng/well of a plasmid encoding Renilla luciferase. Experiments conducted with accessory factors included 5 ng/well of plasmids encoding RTP1S (Gene ID: 132112) and RTP2 (Gene ID: 344892). Inducibly expressed ORs were transfected with 1 ug/ml doxycycline (Sigma-Aldrich) added to the transfection media. 10-100 mM odorant stocks were established in DMSO or ethanol. 24 h after transfection, transfection medium was removed and replaced with 25 ul/well of the appropriate concentration of odorant diluted from the stocks into CD293 (Thermo Fisher Scientific). Four hours after odorant stimulation, the Dual-Glo Luciferase Assay kit was administered according to the manufacturer's instructions. Luminescence was measured using the M1000 plate reader (Tecan). All luminescence values were normalized to Renilla luciferase activity to control for transfection efficiency in a given well. Data were analyzed with Microsoft Excel and R.

2. Odorant-Receptor Activation Luciferase Assay (Integrated)

HEK293T and HEK293T derived cells integrated with the combined receptor/reporter plasmids were plated at a density of 7333 cells/well in 100 uL DMEM in poly-D-lysine coated 96-well plates. 24 hours later, 1 ug/ml doxycycline was added to the well medium. Odorant stimulation, luciferase reagent addition, and luminescence measurements were carried out in the same manner as the transient assays. Constitutively expressed ORs were assayed in the same manner without doxycycline addition. Data were analyzed with Microsoft Excel and R.

3. Odor Stimulation and RNA Extraction for Pilot-Scale Multiplexed Odorant Screening

HEK293T and HEK293T derived cells transposed with the combined receptor/reporter plasmid were plated at a density of 200 k cells/well in a 6 well plate in 2 mL DMEM. 24 hours later, 1 ug/ml doxycycline was added to the well medium. 10-100 mM odorant stocks were established in DMSO or ethanol. 24 hours after doxycycline addition, odorants were diluted in OptiMEM and media was aspirated and replaced with 1 mL of the odorant-OptiMEM solution. 3 hours after odor stimulation, odor media was aspirated and 600 uL of buffer RLT (Qiagen) was added to each well. Cells were lysed with the Qiashredder Tissue and Cell Homogenizer (Qiagen) and RNA was purified using the RNEasy MiniPrep Kit (Qiagen) with the optional on-column DNAse step according to the manufacturer's protocol.

4. Pilot Scale Library Preparation and RNA-Seq

5 ug of total RNA per sample was reverse transcribed with Superscript IV (Thermo-Fisher) using a gene specific primer for the barcoded reporter gene (OL003). The reaction conditions are as follows: annealing: [65° C. for 5 min, 0° C. for 1 min] extension: [52° C. for 60 min, 80° C. for 10 min]0.10% of the cDNA library volumes were amplified for 5 cycles (OL004F and R) using HiFi Master Mix (Kapa Biosystems). The reaction and cycling conditions are optimized as follows: 95° C. for 3 minutes, 5 cycles of 98° C. for 20 seconds, 59° C. for 15 seconds, and 72° C. for 10 seconds, followed by an extension of 72° C. for 1 minute. The PCR products were purified using the DNA Clean & Concentrator kit (Zymo Research) into 10 ul and 1 ul of each sample was amplified (OL005F and R) using the SYBR FAST qPCR Master mix (Kapa Biosystems) with a CFX Connect Thermocycler (Biorad) to determine the number of PCR cycles necessary for library amplification. The reaction and cycling conditions are optimized as follows: 95° C. for 3 minutes, 40 cycles of 95° C. for 3 seconds and 60° C. for 20 seconds. After qPCR, 5 ul of the pre-amplified cDNA libraries were amplified a second time at the same cycling conditions as the first amplification with the same primers used for qPCR for 4 cycles greater than the previously determined Cq. The PCR products were then gel isolated from a 1% agarose gel with the Zymoclean Gel DNA Recovery Kit (Zymo Research). Library concentrations were quantified using a Tape Station 2200 (Agilent) and loaded equimolar onto a Hi-Seq 3000 with a 20% PhiX spike-in and sequenced with custom primers: Read 1 (OL003) and i7 Index (OL006).

5. OR Library Cloning

The backbone plasmid (all genetic elements except the OR and barcode) was created using isothermal assembly with the Gibson Assembly Hifi Mastermix (SGI-DNA). A short fragment was amplified with a primer containing 15 random nucleotides to create the barcode sequence (OL007F and R) using HiFi Master Mix. The reaction and cycling conditions are optimized as follows: 95° C. for 3 minutes, 35 cycles of 98° C. for 20 seconds, 60° C. for 15 seconds, and 72° C. for 20 seconds, followed by an extension of 72° C. for 1 minute. The amplicon and the backbone plasmid were digested with restriction enzymes MluI and AgeI (New England Biolabs) and ligated together with T4 DNA ligase (New England Biolabs). DH5α E. coli competent cells (New England Biolabs) were transformed directly into liquid culture with antibiotic to maintain the diversity of the barcode library.

OR genes were amplified individually with primers (OL008) adding homology to the barcoded backbone plasmid using HiFi Master Mix. The reaction and cycling conditions are optimized as follows: 95° C. for 3 minutes, 35 cycles of 98° C. for 20 seconds, 61° C. for 15 seconds, and 72° C. for 30 seconds, followed by an extension of 72° C. for 1 minute. The amplified ORs were purified with DNA Clean and Concentrator and pooled together. The barcoded backbone plasmid was digested with NdeI and SbfI and the OR amplicon pool was cloned into it using isothermal assembly with the Gibson Assembly Hifi Mastermix. DH5α E. coli competent cells were transformed with the assembly and antibiotic resistant clones were picked and grown up in 96-well plates overnight. The plasmid DNA was prepped with the Zyppy −96 Plasmid Miniprep Kit (Zymo Research). Plasmids were Sanger sequenced (OL109-111) both to associate the barcode with the reporter gene and identify error-free ORs.

6. OR Library Genomic Integration

HEK293T cells and HEK293T derived cells were seeded at a density of 350 k cells/well in a 6-well plate in 2 ml DMEM. 24 hours after seeding, cells were transfected with plasmids encoding receptor/reporter transposon and the Super PiggyBac Transposase (Systems Bioscience) according to the manufacturer's instructions. 1 ug of transposon DNA and 200 ng of transposase DNA were transfected per well with Lipofectamine 3000. 3 days after transfection cells were passaged 1:10 into a 6-well plate and one day after passaging 8 ug/ml blasticidin were added to the cells. Cells were grown with selection for 7-10 days. The OR library was transposed individually and pooled together at equal cell numbers.

7. Accessory Factor Cell Line Generation

HEK293T derived cells were transposed with plasmids encoding the accessory factor genes RTP1S, RTP2, Gα olf (Gene ID: 2774), and Ric8b (Gene ID: 237422) inducibly driven by the Tet-On promoter pooled equimolar according to the transposition protocol in the OR Library Integration section. Cells were selected with 2 ug/ml puromycin (Thermo Fisher). After selection, cells were seeded in a 96-well plate at a density of 0.5 cells/well. Wells were examined for single colonies after 3 days and expanded to 24-well plates after 7 days. Clones were screened for accessory factor expression by screening them for robust activation of Olfr62 and OR7D4 with a transient luciferase assay (FIG. 11). The clone with the highest fold activation for both receptors and no salient growth defects was established for the multiplexed screen.

8. Transposon Copy Number Verification

gDNA was purified from cells transposed with the OR reporter vector and from cells containing the single copy landing pad with the Quick-gDNA Miniprep kit. 50 ng of gDNA was amplified with primers annealing to the regions of the exogenous DNA from each sample using the SYBR FAST qPCR Master Mix (Kapa Biosystems) on a CFX Connect Thermocycler using the manufacturer's protocol. The reaction and cycling conditions are optimized as follows: 95° C. for 3 minutes, 40 cycles of 95° C. for 3 seconds and 60° C. for 20 seconds. Cq values for the transposed ORs were normalized to the single copy landing pad to determine copy number.

9. Lentiviral Transduction

Lentiviral vector was produced by transient transfection of 293T cells with lentiviral transfer plasmid, pCMVAR8.91 and pCAGGS-VSV-G using Mirus TransIT-293. HEK293T cells were transduced to express the m2rtTA transcription factor (Tet-On) at 50% confluency and seeded one day prior to transduction. Clones were isolated by seeding cells in a 96-well plate at a density of 0.5 cells/well. Wells were examined for single colonies after 7 days and expanded to 24 well plates. Clones were assessed for m2rtTA expression by screening for robust activation of MOR42-3 (Gene ID: 257926) with a transient luciferase assay.

10. High-Throughput Odorant Screening

The OR library cell line was thawed from a liquid nitrogen frozen stock into a T-225 flask (Corning) three days before seeding into a 96-well plate for screening. The library was seeded at 6,666 cells per well in 100 ul of DMEM. 24 hours later a working concentration of 1 ug/ml of doxycycline in DMEM was added to the wells. 24 hours after induction, the media was removed from each plate and replaced with 25 ul of odorant diluted in OptiMEM. Each odor was added at three different concentrations (10 uM, 100 uM, 1 mM) in triplicate with the same amount of final DMSO (1%). Each plate contained two control odorants at a three concentration (10 uM, 100 uM, 1 mM) in triplicate and three wells containing 1% DMSO dissolved in media. The library was incubated with odorants for three hours in a cell culture incubator with the lids removed.

After odor incubation, media was pipetted out of the plates and cells were lysed by adding 25 uL of ice-cold Cells-to-cDNA II Lysis Buffer (Thermo Fisher) and pipetting up and down to homogenize and lyse cells. The lysate was then heated to 75° C. for 15 minutes and flash frozen with liquid nitrogen and kept at −80 C until further processing. Then 0.5 uL DNase I (New England Biolabs) was added to lysate, and incubated at 37° C. for 15 minutes. To anneal the RT primer, 5 ul of lysate from each well was combined with 2.5 ul of 10 mM dNTPs (New England Biosciences), 1 ul of 2 uM gene specific RT primer (OL003), and 1.5 ul of H2O. The reaction was heated to 65° C. for 5 min and cooled back down to 0° C. After annealing, 1 ul of M-MuLV Reverse Transcriptase (Enzymatics), 1 ul of buffer, and 0.25 ul of RNase Inhibitor (Enzymatics) were added to each reaction. Reactions were incubated at 42° C. for 60 min and the RT enzyme was heat inactivated at 85° C. for 10 min.

For each batch, qPCR was performed on a few wells (OL005F and OL013) with SYBR FAST qPCR Mastermix to determine the number of cycles necessary for PCR based library preparation. The reaction and cycling conditions are optimized as follows: 95° C. for 3 minutes, 40 cycles of 95° C. for 3 seconds and 60° C. for 20 seconds. After qPCR, 5 ul of each RT reaction was combined with 0.4 ul of 10 uM primers containing sequencing adaptors (OL005F and OL013), 10 ul of NEB-Next Q5 Mastermix (New England Biosciences) and 4.2 ul H2O, the PCR was carried out according to the manufacturer's protocol. The forward primer contains the P7 adaptor sequence and an index identifying the well in the assay and the reverse primer contains the P5 adaptor sequence and an index identifying the plate in the assay. PCR products were pooled together by plate and purified with the DNA Clean and Concentrator Kit. Library concentrations were quantified using a Tape Station 2200 and a Qubit (Thermo Fisher). The libraries were sequenced with two index reads and a single end 75-bp read on a NextSeq 500 in high-output mode (Illumina).

11. Analysis of Next-Generation Sequencing Data

Samples were identified via indexing by their PCR indexes adapters unique for each well (5′ end) and unique for each plate (3′ end). The well barcodes followed the 7 bp indexing scheme in (Illumina Sequencing Library Preparation for Highly Multiplexed Target Capture and Sequencing Matthias Meyer, Martin Kircher, Cold Spring Harb Protoc; 2010; doi:10.1101/pdb.prot5448). The plate indexing scheme followed the Illumina indexing scheme. Sequencing data was demultiplexed and 15 bp barcode sequences were counted with only exact matches by custom python and bash scripts.

12. Statistical Methods for Calling Hits

Count data was then analyzed using the differential expression package EdgeR. To filter out ORs with low representation, we set a cutoff that an OR had to contain at least 0.5% of the reads from more than 399 of the 1954 test samples. This filtered out 3 of 42 ORs which were underrepresented in the cell library (MOR172-1, MOR176-1 and MOR181-1). Normalization factors were determined using the EdgeR package function calcNormFactors, and glmFit was used with the dispersion set to the tagwise dispersion since only 40 ORs were present in the library and trended dispersion values did fit the data well. By fitting a generalized linear model to the count data to determine if odorants stimulated specific ORs, we were able to determine both the mean activation for each OR-odorant interaction and the p-value. We then corrected this p-value for multiple hypothesis testing using the built in p.adjust function with the Benjamini & Hochberg correction yielding a False Discovery Rate (FDR). We set a conservative cutoff of 1% to determine interacting odorant-OR pairs. For each interaction between an odorant and an OR, we further required that an OR-odorant interaction was beyond the cutoff in two different concentrations of odorant or in just the 1000 uM concentration.

13. Molecular Autoencoder

We used an autoencoder as described in Gómez-Bombarelli et al. to visualize OR-chemical interactions in the context of chemical space. Following the authors advice, we used a reimplementation of autoencoder as the original implementation requires a defunct Python package. This model comes pre-trained to a validation accuracy of 0.99 on the entire ChEMBL 23 database with the exception of molecules whose SMILES are longer than 120 characters. We used this pretrained model to generate the latent representations of both our 168 chemicals (for which we could find SMILES representations) and 250,000 randomly sampled chemicals from ChEMBL 23. We then used scikit-learn to perform principal component analysis to project the resulting matrix onto two dimensions.

### Example 3—ADRB2 Variant Screen

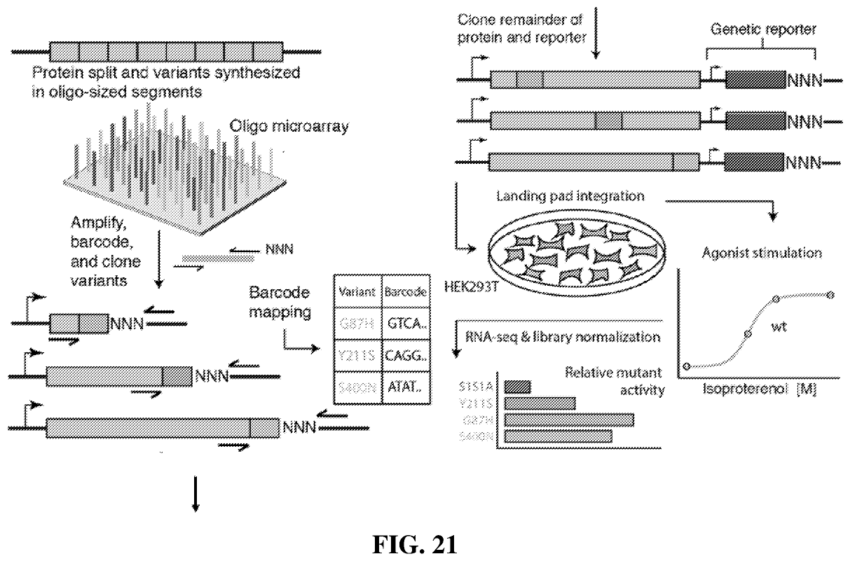

Overview of creation and functional assessment of the mutant library. We synthesize the mutant sequences on oligonucleotide microarrays, however the length limit for each oligo is ˜230 nt and ADRB2 is ˜1200 nt long. To cover the length of the protein we had to segment it into 8 parts, synthesize each mutant eighth and clone into a separate background vectors. When amplifying and cloning the variant segment, we attached a 15 nt random barcode to each sequence. Upon cloning, we mapped each barcode to each variant with next-gen sequencing. Afterwards, we cloned in the remainder of the protein and translocated the barcode to the 3′ UTR of a cyclic AMP Response Element (CRE) reporter gene that expresses upon Gs signaling. From there, we integrated the library at a defined genomic locus in AADRB2 HEK293T cells at single copy per cell (essential to prevent crosstalk between mutants in the multiplexed assay) using serine recombinase technology. After integration, we stimulated the library cell line with various isoproterenol concentrations and performed RNA-seq on the barcode sequences. The relative abundance of each barcode can be inferred as the relative activity of each B2 variant after normalization for representation. This is shown in FIG. 21.

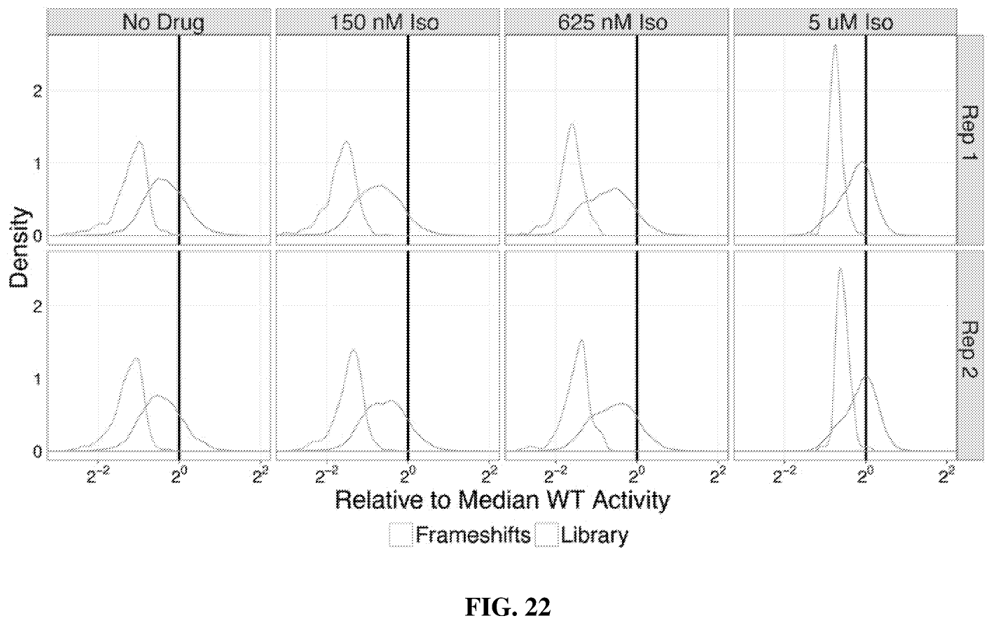

In FIG. 22, we show the the distributions activity relative to the median wild-type signal for both frameshifts (a common error mode of oligonucleotide microarray synthesis) and our single mutant library across two biological replicates. To build our variant distribution, we average the measurements of every barcode associated with a given variant. To build the frameshift distribution, we average the measurements of every barcode associated with an indel at a particular codon (excluding the C-terminus). As expected, frameshifts have a more deleterious effect than the average missense mutation. We also see that at high Isoproterenol concentrations, a higher proportion of our missense mutations approach wild-type levels of activity.

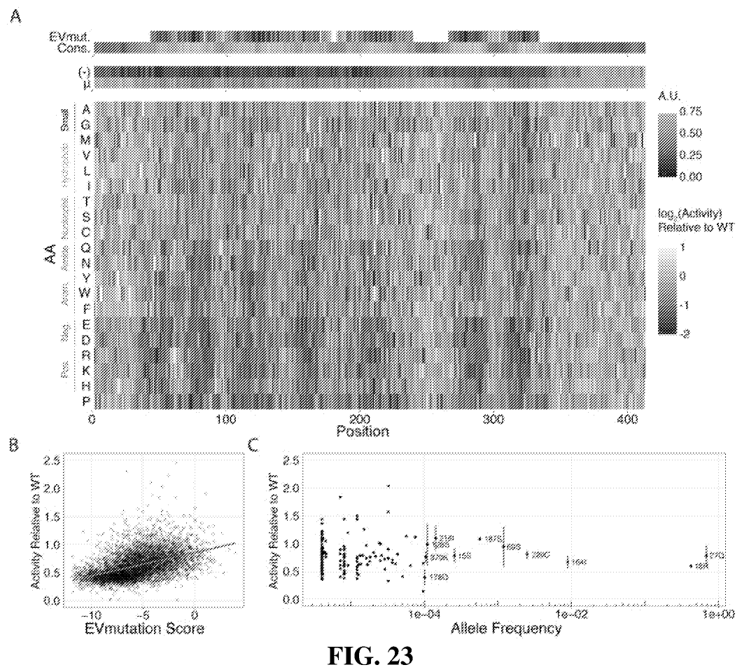

In FIG. 23 we show the variant activity landscape for β2 at 0.625 uM Isoproterenol. The mutational landscape reveals general trends of β2 structure and function. For example, we see that transmembrane domains are more sensitive to proline and charged residue substitutions than the termini or intracellular loop 3 (mutational tolerance is the average effect of all mutations). We also see that the effects of frameshifts are greatly diminished in the C-terminus. We see mutational data is correlated with EV mutation Score and we can also see how rare variants affect function from GNOMAD data.

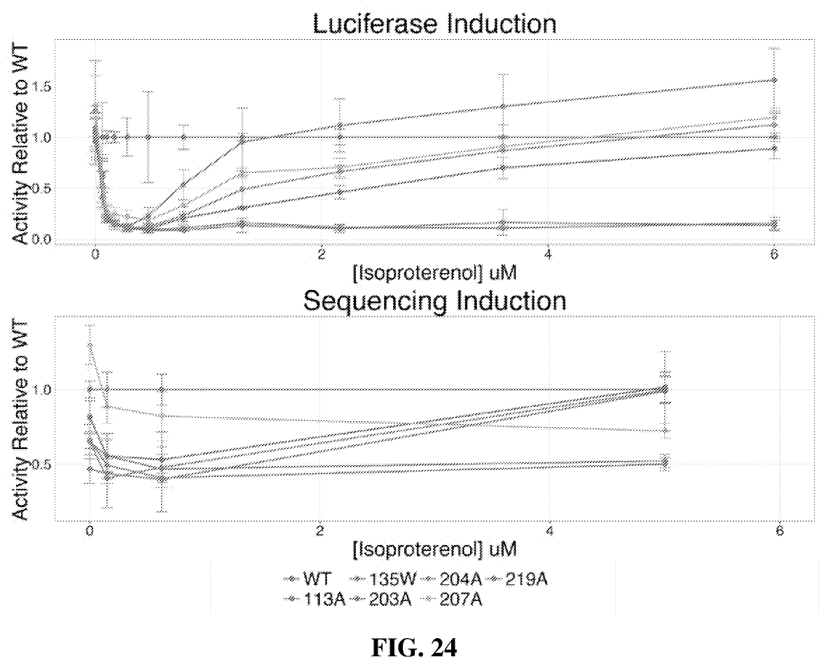

In FIG. 24 we show the comparison between missense variants assayed individually with a luciferase reporter compared to the multiplexed sequencing approach. Mutant activity relative to WT is mostly recapitulated. The multiplexed assay can distinguish between completely dead mutants and partially deleterious mutants over the range of isoproterenol stimulation.

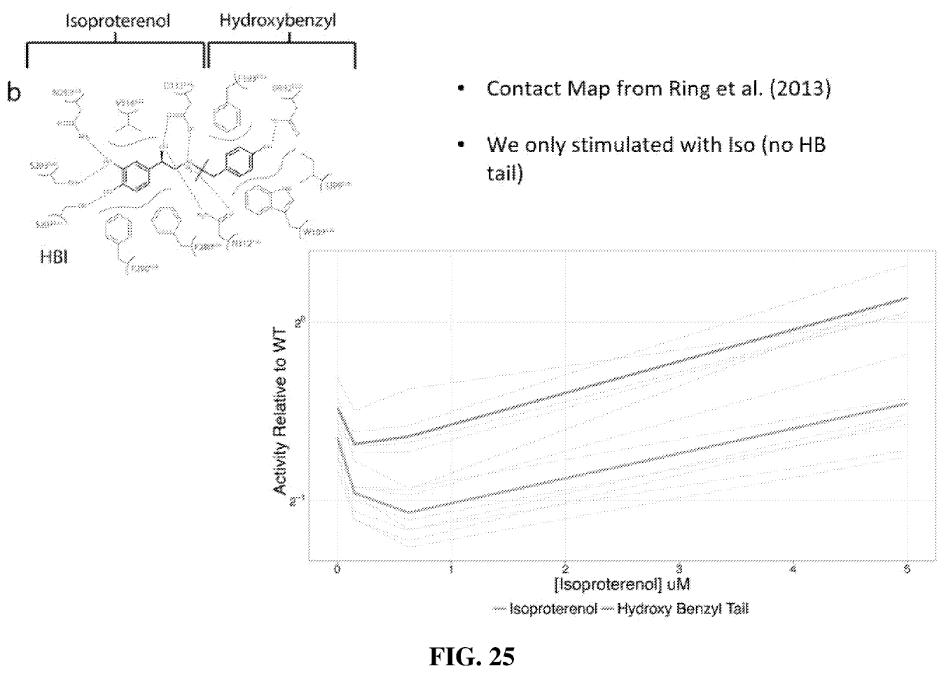

We looked at the mutational tolerance (avg. of all substitutions) of the ligand binding pocket of β2 as annotated from Ring et al.'s contact map of Hydroxybenzyl Isoproterenol with the receptor. In our assay, we stimulated solely with isoproterenol, and we see that mutations to the residues interacting with isoproterenol are significantly less tolerant to mutation relative to residues interacting with the hydroxybenzyl tail. This is shown in FIG. 25.

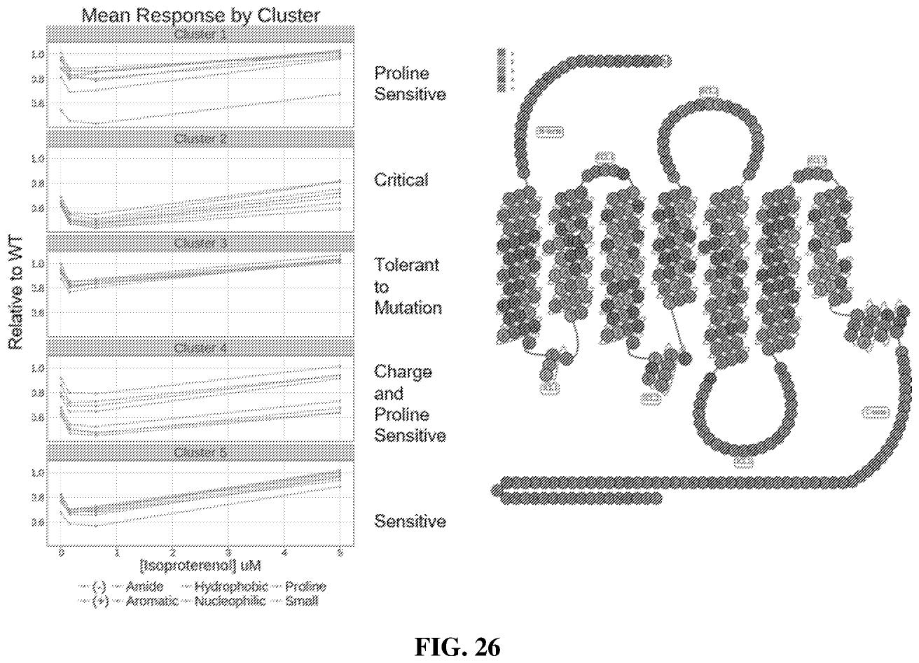

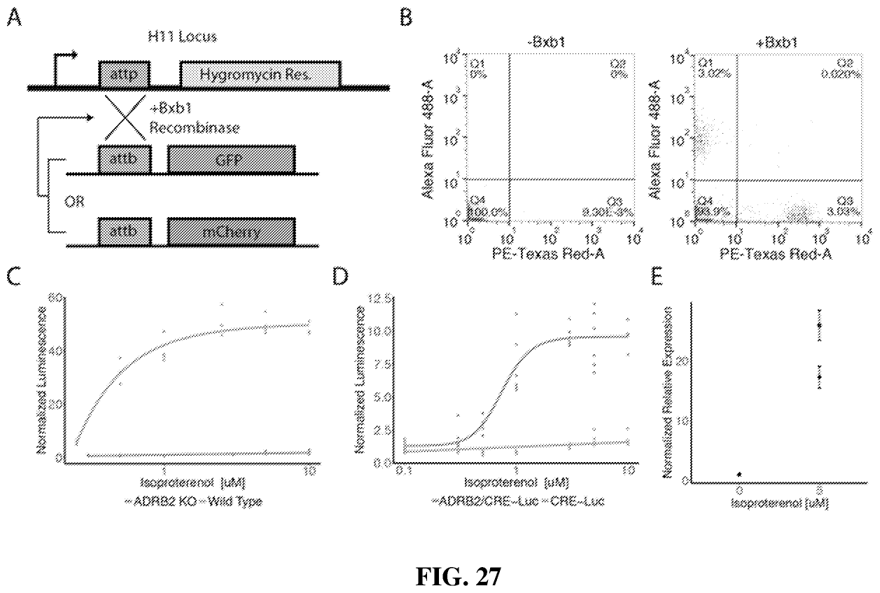

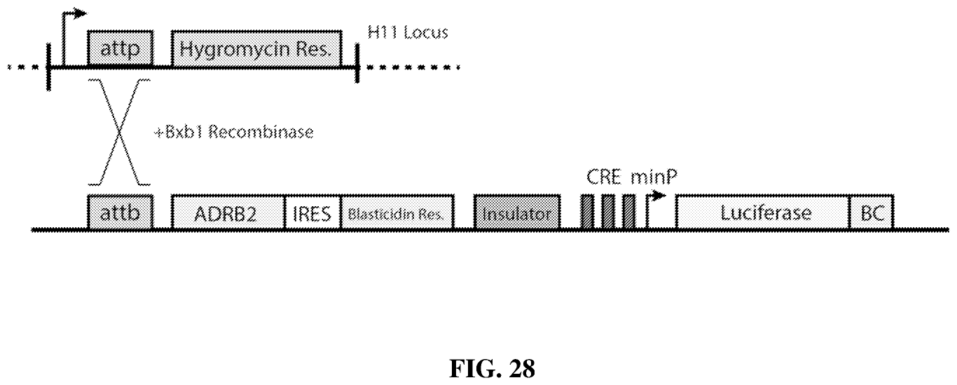

We also found that that simple algorithms such as k-means clustering could group our data into distinct classes that map onto the structure of β2 in a functionally relevant manner. In this specific example, we grouped the amino acid mutations together into functional classes and averaged their signal. Importantly, we did not provide any spatial information to the algorithm. We believe that future deep mutational scans could be a powerful method to investigate protein structure. This is shown in FIG. 26.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references and the publications referred to throughout the specification, to the extent that they provide exemplary procedural or other details supplementary to those set forth herein, are specifically incorporated herein by reference.
1. Roth, B. L., Sheffler, D. J. & Kroeze, W. K. Magic shotguns versus magic bullets: selectively non-selective drugs for mood disorders and schizophrenia. Nat. Rev. Drug Discov. 3, 353-359 (2004).
2. Reddy, A. S. & Zhang, S. Polypharmacology: drug discovery for the future. Expert Rev. Clin. Pharmacol. 6, 41-47 (2013).
3. Fang, J., Liu, C., Wang, Q., Lin, P. & Cheng, F. In silico polypharmacology of natural products. Brief. Bioinform. (2017). doi: 10.1093/bib/bbx045
4. Anighoro, A., Bajorath, J. & Rastelli, G. Polypharmacology: challenges and opportunities in drug discovery. J. Med. Chem. 57, 7874-7887 (2014).
5. Malnic, B., Hirono, J., Sato, T. & Buck, L. B. Combinatorial receptor codes for odors. Cell 96, 713-723 (1999).
6. Buck, L. & Axel, R. A novel multigene family may encode odorant receptors: a molecular basis for odor recognition. Cell 65, 175-187 (1991).
7. Hauser, A. S., Attwood, M. M., Rask-Andersen, M., Schioth, H. B. & Gloriam, D. E. Trends in GPCR drug discovery: new agents, targets and indications. Nat. Rev. Drug Discov. 16, 829-842 (2017).
8. Niimura, Y., Matsui, A. & Touhara, K. Extreme expansion of the olfactory receptor gene repertoire in African elephants and evolutionary dynamics of orthologous gene groups in 13 placental mammals. Genome Res. 24, 1485-1496 (2014).
9. Peterlin, Z., Firestein, S. & Rogers, M. E. The state of the art of odorant receptor deorphanization: a report from the orphanage. J. Gen. Physiol. 143, 527-542 (2014).
10. Lu, M., Echeverri, F. & Moyer, B. D. Endoplasmic reticulum retention, degradation, and aggregation of olfactory G-protein coupled receptors. Traffic 4, 416-433 (2003).
11. Saito, H., Chi, Q., Zhuang, H., Matsunami, H. & Mainland, J. D. Odor coding by a Mammalian receptor repertoire. Sci. Signal. 2, ra9 (2009).
12. Mainland, J. D. et al. The missense of smell: functional variability in the human odorant receptor repertoire. Nat. Neurosci. 17, 114-120 (2014).
13. Botvinik, A. & Rossner, M. J. Linking cellular signalling to gene expression using EXT-encoded reporter libraries. Methods Mol. Biol. 786, 151-166 (2012).
14. Galinski, S., Wichert, S. P., Rossner, M. J. & Wehr, M. C. Multiplexed profiling of GPCR activities by combining split TEV assays and EXT-based barcoded readouts. Sci. Rep. 8, 8137 (2018).
15. Zhuang, H. & Matsunami, H. Synergism of accessory factors in functional expression of mammalian odorant receptors. J. Biol. Chem. 282, 15284-15293 (2007).
16. Shepard, B. D., Natarajan, N., Protzko, R. J., Acres, O. W. & Pluznick, J. L. A cleavable N-terminal signal peptide promotes widespread olfactory receptor surface expression in HEK293T cells. PLoS One 8, e68758 (2013).
17. Saito, H., Kubota, M., Roberts, R. W., Chi, Q. & Matsunami, H. RTP family members induce functional expression of mammalian odorant receptors. Cell 119, 679-691 (2004).
18. Li, X. et al. piggyBac transposase tools for genome engineering. Proc. Natl. Acad. Sci. U.S.A. 110, E2279-87 (2013).
19. McCarthy, D. J., Chen, Y. & Smyth, G. K. Differential expression analysis of multifactor RNA-Seq experiments with respect to biological variation. Nucleic Acids Res. 40, 4288-4297 (2012).
20. Zhuang, H. & Matsunami, H. Evaluating cell-surface expression and measuring activation of mammalian odorant receptors in heterologous cells. Nat. Protoc. 3, 1402-1413 (2008).
21. Gómez-Bombarelli, R. et al. Automatic Chemical Design Using a Data-Driven Continuous Representation of Molecules. ACS Cent Sci 4, 268-276 (2018).
22. Antebi, Y. E. et al. Combinatorial Signal Perception in the BMP Pathway. Cell 170, 1184-1196.e24 (2017).

1-114. (canceled)

115. A mammalian cell comprising: a) a nucleic acid encoding a heterologous receptor operatively coupled to an inducible promoter; and b) a nucleic acid comprising the inducible reporter comprising a receptor-responsive element, wherein the expression of the reporter is dependent on the activation of the activity of the receptor encoded by the heterologous receptor, and wherein the reporter comprises a barcode comprising an index region that is unique to the heterologous receptor.

116. The mammalian cell of claim 115, wherein the inducible promoter comprises a reverse tetracycline-controlled transactivator (rtTA).

117. The mammalian cell of claim 115, wherein the heterologous receptor or the inducible reporter is integrated into the mammalian cell's genome.

118. The mammalian cell of claim 117, wherein the integration is into a safe harbor locus.

119. The mammalian cell of claim 118, wherein the integration is into the H11 safe harbor locus.

120. The mammalian cell of claim 115, wherein the heterologous receptor and the inducible reporter are integrated into the mammalian cell's genome.

121. The mammalian cell of claim 120, wherein the integration is into a safe harbor locus.

122. The mammalian cell of claim 121, wherein the integration is into the H11 safe harbor locus.

123. The mammalian cell of claim 120, wherein a single copy of the heterologous receptor or a single copy of the inducible reporter is incorporated into the mammalian cell's genome.

124. The mammalian cell of claim 123, wherein the single copy of the heterologous receptor or the single copy of the inducible reported is incorporated into the mammalian cell's genome using a Bxb1 attp recombinase site.

125. The mammalian cell of claim 115, wherein the barcode and/or index region comprises at least 10 nucleotides.

126. The mammalian cell of claim 115, wherein the heterologous receptor is flanked at the 5′ and 3′ ends by insulator sequences.

127. The mammalian cell of claim 115, wherein the reporter is flanked at the 5′ and 3′ ends by insulator sequences.

128. A nucleic acid comprising a) a heterologous receptor gene operatively coupled to an inducible promoter; and b) an inducible reporter gene comprising a receptor-responsive element, wherein the expression of the reporter gene is dependent on the activation of the activity of the receptor encoded by the heterologous receptor gene, and wherein the reporter comprises a barcode comprising an index region that is unique to the heterologous receptor.

129. The nucleic acid of claim 128, wherein the inducible promoter gene comprises a reverse tetracycline-controlled transactivator (rtTA).

130. The nucleic acid of claim 128, wherein the nucleic acid further comprises a recombination site.

131. The nucleic acid of claim 130, wherein the recombination site is a Bxb1 attp recombination site.

132. The nucleic acid of claim 128, wherein the barcode and/or index region comprises at least 10 nucleotides.

133. The nucleic acid of claim 128, wherein the heterologous receptor gene is flanked at the 5′ and 3′ ends by insulator sequences.

134. The nucleic acid of claim 128, wherein the reporter is flanked at the 5′ and 3′ ends by insulator sequences.


SEQUENCE LISTING (189 sequences)

SEQ ID NO: 1 - DNA, 8 bp, Unknown
  Description of Unknown CRE motif sequence
tgacgtca

SEQ ID NO: 2 - DNA, 316 bp, Artificial Sequence
  Description of Artificial Sequence Synthetic polynucleotide
cgtcgtgacgtcagacagaccacgcgatcgctcgagtccgccggtcaatccggtgacgtcacgggcctcttcgctattacgccagctggcgaaagggggttgacgtcacattaaatcggccaacgcgcggggagaggcggtgacgtcaacaggcatcgtggtgtcacgctcgtcgtgacgtcagtcgctttaactggccctggctttggcagcctgtagcctgacgtcagagagcctgacgtcagagagcggagactctagagggtatataatggaagctcgaattccagcttggcattccggtactgttggtaaa

SEQ ID NO: 3 - DNA, 232 bp, Artificial Sequence
  Description of Artificial Sequence Synthetic polynucleotide
gagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaa

SEQ ID NO: 4 - protein, 15 aa, Artificial Sequence
  Description of Artificial Sequence Synthetic peptide
GLNDIFEAQKIEWHE

SEQ ID NO: 5 - protein, 26 aa, Artificial Sequence
  Description of Artificial Sequence Synthetic peptide
KRRWKKNFIAVSAANRFKKISSSGAL

SEQ ID NO: 6 - protein, 6 aa, Artificial Sequence
  Description of Artificial Sequence Synthetic peptide
EEEEEE

SEQ ID NO: 7 - protein, 13 aa, Artificial Sequence
  Description of Artificial Sequence Synthetic peptide
GAPVPYPDPLEPR

SEQ ID NO: 8 - protein, 8 aa, Artificial Sequence
  Description of Artificial Sequence Synthetic peptide
DYKDDDDK

SEQ ID NO: 9 - protein, 9 aa, Artificial Sequence
  Description of Artificial Sequence Synthetic peptide
YPYDVPDYA

SEQ ID NO: 10 - protein, 6 aa, Artificial Sequence
  Description of Artificial Sequence Synthetic 6xHis tag
HHHHHH

SEQ ID NO: 11 - protein, 10 aa, Artificial Sequence
  Description of Artificial Sequence Synthetic peptide
EQKLISEEDL

SEQ ID NO: 12 - protein, 18 aa, Artificial Sequence
  Description of Artificial Sequence Synthetic peptide
TKENPRSNQEESYDDNES

SEQ ID NO: 13 - protein, 15 aa, Artificial Sequence
  Description of Artificial Sequence Synthetic peptide
KETAAAKFERQHMDS

SEQ ID NO: 14 - protein, 38 aa, Artificial Sequence
  Description of Artificial Sequence Synthetic polypeptide
MDEKTTGWRGGHVVEGLAGELEQLRARLEHHPQGQREP

SEQ ID NO: 15 - protein, 13 aa, Artificial Sequence
  Description of Artificial Sequence Synthetic peptide
SLAELLNAGLGGS

SEQ ID NO: 16 - protein, 8 aa, Artificial Sequence
  Description of Artificial Sequence Synthetic peptide
TQDPSRVG

SEQ ID NO: 17 - protein, 8 aa, Artificial Sequence
  Description of Artificial Sequence Synthetic peptide
WSHPQFEK

SEQ ID NO: 18 - protein, 6 aa, Artificial Sequence
  Description of Artificial Sequence Synthetic peptide
CCPGCC

SEQ ID NO: 19 - protein, 14 aa, Artificial Sequence
  Description of Artificial Sequence Synthetic peptide
GKPIPNPLLGLDST

SEQ ID NO: 20 - protein, 11 aa, Artificial Sequence
  Description of Artificial Sequence Synthetic peptide
YTDIEMNRLGK

SEQ ID NO: 21 - protein, 8 aa, Artificial Sequence
  Description of Artificial Sequence Synthetic peptide
DLYDDDDK

SEQ ID NO: 22 - protein, 16 aa, Artificial Sequence
  Description of Artificial Sequence Synthetic peptide
TDKDMTITFTNKKDAE

SEQ ID NO: 23 - protein, 13 aa, Artificial Sequence
  Description of Artificial Sequence Synthetic peptide
AHIVMVDAYKPTK

SEQ ID NO: 24 - protein, 12 aa, Artificial Sequence
  Description of Artificial Sequence Synthetic peptide
KLGDIEFIKVNK

SEQ ID NO: 25 - DNA, 20 bp, Artificial Sequence
  Description of Artificial Sequence Synthetic primer
ccctttaatcagatgcgtcg

SEQ ID NO: 26 - DNA, 20 bp, Artificial Sequence
  Description of Artificial Sequence Synthetic primer
ctgcctgcttcaccaccttc

SEQ ID NO: 27 - DNA, 33 bp, Artificial Sequence
  Description of Artificial Sequence Synthetic primer
aagtgccttcctgccctttaatcagatgcgtcg

SEQ ID NO: 28 - DNA, 21 bp, Artificial Sequence
  Description of Artificial Sequence Synthetic primer
cgccgaagtgaaaaccaccta

SEQ ID NO: 29 - DNA, 21 bp, Artificial Sequence
  Description of Artificial Sequence Synthetic primer
aagtgccttcctgccctttaa

SEQ ID NO: 30 - DNA, 50 bp, Artificial Sequence
  Description of Artificial Sequence Synthetic primer
  modified_base (25)..(32): a, c, t or g
caagcagaagacggcatacgagatnnnnnnnncgaagtgaaaaccaccta

SEQ ID NO: 31 - DNA, 50 bp, Artificial Sequence
  Description of Artificial Sequence Synthetic primer
aatgatacggcgaccaccgagatctacacaagtgccttcctgccctttaa

SEQ ID NO: 32 - DNA, 35 bp, Artificial Sequence
  Description of Artificial Sequence Synthetic primer
cgggtttcttggccttgtaggtggttttcacttcg

SEQ ID NO: 33 - DNA, 47 bp, Artificial Sequence
  Description of Artificial Sequence Synthetic primer
  modified_base (13)..(27): a, c, t, g, unknown or other
ggaataacgcgtnnnnnnnnnnnnnnncgacgcatctgattaaaggg

SEQ ID NO: 34 - DNA, 33 bp, Artificial Sequence
  Description of Artificial Sequence Synthetic primer
ggaaggaccggttctagtcaaggcactatacat

SEQ ID NO: 35 - DNA, 55 bp, Artificial Sequence
  Description of Artificial Sequence Synthetic primer
tgctcctggccctgctgaccctaggcctggctcatatgaatggcacagaaggccc

SEQ ID NO: 36 - DNA, 55 bp, Artificial Sequence
  Description of Artificial Sequence Synthetic primer
agtcggccctgctgaggagtctttccacctgcaggtcttatcatgtctgctcgaa

SEQ ID NO: 37 - DNA, 18 bp, Artificial Sequence
  Description of Artificial Sequence Synthetic primer
cttctacgtgcccttctc

SEQ ID NO: 38 - DNA, 20 bp, Artificial Sequence
  Description of Artificial Sequence Synthetic primer
cctgcaggtcttatcatgtc

SEQ ID NO: 39 - DNA, 19 bp, Artificial Sequence
  Description of Artificial Sequence Synthetic primer
tacaggcggaatggacgag

SEQ ID NO: 40 - DNA, 21 bp, Artificial Sequence
  Description of Artificial Sequence Synthetic primer
aagtgaaaaccacctacaagg

SEQ ID NO: 41 - DNA, 20 bp, Artificial Sequence
  Description of Artificial Sequence Synthetic primer
ccctttaatcagatgcgtcg

SEQ ID NO: 42 - DNA, 58 bp, Artificial Sequence
  Description of Artificial Sequence Synthetic primer
  modified_base (30)..(37): a, c, t or g
aatgatacggcgaccaccgagatctacacnnnnnnnnaagtgccttcctgccctttaa

SEQ ID NO: 43 - DNA, 23 bp, Artificial Sequence
  Description of Artificial Sequence Synthetic primer
tgggcagttccaggcttatagtc

SEQ ID NO: 44 - DNA, 24 bp, Artificial Sequence
  Description of Artificial Sequence Synthetic primer
gggcgtacttggcatatgatacac

SEQ ID NO: 45 - DNA, 6 bp, Artificial Sequence
  Description of Artificial Sequence Synthetic oligonucleotide
atcacg

SEQ ID NO: 46 - DNA, 6 bp, Artificial Sequence
  Description of Artificial Sequence Synthetic oligonucleotide
cgatgt

SEQ ID NO: 47 - DNA, 6 bp, Artificial Sequence
  Description of Artificial Sequence Synthetic oligonucleotide
ttaggc

SEQ ID NO: 48 - DNA, 6 bp, Artificial Sequence
  Description of Artificial Sequence Synthetic oligonucleotide
tgacca

SEQ ID NO: 49 - DNA, 6 bp, Artificial Sequence
  Description of Artificial Sequence Synthetic oligonucleotide
acagtg

SEQ ID NO: 50 - DNA, 6 bp, Artificial Sequence
  Description of Artificial Sequence Synthetic oligonucleotide
gccaat

SEQ ID NO: 51 - DNA, 6 bp, Artificial Sequence
  Description of Artificial Sequence Synthetic oligonucleotide
cagatc

SEQ ID NO: 52 - DNA, 6 bp, Artificial Sequence
  Description of Artificial Sequence Synthetic oligonucleotide
acttga

SEQ ID NO: 53 - DNA, 6 bp, Artificial Sequence
  Description of Artificial Sequence Synthetic oligonucleotide
gatcag

SEQ ID NO: 54 - DNA, 6 bp, Artificial Sequence
  Description of Artificial Sequence Synthetic oligonucleotide
tagctt

SEQ ID NO: 55 - DNA, 6 bp, Artificial Sequence
  Description of Artificial Sequence Synthetic oligonucleotide
ggctac

SEQ ID NO: 56 - DNA, 6 bp, Artificial Sequence
  Description of Artificial Sequence Synthetic oligonucleotide
cttgta

SEQ ID NO: 57 - DNA, 6 bp, Artificial Sequence
  Description of Artificial Sequence Synthetic oligonucleotide
agtcaa

SEQ ID NO: 58 - DNA, 6 bp, Artificial Sequence
  Description of Artificial Sequence Synthetic oligonucleotide
agttcc

SEQ ID NO: 59 - DNA, 6 bp, Artificial Sequence
  Description of Artificial Sequence Synthetic oligonucleotide
atgtca

SEQ ID NO: 60 - DNA, 6 bp, Artificial Sequence
  Description of Artificial Sequence Synthetic oligonucleotide
ccgtcc

SEQ ID NO: 61 - DNA, 6 bp, Artificial Sequence
  Description of Artificial Sequence Synthetic oligonucleotide
gtagag

SEQ ID NO: 62 - DNA, 6 bp, Artificial Sequence
  Description of Artificial Sequence Synthetic oligonucleotide
gtccgc

SEQ ID NO: 63 - DNA, 6 bp, Artificial Sequence
  Description of Artificial Sequence Synthetic oligonucleotide
gtgaaa

SEQ ID NO: 64 - DNA, 6 bp, Artificial Sequence
  Description of Artificial Sequence Synthetic oligonucleotide
gtggcc

SEQ ID NO: 65 - DNA, 6 bp, Artificial Sequence
  Description of Artificial Sequence Synthetic oligonucleotide
gtttcg

SEQ ID NO: 66 - DNA, 6 bp, Artificial Sequence
  Description of Artificial Sequence Synthetic oligonucleotide
cgtacg

SEQ ID NO: 67 - DNA, 6 bp, Artificial Sequence
  Description of Artificial Sequence Synthetic oligonucleotide
gagtgg

SEQ ID NO: 68 - DNA, 6 bp, Artificial Sequence
  Description of Artificial Sequence Synthetic oligonucleotide
ggtagc

SEQ ID NO: 69 - DNA, 6 bp, Artificial Sequence
  Description of Artificial Sequence Synthetic oligonucleotide
actgat

SEQ ID NO: 70 - DNA, 6 bp, Artificial Sequence
  Description of Artificial Sequence Synthetic oligonucleotide
atgagc

SEQ ID NO: 71 - DNA, 6 bp, Artificial Sequence
  Description of Artificial Sequence Synthetic oligonucleotide
attcct

SEQ ID NO: 72 - DNA, 6 bp, Artificial Sequence
  Description of Artificial Sequence Synthetic oligonucleotide
caaaag

SEQ ID NO: 73 - DNA, 6 bp, Artificial Sequence
  Description of Artificial Sequence Synthetic oligonucleotide
caacta

SEQ ID NO: 74 - DNA, 6 bp, Artificial Sequence
  Description of Artificial Sequence Synthetic oligonucleotide
caccgg

SEQ ID NO: 75 - DNA, 6 bp, Artificial Sequence
  Description of Artificial Sequence Synthetic oligonucleotide
cacgat

SEQ ID NO: 76 - DNA, 6 bp, Artificial Sequence
  Description of Artificial Sequence Synthetic oligonucleotide
cactca

SEQ ID NO: 77 - DNA, 6 bp, Artificial Sequence
  Description of Artificial Sequence Synthetic oligonucleotide
caggcg

SEQ ID NO: 78 - DNA, 6 bp, Artificial Sequence
  Description of Artificial Sequence Synthetic oligonucleotide
catggc

SEQ ID NO: 79 - DNA, 7 bp, Artificial Sequence
  Description of Artificial Sequence Synthetic oligonucleotide
cctgcga

SEQ ID NO: 80 - DNA, 8 bp, Artificial Sequence
  Description of Artificial Sequence Synthetic oligonucleotide
ctctctat

SEQ ID NO: 81 - DNA, 7 bp, Artificial Sequence
  Description of Artificial Sequence Synthetic oligonucleotide
tgcagag

SEQ ID NO: 82 - DNA, 8 bp, Artificial Sequence
  Description of Artificial Sequence Synthetic oligonucleotide
tatcctct

SEQ ID NO: 83 - DNA, 7 bp, Artificial Sequence
  Description of Artificial Sequence Synthetic oligonucleotide
acctagg

SEQ ID NO: 84 - DNA, 8 bp, Artificial Sequence
  Description of Artificial Sequence Synthetic oligonucleotide
gtaaggag

SEQ ID NO: 85 - DNA, 7 bp, Artificial Sequence
  Description of Artificial Sequence Synthetic oligonucleotide
ttgatcc

SEQ ID NO: 86 - DNA, 8 bp, Artificial Sequence
  Description of Artificial Sequence Synthetic oligonucleotide
actgcata

SEQ ID NO: 87 - DNA, 7 bp, Artificial Sequence
  Description of Artificial Sequence Synthetic oligonucleotide
atcttgc

SEQ ID NO: 88 - DNA, 8 bp, Artificial Sequence
  Description of Artificial Sequence Synthetic oligonucleotide
aaggagta

SEQ ID NO: 89 - DNA, 7 bp, Artificial Sequence
  Description of Artificial Sequence Synthetic oligonucleotide
tctccat

SEQ ID NO: 90 - DNA, 8 bp, Artificial Sequence
  Description of Artificial Sequence Synthetic oligonucleotide
ctaagcct

SEQ ID NO: 91 - DNA, 7 bp, Artificial Sequence
  Description of Artificial Sequence Synthetic oligonucleotide
catcgag

SEQ ID NO: 92 - DNA, 8 bp, Artificial Sequence
  Description of Artificial Sequence Synthetic oligonucleotide
cgtctaat

SEQ ID NO: 93 - DNA, 7 bp, Artificial Sequence
  Description of Artificial Sequence Synthetic oligonucleotide
ttcgagc

SEQ ID NO: 94 - DNA, 8 bp, Artificial Sequence
  Description of Artificial Sequence Synthetic oligonucleotide
tctctccg

SEQ ID NO: 95 - DNA, 7 bp, Artificial Sequence
  Description of Artificial Sequence Synthetic oligonucleotide
agttggt

SEQ ID NO: 96 - DNA, 8 bp, Artificial Sequence
  Description of Artificial Sequence Synthetic oligonucleotide
ctagtcga

SEQ ID NO: 97 - DNA, 7 bp, Artificial Sequence
  Description of Artificial Sequence Synthetic oligonucleotide
gtaccgg

SEQ ID NO: 98 - DNA, 8 bp, Artificial Sequence
  Description of Artificial Sequence Synthetic oligonucleotide
agctagaa

SEQ ID NO: 99 - DNA, 7 bp, Artificial Sequence
  Description of Artificial Sequence Synthetic oligonucleotide
cggagtt

SEQ ID NO: 100 - DNA, 8 bp, Artificial Sequence
  Description of Artificial Sequence Synthetic oligonucleotide
actctagg

SEQ ID NO: 101 - DNA, 7 bp, Artificial Sequence
  Description of Artificial Sequence Synthetic oligonucleotide
acttcaa

SEQ ID NO: 102 - DNA, 8 bp, Artificial Sequence
  Description of Artificial Sequence Synthetic oligonucleotide
tcttacgc

SEQ ID NO: 103 - DNA, 7 bp, Artificial Sequence
  Description of Artificial Sequence Synthetic oligonucleotide
tgatagt

SEQ ID NO: 104 - DNA, 8 bp, Artificial Sequence
  Description of Artificial Sequence Synthetic oligonucleotide
cttaatag

SEQ ID NO: 105 - DNA, 7 bp, Artificial Sequence
  Description of Artificial Sequence Synthetic oligonucleotide
gatccaa

SEQ ID NO: 106 - DNA, 7 bp, Artificial Sequence
  Description of Artificial Sequence Synthetic oligonucleotide
caggtcg

SEQ ID NO: 107 - DNA, 7 bp, Artificial Sequence
  Description of Artificial Sequence Synthetic oligonucleotide
cgcatta

SEQ ID NO: 108 - DNA, 7 bp, Artificial Sequence
  Description of Artificial Sequence Synthetic oligonucleotide
ggtacct

SEQ ID NO: 109 - DNA, 7 bp, Artificial Sequence
  Description of Artificial Sequence Synthetic oligonucleotide
ggacgca

SEQ ID NO: 110 - DNA, 7 bp, Artificial Sequence
  Description of Artificial Sequence Synthetic oligonucleotide
gagattc

SEQ ID NO: 111 - DNA, 7 bp, Artificial Sequence
  Description of Artificial Sequence Synthetic oligonucleotide
gagcatg

SEQ ID NO: 112 - DNA, 7 bp, Artificial Sequence
  Description of Artificial Sequence Synthetic oligonucleotide
gttgcgt

SEQ ID NO: 113 - DNA, 7 bp, Artificial Sequence
  Description of Artificial Sequence Synthetic oligonucleotide
ccaatgc

SEQ ID NO: 114 - DNA, 7 bp, Artificial Sequence
  Description of Artificial Sequence Synthetic oligonucleotide
cgagatc

SEQ ID NO: 115 - DNA, 7 bp, Artificial Sequence
  Description of Artificial Sequence Synthetic oligonucleotide
catattg

SEQ ID NO: 116 - DNA, 7 bp, Artificial Sequence
  Description of Artificial Sequence Synthetic oligonucleotide
gacgtca

SEQ ID NO: 117 - DNA, 7 bp, Artificial Sequence
  Description of Artificial Sequence Synthetic oligonucleotide
tggcatc

SEQ ID NO: 118 - DNA, 7 bp, Artificial Sequence
  Description of Artificial Sequence Synthetic oligonucleotide
gtaattg

SEQ ID NO: 119 - DNA, 7 bp, Artificial Sequence
  Description of Artificial Sequence Synthetic oligonucleotide
cctatct

SEQ ID NO: 120 - DNA, 7 bp, Artificial Sequence
  Description of Artificial Sequence Synthetic oligonucleotide
caatcgg

SEQ ID NO: 121 - DNA, 7 bp, Artificial Sequence
  Description of Artificial Sequence Synthetic oligonucleotide
gcggcat

SEQ ID NO: 122 - DNA, 7 bp, Artificial Sequence
  Description of Artificial Sequence Synthetic oligonucleotide
agtactg

SEQ ID NO: 123 - DNA, 7 bp, Artificial Sequence
  Description of Artificial Sequence Synthetic oligonucleotide
tactatt

SEQ ID NO: 124 - DNA, 7 bp, Artificial Sequence
  Description of Artificial Sequence Synthetic oligonucleotide
ccggatg

SEQ ID NO: 125 - DNA, 7 bp, Artificial Sequence
  Description of Artificial Sequence Synthetic oligonucleotide
accatga

SEQ ID NO: 126 - DNA, 7 bp, Artificial Sequence
  Description of Artificial Sequence Synthetic oligonucleotide
cggttct

SEQ ID NO: 127 - DNA, 7 bp, Artificial Sequence
  Description of Artificial Sequence Synthetic oligonucleotide
tattcca

SEQ ID NO: 128 - DNA, 7 bp, Artificial Sequence
  Description of Artificial Sequence Synthetic oligonucleotide
cctcctg

SEQ ID NO: 129 - DNA, 7 bp, Artificial Sequence
  Description of Artificial Sequence Synthetic oligonucleotide
aggtatt

SEQ ID NO: 130 - DNA, 7 bp, Artificial Sequence
  Description of Artificial Sequence Synthetic oligonucleotide
gcattcg

SEQ ID NO: 131 - DNA, 7 bp, Artificial Sequence
  Description of Artificial Sequence Synthetic oligonucleotide
ttgcgaa

SEQ ID NO: 132 - DNA, 7 bp, Artificial Sequence
  Description of Artificial Sequence Synthetic oligonucleotide
ttgaatt

SEQ ID NO: 133 - DNA, 7 bp, Artificial Sequence
  Description of Artificial Sequence Synthetic oligonucleotide
ctgcgcg

SEQ ID NO: 134 - DNA, 7 bp, Artificial Sequence
  Description of Artificial Sequence Synthetic oligonucleotide
agacctt

SEQ ID NO: 135 - DNA, 7 bp, Artificial Sequence
  Description of Artificial Sequence Synthetic oligonucleotide
gtccagt

SEQ ID NO: 136 - DNA, 7 bp, Artificial Sequence
  Description of Artificial Sequence Synthetic oligonucleotide
acctgct

SEQ ID NO: 137 - DNA, 7 bp, Artificial Sequence
  Description of Artificial Sequence Synthetic oligonucleotide
ccggtac

SEQ ID NO: 138 - DNA, 7 bp, Artificial Sequence
  Description of Artificial Sequence Synthetic oligonucleotide
cttgacc

SEQ ID NO: 139 - DNA, 7 bp, Artificial Sequence
  Description of Artificial Sequence Synthetic oligonucleotide
catcatt

SEQ ID NO: 140 - DNA, 7 bp, Artificial Sequence
  Description of Artificial Sequence Synthetic oligonucleotide
tctgact

SEQ ID NO: 141 - DNA, 7 bp, Artificial Sequence
  Description of Artificial Sequence Synthetic oligonucleotide
tctagtt

SEQ ID NO: 142 - DNA, 7 bp, Artificial Sequence
  Description of Artificial Sequence Synthetic oligonucleotide
gccatag

SEQ ID NO: 143 - DNA, 7 bp, Artificial Sequence
  Description of Artificial Sequence Synthetic oligonucleotide
accgtcg

SEQ ID NO: 144 - DNA, 7 bp, Artificial Sequence
  Description of Artificial Sequence Synthetic oligonucleotide
cttggtt

SEQ ID NO: 145 - DNA, 7 bp, Artificial Sequence
  Description of Artificial Sequence Synthetic oligonucleotide
tacgccg

SEQ ID NO: 146 - DNA, 7 bp, Artificial Sequence
  Description of Artificial Sequence Synthetic oligonucleotide
ggactgc

SEQ ID NO: 147 - DNA, 7 bp, Artificial Sequence
  Description of Artificial Sequence Synthetic oligonucleotide
gcgcgag

SEQ ID NO: 148 - DNA, 7 bp, Artificial Sequence
  Description of Artificial Sequence Synthetic oligonucleotide
gtcgcag

SEQ ID NO: 149 - DNA, 7 bp, Artificial Sequence
  Description of Artificial Sequence Synthetic oligonucleotide
catacgt

SEQ ID NO: 150 - DNA, 7 bp, Artificial Sequence
  Description of Artificial Sequence Synthetic oligonucleotide
tcagtat

SEQ ID NO: 151 - DNA, 7 bp, Artificial Sequence
  Description of Artificial Sequence Synthetic oligonucleotide
ctaagta

SEQ ID NO: 152 - DNA, 7 bp, Artificial Sequence
  Description of Artificial Sequence Synthetic oligonucleotide
ttagctt

SEQ ID NO: 153 - DNA, 7 bp, Artificial Sequence
  Description of Artificial Sequence Synthetic oligonucleotide
cgccgtc

SEQ ID NO: 154 - DNA, 7 bp, Artificial Sequence
  Description of Artificial Sequence Synthetic oligonucleotide
gtcttct

SEQ ID NO: 155 - DNA, 7 bp, Artificial Sequence
  Description of Artificial Sequence Synthetic oligonucleotide
gccggac

SEQ ID NO: 156 - DNA, 7 bp, Artificial Sequence
  Description of Artificial Sequence Synthetic oligonucleotide
aagctga

SEQ ID NO: 157 - DNA, 7 bp, Artificial Sequence
  Description of Artificial Sequence Synthetic oligonucleotide
gcgctct

SEQ ID NO: 158 - DNA, 7 bp, Artificial Sequence
  Description of Artificial Sequence Synthetic oligonucleotide
cgtaggc

SEQ ID NO: 159 - DNA, 7 bp, Artificial Sequence
  Description of Artificial Sequence Synthetic oligonucleotide
atgatta

SEQ ID NO: 160 - DNA, 7 bp, Artificial Sequence
  Description of Artificial Sequence Synthetic oligonucleotide
gcaggtt

SEQ ID NO: 161 - DNA, 7 bp, Artificial Sequence
  Description of Artificial Sequence Synthetic oligonucleotide
aatcgtc

SEQ ID NO: 162 - DNA, 7 bp, Artificial Sequence
  Description of Artificial Sequence Synthetic oligonucleotide
cggccta

SEQ ID NO: 163 - DNA, 7 bp, Artificial Sequence
  Description of Artificial Sequence Synthetic oligonucleotide
ctatgcc

SEQ ID NO: 164 - DNA, 7 bp, Artificial Sequence
  Description of Artificial Sequence Synthetic oligonucleotide
ggttgaa

SEQ ID NO: 165 - DNA, 7 bp, Artificial Sequence
  Description of Artificial Sequence Synthetic oligonucleotide
gagttaa

SEQ ID NO: 166 - DNA, 7 bp, Artificial Sequence
  Description of Artificial Sequence Synthetic oligonucleotide
tagacta

SEQ ID NO: 167 - DNA, 7 bp, Artificial Sequence
  Description of Artificial Sequence Synthetic oligonucleotide
tcatgca

SEQ ID NO: 168 - DNA, 7 bp, Artificial Sequence
  Description of Artificial Sequence Synthetic oligonucleotide
gcttatt

SEQ ID NO: 169 - DNA, 7 bp, Artificial Sequence
  Description of Artificial Sequence Synthetic oligonucleotide
caaggct

SEQ ID NO: 170 - DNA, 7 bp, Artificial Sequence
  Description of Artificial Sequence Synthetic oligonucleotide
aggttgg

SEQ ID NO: 171 - DNA, 7 bp, Artificial Sequence
  Description of Artificial Sequence Synthetic oligonucleotide
cttctgc

SEQ ID NO: 172 - DNA, 7 bp, Artificial Sequence
  Description of Artificial Sequence Synthetic oligonucleotide
taattct

SEQ ID NO: 173 - DNA, 7 bp, Artificial Sequence
  Description of Artificial Sequence Synthetic oligonucleotide
gatgctg

SEQ ID NO: 174 - DNA, 7 bp, Artificial Sequence
  Description of Artificial Sequence Synthetic oligonucleotide
cctagaa

SEQ ID NO: 175 - DNA, 7 bp, Artificial Sequence
  Description of Artificial Sequence Synthetic oligonucleotide
ctagagg

SEQ ID NO: 176 - DNA, 7 bp, Artificial Sequence
  Description of Artificial Sequence Synthetic oligonucleotide
tatccgg

SEQ ID NO: 177 - DNA, 7 bp, Artificial Sequence
  Description of Artificial Sequence Synthetic oligonucleotide
aggcggc

SEQ ID NO: 178 - DNA, 7 bp, Artificial Sequence
  Description of Artificial Sequence Synthetic oligonucleotide
ggtcgtt

SEQ ID NO: 179 - DNA, 7 bp, Artificial Sequence
  Description of Artificial Sequence Synthetic oligonucleotide
ccgctgg

SEQ ID NO: 180 - DNA, 7 bp, Artificial Sequence
  Description of Artificial Sequence Synthetic oligonucleotide
ggaacta

SEQ ID NO: 181 - DNA, 7 bp, Artificial Sequence
  Description of Artificial Sequence Synthetic oligonucleotide
attgcca

SEQ ID NO: 182 - DNA, 7 bp, Artificial Sequence
  Description of Artificial Sequence Synthetic oligonucleotide
atatacg

SEQ ID NO: 183 - DNA, 7 bp, Artificial Sequence
  Description of Artificial Sequence Synthetic oligonucleotide
gattagc

SEQ ID NO: 184 - DNA, 7 bp, Artificial Sequence
  Description of Artificial Sequence Synthetic oligonucleotide
agaagtc

SEQ ID NO: 185 - DNA, 7 bp, Artificial Sequence
  Description of Artificial Sequence Synthetic oligonucleotide
atagtac

SEQ ID NO: 186 - DNA, 7 bp, Artificial Sequence
  Description of Artificial Sequence Synthetic oligonucleotide
gatctcg

SEQ ID NO: 187 - DNA, 7 bp, Artificial Sequence
  Description of Artificial Sequence Synthetic oligonucleotide
ggctgcg

SEQ ID NO: 188 - protein, 10 aa, Artificial Sequence
  Description of Artificial Sequence Synthetic peptide
  MISC_FEATURE (1)..(10): This sequence may encompass 5-10 residues
HHHHHHHHHH

SEQ ID NO: 189 - protein, 413 aa, Unknown [sequence omitted]